(19) United States
(12) Patent Application Publication
(10) Pub. No.: US 2011/0151490 A1
(43) Pub. Date: Jun. 23, 2011

(54) Compositions for the Detection and Treatment of Colorectal Cancer

(75) Inventors: Jeffrey D. Hillman, Gainesville, FL (US); Manohar John, Newberry, FL (US)

(73) Assignee: Oragenics, Inc., Alachua, FL (US)

(21) Appl. No.: 13/054,667

(22) PCT Filed: Jul. 17, 2009
(86) PCT No.: PCT/US09/50938

§ 371 Date: Mar. 2, 2011

Related U.S. Application Data
(60) Provisional application No. 61/081,926, filed on Jul. 18, 2008.

(51) Int. Cl. G01N 33/53; C07K 16/18; C07K 16/22; C07K 16/26

(52) U.S. Cl. 435/7.92; 530/389.2; 530/389.1; 530/389.3; 530/388.1

(57) ABSTRACT

The invention provides methods of identifying proteins and polypeptides and their cognate polynucleotides that are expressed by cells under one environmental condition and not under a second environmental condition. The invention also provides compositions for the treatment and detection of cancer, including colorectal cancer.

### PRIORITY

This application claims the benefit of U.S. Ser. No. 61/081,926, filed Jul. 18, 2008, which is incorporated herein by reference in its entirety.

### GOVERNMENT INTEREST

This invention was supported, in part, by NIH/NCI/SBIR grant number 1R43CA124006-01A1. The government of the United States has certain rights to the invention.

### BACKGROUND OF THE INVENTION

The identification of proteins, polypeptide and other cellular constituent that are made when a cell undergoes a change from one state or condition to another can be important because such molecules are very likely to serve as indicators that the change is or has taken place. In the case where one condition is health and the second condition is a disease state, identification of such “change mediated” proteins, polypeptides or other cellular components should provide excellent targets for the development of new diagnostics, and likewise may provide targets for various types of antibiotherapies (e.g., vaccines) to aid in the treatment of the disease.

In certain instances, change mediated molecules may be shed from the diseased tissue and enter into bodily fluids that are relatively easily recovered. The identification of the presence of cellular constituents shed from diseased (e.g., cancerous) tissue in bodily fluids can be important because such shed proteins are very likely candidates to serve as ideal diagnostic targets that are pathogenomonic of active disease. For example, polypeptides that are differentially expressed in cancerous cells, such as colorectal cancer cells, and polypeptides that specifically expressed in cancerous cells and that are shed from cancerous cells into bodily fluids can be used to provide a precise and accurate diagnosis of cancer, for screening of anti-cancer compounds, for the development of therapeutic compositions, and other uses.

### SUMMARY OF THE INVENTION

One embodiment of the invention provides a method of detecting cancer or a predisposition to developing cancer in a subject. The method comprises determining an expression level of a cancer-associated polynucleotide, protein, or polypeptide selected from the group consisting of Titin; HBA1; Insulin-like growth factor 1 receptor (IGF1R); Isoform 3 of zonadhesin precursor; latent transforming growth factor beta binding protein 4 (LTBP4); ASXL1 (additional sex combs like 1); beta globin (HBB); BMP15-bone morphogenetic protein; TRIM49; DNAJ homolog subfamily B member 11 precursor; uncharacterized hematopoietic stem/progenitor cells protein MDS027; uncharacterized protein ALB; isoform 3 of sushi, nidogen and EGF-like domain-containing protein 1 precursor; isoform 2 of peripherin; mitochondrial 28S ribosomal protein S22; translation initiation factor EIF-2B subunit epsilon; estradiol 17-beta-dehydrogenase 1; XRCC6BP1; brain-specific angiogenesis inhibitor 1 precursor; isoform 2 of ring finger and CCCH-type zinc finger domain-containing protein 2; hemoglobin subunit beta; isoform 1 of far upstream element-binding protein 1; GALECTIN-3; lysozyme C precursor; actin, alpha skeletal muscle; isoform M2 of pyruvate kinase isozymes M1/M2; AGR2; neutrophil defensin 1 precursor; myeloblastin precursor; uncharacterized protein PSME2; tubulin beta-2C chain; thiosulfate sulfurtransferase; heat shock 70 kDa protein 1; Ig kappa chain V-III region sie; macrophage migration inhibitory factor; isoform 1 of ATP synthase subunit D, mitochondrial; uncharacterized protein ENSP00000374051; isocitrate dehydrogenase [NADP] cytoplasmic; hemoglobin subunit delta; isoform 1 of splicing factor, arginine/serine-rich 7; isoform 1 of mRNA-capping enzyme; LON protease homolog, mitochondrial precursor; signal recognition particle 54 kDa protein; isoform long of galectin-9; integrin-linked protein kinase; bifunctional aminoacyl-tRNA synthetase; isoform 1 of zinc finger protein 207; inorganic pyrophosphatase; calponin-2; isoform 1 of muscleblind-like protein 3; cathepsin G precursor; zinc finger and BTB domain-containing protein 34; adenine phosphoribosyltransferase; 40S ribosomal protein S9; TALIN-1; leucine-rich repeat-containing protein 59; ATP synthase subunit alpha, mitochondrial precursor; isoform 7 of protein transport protein SEC31A; dihydroxyacetone kinase; protein similar to heterogeneous nuclear ribonucleoproteins C1/C2 (HNRNP C1/HNRNP C2) isoform 4; 18 kDa protein (e.g., UNIPARC Accession Number IP100796554; cold agglutinin FS-1 L-chain; isoform 1 of heterogeneous nuclear ribonucleoprotein d0; DAZAP1/MEF2D fusion protein; POTE2; Keratin 18 (KRT18); PSME4 Isoform 1 of Proteasome activator complex subunit; Mitogen-activated protein kinase-activated protein kinase (MAPKAPK33); Complement component 1, s subcomponent (C1S); Lysozyme C precursor (LYZ); Keritin Type Cytoskeletal 20 (KRT20); RNASE3; Aldehyde dehydrogenase X, mitochondrial precursor (ALDH1B1); CDNA FLJ25506 fis, clone CBR05185; Isoform B of fibulin-1 precursor (FBLN1); Nucleobindin 1 (NUCB1); Histone cluster 2, H2ba (HIST2H2BA); Tripartite motif-containing 28 (TRIM28); Peroxisomal D3, D2 enoyl-CoA isomerase (PECI); Peptidylprolyl isomerase B (PPIB); Similar to 40S ribosomal protein S17; Eukaryotic translation elongation factor 1 gamma (EEF1G); Keratin 8 (KRT8); Fibulin 2 (FBLN2); VIM; Fibrinogen alpha chain (FGA); Annexin A2 (ANXA2); H2A histone family, member J (H2AFJ); Actin alpha, cardiac muscle 1 (ACTC1); Keratin 19 (KRT19); Immunoglobin lambda locus (IGL@protein); Immunoglobulin heavy constant mu (IGHM); EGF-containing fibulin-like extracellular matrix protein 1 (EFEMP1); Tripartite motif-containing protein 34; Isoform 3 of AP1-subunit Gamma Binding Protein 1; Proflin-1; Histone H4; Hemoglobin subunit alpha; Transgelin); Lumican precursor; Hemoglobin Beta; Fibrinogen Beta Chain Precursor; Immunoglobulin kappa constant (IGKC); Uncharacterized Protein ALB; ApoA1; C4A; C3 187 kDa protein; Actin, Cytoplasmic 1 (actin beta); Hemoglobin beta; Hemoglobin subunit alpha; POTE-2 alpha actin; SLC4A10; Ribonuclease P Protein Subunit P20 (POP7); Nuclear RNA export factor 1 (NXF1); UVEAL Autoantigen With Coiled-Coil Domains And Ankyrin Repeats, UACA; Uncharacterized Protein C13ORF27; Isoform 3 of Sushi, Nidogen And EGF-Like Domain-Containing Protein 1 Precursor; Isoform 1 Of Dynein Heavy Chain 10, Axonemal (DNAH10); Gap junction alpha-1 protein (GJA1/Connexion 43); Isoform 1 Of Kinesin-Like Protein KIF25 (KIF25); GAPDH-Glyceraldehyde-3-Phosphate Dehydrogenase; Uncharacterized Protein ALB; Galectin-3, LGALS3; Similar to NAC-Alpha Domain-Containing Protein 1 (NACAD); Acetyl-CoA Acetyltransferase, Mitochondrial, ACAT1; KH-Type Splicing Regulatory Protein, FUBP2; Profilin 1 (PFN1); Chloride Intracellular Channel Protein 1, CLIC1; Zinc Finger Protein 831; Endoplasmin; Ribosomal Protein S10 (RPS10); Splicing Factor, Arginine/Serine-Rich 3; ACTA2 Protein (alpha actin, smooth muscle); Isoform 1 of Sodium Channel Protein Type 8 Subunit Alpha, SCN8A; Isoform Long of Galectin-9; T-Complcx Protein 1 Subunit Epsilon, CCT5; Alpha-Enolase, Lung Specific; Proto-Oncogene Serine/Threonine-Protein Kinase MOS; Isoform 1 Of Beta-Adducin (ADD2); Apolipoprotein E (APOE); Ubiquilin-4 (UBQLN4) (ataxin-1 ubiquitin-like interacting protein); Sumo-Conjugating Enzyme UB21 (UBC9 homolog in yeast); Myosin-15 (MYH15); FLJ93091, Homo Sapiens UMP-CMP Kinase (UMP-CMPK); Intelectin-1 (ITLN1); Apolipoprotein A-IV (APOA4); Mitochondrial pyruvate dehydrogenase (lipoamide) alpha 1 (PDHA1); Leucine-Rich Repeat-Containing Protein 59 (LRRC59); 60S Ribosomal Protein L37A (RPL37A); Uridine-Cytidine Kinase 1-like 1 (UCKL1); Aldehyde Dehydrogenase 9A1 (ALDH9A1); Isoform 3 of Thioredoxin Reductase 1, Cytoplasmic (TXNRD1); Nuclear Receptor Subfamily 2 Group E Member 1 (NR2E1); Cation Channel Sperm-Associated Protein 3 (CATSPER3); Transmembrane EMP24 Domain-Containing Protein 1 (TMED1); Protein FAM154A (FAM154A); Sand Isoform 1 of Transcriptional Repressor NF-X1 (NFX1); or any combinations thereof (“the polypeptides of the invention”) in a biological sample from the subject. An increase of the expression level of the cancer-associated polynucleotide in the biological sample, such as a bodily fluid, as compared to a control sample indicates that the subject has cancer or has a predisposition to developing cancer. The protein or polypeptide can comprise an amino acid sequence set forth as SEQ ID NO:1-157. The cancer can be colorectal cancer. The method can further comprise determining the expression level of one or more or two or more of the cancer-associated proteins or polypeptides. The expression level of the cancer-associated proteins or polypeptides can be determined by a method selected from group consisting of: (a) detecting the presence of the protein or polypeptide (b) detecting the biological activity of the protein or polypeptide encoded by the cancer-associated polynucleotide, and (c) detecting mRNA of the cancer-associated polynucleotide. The biological sample can comprise cells, cell extracts, tissue, bodily fluid, and bodily fluid substantially lacking cells (e.g., less than about 1, 5, or 10% cells) such as serum, urine, tears, milk, seminal fluid, prostatic fluid, lung lavage fluid, and saliva. The level of the cancer-associated protein or polypeptide can be determined by detecting its level in the biological sample using an antibody that binds to epitopes of the protein or polypeptide specific to the change mediated protein or polypeptide or by other means known in the art.

Another embodiment of the invention provides an isolated antibody or antigen-binding fragment thereof that specifically binds to a protein or polypeptide of the invention or any combinations thereof. A protein or polypeptide of the invention can comprise an amino acid sequence set forth as SEQ ID NO:1-157. The antibody can be a monoclonal antibody, a polyclonal antibody, a single-chain antibody, a monospecific single-chain antibody, a bispecific single-chain antibody, a bivalent single-chain antibody, a tetravalent single-chain antibody, a chimeric antibody, an antigen-binding fragment of an antibody, or a humanized antibody.

Even another embodiment of the invention provides a method of screening for anti-cancer compounds. The method comprises comparing the level of a change mediated protein or polypeptide expression product in a first biological sample in the presence of a test compound to the level of the change mediated protein or polypeptide expression product in a second biological sample in the absence of the test compound. The change mediated expression product comprises a protein or polypeptide of the invention or mRNA encoding the polypeptide of the invention or any combinations thereof. A test compound that decreases the level of the expression product in the first biological sample as compared to the second biological sample is identified as an anti-cancer agent. The protein or polypeptide can comprise an amino acid sequence set forth as SEQ ID NO:1-157.

Yet another embodiment of the invention provides a method of screening for a compound for treating or preventing cancer. The method comprises (a) contacting a candidate compound with a cell expressing a protein or polypeptide of the invention or any combinations thereof and (b) selecting a compound that reduces the expression level of the protein or polypeptide. The protein or polypeptide can comprise an amino acid sequence set forth as SEQ ID NO:1-157.

Another embodiment of the invention provides a kit for the detection of cancer in a mammal. The kit comprises (a) an antibody or antigen-binding fragment thereof, wherein in the antibody or antigen-binding fragment thereof specifically binds an epitope of the protein or polypeptide of the invention and (b) one or more reagents for detecting a binding reaction between the antibody and the protein or polypeptide. The protein or polypeptide can comprise an amino acid sequence set forth as SEQ ID NO:1-157 or any combinations thereof.

Still another embodiment of the invention provides a kit for detecting cancer cells in a biological sample comprising at least one polynucleotide primer or probe wherein the polynucleotide primer or probe is specific for a polynucleotide that encodes a protein or polypeptide of the invention. The protein or polypeptide can comprise an amino acid sequence set forth as SEQ ID NO:1-157 or any combinations thereof. The kit can comprise at least two polynucleotide primers specific for the polynucleotide that encodes a protein or polypeptide of the invention.

Yet another embodiment of the invention provides a fusion protein comprising at least two proteins or polypeptides of the invention or any combinations thereof. At least two proteins or polypeptides can he selected from the group consisting of an amino acid sequence set forth as SEQ ID NO:1-157.

Even another embodiment of the invention provides a composition comprising a first component selected from the group consisting of physiologically acceptable carriers and immunostimulants, and a second component selected from the group consisting of a protein or polypeptide of the invention or any combinations thereof; a polynucleotide that encodes the protein or polypeptide of the invention or any combinations thereof; an antibody according of the invention or any combinations thereof; and a fusion protein of the invention or any combinations thereof. The protein or polypeptide can comprise an amino acid sequence set forth as SEQ ID NO:1-157 or any combinations thereof.

Another embodiment of the invention provides a colorectal cancer reference expression profile, comprising a pattern of protein or polypeptide expression of two or more proteins or polypeptides of the invention set forth as SEQ ID NO:1-157 or any combinations thereof.

Another embodiment of the invention provides a colorectal cancer reference expression profile, comprising a pattern of polynucleotide expression of two or more polynucleotides that encode proteins or polypeptides of the invention or any combinations thereof. The polypeptides of the invention can comprise amino acid sequences set forth as SEQ ID NO:1-157.

Yet another embodiment of the invention provides an array comprising two or more polynucleotides that specifically hybridize to two or more polynucleotides that encode a protein or polypeptide of the invention or two or more polypeptides of the invention or any combinations thereof. The polypeptides of the invention can comprise amino acid sequences set forth as SEQ ID NO:1-157.

Still another embodiment of the invention provides a composition for treating cancer. The composition comprises a pharmaceutically effective amount of an antibody or antigen-binding fragment thereof that specifically binds to a protein or polypeptide of the invention or any combinations thereof. The protein or polypeptide of invention can comprise an amino acid sequence set forth as SEQ ID NO:1-157.

Even another embodiment of the invention provides a composition for treating cancer. The composition comprises a pharmaceutically effective amount of a polypeptide of the invention or a polynucleotide encoding the polypeptide of the invention. The polypeptide of the invention can comprise an amino acid sequence set forth as SEQ ID NO:1-157.

Another embodiment of the invention provides a method for treating cancer in a subject or stimulating an immune response, such as an anti-tumor immune response or any other type of immune response in a subject. The method comprises (a) administering to the subject a pharmaceutically effective amount of a protein or polypeptide of the invention (b) administering to the subject a pharmaceutically effective amount of a polynucleotide, or fragment thereof, that encodes the polypeptide of the invention; or (c) administering to the subject a pharmaceutically effective amount of an antibody or antigen-binding fragment thereof that specifically binds to the protein or polypeptide of the invention. The protein or polypeptide of the invention can comprise an amino acid sequence set forth as SEQ ID NO:1-157. The cancer can be colorectal cancer.

Still another embodiment of the invention provides a method of isolating a change mediated protein or polypeptide, and its cognate gene or polynucleotide, expressed by a first host under a first environmental condition and not under a second environmental condition. The method comprises the steps of:
(a) obtaining a cell, tissue or fluid sample from the first host under the first environmental condition and optionally storing it under conditions (e.g., frozen) to preserve proteins, polypeptides, and other components of potential interest (i.e., change mediated) in the sample;
(b) immunizing an animal, optionally one that is phylogenetically distant from the first host and optionally using strong adjuvants, with the sample from (a) to elicit a strong, broad antibody response, resulting in an immunized animal;
(c) collecting antibodies from the immunized animal and optionally purifying the antibodies;
(d) adsorbing the antibodies with tissue, homogenized tissue, cells, cell extracts or fluid samples from a second host (optionally of the same species or same individual host as used in (a)) under the second environmental condition;
(e) isolating unadsorbed antibodies; and
(f) using the unadsorbed antibodies to isolate proteins, polypeptides or other constituents (e.g., lipids, carbohydrates, or glycoproteins) present in the cell, tissue or fluid sample of the first host under the first environmental condition and not present in the cell, tissue or fluid of the host under the second environmental condition; and optionally
(g) identifying the isolated protein, polypeptide or other component.
   The first environmental condition can be a disease, a cancer, or an autoimmune disease. The second environmental condition can be normal condition, healthy condition, non-diseased condition or an environmental condition that is different from the first environmental condition. Cells and tissue can be from any part of the host. In the case where the host is an animal, the bodily fluid can be urine, tears, plasma, milk, lavage fluid, prostatic fluid, seminal fluid, saliva, scrum, sputum, and pleural effusion. The bodily fluid from a plant can be extracted from phloem or xylum. The bodily fluid can substantially lack cells. Where the first host is an animal, the immunized animal can be the same species as the first host animal or a different type of animal than the first host animal. The method can further comprise isolating proteins, peptides or other components of interest directly from homogenates or extracts of cells or tissues taken from the host under the first condition. Proteins or peptide can alternatively be captured using the unadsorbed antibodies from a library constructed using DNA or mRNA obtained from the host under the first environmental condition, wherein the library is an expression library or display library. “Probing a library” can comprise:
(a) immobilizing the unadsorbed antibodies on a solid support;
(b) adding cell or tissue homogenates or fluids of the first host under the first condition or expressed proteins of the genomic expression library or surface display library made from the DNA or RNA of the host under the first environmental condition;
(c) washing unbound proteins or members of the phage library from the solid support;
(d) recovering proteins and polypeptides or members of the surface display library that are bound to the solid support; and
(e) identifying the specifically captured proteins and polypeptides, or, in the case of surface display library probing, the gene or polynucleotide responsible for expressing the cognate protein or polypeptide that was captured by the antibody(ies).
   The solid support can be blocked with a blocking agent before the homogenate, fluid, or library is added to decrease non-specific binding. The solid support can be selected from the group consisting of nitrocellulose, nylon, polystyrene, polyvinylchloride, latex, fiberglass, glass, microsphere, liposome, sepharose, sephadex, and a magnetic particle. The antibodies can be derived from an immunized animal selected from the group consisting of humans, baboons, chimpanzees, macaques, cattle, sheep, pigs, horses, goats, dogs, cats, rabbits, guinea pigs, rats, mice, chickens, ducks, and fish. The cells, tissues, and bodily fluid samples can be frozen immediately after it is obtained from the host under the first environmental condition. The cells, tissues, and fluid samples from the second host under the second environmental condition can be frozen immediately after they are obtained to minimize degradation of molecules needed to adsorb and remove non-change mediated components. Identification of the captured proteins, polypeptides or other components (e.g., lipids, carbohydrates, or glycoproteins) can be performed using conventional methods known in the art such as mass spectroscopy in association with separation methods (e.g., GeLC-MS/MS).

Also provided is a method of confirming and validating the specifically expressed nature of the isolated protein/polypeptide as expressed by the host in response to the disease or change mediated condition. The method comprises:
(a) purifying the natural or recombinant protein or polypeptide;
(b) producing non-cross reactive (e.g., monoclonal) antibodies to the polypeptide;
(c) probing cells, cell extracts, bodily fluids, or tissue of the host under the first and second environmental conditions with the antibodies of (b); and
(d) demonstrating relative reactivity of the antibody(ies) with samples from the first but not the second environmental condition.
   whereby the identified protein or polypeptide and its cognate polynucleotide is confirmed as being a change mediated molecule expressed under the first environmental condition but not the second if the antibodies specifically bind with the cells, cell extracts, bodily fluids, or tissue obtained from the host under the first condition but not the second.

### BRIEF DESCRIPTION OF THE DRAWING

FIG. 1 shows an assessment of reactivity of polyclonal egg antibodies (YPAbs) raised in chickens using homogenates of stage IV human colon cancer tissue with pooled sera of patients diagnosed with stage IV colon cancer by dot immunoblot assay. Differential reactivity of spotted pooled sera from stage IV colon cancer patients was compared with spots of control serum from age, gender and ethnicity-matched healthy patients (spot 4), BSA (spot 3), and homogenates of healthy tissue (spot 1). A homogenate of stage IV cancer tissue was the positive control (spot 2).

### DETAILED DESCRIPTION OF THE INVENTION

As used in this specification and the appended claims, the singular forms “a,” “an” and “the” include plural references unless the content clearly dictates otherwise.

Identification of Proteins that are Differentially Regulated in Cancer Cells

Proteomics-based Change Mediated Antigen Technology (PCMAT) is a method for identifying proteins and polypeptides and their cognate genes or polynucleotides that are specifically expressed when a cell undergoes a change (e.g., change from a normal, healthy cell to a diseased cell) or is exposed to a change in environmental conditions (e.g., change of a plant cell going from moist to arid conditions). PCMAT can be used to identify proteins and polypeptides and their cognate genes or polynucleotides that are up-regulated or are specifically expressed in cells when the cells become diseased or cancerous.

By “specifically expressed” is meant that the protein or polypeptide is expressed to a greater or lesser extent under a first environmental condition as compared to a second environmental condition. For example, the protein or polypeptide might be expressed under a first environmental condition but not expressed under a second environmental condition. Alternatively, the protein or polypeptide might be expressed to a greater extent, for example 10%, 20%, 50%, 100%, 200%, or more, in the first environmental condition as compared to the second environmental condition.

First environmental conditions include, but are not limited to, a disease condition (such as, for example, a viral disease, a bacterial disease, a fungal disease, a disease caused by a prion, a disease caused by a protozoan, a parasitic disease, cancer, an autoimmune disease (e.g., arthritis, chronic inflammatory bowel disease, or diabetes), heat, cold, exposure to toxic chemicals, exposure to drugs, exposure to chemotherapy drugs or regimens, exposure to stress, exposure to toxic metals, exposure to radiation, exposure to toxins, exposure to antibiotics, exposure to chemicals meant to kill or slow the growth of the microbe such as bactericides, viricides, and bacteriostatic or viristatic agents, low oxygen conditions, high oxygen conditions, low pH conditions, high pH conditions, exposure to iron, exposure to low levels of nutrients, and exposure to high levels of nutrients.

A second environmental condition can be, for example, normal conditions, healthy conditions, non-diseased conditions, and/or the absence of the first environmental conditions. In one embodiment of the invention, a first environmental condition can be one stage or phase of a disease (e.g., early, middle, late, chronic, treated, untreated, treatment for a certain amount of time, remission) and the second environmental condition can be a second, different stage of a disease (e.g., early, middle, late, chronic, treated, untreated, treatment for a certain amount of time, remission).

One embodiment of the invention provides a method for isolating a protein, polypeptide or other component of a cell (e.g., lipid, carbohydrate, or glycoprotein) that is expressed under a first environmental condition (e.g., a diseased condition) and not under a second environmental condition (e.g., a healthy or non-diseased condition). In general, the method comprises obtaining a first sample from a host in a first condition (e.g., a diseased condition) and immunizing a second animal with the host sample. Antibodies from the immunized animal are collected and adsorbed with host samples collected from a second host under a second environment condition (e.g., healthy conditions). The second host can be the same individual first host under the second conditions (e.g. healthy tissues or cells from the first host) or a different host of the same or different species as the first host. Unadsorbed antibodies are collected and used to collect differentially expressed proteins, polypeptides or other components directly from diseased tissue or fluid of the first host or from an expression or display library of the host's DNA or RNA or similar DNA or RNA.

The host exposed to the first environmental condition can be any type of organism, for example, a mammal, such as a human, baboon, chimpanzee, macaque, cattle, sheep, pig, horse, goat, dog, cat, rabbit, guinea pig, rat, or mouse. An animal can also be, for example, a chicken, duck, insect, or fish. The host can also be a member of the plant or microbial kingdom.

In the case where the host is from the animal kingdom, the sample collected from a host in the first environmental condition can be, for example, cells, cell extracts, tissue, bodily fluid, bodily fluid substantially lacking cells (e.g., less than about 1, 5, or 10% cells), serum, urine, tears, milk, seminal fluid, prostatic fluid, lung lavage fluid, saliva, mucosal cells, tumor cells, cancer cells, a biopsy sample, a lavage sample, sputum, plasma, blood, a fecal sample, a lymph node sample, bone marrow, colon tissue, rectal tissue, or a pleural effusion sample. Where the host is a plant, the sample can be from, e.g., cells, tissues, cell extracts, fluid extracted from phloem, fluid extracted from xylum. Wherein the host is a microbe, bacterium, virus or prion the sample can be cells or cell extracts, or cells or tissues of a host infected or colonized by the microbe.

Samples from animal host in a first environmental condition can be collected and processed immediately for immunization or are quickly frozen for later processing to preserve as closely as possible all of the potential epitopes that were present in the host animal sample at the moment the sample was taken. Individual samples or pooled samples collected at different time intervals or from different sampling sites or from different animals exposed to the same first environmental condition or similar environmental conditions can be used to immunize an animal to obtain an antibody response.

Antibodies from the immunized animal are collected. The immunized animal can be any type of animal capable of mounting a humoral immune response, for example, a mammal, such as a human, baboon, chimpanzee, macaque, cattle, sheep, pig, horse, goat, dog, cat, rabbit, guinea pig, rat, or mouse. An animal can also be, for example, a chicken, duck, insect, or fish. In one embodiment, the immunized animal is the same species as the first host animal. In another embodiment, the immunized animal is a different species from the first host animal. In another embodiment, the immunized animal is a different species from the first host animal wherein the immunized animal is distantly related to the first host animal (e.g., the first host animal is a human and the immunized animal is a chicken).

In the case where a bodily fluid is used as the immunogen, the fluid sample does not need to come from the site of the first environmental condition. That is, the bodily fluid does not need to be collected from the direct site of the diseased tissue or cancerous lesion, but instead can be, e.g., scrum drawn from a site away from the diseased tissue or cancerous lesion.

The immunization of animals with an antigen sample for the production of antibodies is well known in the art. See e.g., Antibody Techniques, Malik &Lillehoj, eds., Academic Press (1994); Antibodies: A Laboratory Manual, Harlow & Lane, eds., Cold Spring Harbor Laboratories (1988). A sample can be homogenized before administration to an animal. Administration can be by, for example, intramuscular, interperitoneal, subcutaneous, intradermal, intravenous, or nasal/inhalation, or combinations thereof.

The administration of the sample to the animal can be combined with an adjuvant. Alternatively, an adjuvant can be administered to the animal separately. An adjuvant can enhance an immune response to an antigen. An adjuvant can be, for example, complete Freund's adjuvant (CFA), Incomplete Freund's Adjuvant (IFA), montanide ISA (incomplete Seppic adjuvant), Ribi Adjuvant System (RAS), TiterMax®, Syntex Adjuvant Formulation (SAF), aluminum salt adjuvants, nitrocellulose-adsorbed antigen, encapsulated or entrapped antigens, immune-stimulating complexes (ISCOMs), for example Quil A or QS-21, and Gerbu® adjuvant. One of skill in the art can choose an appropriate adjuvant for a particular sample.

Booster administrations of the host samples from a first environmental condition can be given to the animal at, for example, 2 weeks, 1 month, two months, or three months after the immunization.

After an immune response occurs in the animal, an antibody sample is collected from the immunized animal. The sample can comprise, for example, the serum of an immunized animal. The animal's serum will contain antibodies, including antibodies specific for antigens expressed under the first environmental condition by the host animal (e.g., a diseased condition). Antibodies collected from an individual immunized animal can be used or antibodies pooled from two or more animals can be used. For example, antibodies collected from about 2, 5, 25, 100, 500, or 1,000 animals can be pooled.

Antibodies that bind to antigens that are produced under a second environmental condition, e.g., a healthy or non-disease condition are subtracted from the sample of antibodies. The result is an “unadsorbed antibody” sample. The antibodies are collected from the immunized animal and adsorbed with an animal host sample comparable to the one used to produce the antibodies, except that this sample is obtained from a host animal that is in the second environmental condition (e.g., healthy, normal or a condition that differs from the first environmental condition). The animal host sample (i.e., a host sample collected from a host animal in the second environmental condition) can be, for example, cells, cell extracts, tissue, bodily fluid, bodily fluid substantially lacking cells (e.g., less than about 1, 5, or 10% cells), serum, urine, tears, milk, seminal fluid, prostatic fluid, lung lavage fluid, saliva, mucosal cells, tumor cells, cancer cells, a biopsy sample, a lavage sample, sputum, plasma, blood, a fecal sample, a lymph node sample, bone marrow, colon tissue, rectal tissue, a pleural effusion sample, microbial or plant cells, tissues, or cell extracts. The adsorption removes antibodies that are reactive with proteins and other cell components made by the host in the second environmental condition (e.g., in the absence of disease). Unadsorbed antibodies that are reactive with antigens expressed by the animal host under the first environmental condition are recovered and used to capture proteins, polypeptides and other components specifically expressed by the host under the first environmental condition. The source of the proteins and polypeptides can be extracts of the tissues or bodily fluids from the animal in the first environmental condition. Alternatively, an expression or display library of the host's DNA or RNA can be used as the source of proteins. Proteins specifically captured by the adsorbed antibodies are eluted, concentrated and identified by proteomic methods known to persons skilled in the art (e.g., GeLC-MS/MS). In the case where surface display libraries are used, the cloned genetic fragment encoding the displayed protein is sequenced and the protein expressed by this fragment is deduced.

The adsorption step can be performed by, for example, contacting the antibody sample with host samples from the second environmental condition that are immobilized on a solid support, such as a nitrocellulose membrane or latex beads. See, Brady & Daphtary, J. Infect. Dis. 158:965-972 (1988). Optionally, the host sample from the second environmental condition can be denatured (e.g., by heating) before use to expose additional immunoreactive epitopes. Two or more successive adsorptions can be performed using the same or different adsorption methodologies.

All or substantially all of the antibodies in the antibody sample whose corresponding antigens are derived from a host under a second environmental condition will bind to these antigens to form immune complexes. However, antibodies directed against antigens that are specifically expressed under the first environmental condition will remain uncomplexed since their corresponding antigens are not present in the host sample under the second environmental condition. The uncomplexed antibodies comprise the unadsorbed antibody sample.

Polypeptides can be expressed from polynucleotides of the invention. The polypeptides can then be used to generate antibodies that specifically bind to an immunological epitope present in the polypeptides of the invention. Antibodies of the invention are antibody molecules that specifically bind to a polypeptide of the invention or fragment thereof. An antibody of the invention can be a polyclonal antibody, a monoclonal antibody, a single chain antibody (scFv), or an antigen-binding fragment of an antibody.

Antigens induced under a first environmental condition can be directly verified as actually expressed by the animal host in response to a first environmental condition by directly probing biological samples taken from, e.g., disease sites or from bodily fluid samples by any method known in the art. For example, monoclonal antibodies generated against a change mediated protein can be raised and tested for their specificity and cross reactivity to other proteins or polypeptides that are known to be or may be present in the test sample. Monoclonal antibodies that show appropriate specificity for epitopes on change mediated proteins or polypeptides can be labeled by various methods and tested for their reactivity with appropriate biological samples including tissues or bodily fluids from the host in both environmental conditions one and two. The labeled antibodies will react with the biological sample from the host in condition one (i.e., diseased), but will not react with the biological sample from the host in condition two (i.e., healthy or non-diseased). These results provide direct evidence that the host specifically expresses the antigen of interest under a first environmental condition, and that the change mediated protein or polypeptide so identified has potential for use in diagnosis, prevention, and therapy of the disease condition.

Samples taken at regular intervals throughout the course of disease will assure the presence of proteins and other potentially important cell components that can be transiently expressed. The more samples that are taken, the better the likelihood that the entire array of specifically expressed components will be obtained. The samples obtained in different time stages of disease or first conditions can be combined for immunization. Alternatively, they can be used to separately immunize animals to determine the approximate time during the disease that a particular protein or other cell component is expressed.

For example, comparing proteins and polypeptides of a animal host that are expressed under a first environmental condition at different stages of disease can comprise immunizing an animal with a first sample comprising one or more animal host samples under a first environmental condition, wherein each of the one or more host samples is in about the same stage of disease progression or treatment phase (e.g., early, middle, late, chronic, treated, untreated, treatment for a certain amount of time, remission). The stage or treatment phase of the first condition can be ascertained by, for example, by a medical professional. Antibodies from the immunized animal are collected and adsorbed with a host sample under a second environmental condition (e.g., a healthy or normal condition). Unadsorbed antibodies are collected and used as described above to identify change mediated proteins and polypeptides that are expressed throughout the entire timecourse of the disease, with and without remission, and with and without treatment).

An animal is immunized with a second host sample comprising one or more host samples under the first environmental condition, wherein each of host samples is in about the same stage of disease progression or same treatment phase, wherein the stage or phase is different from the stage or phase a described above. Antibodies from the immunized animal are collected and adsorbed with host samples under a second environmental condition. Unadsorbed antibodies are collected and used as described above to identify change mediated proteins and polypeptides that are specifically expressed at particular stages of the disease or those that are specifically expressed in response to remission or treatment.

### Colorectal Cancer Application of PCMAT Techniques

PCMAT and variations of PCMAT were used herein to identify polynucleotides that are expressed when healthy colorectal cells become cancerous colorectal cells. Adenocarcinoma tissues were obtained from Asterand XpressBank (Detroit, Mich.). The samples were harvested and quick frozen to preserve intact any potential antigen that was present at the time of harvest. The identity of the diseased tissue and staging were performed by clinical and histopathological examination. Integrity of the tissue sample was confirmed by RNA profile. From a potential list of approximately 200 available tissue samples, 4 samples were selected based on the following criteria: adenocarcinoma was the principal diagnosis; stages 1, 2, 3 and 4 were represented based on the AJCC/UICC classification scheme; the RNA profile indicated that minimal degradation of the tissue had occurred during the period following harvesting and quick freezing; the diseased tissue was from the large bowel; paired (homologous), healthy tissue (confirmed by clinical and histopathological examination) was available; and the samples represented both males and females.

Each of the 4 cancerous tissue samples (stage 1, 2, 3 and 4) was processed independently and subjected to PCMAT, which identified proteins and polypeptides that are specifically expressed in the adenocarcinoma samples relative to the proteins that are expressed in healthy bowel tissue.

Briefly, the adenocarcinoma samples were homogenized in PBS and samples from each cancer stage were individually used to immunize appropriate animals. Chickens, which are phylogenetically distant from humans, were chosen for this step to optimize the strength and breadth of the immune response. A strong adjuvant (Complete Freund's Adjuvant) was also used for this purpose. Colorectal cancer stage-specific polyclonal immunoglobulin (PAbs) was obtained from egg yolks of immunized animals. To selectively enrich for immunoglobulin directed against protein antigens unique to colon carcinoma tissues and concomitantly deplete immunoglobulin directed against protein antigens expressed by cells comprising both healthy and cancerous tissues, homogenates of healthy, autologous bowel tissue were prepared as for the diseased tissue. Antibodies reactive with proteins expressed by healthy bowel tissue were depleted from the immunoglobulin by adsorption using the UltraBind affinity membranes with covalently coupled proteins from healthy tissues. The procedure was repeated until essentially no reactivity was observed in ELISA or western blots between the adsorbed immunoglobulin and the paired healthy tissue homogenates. Immunoglobulin depleted of antibodies reactive with constitutively expressed protein antigens from healthy tissues were subjected to another round of adsorption with whole cells and lysates of the Escherichia coli host strain/pET30 grown with inducer (1 mM IPTG) to remove any antibodies reactive or cross-reactive with contaminants in the cDNA libraries.

Change mediated proteins were captured using the unadsorbed antibodies remaining after the adsorption steps using two different sources. The first source was the homogenates of diseased tissue (stages 1, 2, 3, and 4) used to immunize the animals. The second source was a normalized cDNA library, NCI_CGAP_Col4, which was obtained from the I.M.A.G.E. consortium. This library reportedly was constructed using cDNA generated by reverse transcription of mRNA isolated from moderately differentiated colon adenocarcinoma, and cloning into the shuttle vector, pCMV-SPORT6.

Adsorbed immunoglobulin preparations were covalently bound to M-280 Tosyl-activated Dynabeads according to the manufacturer's (Dynal Biotech) directions to create “charged” magnetic beads. For immunocapture, 5 ml of previously prepared diseased tissue homogenates or cDNA expression library fractions containing recombinant proteins at a concentration of 1 mg/ml were incubated with 0.5 ml of charged beads for 2 h at 4° C. with tilt rotation. Following immunocapture, charged beads were washed with 10 bead volumes of wash buffer (PBS-0.2% NOG).

Specifically bound proteins were eluted three times with 1M acetic acid. All wash and elution fractions were collected for analysis. Following elution, the specifically bound proteins were immediately neutralized by addition of 10 volumes of 0.2 M Na₂PO₄ (pH 7.4) and stored at 4° C. in the presence of 0.02% sodium azide until further use. A negative control consisted of an identical volume of beads charged with preimmune immunoglobulin and treated as above to capture non-specifically bound proteins. Eluates from charged columns treated with soluble lysates from the cDNA library, and homogenates of the tumors clearly demonstrated the presence of proteins that were absent in the negative controls.

Proteins specifically bound by columns charged with adsorbed immunoglobulin were identified by GeLC-MS/MS at the University of Florida Interdisciplinary Center for Biotechnology Research (ICBR). Specifically bound recombinant proteins eluted from charged columns above were concentrated, fractionated on 1D SDS-PAGE, and digested in-gel with trypsin prior to tandem MS/MS. Fractions of the 1D-lane were reduced, alkylated, and digested with trypsin (Promega). The enzymatically-digested samples were separated using a C18 Pep Map HPLC column with elution using a formic acid gradient. GeLC-MS/MS analysis was carried out on a hybrid quadrupole-TOF mass spectrometer (QSTAR, Applied Biosystems, Framingham, Mass.). All MS/MS samples were analyzed using Mascot version 2.0.01 (Matrix Science, London, UK) and Scaffold (version Scaffold-01-06-03, Proteome Software Inc., Portland, Oreg.). Change mediated antigens identified were analyzed via bioinformatics by querying the human genomic sequence database.

Proteins and their cognate polynucleotides that are upregulated in stage 1 cancerous cells were identified. The identified polypeptides and proteins are as follows: Titin (also known as TTN rhabdomyosarcoma antigen MU-RMS 40) (e.g., GenBank Accession Number Q8WZ42-2 (SEQ ID NO:1)); HBA1 (e.g., GenBank Accession Number P69905 (SEQ ID NO:2)); Insulin-like growth factor 1 receptor (IGF1R) (e.g., GenBank Accession Number P08069 (SEQ ID NO:3)); Isoform 3 or zonadhesin precursor (e.g., GenBank Accession Number Q9Y493-1 (SEQ ID NO:4)); latent transforming growth factor beta binding protein 4 (LTBP4) (e.g., UniProt Accession Number A6NCG8 (SEQ ID NO:5)); ASXL1 (additional sex combs like 1) (e.g., GenBank Accession Number Q8IXJ9-1 (SEQ ID NO:6)); beta globin (HBB) (e.g., GenBank Accession Number P68871 (SEQ ID NO:7)); BMP15-bone morphogenetic protein (e.g., GenBank Accession Number NM_{—}005448.1 (see also, UniProt Accession Number O95972) (SEQ ID NO:8)); TRIM49 (also known as RNF18; tripartite motif-containing 49) (e.g., GenBank Accession Number Q9NS80 (SEQ ID NO:9)); DNAJ homolog subfamily B member 11 precursor (e.g., GenBank Accession Number Q9UBS4 (SEQ ID NO:10)); uncharacterized hematopoietic stem/progenitor cells protein MDS027 (also known as MDS027 hHBrk1 HSPC300) GenBank Accession Number Q9NZ47 (SEQ ID NO:11)); uncharacterized protein ALB (e.g., UniProt Accession Number A6NBZ8 (SEQ ID NO:12)); isoform 3 of sushi, nidogen and EGF-like domain-containing protein 1 precursor (e.g., GenBank Accession Number Q8TER0-4 (SEQ ID NO:13)); isoform 2 of peripherin (e.g., GenBank Accession Number P41219-2 (SEQ ID NO:14)); mitochondrial 28S ribosomal protein S22 (e.g., GenBank Accession Number P82650 (SEQ ID NO:15)); translation initiation factor EIF-2B subunit epsilon (e.g., GenBank Accession Number Q13144 (SEQ ID NO:16)); estradiol 17-beta-dehydrogenase 1 (e.g., GenBank Accession Number P14061 (SEQ ID NO:17)); XRCC6BP1 (e.g., GenBank Accession Number Q8N4L5 (SEQ ID NO:18)); brain-specific angiogenesis inhibitor 1 precursor (e.g., GenBank Accession Number O14514 (SEQ ID NO:19)); isoform 2 of ring finger and CCCH-type zinc finger domain-containing protein 2 (e.g., GenBank Accession Number Q91-HBD1-2 (SEQ ID NO:20)); hemoglobin subunit beta (e.g., GenBank Accession Number P68871 (SEQ ID NO:21)); isoform 1 of far upstream element-binding protein 1 (e.g., GenBank Accession Number Q96AE4-1 (SEQ ID NO:22)); GALECTIN-3 (e.g., GenBank Accession Number P17931 (SEQ ID NO:23)); lysozyme C precursor (e.g., GenBank Accession Number P61626 (SEQ ID NO:24)); actin, alpha skeletal muscle (e.g., GenBank Accession Number P68133 (SEQ ID NO:25)); isoform M2 of pyruvate kinase isozymes M1/M2 (e.g., GenBank Accession Number P14618-1 (SEQ ID NO:26)); AGR2 (e.g., GenBank Accession Number O95994 (SEQ ID NO:27)); neutrophil defensin 1 precursor (e.g., GenBank Accession Number P59665 (SEQ ID NO:28)); myeloblastin precursor (e.g., GenBank Accession Number P24158 (SEQ ID NO:29)); uncharacterized protein PSME2 (e.g., GenBank Accession Number Q9UL46 (SEQ ID NO:30)); tubulin beta-2C chain (e.g., UniProt Accession Number P68371 (SEQ ID NO:31)); thiosulfate sulfurtransferase (e.g., GenBank Accession Number Q16762 (SEQ ID NO:32)); heat shock 70 kDa protein 1(e.g., GenBank Accession Number P08107 (SEQ ID NO:33)); Ig kappa chain V-III region sie (e.g., GenBank Accession Number P01620 (SEQ ID NO:34)); macrophage migration inhibitory factor (e.g., GenBank Accession Number P14174 (SEQ ID NO:35)); isoform 1 of ATP synthase subunit D, mitochondrial (e.g., GenBank Accession Number O75947-1 (SEQ ID NO:36)); uncharacterized protein ENSP00000374051 (e.g., GenBank Accession Number A6NGM3 (SEQ ID NO:37)); isocitrate dehydrogenase [NADP] cytoplasmic (e.g., UniProt Accession Number O75874 (SEQ ID NO:38)); hemoglobin subunit delta (e.g., GenBank Accession Number P02042 (SEQ ID NO:39)); isoform 1 of splicing factor, arginine/serine-rich 7 (e.g., GenBank Accession Number Q16629-1 (SEQ ID NO:40)); isoform 1 of mRNA-capping enzyme (e.g., GenBank Accession Number O60942-1 (SEQ ID NO:41)); LON protease homolog, mitochondrial precursor (e.g., GenBank Accession Number P36776 (SEQ ID NO:42)); signal recognition particle 54 kDa protein (e.g., GenBank Accession Number P61011 (SEQ ID NO:43)); isoform long of galectin-9 (e.g., GenBank Accession Number O00182-1 (SEQ ID NO:44)); integrin-linked protein kinase (e.g., GenBank Accession Number Q13418 (SEQ ID NO:45)); bifunctional aminoacyl-tRNA synthetase (e.g., GenBank Accession Number P07814 (SEQ ID NO:46)); isoform 1 of zinc finger protein 207 (e.g., GenBank Accession Number O43670-1 (SEQ ID NO:47)); inorganic pyrophosphatase (e.g., GenBank Accession Number Q15181 (SEQ ID NO:48)); calponin-2 (e.g., GenBank Accession Number Q99439 (SEQ ID NO:49)); isoform 1 of muscleblind-like protein 3 (e.g., GenBank Accession Number Q9NUK0-1 (SEQ ID NO:50)); cathepsin G precursor (e.g., GenBank Accession Number P08311 (SEQ ID NO:51)); zinc finger and BTB domain-containing protein 34 (e.g., GenBank Accession Number Q8NCN2 (SEQ ID NO:52)); adenine phosphoribosyltransferase (e.g., GenBank Accession Number P07741 (SEQ ID NO:53)); 40S ribosomal protein S9 (e.g., GenBank Accession Number P46781 (SEQ ID NO:54)); TALIN-1 (e.g., GenBank Accession Number Q9Y490 (SEQ ID NO:55)); leucine-rich repeat-containing protein 59 (e.g., GenBank Accession Number Q96AG4 (SEQ ID NO:56)); ATP synthase subunit alpha, mitochondrial precursor (e.g., GenBank Accession Number P25705 (SEQ ID NO:57)); isoform 7 of protein transport protein SEC31A (e.g., GenBank Accession Number O94979-7 (SEQ ID NO:58)); dihydroxyacetone kinase (e.g., GenBank Accession Number Q3LXA3 (SEQ ID NO:59)); protein similar to heterogeneous nuclear ribonucleoproteins C1/C2 (HNRNP C1/HNRNP C2) isoform 4 (e.g., ENSEMBL Accession Number ENST0000342709 (see also, GenBank Accession No. NM_{—}004500.3 and UNIPARC Accession Number IP100868835) (SEQ ID NO:60)); 18 kDa protein (e.g., UNIPARC Accession Number IP100796554 (SEQ ID NO:61)); cold agglutinin FS-1 L-chain (e.g., GenBank Accession Number A2NB45 (SEQ ID NO:62)); isoform 1 of heterogeneous nuclear ribonucleoprotein d0 (e.g., UniProt Accession Number Q14103-1 (SEQ ID NO:63)); DAZAP1/MEF2D fusion protein (e.g., GenBank Accession Number Q51RN2 (SEQ ID NO:64)).

Proteins and their cognate polynucleotides that are upregulated in stage IV cancerous cells were also identified. The polynucleotides encode the following polypeptides: POTE2 (also known as ANKRD26-like family C, member 1A) (e.g., GenBank Accession Number NP_{—}001077007 (SEQ ID NO: 65)); keratin 18 (KRT18) (e.g., GenBank Accession Number NP_{—}000215 (SEQ ID NO: 66)); PSME4 Isoform 1 of Proteasome activator complex subunit (also known as prosome macropain, activator subunit 4) (e.g., GenBank Accession Number NP_{—}055429 (SEQ ID NO: 67)); Mitogen-activated protein kinase-activated protein kinase (MAPKAPK33) (e.g., GenBank Accession Number NP_{—}004626 (SEQ ID NO: 68)); Complement component 1, s subcomponent (C1S) (e.g., GenBank Accession Number NP_{—}001725 (SEQ ID NO: 69)); Lysozyme C precursor (LYZ) (e.g., GenBank Accession Number NP_{—}000230 (SEQ ID NO: 70)); Keritin Type Cytoskeletal 20 (KRT20) (e.g., GenBank Accession Number NP_{—}061883 (SEQ ID NO: 71)); RNASE3 (also known as ECP RNS3, ribonuclease, RNase A family 3) (e.g., GenBank Accession Number NP_{—}002926 (SEQ ID NO: 72)); Aldehyde dehydrogenase X, mitochondrial precursor (ALDH1B1) (e.g., GenBank Accession Number NP_{—}000683 (SEQ ID NO: 73)); CDNA FLJ25506 fis, clone CBR05185 (e.g., GenBank Accession Number Q8N716 (SEQ ID NO: 74)); Isoform B of fibulin-1 precursor (FBLN1) (e.g., GenBank Accession Number P23142-2 (SEQ ID NO: 75)); Nucleobindin 1 (NUCB1) (e.g., GenBank Accession Number NP_{—}006175 (SEQ ID NO: 76)); Historic cluster 2, H2ba (HIST2H2BA) (e.g., GenBank Accession Number NP_{—}001019770 (SEQ ID NO: 77)); Tripartite motif-containing 28 (TRIM28) (e.g., GenBank Accession Number NP_{—}005753 (SEQ ID NO: 78)); Peroxisomal D3, D2 enoyl-CoA isomerase (PECI) (e.g., GenBank Accession Number NP_{—}006108 (SEQ ID NO: 79)); Peptidylprolyl isomerase B (PPIB) (e.g., GenBank Accession Number NP_{—}000933 (SEQ ID NO: 80)); Similar to 40S ribosomal protein S17 (e.g., GenBank Accession Number IP00743305 (SEQ ID NO: 81)); Eukaryotic translation elongation factor 1 gamma (EEF1G) (e.g., GenBank Accession Number IP100747497 (SEQ ID NO: 82)); Keratin 8 (KRT8) (e.g., GenBank Accession Number NP_{—}002264 (SEQ ID NO: 83)); Fibulin 2 (FBLN2) (e.g., GenBank Accession Number NP_{—}001989 (SEQ ID NO: 84)); VIM (e.g., GenBank Accession Number NP_{—}003371 (SEQ ID NO: 85)); Fibrinogen alpha chain (FGA) (e.g., GenBank Accession Number NP_{—}000499 (SEQ ID NO: 86)); Annexin A2 (ANXA2) (e.g., GenBank Accession Number NP_{—}001002858 (SEQ ID NO: 87)); H2A histone family, member J (H2AFJ) (e.g., GenBank Accession Number NP_{—}808760 (SEQ ID NO: 88)); Actin alpha, cardiac muscle 1 (ACTC1) (e.g., GenBank Accession Number NP_{—}005150 (SEQ ID NO: 89)); Keratin 19 (KRT19) (e.g., GenBank Accession Number NP_{—}002267 (SEQ ID NO: 90)); Immunoglobin lambda locus (IGL@protein) (e.g., GenBank Accession Number Q6PIQ7 (SEQ ID NO: 91)); immunoglobulin heavy constant mu (IGHM) (e.g., GenBank Accession Number Q8WUK1 (SEQ ID NO: 92)); EGF-containing Fibulin-like extracellular matrix protein 1 (EFEMP1) (e.g., GenBank Accession Number Q12805-3 (SEQ ID NO: 93)); Tripartite motif-containing protein 34 (e.g., GenBank Accession Number NP_{—}067629 (SEQ ID NO: 94)); Isoform 3 of AP1-subunit Gamma Binding Protein 1 (e.g., GenBank Accession Number NP_{—}542117 (SEQ ID NO: 95)); Proflin-1 (e.g., GenBank Accession Number NP_{—}005013 (SEQ ID NO:96)); Histone H4 (e.g., GenBank Accession Number NP_{—}001029249 (SEQ ID NO: 97)); Hemoglobin subunit alpha (e.g., GenBank Accession Number NP_{—}000549 (SEQ ID NO: 98)); Transgelin (also known as TAGLN) (e.g., GenBank Accession Number NP_{—}001001522 (SEQ ID NO: 99)); Lumican precursor (e.g., GenBank Accession Number NP_{—}002336 (SEQ ID NO: 100)); Hemoglobin Beta (also known as HBD CD113t) (e.g., GenBank Accession Number NP_{—}000509 (SEQ ID NO: 101)); Fibrinogen Beta Chain Precursor (e.g., GenBank Accession Number NP_{—}005132 (SEQ ID NO: 102)); immunoglobulin kappa constant (IGKC) (e.g., GenBank Accession Number Q6GMX8 (SEQ ID NO: 103)); Uncharacterized Protein ALB (also known as albumin) (e.g., GenBank Accession Number Q56G89 (SEQ ID NO: 104)).

In another example, PCMAT was used to identify proteins that are shed into body fluids during a diseased state, namely stage IV colorectal bowel cancer. See Example 1. This study used the YPAbs (polyclonal IgY antibodies) raised in chickens against adjuvanted homogenates of stage IV human colon cancer tissue. The YPAbs evoked from the stage IV tumor tissue were adsorbed with sera from healthy subjects bound to a solid support. After confirmation using western and dot blots that no remaining antibodies reactive with antigens present in healthy serum was established, the remaining unadsorbed antibodies were bound to a solid support resin to create a charged column as described above. Serum from patients with stage IV colorectal cancer was passed through the column, and non-specifically bound proteins and peptides were removed by washing. Specifically bound proteins were removed using acetic acid, which were identified by GeLC-MS/MS as described above. Stage II tumor tissue was used in the same manner to identify SEQ ID NOs:108-157 and are as follows: Actin, Cytoplasmic 1 (actin beta) (e.g., GenBank Accession Number NP_{—}001092 (SEQ ID NO:108)); Hemoglobin beta (e.g., GenBank Accession Number O95408 (SEQ ID NO:109)); Hemoglobin subunit alpha (e.g., GenBank Accession Number P69905 (SEQ ID NO:110)); POTE-2 alpha actin (e.g., GenBank Accession Number A5A3E0 (SEQ ID NO:111)); SLC4A10 (e.g., GenBank Accession Number Q6U841 (SEQ ID NO:112)); Ribonuclease P Protein Subunit P20 (POP7) (e.g., GenBank Accession Number O75817 (SEQ ID NO:113)); Nuclear RNA export factor 1 (NXF1) (e.g., GenBank Accession Number Q59E96 (SEQ ID NO:114)); UVEAL Autoantigen With Coiled-Coil Domains And Ankyrin Repeats, UACA (e.g., GenBank Accession Number Q05DB3 (SEQ ID NO:115)); Uncharacterized Protein C13ORF27 (e.g., GenBank Accession Number Q5JUR7 (SEQ ID NO:116)); Isoform 3 of Sushi, Nidogen And EGF-Like Domain-Containing Protein 1 Precursor (e.g., GenBank Accession Number Q8TER0 (SEQ ID NO:117)); Isoform 1 Of Dynein Heavy Chain 10, Axonemal (DNAH10): (e.g., GenBank Accession Number Q8IVF4 (SEQ ID NO:118)); Gap junction alpha-1 protein (GJA1/Connexion 43) (e.g., GenBank Accession Number P17302 (SEQ ID NO:119)); Isoform 1 Of Kinesin-Like Protein KIF25 (KIF25) (e.g., GenBank Accession Number Q5SZU8 (SEQ ID NO:120)); GAPDH-Glyceraldehyde-3-Phosphate Dehydrogenase (e.g., GenBank Accession Number P04406 (SEQ ID NO:121)); Uncharacterized Protein ALB (e.g., GenBank Accession Number P02768 (SEQ ID NO:122)); Galectin-3, LGALS3 (e.g., GenBank Accession Number NP_{—}002297 (SEQ ID NO:123)); Similar to NAC-Alpha Domain-Containing Protein 1 (NACAD) (e.g., GenBank Accession Number O15069 (SEQ ID NO:124)); Acetyl-CoA Acetyltransferase, Mitochondrial, ACAT1 (e.g., GenBank Accession Number NP_{—}000010 (SEQ ID NO:125)); KH-Type Splicing Regulatory Protein, FUBP2 (e.g., GenBank Accession Number NP_{—}003676 (SEQ ID NO:126)); Profilin 1 (PFN1) (e.g., GenBank Accession Number NP_{—}005013 (SEQ ID NO:127)); Chloride Intracellular Channel Protein 1, CLIC1 (e.g., GenBank Accession Number NP_{—}001279 (SEQ ID NO:128)); Zinc Finger Protein 831 (e.g., GenBank Accession Number NP_{—}848552 (SEQ ID NO:129)); Endoplasmin (e.g., GenBank Accession Number NP_{—}003290 (SEQ ID NO:130)); Ribosomal Protein S10 (RPS10) (e.g., GenBank Accession Number P46783 (SEQ ID NO:131)); Splicing Factor, Arginine/Serine-Rich 3 (e.g., GenBank Accession Number NP_{—}003008 (SEQ ID NO:132)); ACTA2 Protein (alpha actin, smooth muscle) (e.g., GenBank Accession Number P62736 (SEQ ID NO:133)); Isoform 1 of Sodium Channel Protein Type 8 Subunit Alpha, SCN8A (e.g., GenBank Accession Number NP_{—}055006 SEQ ID NO:134)); Isoform Long of Galectin-9 GenBank Accession Number NP_{—}033665 SEQ ID NO:135)); T-Complex Protein 1 Subunit Epsilon, CCT5 (e.g., GenBank Accession Number NP_{—}036205 (SEQ ID NO:136)); Alpha-Enolase, Lung Specific (e.g., GenBank Accession Number CAA47179 (SEQ ID NO:137)); Proto-Oncogene Serine/Threonine-Protein Kinase MOS (e.g., GenBank Accession Number NP_{—}005363 (SEQ ID NO:138)); Isoform 1 Of Beta-Adducin (ADD2) (e.g., GenBank Accession Number NP_{—}001608 (SEQ ID NO:139)); Apolipoprotein E (APOE) (e.g., GenBank Accession Number NP_{—}000032 SEQ ID NO:140)); Ubiquitin-4 (UBQLN4) (ataxin-1 ubiquitin-like interacting protein) (e.g., GenBank Accession Number NP_{—}064516 (SEQ ID NO:141)); Sumo-Conjugating Enzyme UB21 (UBC9 homolog in yeast) (e.g., GenBank Accession Number NP_{—}003336 (SEQ ID NO:142)); Myosin-15 (MYH15) (e.g., GenBank Accession Number NP_{—}055796 (SEQ ID NO:143)); FLJ93091, Homo Sapiens UMP-CMP Kinase (UMP-CMPK) (e.g., GenBank Accession Number NP_{—}057392 (SEQ ID NO:144)); Intelectin-1 (ITLN1) (e.g., GenBank Accession Number NP_{—}060095 (SEQ ID NO:145)); Apolipoprotein A-IV (APOA4) (e.g., GenBank Accession Number Q13784 (SEQ ID NO:146)); Mitochondrial pyruvate dehydrogenase (lipoamide) alpha 1 (PDHA1) (e.g., GenBank Accession Number P08559 (SEQ ID NO:147)); Leucine-Rich Repeat-Containing Protein 59 (LRRC59) (e.g., GenBank Accession Number NP_{—}060979 (SEQ ID NO:148)); 60S Ribosomal Protein L37A (RPL37A) (e.g., GenBank Accession Number NP_{—}000989 (SEQ ID NO:149)); Uridine-Cytidine Kinase 1-like 1 (UCKL1) (e.g., GenBank Accession Number Q53HM1 (SEQ ID NO:150)); Aldehyde Dehydrogenase 9A1 (ALDH9A1) (e.g., GenBank Accession Number NP_{—}000687 (SEQ ID NO:151)); Isoform 3 Of Thioredoxin Reductase 1, Cytoplasmic (TXNRD1) (e.g., GenBank Accession Number Q16881 (SEQ ID NO:152)); Nuclear Receptor Subfamily 2 Group E Member 1 (NR2E1) (e.g., GenBank. Accession Number NP_{—}003260 (SEQ ID NO:153)); Cation Channel Sperm-Associated Protein 3 (CATSPER3) (e.g., GenBank Accession Number NP_{—}821138 (SEQ ID NO:154)); Transmembrane EMP24 Domain-Containing Protein 1 (TMED1) (e.g., GenBank Accession Number NP_{—}006849 (SEQ ID NO:155)); Protein FAM154A (FAM154A) (e.g., GenBank Accession Number NP_{—}714918 (SEQ ID NO:156)); Isoform 1 of Transcriptional Repressor NF-X1 (NFX1) (e.g., GenBank Accession Number NP_{—}002495 (SEQ ID NO:157)).

Shed change mediated proteins and their cognate polynucleotides that are upregulated in stage IV cancerous cells were identified. The polynucleotides encode the polypeptides shown in SEQ ID NOs:105-107 (ApoA1 e.g., GenBank Accession Number P02647 (SEQ ID NO:105); C4A (e.g., GenBank Accession Number P0C0L4 (SEQ ID NO:106); and C3 187 kDa protein (e.g., GenBank Accession Number P01024 (SEQ ID NO:107)).

In general, PCMAT has a number of outstanding attributes, including its speed (the entire biomarker discovery portion of the project can be performed in less than 6 months), cost efficiency, and, most importantly, its sensitivity. In general, chickens serve as an excellent host in which to raise high titer, broadly reactive antibodies: they tolerate very strong adjuvants extremely well, they are phylogenetically distant from humans, which makes them more likely to respond to human immunogens in cancer studies, they have a very large immune repertoire, and enormous amounts of purified IgY (essentially identical to IgG) can be readily obtained from their eggs. The use of strong adjuvants helps to assure that even low abundance proteins will elicit an antibody response and will be recovered. Another aspect of PCMAT that promotes sensitivity is that the size of the charged column and the amount of the body fluid that can be passed through it can be substantial. Again, this promotes the likelihood of finding low abundance proteins. Finally, the subtraction step in which fluids from healthy subjects are used to remove antibodies reactive with background proteins results in a tremendously increased signal to noise ratio. The need for sensitivity as provided by PCMAT cannot be overstated. It is highly likely that cancerous proteins that are shed into body fluids are of relatively low-abundance, and therefore missed by strategies that are currently in use. The use of PCMAT to find cancerous shed proteins presents a unique opportunity for the identification of novel target for the development of diagnostics for cancer.

All of these polypeptides are referred to herein as “the polypeptides of the invention” or “cancer-associated antigens or polypeptides.” The polynucleotides that encode the polypeptides of the invention are referred to herein as “the polynucleotides of the invention” or “cancer-associated polynucleotides.”

### Polypeptides

A polypeptide is a polymer of three or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, and/or chemicals used in synthesis of the polypeptide has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure.

A polypeptide of the invention can comprise at least 1, 2, 3, 4, 5, 10, 25, 100, 500, 1,000 or more non-naturally occurring amino acids immediately contiguous with one or both of the amino and carboxy termini of the polypeptide.

Polypeptides of the invention can either be full-length polypeptides or proteins or fragments of polypeptides or proteins. For example, fragments of polypeptides of the invention can comprise about 5, 10, 15, 20, 30, 40, 50, 60, 70, 80, 90, 100, 250, 500, 750, 1,000, 2,000, 3,000, 4,000, 5,000 or more contiguous amino acids of polypeptides of the invention or any value or range between 5 and 5,000. Examples of polypeptides of the invention include those shown in SEQ ID NOs:1-157. Variant polypeptides are at least about 80, or about 85% 90, 91, 92, 93, 94, 95, 96, 97, 98, 99% or more identical to the polypeptide sequences shown in SEQ ID NOs:1-157. Variant polypeptides have one or more conservative amino acid variations or other minor modifications and retain biological activity, i.e., are biologically functional equivalents. A biologically active equivalent has substantially equivalent function when compared to the corresponding wild-type polypeptide.

Percent sequence identity has an art recognized meaning and there are a number of methods to measure identity between two polypeptide or polynucleotide sequences. See, e.g., Lesk, Ed., Computational Molecular Biology, Oxford University Press, New York, (1988); Smith, Ed., Biocomputing: Informatics And Genuine Projects, Academic Press, New York, (1993); Griffin & Griffin, Eds., Computer Analysis Of Sequence Data, Part I, Humana Press, New Jersey, (1994); von Heinje, Sequence Analysis In Molecular Biology, Academic Press, (1987); and Gribskov & Devereux, Eds., Sequence Analysis Primer, M Stockton Press, New York, (1991). Methods for aligning polynucleotides or polypeptides are codified in computer programs, including the GCG program package (Devereux et al., Nuc. Acids Res. 12:387 (1984)), BLASTP, BLASTN, FASTA (Atschul et al., J. Molec. Biol. 215:403 (1990)); and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman (Adv. App. Math., 2:482-489 (1981)). For example, the computer program ALIGN which employs the FASTA algorithm can be used, with an affine gap search with a gap open penalty of −12 and a gap extension penalty of −2.

When using any of the sequence alignment programs to determine whether a particular sequence is, for instance, about 95% identical to a reference sequence, the parameters are set such that the percentage of identity is calculated over the full length of the reference polynucleotide and that gaps in identity of up to 5% of the total number of nucleotides in the reference polynucleotide are allowed.

Variants can generally be identified by modifying one of the polypeptide sequences of the invention, and evaluating the properties of the modified polypeptide to determine if it is a biological equivalent. A variant is a biological equivalent if it reacts substantially the same as a polypeptide of the invention in an assay such as an immunohistochemical assay, an enzyme-linked immunosorbent assay (ELISA), a radioimmunoassay (RIA), immunoenzyme assay or a western blot assay, e.g. has 90-110% of the activity of the original polypeptide. In one embodiment, the assay is a competition assay wherein the biologically equivalent polypeptide is capable of reducing binding of the polypeptide of the invention to a corresponding reactive antigen or antibody by about 80, 95, 99, or 100%. An antibody that specifically binds a corresponding wild-type polypeptide also specifically binds the variant polypeptide. Variant polypeptides of the invention can comprise about 1, 2, 3, 4, 5, 10, 20, 30, 40, 50, 60, 70, 80, 100, 200 or more conservative amino acid substitutions or any value or range of substitutions between about 1 and about 200.

A conservative substitution is one in which an amino acid is substituted for another amino acid that has similar properties, such that one skilled in the art of peptide chemistry would expect the secondary structure and hydropathic nature of the polypeptide to be substantially unchanged. Conservative substitutions include swaps within groups of amino acids such as replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

A polypeptide of the invention can further comprise a signal (or leader) sequence that co-translationally or post-translationally directs transfer of the protein. The polypeptide can also comprise a linker or other sequence for case of synthesis, purification or identification of the polypeptide (e.g., poly-His), or to enhance binding of the polypeptide to a solid support. A polypeptide of the invention can further comprise a signal (or leader) sequence that co-translationally or post-translationally directs transfer of the protein. The polypeptide can also comprise a linker or other sequence for ease of synthesis, purification or identification of the polypeptide (e.g., poly-His), or to enhance binding of the polypeptide to a solid support. For example, a polypeptide can be conjugated to an immunoglobulin Fc region or bovine serum albumin.

A polypeptide can be covalently or non-covalently linked to an amino acid sequence to which the polypeptide is not normally associated with in nature. A polypeptide can also be covalently or non-covalently linked to compounds or molecules other than amino acids. For example, a polypeptide can be linked to an indicator reagent, an amino acid spacer, an amino acid linker, a signal sequence, a stop transfer sequence, a transmembrane domain, a protein purification ligand, or a combination thereof. In one embodiment of the invention a protein purification ligand can be one or more amino acid residues at, for example, the amino terminus or carboxy terminus of a polypeptide of the invention. An amino acid spacer is a sequence of amino acids that are not usually associated with a polypeptide of the invention in nature. An amino acid spacer can comprise about 1, 5, 10, 20, 100, 500, 1,000 or more amino acids.

If desired, a polypeptide can be a fusion protein, which can also contain other amino acid sequences, such as amino acid linkers, amino acid spacers, signal sequences, TMR stop transfer sequences, transmembrane domains, as well as ligands useful in protein purification, such as glutathione-S-transferase, histidine tag, and staphylococcal protein A, or combinations thereof. More than one polypeptide of the invention can be present in a fusion protein. Fragments of polypeptides of the invention can be present in a fusion protein of the invention. A fusion protein of the invention can comprise one or more of SEQ ID NOs:1-157, fragments thereof, or combinations thereof.

Polypeptides of the invention can be in a multimeric form. That is, a polypeptide can comprise one or more copies of SEQ ID NOs:1-157 or a combination thereof. A multimeric polypeptide can be a multiple antigen peptide (MAP). See e.g., Tam, J. Immunol. Methods, 196:17-32 (1996).

Polypeptides of the invention can comprise an antigen that is recognized by an antibody. The antigen can comprise one or more epitopes (i.e., antigenic determinants). An epitope can be a linear epitope, sequential epitope or a conformational epitope. Epitopes within a polypeptide of the invention can be identified by several methods. See, e.g., U.S. Pat. No. 4,554,101; Jameson & Wolf, CABIOS 4:181-186 (1988). For example, a polypeptide of the invention can be isolated and screened. A series of short peptides, which together span an entire polypeptide sequence, can be prepared by proteolytic cleavage. By starting with, for example, 100-mer polypeptide fragments, each fragment can be tested for the presence of epitopes recognized in an ELISA. For example, in an ELISA assay a polypeptide, such as a 100-mer polypeptide fragment, is attached to a solid support, such as the wells of a plastic multi-well plate. A population of antibodies are labeled, added to the solid support and allowed to bind to the unlabeled antigen, under conditions where non-specific absorption is blocked, and any unbound antibody and other proteins are washed away. Antibody binding is detected by, for example, a reaction that converts a colorless substrate into a colored reaction product. Progressively smaller and overlapping fragments can then be tested from an identified 100-mer to map the epitope of interest.

A polypeptide of the invention can be produced recombinantly. A polynucleotide encoding a polypeptide of the invention can be introduced into a recombinant expression vector that can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system. A polypeptide can also be chemically synthesized or obtained from cancerous cells.

An immunogenic polypeptide of the invention can comprise an amino acid sequence shown in SEQ ID NOs:1-157. An immunogenic polypeptide can elicit antibodies or other immune responses (e.g., T-cell responses of the immune system) that recognize epitopes of polypeptides having SEQ ID NOs:1-157. An immunogenic polypeptide of the invention can also be a fragment of a polypeptide that has an amino acid sequence shown in SEQ NOs:1-157. An immunogenic polypeptide fragment of the invention can be about 5, 10, 15, 20, 30, 40, 50, 60, 70, 80, 90, 100, 250, 500, 750, 1,000, 2,000, 3,000, 4,000, 5,000 or more or any value or range between about 5 and about 5,000 amino acids in length.

### Polynucleotides

Polynucleotides of the invention contain less than an entire genome and can be single- or double-stranded nucleic acids. A polynucleotide can be RNA, mRNA, DNA, cDNA, genomic DNA, chemically synthesized RNA or DNA or combinations thereof. The polynucleotides can be purified free of other components, such as proteins, lipids and other polynucleotides. For example, the polynucleotide can be 50%, 75%, 90%, 95%, 96%, 97%, 98%, 99%, or 100% purified. The polynucleotides of the invention encode the polypeptides described above. In one embodiment of the invention the polynucleotides encode polypeptides of the invention and polypeptides shown in SEQ ID NOs:1-157, the complements thereof, or combinations thereof. Polynucleotides of the invention can comprise other nucleotide sequences, such as sequences coding for linkers, signal sequences, TMR stop transfer sequences, transmembrane domains, or ligands useful in protein purification such as glutathione-S-transferase, histidine tag, and staphylococcal protein A.

Polynucleotides of the invention can be isolated. An isolated polynucleotide is a polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. A nucleic acid molecule existing among hundreds to millions of other nucleic acid molecules within, for example, cDNA or genomic libraries, or gel slices containing a genomic DNA restriction digest are not to be considered an isolated or purified polynucleotide.

Polynucleotides of the invention can also comprise fragments that encode immunogenic polypeptides. Polynucleotides of the invention can encode full-length polypeptides or proteins, polypeptide fragments, and variant or fusion polypeptides.

Degenerate nucleotide sequences encoding polypeptides of the invention, as well as homologous nucleotide sequences that are at least about 80, or about 85, 90, 95, 96, 97, 98, 99% or more identical to the polynucleotide sequences of the invention and the complements thereof are also polynucleotides of the invention. Percent sequence identity can be calculated as described in the “Polypeptides” section. Degenerate nucleotide sequences are polynucleotides that encode a polypeptide of the invention or fragments thereof, but differ in nucleic acid sequence from the wild-type polynucleotide sequence, due to the degeneracy of the genetic code. Complementary DNA (cDNA) molecules, species homologs, and variants of polynucleotides that encode biologically functional polypeptides of the invention also are polynucleotides of the invention. Polynucleotides of the invention can be isolated from nucleic acid sequences present in, for example, a biological sample, such as blood, serum, saliva, or tissue from an individual patient. Polynucleotides can also he synthesized in the laboratory, for example, using an automatic synthesizer. An amplification method such as PCR can be used to amplify polynucleotides from either genomic DNA or cDNA encoding the polypeptides.

Polynucleotides of the invention can comprise coding sequences for naturally occurring polypeptides or can encode altered sequences that do not occur in nature. If desired, polynucleotides can be cloned into an expression vector comprising expression control elements, including for example, origins of replication, promoters, enhancers, or other regulatory elements that drive expression of the polynucleotides of the invention in host cells. An expression vector can be, for example, a plasmid, such as pBR322, pUC, or ColE1, or an adenovirus vector, such as an adenovirus Type 2 vector or Type 5 vector. Optionally, other vectors can be used, including but not limited to Sindbis virus, simian virus 40, alphavirus vectors, poxvirus vectors, and cytomegalovirus and retroviral vectors, such as murine sarcoma virus, mouse mammary tumor virus, Moloney murine leukemia virus, and Rous sarcoma virus. Minichromosomes such as MC and MC1, bacteriophages, phagemids, yeast artificial chromosomes, bacterial artificial chromosomes, virus particles, virus-like particles, cosmids (plasmids into which phage lambda cos sites have been inserted) and replicons (genetic elements that are capable of replication under their own control in a cell) can also be used.

Methods for preparing polynucleotides operably linked to an expression control sequence and expressing them in a host cell are well-known in the art. See, e.g., U.S. Pat. No. 4,366,246. A polynucleotide of the invention is operably linked when it is positioned adjacent to or close to one or more expression control elements, which direct transcription and/or translation of the polynucleotide.

Polynucleotides of the invention can be used, for example, as probes or primers, for example PCR primers, to detect the presence of polynucleotides in a sample, such as a biological sample. The ability of such probes and primers to specifically hybridize to polynucleotides of the invention will enable them to be of use in detecting the presence of complementary sequences in a given sample. Polynucleotide probes and primers of the invention can hybridize to complementary sequences in a sample such as a biological sample, including saliva, sputum, blood, urine, feces, cerebrospinal fluid, amniotic fluid, wound exudate, or tissue. Polynucleotides from the sample can be, for example, subjected to gel electrophoresis or other size separation techniques or can be immobilized without size separation. The polynucleotide probes or primers can be labeled. Suitable labels and methods for labeling probes and primers are known in the art, and include, for example, radioactive labels incorporated by nick translation or by kinase, biotin labels, fluorescent labels, chemiluminescent labels, bioluminescent labels, metal chelator labels and enzyme labels. Polynucleotides from a sample are contacted with the probes or primers under hybridization conditions of suitable stringencies.

Depending on the application, varying conditions of hybridization can be used to achieve varying degrees of selectivity of the probe or primer towards the target sequence. For applications requiring high selectivity, relatively stringent conditions can be used, such as low salt and/or high temperature conditions, such as provided by a salt concentration of from about 0.02 M to about 0.15 M salt, or any value or range between about 0.02M to about 0.15 M salt, at temperatures of from about 50° C. to about 70° C., or any value or range between about 50° C. to about 70° C. For applications requiring less selectivity, less stringent hybridization conditions can be used. For example, salt conditions from about 0.14 M to about 0.9M salt or any value or range between about 0.14 M to about 0.9M salt, at temperatures ranging from about 20° C. to about 55° C. or any value or range between about 20° C. to about 55° C. The presence of a hybridized complex comprising the probe or primer and a complementary polynucleotide from the test sample can indicate the presence of cancer in the sample.

### Antibodies

Antibodies of the invention are antibody molecules that specifically and stably bind to a polypeptide of the invention or fragment thereof. An antibody of the invention can be a polyclonal antibody, a monoclonal antibody, a single chain antibody (scFv), a monospecific single-chain antibody, a bispecific single-chain antibody, a bivalent single-chain antibody, a tetravalent single-chain antibody, a chimeric antibody, a humanized antibody, or an antigen-binding fragment of an antibody. Antigen-binding fragments of antibodies are a portion of an intact antibody comprising the antigen binding site or variable region of an intact antibody, wherein the portion is free of the constant heavy chain domains of the Fc region of the intact antibody. Examples of antigen-binding antibody fragments include Fab, Fab′, Fab′-SH, F(ab′)₂ and Fᵥ fragments.

An isolated antibody is substantially separated from its natural environment. For instance, an isolated antibody is substantially separated from the biological source from which it is derived. A purified antibody is substantially free of other material that associates with the antibody in its natural environment. For instance, a purified antibody is substantially free of cellular material or other proteins or antibodies from the cell or tissue from which it is derived. The term refers to preparations where the isolated antibody is at least about 70% to 80% (w/w) pure, more preferably, at least about 80%-90% (w/w) pure, even more preferably about 90-95% pure; and, most preferably at least about 95%, 96%, 97%, 98%, 99%, or 100% (w/w) pure.

An antibody of the invention can be any antibody class and any subtype, including for example, IgG (IgG1, IgG2, IgG4), IgM, IgA, IgD, IgE, and IgY. An antibody or antigen-binding fragment thereof binds to an epitope of a polypeptide of the invention. An antibody can be made in vivo in suitable laboratory animals or in vitro using recombinant DNA techniques. Means for preparing and characterizing antibodies are well know in the art. See, e.g., Dean, Methods Mol. Biol. 80:23-37 (1998); Dean, Methods. Mol. Biol. 32:361-79 (1994); Baileg, Methods Mol. Biol. 32:381-88 (1994); Gullick, Methods Mol. Biol. 32:389-99 (1994); Drenckhahn et al. Methods Cell. Biol. 37:7-56 (1993); Morrison, Ann. Rev. Immunol. 10:239-65 (1992); Wright et al. Crit. Rev. Immunol. 12:125-68 (1992). For example, polyclonal antibodies can be produced by administering a polypeptide of the invention to an animal, such as a human or other primate, mouse, rat, rabbit, guinea pig, goat, pig, dog, cow, sheep, donkey, chicken, or horse. Scrum from the immunized animal is collected and the antibodies are purified from the plasma by, for example, precipitation with ammonium sulfate, followed by chromatography, such as affinity chromatography. Techniques for producing and processing polyclonal antibodies are known in the art.

“Specifically binds” or “specific for” means that a first antigen, e.g., a polypeptide of the invention, recognizes and binds to an antibody of the invention with greater affinity than other, non-specific molecules. A non-specific molecule is an antigen that shares no common epitope with the first antigen. In this case, polypeptides of the invention would not generally be desirable choices for non-specific control molecules. For example, an antibody raised against a first antigen (e.g., a polypeptide) to which it binds more efficiently than to a non-specific antigen can be described as specifically binding to the first antigen. In a preferred embodiment, an antibody or antigen-binding portion thereof specifically binds to a polypeptide of the invention, such as SEQ ID NOs:1-157 or fragments thereof when it binds with a binding affinity Kₐ of 10⁷ l/mol or more. Specific binding can be tested using, for example, an enzyme-linked immunosorbant assay (ELISA), a radioimmunoassay (RIA), or a western blot assay using methodology well known in the art.

Additionally, monoclonal antibodies directed against epitopes present on a polypeptide of the invention can also be readily produced. For example, normal B cells from a mammal, such as a mouse, which was immunized with a polypeptide of the invention can be fused with, for example, HAT-sensitive mouse myeloma cells to produce hybridomas. Hybridomas producing antibodies can be identified using RIA or ELISA and isolated by cloning in semi-solid agar or by limiting dilution. Clones producing polypeptide-specific antibodies are isolated by another round of screening. Monoclonal antibodies can be screened for specificity using standard techniques, for example, by binding a polypeptide of the invention to a microtiter plate and measuring binding of the monoclonal antibody by an ELISA assay. Techniques for producing and processing monoclonal antibodies are known in the art. See e.g., Kohler & Milstein, Nature, 256:495 (1975). Particular isotypes of a monoclonal antibody can be prepared directly, by selecting from the initial fusion, or prepared secondarily, from a parental hybridoma secreting a monoclonal antibody of a different isotype by using a sib selection technique to isolate class-switch variants. See Steplewski et al., P.N.A.S. U.S.A. 82:8653 1985; Spria et al., J. Immunolog. Meth. 74:307, 1984. Monoclonal antibodies of the invention can also be recombinant monoclonal antibodies. See, e.g., U.S. Pat. No. 4,474,893; U.S. Pat. No. 4,816,567. Antibodies of the invention can also be chemically constructed. See, e.g., U.S. Pat. No. 4,676,980.

Antibodies of the invention can be chimeric (see, e.g., U.S. Pat. No. 5,482,856), humanized (see, e.g., Jones et al., Nature 321:522 (1986); Reichmann et al., Nature 332:323 (1988); Presta, Curr. Op. Struct. Biol. 2:593 (1992)), or human antibodies. Human antibodies can be made by, for example, direct immortilization, phage display, transgenic mice, or a Trimera methodology, see e.g., Reisener et al., Trends Biotechnol. 16:242-246 (1998).

Antibodies that specifically bind antigens (e.g., polypeptides of the invention), are particularly useful for detecting the presence of cancer-associated antigens in a sample, such as a serum, blood, urine, tissue, or saliva sample from an animal suspected of having cancer, such as a human. An immunoassay for cancer-associated antigens can utilize one antibody or several antibodies. An immunoassay for cancer-associated antigens can use, for example, a monoclonal antibody directed towards one epitope of a polypeptide of the invention, a combination of monoclonal antibodies directed towards epitopes of one polypeptide of the invention, monoclonal antibodies directed towards epitopes of different polypeptides of the invention, polyclonal antibodies directed towards the same antigen from a polypeptide of the invention, polyclonal antibodies directed towards different antigens, or a combination of monoclonal and polyclonal antibodies. Immunoassay protocols can be based upon, for example, competition, direct reaction, or sandwich type assays using, for example, labeled antibody. Antibodies of the invention can be labeled with any type of label known in the art, including, for example, fluorescent, chemiluminescent, radioactive, enzyme, colloidal metal, radioisotope and bioluminescent labels.

Antibodies of the invention include antibodies and antigen-binding fragments thereof that (a) compete with a reference antibody for binding to polypeptides of the invention, such as SEQ ID NOs:1-157 or antigen binding fragments thereof; (b) binds to the same epitope of polypeptides of the invention, such as SEQ ID NOs:1-157 or antigen binding fragments thereof as a reference antibody; (c) binds to polypeptides of the invention, such as SEQ ID NOs:1-157 or antigen binding fragments thereof with substantially the same K_{d} as a reference antibody; and/or (d) binds to polypeptides of the invention such as SEQ ID NOs:1-157 or fragments thereof with substantially the same off rate as a reference antibody, wherein the reference antibody is an antibody or antigen-binding fragment thereof that specifically binds to a polypeptide of the invention, such as SEQ ID NOs:1-157 or antigen-binding fragments thereof with a binding affinity Kₐ of 10⁷ l/mol or more.

Antibodies of the invention or antigen-binding fragments thereof can be bound to a support and used to detect the presence of cancer-associated antigens. Supports include, for example, glass, polystyrene, polypropylene, polyethylene, dextran, nylon, amylases, natural and modified celluloses, polyacrylamides, agaroses and magletite.

Antibodies of the invention can further be used to isolate cancer-associated antigens by immunoaffinity columns. The antibodies can be affixed to a solid support by, for example, adsorbtion or by covalent linkage so that the antibodies retain their immunoselective activity. Optionally, spacer groups can be included so that the antigen binding site of the antibody remains accessible. The immobilized antibodies can then be used to bind cancer-associated antigens from a sample, such as a biological sample including saliva, serum, sputum, blood, urine, feces, cerebrospinal fluid, amniotic fluid, wound exudate, or tissue. The bound cancer-associated antigens are recovered from the column matrix by, for example, a change in pH.

Antibodies of the invention can also be used in immunolocalization studies to analyze the presence and distribution of a polypeptide of the invention during various cellular events or physiological conditions. Antibodies can also be used to identify molecules involved in passive immunization and to identify molecules involved in the biosynthesis of non-protein antigens. Identification of such molecules can be useful in vaccine development. Antibodies of the invention, including, for example, monoclonal antibodies and single chain antibodies, can be used to monitor the course of amelioration of a cancer. Stage IV polynucleotide of the invention (i.e., polynucleotides that encode SEQ ID NOs:65-107) are particularly useful in this method, however, Stage I (i.e., polynucleotides that encode SEQ ID NOs:1-64) and Stage II (i.e., polynucleotides that encode SEQ ID NOs:108-157) can be used in this method. By measuring the increase or decrease of antibodies to cancer-associated antigens in a test sample from an animal, it can be determined whether a particular therapeutic regiment aimed at ameliorating the cancer is effective. Antibodies can be detected and/or quantified using for example, direct binding assays such as RIA, ELISA, or western blot assays.

### Methods of Detection of Cancer

Methods of detecting cancer, a predisposition to developing cancer, or a susceptibility to developing cancer in a subject are provided herein. A predisposition to cancer means that a subject is susceptible to cancer, such as colorectal cancer, or is more likely to develop cancer than a normal individual or a normal population of individuals. A subject can be a mammal such as a human, non-human primate, mouse, rat, dog, cat, sheep, pig, horse, or cow. One hundred seven polypeptides that were specifically expressed (i.e., the polypeptides are expressed in cancerous tissues, but are not expressed or are expressed at low levels in healthy tissues) in colon cancer tissues were identified. These polypeptides are cancer-associated polypeptides and are encoded by cancer-associated polynucleotides. The stage I polypeptides and polynucleotides are especially useful for early diagnosis. An expression level of one or more of the cancer-associated polynucleotides that encode polypeptides of the invention can be determined in a biological sample from a subject, wherein an increase of the expression level of the cancer-associated polynucleotides compared to a normal control expression level of the polynucleotide indicates that the subject has cancer or is at risk of developing cancer. A comparison to a normal control expression level is not necessary since the polynucleotides of the invention are not expressed or are expressed at low levels in healthy cells and tissues.

In general, PCMAT can be applied to a wide variety of cancers. The cancer can be colon cancer (also known as, and referred to herein also as colorectal or large bowel cancer), adenocarcinoma, carcinoma, sarcoma, lymphoma, leukemia, prostrate cancer, gastric cancer, lung cancer, bladder cancer, melanoma, pancreatic cancer, breast cancer, endometrial cancer, ovarian cancer, anal cancer, skin cancer, osteosarcoma, brain tumor, gastrointestinal cancer, esophageal cancer, bile duct cancer, eye cancer, gall bladder cancer, glioma, head and neck cancer, liver cancer, kidney cancer, laryngeal cancer, lip and oral cancer, mesothelioma, small intestinal cancer, testicular cancer, thyroid cancer, urethral cancer, uterine cancer, vaginal cancer, vulvar cancer, penile cancer, or any combination or subset thereof. The biological sample can be, for example, mucosal cells, tumor cells, cancer cells, a biopsy sample, a lavage sample, a sputum sample, a serum sample, a gastric secretion sample, a plasma sample, a blood sample, a fecal sample, a lymph node sample, a bone marrow sample, a urine sample, a tissue sample, a colorectal tissue sample, a pleural effusion sample, cells, cell extracts, bodily fluid, bodily fluids that are substantially lacking cells (e.g., less than about 1, 5, or 10% cells, tears, milk, seminal fluid, prostatic fluid, lung lavage fluid, or saliva.

The expression level of cancer-associated protein or polypeptide can be determined by detecting the polypeptide encoded by the cancer-associated polynucleotide. The level of the polypeptide expression can be detected using an immunoassay such as an ELISA, an immunohistochemical assay, an immunocytochemical assay, and a flow cytometry assay of antibody-labeled cells. The level of the polypeptide expression can be detected by, e.g., using an antibody that specifically binds to the polypeptide. The expression level of cancer-associated proteins and polypeptides can also be determined by detecting the biological activity of the polypeptides encoded by the cancer-associated polynucleotides. Methods of detecting the biological activity of polypeptides are well known in the art.

The expression level of a polynucleotide of the invention (i.e., “cancer-associated polynucleotide”) can be determined by detecting mRNA expression levels of the cancer-associated polynucleotide. The expression level of a cancer-associated polynucleotide can be determined by detecting hybridization of a cancer-associated polynucleotide probe to a polynucleotide transcript of a patient-derived biological sample. Hybridization can be detected using, for example a polynucleotide array. For example, probes for detecting RNA sequences corresponding to the cancer-associated polynucleotides of the invention can be used in, e.g., northern blot hybridization assays. Alternatively, polynucleotides of the invention can be used to construct primers that specifically amplify polynucleotide sequences in, e.g., amplification-based detection methods such as reverse-transcription based polymerase chain reaction (RT-PCR), polymerase chain reaction amplification (PCR), ligase chain reaction amplification (LCR), strand displacement amplification (SDA), and nucleic acid sequence based amplification (NASBA).

The expression level of one or more of the cancer-associated polynucleotides of the invention in the test sample can be compared to expression levels of the cancer-associated polynucleotides in a control sample. The control sample can be, e.g., a cancerous sample or non-cancerous sample (e.g., healthy tissue, such as healthy colorectal tissue).

Where the control sample is non-cancerous, a similar protein or polynucleotide expression level in the test sample and control sample indicates the test sample is non-cancerous. A test sample can be compared to multiple control samples. Thus, a test sample can be compared to a second control sample that contains, e.g., cancerous cells, as well as a second control that contains, e.g., non-cancerous cells.

Proteins, polypeptides and polynucleotides of the invention can be used to test a putative therapeutic or prophylactic anti-cancer agent, such as an anti-colorectal cancer agent, in a test sample from a specific subject to determine if the agent is a suitable anti-cancer agent in the specific subject. To identify an anti-cancer agent that is appropriate for a specific subject, a test sample, such as a cancerous cell or tumor sample is obtained from the subject and is exposed to the anti-cancer agent. The expression of one or more of polynucleotides of the invention is determined. The pattern of cancer-associated polynucleotide expression of the test sample can be measured and compared to one or more control profiles, e.g. a colorectal cancer reference expression profile or a non-colorectal cancer reference expression profile. Preferably, the cell population is contacted ex vivo with the agent or activated form of the anti-cancer agent.

Expression of the cancer-associated polypeptide or polynucleotides in the test sample is then compared to the expression of the cancer-associated polypeptide or polynucleotide in a control sample. The control sample can be cells whose cancer state is known. If the control sample is non-cancerous, a similar gene expression profile between the test sample and the control sample indicates the anti-cancer agent is suitable for treating cancer in the subject. A difference in expression between polypeptide or polynucleotide expression in the test sample and those in the control sample indicates that the anti-cancer agent is not suitable for treating cancer in the subject. A decrease in expression of one or more of the cancer-associated polypeptide or polynucleotides in a test sample relative to a control sample from cancerous tissues is indicative that the agent is therapeutic.

Polypeptides or polynucleotides of the invention can also be used to identify candidate therapeutic agents for treating a cancer, such as colorectal cancer. A candidate therapeutic agent is screened to determine if it converts an expression profile of cancer-associated polypeptide or polynucleotides characteristic of a cancer state, such as a colorectal cancer state, to a pattern indicative of a non-cancerous state.

A cancerous sample is exposed to a test agent or a combination of test agents (sequentially or simultaneously) and the expression of one or more cancer-associated polypeptide or polynucleotides in the sample is measured. The expression of the cancer-associated polypeptide or polynucleotides in the test sample is compared to expression level of the cancer-associated polypeptide or polynucleotides in a control sample that is not exposed to the test agent. Therapeutic test agents will decrease the expression of cancer-associated polypeptide or polynucleotides that are up-regulated in cancer cells.

The control sample can be cancerous cells, such as cancerous colorectal cancer cells. A decrease in expression of the cancer-associated polypeptide or polynucleotides in the presence of the test agent from the expression profile of the control sample in the absence of the test agent indicates the test agent is a candidate therapeutic agent for treating cancer, such as colorectal cancer.

Also provided is a method of assessing the prognosis of a subject with cancer, such as colorectal cancer, by comparing the expression of one or more polypeptide or polynucleotides of the invention in a test sample to the expression of the polypeptide or polynucleotides in a control sample derived from patients over a spectrum of disease stages. By comparing polypeptide or polynucleotide expression of one or more polypeptide or polynucleotides of the invention in the test sample and the control samples, or by comparing the pattern of polypeptide or polynucleotide expression over time in test samples derived from the subject, the prognosis of the subject can be assessed. The expression of one or more stage IV polypeptide or polynucleotides (i.e., polypeptide or polynucleotides that encode SEQ ID NOs:65-107) would be indicative of poorer prognosis. The expression of one or more stage I polypeptide or polynucleotides (i.e., polypeptide or polynucleotides that encode SEQ ID NOs:1-64) to the exclusion of expression of one or more stage IV polynucleotides would be indicative of a better prognosis.

The control sample can be a healthy sample or a cancerous sample, such as a colorectal cancer sample. Alternatively, the control sample is a cancer expression profile, such as a colorectal cancer expression profile. When the control sample is cancerous an increase of expression of one or more of the polypeptides of the invention, indicates less favorable prognosis. A decrease in expression of polypeptides or polypeptides of the invention indicates a more favorable prognosis for the subject. Alternatively, when a control sample is a healthy sample, an increase in expression of one or more or the polypeptides or polypeptides of the invention indicates a less favorable prognosis in the subject, while a decrease or similar expression indicates a more favorable prognosis.

The invention also provides a colorectal cancer reference expression profile comprising a pattern of polypeptide or polynucleotide expression levels of two or more of polypeptide or polynucleotides of the invention, optionally, over the course of the disease. The expression profile serves as a control for the diagnosis of colorectal cancer or predisposition for developing the disease, monitoring the course of treatment and assessing prognosis of a subject with the disease.

The invention also provides methods for predicting propensity for high-grade or low-grade metastatic spread of a cancer. The presence and/or level of a polypeptide or polynucleotide expression product in a cancerous sample can be detected and/or quantified and correlated to the propensity of the tumor to metastasize. The expression of one or more stage IV polypeptides or polynucleotides (i.e., polypeptides or polynucleotides that encode SEQ ID NOs:65-107) would be indicative of a higher grade metastatic spread of cancer. The expression of one or more stage I polynucleotides (i.e., polypeptides or polynucleotides that encode SEQ ID NOs:1-64) to the exclusion of expression of one or more stage IV polynucleotides would be indicative of a lower grade metastatic spread of cancer.

The polypeptides and polynucleotides of the invention can also be used to monitor the course of treatment of cancer, such as colorectal cancer. A test sample from a subject undergoing treatment for cancer, such as colorectal cancer is obtained. Test samples can be obtained from the subject at various time points before, during, or after treatment. Expression of one or more of the polypeptides or polynucleotides of the invention in the test sample is determined and compared to a control sample that includes cells having a known cancer state. Preferably, the control sample has not been exposed to the treatment. Stage IV polypeptides or polynucleotides of the invention (i.e., polypeptides of SEQ ID NOs:65-107 or polynucleotides that encode SEQ ID NOs:65-107) are particularly useful in this method, however, stage I (i.e., polypeptides of SEQ ID NOs:1-64 or polynucleotides that encode SEQ ID NOs:1-64) and stage II (i.e., polypeptides of SEQ ID NOs:108-157 or polynucleotides that encode SEQ ID NOs: 108-157) can be used in this method.

Where the control sample contains non-cancerous cells, a similarity in expression between polypeptides or polynucleotides of the invention in the test sample and the control sample indicates that the treatment is efficacious. However, an increase in expression of polypeptides or polynucleotides of the invention in the test sample as compared the control sample indicates the treatment is not efficacious.

Efficacious means that the treatment leads to a decrease in size, prevalence, or metastatic potential of cancer, such as colorectal cancer, in a subject. When treatment is applied prophylactically, efficacious means that the treatment retards, slows, or prevents cancer, such as colorectal cancer, from forming. Efficaciousness can be determined in association with any known method for diagnosing or treating cancer, such as colorectal cancer.

Where the control sample is cancerous, e.g., where the control sample includes cancer cells taken from the subject at the time of diagnosis, but prior to beginning treatment, a similarity in the expression pattern between the test sample and the control sample indicates the treatment is not efficacious. A difference in expression between polypeptide or polynucleotide expression in the test sample (i.e., a decrease in the test sample) and the control sample indicates the treatment is efficacious. Where the control sample contains non-cancerous cells, a decrease in expression of one or more of the polypeptide or polynucleotides of the invention in the test sample as compared to the control sample indicates that the treatment is efficacious.

### Methods of Treatment of Cancer

The invention provides methods for treating cancer, such as colorectal cancer, in a subject or stimulating an immune response in a subject comprising, for example, (a) administering to the subject a pharmaceutically effective amount of a polypeptide of the invention; (b) administering to the subject a pharmaceutically effective amount of a polynucleotide that encodes a polypeptide of the invention; or (c) administering to the subject a pharmaceutically effective amount of an antibody or antigen-binding fragment thereof that specifically binds to a polypeptide of the invention.

The invention also provides methods for inducing anti-tumor immunity in a subject comprising, for example, contacting a polypeptide of the invention with antigen presenting cells, or introducing a polynucleotide encoding the polypeptide or a vector comprising the polynucleotide to antigen presenting cells, and then administering the antigen presenting cells to the subject.

Administration of a therapeutic agent can be prophylactic or therapeutic to a subject at risk of (or susceptible to) a disorder or having a disorder associated with the differentially expressed polynucleotides of the invention. The expression, function, or both, of one or more expression products of the polynucleotides of the invention can be decreased in order to prophylactically or therapeutically treat a subject. Expression can be inhibited or decreased by administering to the subject a polynucleotide, such as an antisense molecule or siRNA molecule that inhibits or decreases the expression of the polynucleotides of the invention.

Antisense molecules and siRNA that correspond to polynucleotides of the invention are useful for the treatment of cancer, such as colorectal cancer. Antisense molecules and siRNA molecules can be entirely complementary to the target sequence or can have a mismatch of one or more nucleotides, so long as the antisense molecules and siRNA molecules can specifically hybridize to the target sequences. For example, the antisense molecules or siRNA molecules include polynucleotides that have a homology to a polynucleotide of the invention or its complement, of at least 80% or higher, more preferably 90% or higher, even more preferably 95% or higher over a span of at least 15 continuous nucleotides. Algorithms known in the art can be used to determine the homology.

Antisense molecules, siRNA molecules and polynucleotides of the invention can be delivered to a subject by standard vectors and/or gene delivery systems. Suitable gene delivery systems include liposomes, receptor-mediated delivery systems, naked DNA, and viral vectors such as herpes viruses, retroviruses, adenoviruses and adeno-associated viruses, among others.

Antisense molecules or siRNA molecules inhibit the expression of a polynucleotide of the invention and is thereby useful for suppressing the biological activity of a polypeptide of the invention. Therefore, a composition comprising an antisense molecule or siRNA molecule targeted to a polynucleotide of the invention is useful in treating a cancer, such as colorectal cancer.

In another embodiment of the invention, the function of one or more expression products of the polynucleotides of the invention can be inhibited by administering a compound that binds to or otherwise inhibits the function of the expression products. The compound can be, e.g., an antibody that specifically binds to an expression product of the polynucleotides of the invention.

Therapeutic compounds that may be utilized include, e.g., (i) a polypeptide or fragments thereof of SEQ ID NOs:1-157; (ii) antibodies or specific binding fragments thereof that specifically bind SEQ ID NOs:1-157; (iii) polynucleotides or fragments thereof that encode SEQ ID NOs:1-157; (iv) antisense molecules specific for polynucleotides (or complements thereof) that encode SEQ ID NOs:1-157 or fragments thereof; (v) siRNA molecules specific for polynucleotides (or complements thereof) that encode SEQ ID NOs:1-157 or fragments thereof; and (vi) modulators (i.e., inhibitors, agonists and antagonists that alter the interaction between a polypeptide of the invention and its binding partner).

Administration of a prophylactic pharmaceutical composition can occur prior to the manifestation of symptoms characteristic of a disease or disorder, such that a disease or disorder is prevented or, alternatively, delayed in its progression.

The present invention also relates to a method of treating or preventing cancer, such as colorectal cancer, in a subject comprising administering to said subject an immunological composition (i.e., a composition that can induce antibodies or other immune responses in a subject) comprising a polypeptide encoded by a polynucleotide of the invention or an immunologically active fragment of said polypeptide, or a polynucleotide encoding the polypeptide or the fragment thereof. Administration of the polypeptide can induce an anti-tumor immunity in a subject. In one embodiment the polypeptides of the invention or fragments thereof may be administered in a form bound to a T cell receptor (TCR) or presented by an antigen presenting cell (APC), such as macrophage, dendritic cell (DC), or B-cell.

In the present invention, an immunological composition against cancer, such as colorectal cancer, can function to induce anti-tumor immunity upon inoculation into a subject. Polypeptides of the invention may induce potent and specific immune response against cancer, such as colorectal cancer. In general, anti-tumor immunity includes immune responses such as induction of cytotoxic lymphocytes against tumors, induction of antibodies that recognize tumors, and induction of anti-tumor cytokine production.

Anti-tumor immunity is induced by administering the immunological composition of this invention, and the induction of anti-tumor immunity enables treatment and prevention of cancer, such as colorectal cancer.

A polypeptide of the invention that has immunological activity or a vector encoding the polypeptide may be combined with an adjuvant. An adjuvant can enhance the immune response against the polypeptide when administered together (or successively) with the polypeptide having immunological activity. The immunological composition is administered systemically or locally. Immunological composition administration may be performed by single administration, or boosted by multiple administrations.

In another aspect the invention includes pharmaceutical, or therapeutic, compositions containing one or more therapeutic compounds described herein. Pharmaceutical formulations may include those suitable for oral, rectal, nasal, topical (including buccal and sub-lingual), vaginal or parenteral (including intramuscular, intraperitoneal, intratumor, sub-cutaneous and intravenous) administration, or for administration by inhalation or insufflation. The formulations may, where appropriate, be conveniently presented in discrete dosage units and may be prepared by any of the methods well known in the art of pharmacy. All such pharmacy methods include the steps of bringing into association the active compound with liquid carriers or finely divided solid carriers or both as needed and then, if necessary, shaping the product into the desired formulation.

Pharmaceutical formulations suitable for oral administration may conveniently be presented as discrete units, such as capsules, cachets or tablets, each containing a predetermined amount of the active ingredient; as a powder or granules; or as a solution, a suspension or as an emulsion. The tablets or capsules may optionally be formulated so as to provide slow or controlled release of the active ingredient therein. The active ingredient may also be presented as a bolus electuary or paste, and be in a pure form, i.e., without a carrier. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration may be presented as a suppository with the usual carriers such as cocoa butter or polyethylene glycol. Formulations for topical administration in the mouth, for example buccally or sublingually, include lozenges, comprising the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents. Liquid sprays are conveniently delivered from pressurized packs.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflators.

When desired, the above described formulations, adapted to give sustained release of the active ingredient, may be employed. The pharmaceutical compositions may also contain other active ingredients such as antimicrobial agents, immunosuppressants or preservatives.

For each of the aforementioned conditions, the compositions may be administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg. The dose employed will depend upon a number of factors, including the age and sex of the subject, the precise disorder being treated, and its severity. Also the route of administration may vary depending upon the condition and its severity.

### Methods for Screening Anti-Cancer Compounds

The invention provides methods for screening for anti-cancer compounds, e.g. anti-colorectal cancer compounds. For example, anti-cancer compounds can be identified by comparing the level of a polypeptide or polynucleotide expression product in a first biological sample (e.g., a cancerous sample) in the presence of a test compound to the level of the polypeptide or polynucleotide expression product in a second biological sample (e.g., a cancerous sample) in the absence of the test compound; wherein the polypeptide or polynucleotide expression product comprises, for example, a polypeptide selected from the group consisting of SEQ ID NOs:1-157 or mRNA encoding the polypeptide. A test compound that decreases the level of the polypeptide or polynucleotide expression product in the first biological sample as compared to the second biological sample is identified as an anti-cancer agent. In one embodiment of the invention, the test compound decreases the level of the polypeptide or polynucleotide expression product by at least about 10%, 20%, 30%, 40%, 50%, 60%, 70%, 80%, 90% (or any value or range between about 10% and about 90%) in the first biological sample as compared to the level of the expression product in the second biological sample.

In one embodiment of the invention, screening for anti-cancer compounds, e.g. anti-colorectal cancer compounds, can comprise comparing the level of biological activity of a polypeptide of the invention in a first biological sample in the presence of a test compound to the level of biological activity in a second biological sample in the absence of the test compound; wherein a test compound that decreases the level of biological activity in the first biological sample as compared to the second biological sample is identified as an anti-cancer agent.

In one embodiment of the invention, screening for anti-cancer compounds, e.g. anti-colorectal cancer compounds can comprise a) contacting a test compound with a polypeptide of the invention; b) detecting the binding activity between the polypeptide and the test compound; and c) selecting a compound that binds to the polypeptide.

In one embodiment of the invention, screening for anti-cancer compounds, e.g. anti-colorectal cancer compounds, can comprise a) contacting a candidate compound with a test cell expressing, one or more of the polypeptides of the invention; and b) selecting a compound that reduces the expression level of one or more polypeptides of the invention. The test cell can comprise a colorectal cancer cell.

In one embodiment of the invention, screening for anti-cancer compounds, e.g. anti-colorectal cancer compounds, can comprise a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of polynucleotides that encode SEQ ID NOs:1-157) measuring the activity of the reporter gene; and c) selecting a compound that reduces the expression level of the reporter gene as compared to a control.

The invention provides kits for use, for example, in diagnostic methods. Components of the kits can include, for example, compounds, reagents, containers and/or equipment. For example, one container within a kit may contain a monoclonal antibody or antigen-binding fragment thereof that specifically binds to a polypeptide of the invention. The antibodies or antigen-binding fragments can be, e.g., attached to a support material. One or more additional containers can contain elements, such as reagents or buffers, to be used in an assay. The kits can also, or alternatively, contain a detection reagent that contains a reporter group suitable for direct or indirect detection of specific antibody binding.

Alternatively, a kit can be used to detect, e.g., the level of mRNA encoding a polypeptide of the invention in a biological sample. Such kits can comprise at least one, two, or more polynucleotide probes or primers, that hybridize to a polynucleotide (or the complement thereof) encoding a polypeptide of the invention. Such polynucleotides can be used, for example, within an amplification assay (e.g., RT-PCR) or hybridization assay. Additional components that can be present in such kits include a second polynucleotide and/or a diagnostic reagent or container to facilitate the detection of a polynucleotide encoding a polypeptide of the invention.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, without changing the ordinary meanings of these terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

All references cited in this disclosure are incorporated herein in their entirety by reference. Furthermore, the content (as of the filing date of this application) of all GenBank, ENSEMBL, UNIPARC, and UniProt Accession Numbers (and data associated therewith) listed herein are incorporated herein by reference in their entirety.

| Titin (also known as TTN rhabdomyosarcoma antigen MU-RMS 40) | |
|---|---|
| (e.g., GenBank Accession Number Q8WZ42-2 | |
| (SEQ ID NO: 1) | |
| 1 | mttqaptftq plqsvvvleg statfeahis gfpvpevswf rdgqvistst lpgvqisfsd |
| 61 | grakltipav tkansgrysl katngsgqat staellvkae tappnfvqrl qsmtvrqgsq |
| 121 | vrlqvrvtgi ptpvvkfyrd gaeiqssldf qisqegdlys lliaeayped sgtysvnatn |
| 181 | svgratstae llvqgeeevp akktktivst aqisesrqtr iekkieahfd arsiatvemv |
| 241 | idgaagqqlp hktphrippk pksrsptpps iaakaqlarq qspspirhsp spvrhvrapt |
| 301 | pspvrsvspa aristspirs vrspllmrkt qastvatgpe vpppwkqegy vassseaemr |
| 361 | ettlttstqi rteerwegry gvqeqvtisg aagaaasvsa sasyaaeava tgakevkqda |
| 421 | dksaavatvv aavdmarvre pvisaveqta qrttttavhi qpaqeqvrke aektavtkvv |
| 481 | vaadkakeqe lksrtkevit tkqeqmhvth eqirketekt fvpkvvisaa kakeqetris |
| 541 | eeitkkqkqv tqeairqete itaasmvvva takstkletv pgaqeetttq qdqmhlsyek |
| 601 | imketrktvv pkvivatpkv keqdlvsrgr egittkreqv qitqekmrke aektalstia |
| 661 | vatakakeqe tilrtretma trqeqiqvth gkvdvgkkae avatvvaavd qarvreprep |
| 721 | ghleesyaqq ttleygyker isaakvaepp qrpasephvv pkavkprviq apsethiktt |
| 781 | dqkgmhissq ikkttdltte rlvhvdkrpr tasphftvsk isvpktehgy easiagsaia |
| 841 | tlqkelsats saqkitksvk aptvkpsetr vraeptplpq fpfadtpdty kseagvevkk |
| 901 | evgvsitgtt vreerfevlh greakvteta rvpapveipv tpptlvsglk nvtviegesv |
| 961 | tlechisgyp sptvtwyred yqiessidfq itfqsgiarl mireafaeds grftcsavne |
| 1021 | agtvstscyl avqvseefek ettavtekft teekrfvesr dvvmtdtslt eeqagpgepa |
| 1081 | apyfitkpvv qklveggsvv fgcqvggnpk phvywkksgv plttgyrykv synkqtgeck |
| 1141 | lvismtfadd ageytivvrn khgetsasas lleeadyell mksqqemlyq tqvtafvqep |
| 1201 | kvgetapgfv yseyekeyek eqalirkkma kdtvvvrtyv edqefhissf eerlikeiey |
| 1261 | riikttleel leedgeekma vdiseseave sgfdlrikny rilegmgvtf hckmsgyplp |
| 1321 | kiawykdgkr ikhgeryqmd flqdgraslr ipvvlpedeg iytafasnik gnaicsgkly |
| 1381 | vepaaplgap tyiptlepvs rirslsprsv srspirmspa rmsparmspa rmsparmspg |
| 1441 | rrleetdesq lerlykpvfv lkpvsfkcle gqtarfdlkv vgrpmpetfw fhdgqqivnd |
| 1501 | ythkvviked gtqsliivpa tpsdsgewtv vaqnragrss isviltveav ehqvkpmfve |
| l561 | klknvnikeg sqlemkvrat gnpnpdivwl knsdiivphk ypkiriegtk geaalkidst |
| 1621 | vsqdsawyta tainkagrdt trckvnveve faepeperkl iiprgtyrak eiaapelepl |
| 1681 | hlrygqeqwe egdlydkekq qkpffkkklt slrlkrfgpa hfecrltpig dptmvvewlh |
| l741 | dgkpleaanr lrminefgyc sldygvaysr dsgiitcrat nkygtdhtsa tlivkdeksl |
| 1801 | veesqlpegr kglqrieele rmahegaltg vttdqkekqk pdivlypepv rvlegetarf |
| 1861 | rcrvtgypqp kvnwylngql irkskrfrvr ydgihyldiv dcksydtgev kvtaenpegv |
| 1921 | iehkvkleiq qredfrsvlr rapeprpefh vhepgklqfe vqkvdrpvdt tetkevvklk |
| 1981 | raerithekv peeseelrsk fkrrteegyy eaitavelks rkkdesyeel lrktkdellh |
| 2041 | wtkelteeek kalaeegkit iptfkpdkie lspsmeapki feriqsqtvg qgsdahfrvr |
| 2101 | vvgkpdpece wykngvkier sdriywywpe dnvcelvird vtaedsasim vkainiaget |
| 2161 | sshafllvqa kqlitftqel qdvvakekdt matfecetse pfvkvkwykd gmevhegdky |
| 2221 | rmhsdrkvhf lsiltidtsd aedyscvlve denvkttakl ivegavvefv kelqdievpe |
| 2281 | sysgeleciv speniegkwy hndvelksng kytitsrrgr qnltvkdvtk edqgeysfvi |
| 2341 | dgkkttcklk mkprpiailq glsdqkvceg divqlevkvs lesvegvwmk dgqevqpsdr |
| 2401 | vhividkqsh mlliedmtke dagnysftip alglstsgrv svysvdvitp lkdvnviegt |
| 2461 | kavleckvsv pdvtsvkwyl ndeqikpddr vqaivkgtkq rlvinrthas degpyklivg |
| 2521 | rvetncnlsv ekikiirglr dltctetqnv vfevelshsg idvlwnfkdk eikpsskyki |
| 2581 | eahgkiyklt vlnmmkddeg kytfyageni tsgkltvagg aiskpltdqt vaesqeavfe |
| 2641 | cevanpdskg ewlrdgkhlp ltnnirsesd ghkrrliiaa tklddigeyt ykvatsktsa |
| 2701 | klkveavkik ktlknltvte tqdavftvel thpnvkgvqw ikngvvlesn ekyaisvkgt |
| 2761 | iyslriknca ivdesvygfr lgrlgasarl hvetvkiikk pkdvtalena tvafevsvsh |
| 2821 | dtvpvkwfhk nveikpsdkh rlvserkvhk lmlqnispsd ageytavvgq leckaklfve |
| 2881 | tlhitktmkn ievpetktas fecevshfnv psmwlkngve iemsekfkiv vqgklhqlii |
| 2941 | mntstedsae ytfvcgndqv satltvtpim itsmlkdina eekdtitfev tvnyegisyk |
| 3001 | wlkngveiks tdkcqmrtkk lthslnirnv hfgdaadytf vagkatstat lyvearhief |
| 3061 | rkhikdikvl ekkramfece vsepditvqw mkddqelqit drikiqkeky vhrllipstr |
| 3121 | msdagkytvv aggnvstakl fvegrdvrir sikkevqvie kqravvefev neddvdahwy |
| 3181 | kdgieinfqv qerhkyvver rihrmfiset rqsdageytf vagrnrssvt lyvnapeppq |
| 3241 | vlqelqpvtv qsgkparfca visgrpqpki swykeeqlls tgfkckflhd ggeytlllie |
| 3301 | afpedaavyt ceakndygva ttsaslsvev pevvspdqem pvyppaiitp lqdtvtsegq |
| 3361 | parfqcrvsg tdlkvswysk dkkikpsrff rmtqfedtyq leiaeayped egtytfvasn |
| 3421 | avgqvsstan lsleapesil herieqeiem emkefsssfl saeeeglhsa elqlskinet |
| 3481 | lellsespvy stkfdsekeg tgpifikevs nadismgdva tlsvtvigip kpkiqwffng |
| 3541 | vlltpsadyk fvfdgddhsl iilftklede geytcmasnd ygkticsayl kinskgeghk |
| 3601 | dtetesavak sleklggpcp phflkelkpi rcaqglpaif eytvvgepap tvtwfkenkq |
| 3661 | lctsvyytii hnpngsgtfi vndpqredsg lyickaenml gestcaaell vlledtdmtd |
| 3721 | tpckakstpe apedfpqtpl kgpavealds eqeiatfvkd tilkaalite enqqlsyehi |
| 3781 | akanelssql plgaqelqsi leqdkltpes treflcings ihfqplkeps pnlqlqivqs |
| 3841 | qktfskegil mpeepetqav lsdtekifps amsieqinsl tveplktlla epegnypqss |
| 3901 | ieppmhsylt svaeevlspk ektvsdtnre qrvtlqkqea qsalilsqsl aeghveslqs |
| 3961 | pdvmisqvny eplvpsehsc teggkilies anplenagqd savrieegks lrfplaleek |
| 4021 | qvllkeehsd nvvmppdqii eskrepvaik kvqevqgrdl lskesllsgi peeqrlnlki |
| 4081 | qicralqaav aseqpglfse wlrniekvev eavnitqepr himcmylvts aksvteevti |
| 4141 | iiedvdpqma nlkmelrdal caiiyeeidi ltaegpriqq gaktslqeem dsfsgsqkve |
| 4201 | pitepevesk ylisteevsy fnvqsrvkyl datpvtkgva savvsdekqd eslkpseeke |
| 4261 | esssesgtee vatvkigeae gglikedgpm ihtplvdtvs eegdivhltt sitnakevnw |
| 4321 | yfenklvpsd ekfkclqdqn tytlvidkvn tedhqgeyvc ealndsgkta tsakltvvkr |
| 4381 | aapvikrkie plevalghla kftceiqsap nvrfqwfkag reiyesdkcs irsskyissl |
| 4441 | eilrtqvvdc geytckasne ygsysctatl tvteaypptf lsrpkslttf vgkaakfict |
| 4501 | vtgtpvieti wqkdgaalsp spnwkisdae nkhilelsnl tiqdrgvysc kasnkfgadi |
| 4561 | cqaeliiidk phfikelepv qsainkkvhl ecqvdedrkv tvtwskdgqk lppgkdykic |
| 4621 | fedkiatlei plaklkdsgt yvctasneag ssscsatvtv reppsfvkkv dpsylmlpge |
| 4681 | sarlhcklkg spviqvtwfk nnkelsesnt vrmyfvnsea ilditdvkve dsgsysceav |
| 4741 | ndvgsdscst eivikeppsf iktlepadiv rgtnallqce vsgtgpfeis wfkdkkqirs |
| 4801 | skkyrlfsqk slvcleifsf nsadvgeyec vvanevgkcg cmathllkep ptfvkkvddl |
| 4861 | ialggqtvtl qaavrgsepi svtwmkgqev iredgkikms fsngvavlii pdvqisfggk |
| 4921 | ytclaeneag sqtsvgeliv kepakiiera eliqvtagdp atleytvagt pelkpkwykd |
| 4981 | grplvaskky risfknnvaq ikfysaelhd sgqytfeisn evgssscett ftvldrdiap |
| 5041 | fftkplrnvd svvngtcrld ckiagslpmr vswfkdgkei aasdryriaf vegtasleii |
| 5101 | rvdmndagnf tcratnsvgs kdssgativq eppsfvtkpg skdvlpgsav clkstfqgst |
| 5161 | pltirwfkgn kelvsggscy itkealessl elylvktsds gtytckvsnv aggvecsanl |
| 5221 | fvkepatfve klepsqllkk gdatqlackv tgtppikitw fandreikes skhrmsfves |
| 5281 | tavlrltdvg iedsgeymce aqneagsdhc ssivivkesp yftkefkpie vlkeydvmll |
| 5341 | aevagtppfe itwfkdntil rsgrkyktfi qdhlvslqil kfvaadagey qcrvtnevgs |
| 5401 | sicsarvtlr eppsfikkie stsslrggta afqatlkgsl pitvtwlkds deiteddnir |
| 5461 | mtfennvasl ylsgievkhd gkyvcqaknd agiqrcsall svkepatite eavsidvtqg |
| 5521 | dpatlqvkfs gtkeitakwf kdgqeltlgs kykisvtdtv silkiistek kdsgeytfev |
| 5581 | qndvgrssck arinvldlii ppsftkklkk mdsikgsfid lecivagshp isiqwfkddq |
| 5641 | eisasekykf sfhdntafle isqlegtdsg tytcsatnka ghnqcsghlt vkeppyfvek |
| 5701 | pqsqdvnpnt rvqlkalvgg tapmtikwfk dnkelhsgaa rsvwkddtst slelfaakat |
| 5761 | dsgtyicqls ndvgtatska tlfvkeppqf ikkpspvlvl rngqsttfec qitgtpkirv |
| 5821 | swyldgneit aiqkhgisfi dglatfqisg arvensgtyv cearndagta scsielkvke |
| 5881 | pptfirelkp vevvkysdve lecevtgtpp fevtwlknnr eirsskkytl tdrvsvfnlh |
| 5941 | itkcdpsdtg eyqcivsneg gscscstrva lkeppsfikk ientttvlks satfqstvag |
| 6001 | sppisitwlk ddqildeddn vyisfvdsva tlqirsvdng hsgrytcqak nesgvercya |
| 6061 | fllvqepaqi vekaksvdvt ekdpmtlecv vagtpelkvk wlkdgkqivp sryfsmsfen |
| 6121 | nvasfriqsv mkqdsgqytf kvendfgsss cdaylrvldq nippsftkkl tkmdkvlgss |
| 6181 | ihmeckvsgs lpisaqwfkd gkeistsaky rlvchersvs levnnleled tanytckvsn |
| 6241 | vagddacsgi ltvkeppsfl vkpgrqqaip dstvefkail kgtppfkikw fkddvelvsg |
| 6301 | pkcfiglegs tsflnlysvd asktgqytch vtndvgsdsc ttmllvtepp kfvkkleask |
| 6361 | ivkagdssrl eckiagspei rvvwfrnehe lpasdkyrmt fidsvaviqm nnlstedsgd |
| 6421 | ficeaqnpag stscstkviv keppvfssfp pivetlknae vslecelsgt ppfevvwykd |
| 6481 | krqlrsskky kiasknfhts ihilnvdtsd igeyhckaqn evgsdtcvct vklkepprfv |
| 6541 | sklnsltvva gepaelqasi egaqpifvqw lkekeevire seniritfve nvatlqfaka |
| 6601 | epanagkyic qikndggmee nmatlmvlep avivekagpm cvtvgetctl eckvagtpel |
| 6661 | svewykdgkl ltssqkhkfs fynkisslri lsverqdagt ytfqvqnnvg kssctavvdv |
| 6721 | sdravppsft rrlkntggvl gascileckv agsspisvaw fhektkivsg akyqttfsdn |
| 6781 | vctlqlnsld ssdmgnytcv aanvagsdec ravltvqepp sfvkepeple vlpgknvtft |
| 6841 | svirgtppfk vnwfrgarel vkgdrcniyf edtvaelelf nidisqsgey tcvvsnnagq |
| 6901 | ascttrlfvk epaaflkrls dhsvepgksi ilestytgtl pisvtwkkdg fnittsekcn |
| 6961 | ivttektcil eilnstkrda gqysceiene agrdvcgalv stleppyfvt elepieaavg |
| 7021 | dsyslqcqva gtpeitvswy kgdtklrptp eyrtyftnnv ativfnkvni ndsgeytcka |
| 7081 | ensigtassk tvfriqerql ppsfarqlkd ieqtvglpvt ltcrlngsap iqvcwyrdgv |
| 7141 | llrddenlqt sfvdnvatlk ilqtdlshsg qyscsasnpl gtasssarlt arepkkspff |
| 7201 | dikpvsidvi agesadfech vtgagpmrit wskdnkeirp ggnytitcvg ntphlrilkv |
| 7261 | gkgdsgqytc qatndvgkdm csaqlsvkep pkfvkkleas kvakqgesiq leckisgspe |
| 7321 | ikvswfrnds elheswkynm sfinsvallt ineasaedsg dyiceahngv gdascstalt |
| 7381 | vkappvftqk pspvgalkgs dvilqceisg tppfevvwvk drkqvrnskk fkitskhfdt |
| 7441 | slhilnleas dvgeyhckat nevgsdtcsc svkfkepprf vkklsdtstl igdavelrai |
| 7501 | vegfqpisvv wlkdrgevir esentrisfi dniatlqlgs peasnsgkyi cqikndagmr |
| 7561 | ecsavltvle pariiekpep mtvttgnpfa lecvvtgtpe isakwfkdgr elsadskhhi |
| 7621 | tfinkvaslk ipcaemsdkg lysfevknsv gksnctvsvh vsdrivppsf irklkdvnai |
| 7681 | lgasvvlecr vsgsapisvg wfqdgneivs gpkcqssfse nvctlnlsll epsdtgiytc |
| 7741 | vaanvagsde csavltvqep psfeqtpdsv evlpgmsltf tsvirgtppf kvkwfkgsre |
| 7801 | lvpgescnis ledfvtelel fevqplesgd ysclvtndag sasctthlfv kepatfvkrl |
| 7861 | adfsvetgsp ivleatytgt ppisyswikd eylisqserc sitmteksti leilestied |
| 7921 | yaqysclien eagqdiceal vsvleppyfi eplehveavi gepatlqckv dgtpeirisw |
| 7981 | ykehtklrsa paykmqfknn vaslvinkvd hsdvgeysck adnsvgavas savlvikerk |
| 8041 | lppffarklk dvhetlyfpv afecringse plqvswykdg vllkddanlq tsfvhnvatl |
| 8101 | gilqtdqshi gqyncsasnp lgtasssakl ilsehevppf fdlkpvsvdl algesgtfkc |
| 8161 | hvtgtapiki twakdnreir pggnykmtlv entatltvlk vgkgdagqyt cyasniagkd |
| 8221 | scsahlgvqe pprfikklep srivkqdeft ryeckiggsp eikvlwykde teiqesskfr |
| 8281 | msfvdsvavl emhnlsveds gdytceahna agsassstsl kvkeppifrk kphpietlkg |
| 8341 | advhlecelq gtppfhvswy kdkrelrsgk kykimsenfl tsihilnvda adigeyqcka |
| 8401 | tndvgsdtcv gsialkappr fvkklsdist vvgkevqlqt tiegaepisv vwfkdkgeiv |
| 8461 | resdniwisy seniatiqfs rvepanagky tcqikndagm qecfatlsvl epativekpe |
| 8521 | sikvttgdtc tlectvagtp elstkwfkdg keltsdnkyk isffnkvsgl kiinvapsds |
| 8581 | gvysfevqnp vgkdsctasl qvsdrtvpps ftrklketng lsgssvvmec kvygsppisv |
| 8641 | swfhegneis sgrkyqttlt dntcaltvnm leesdsgdyt ciatnmagsd ecsapltvre |
| 8701 | ppsfvqkpdp mdvltgtnvt ftsivkgtpp fsyswfkgss elvpgdrcnv sledsvaele |
| 8761 | ifdvdtsqsg eytcivsnea gkasctthly ikapakfvkr indysiekgk plilegtftg |
| 8821 | tppisvtwkk nginvtpsqr cnittteksa ileipsstve dagqyncyie nasgkdscsa |
| 8881 | qilileppyf vkqlepvkvs vgdsaslqcq lagtpeigvs wykydtklrp tttykmhfrn |
| 8941 | nvatlvfnqv dindsgeyic kaensvgevs astfltvqeq klppsfsrql rdvqetvglp |
| 9001 | vvfdcaisgs episvswykd gkplkdspnv qtsfldntat lnifktdrsl agqysctatn |
| 9061 | pigsasssar liltegknpp ffdirlapvd avvgesadfe chvtgtqpik vswakdsrei |
| 9121 | rsggkyqisy lensahltvl kvdkgdsgqy tcyavnevgk dsctaqlnik erlippsftk |
| 9181 | rlsetveete gnsfklegrv agsqpitvaw yknnieiqpt snceitfknn tlvlqvrkag |
| 9241 | mndaglytck vsndagsalc tssivikepk kppvfdqhlt pvtvsegeyv qlschvqgse |
| 9301 | piriqwlkag reikpsdrcs fsfasgtavl elrdvakads gdyvckasnv agsdttkskv |
| 9361 | tikdkpavap atkkaavdgr lffvsepqsi rvvekttatf iakvggdpip nvkwtkgkwr |
| 9421 | qlnqggrvfi hqkgdeakle irdttktdsg lyrcvafneh geiesnvnlq vderkkqeki |
| 9481 | egdlramlkk tpilkkgage eeeidimell knvdpkeyek yarmygitdf rgllqafell |
| 9541 | kqsqeeethr leieeierse rdekefeelv sfiqqrlsqt epvtlikdie nqtvlkdnda |
| 9601 | vfeidikiny peiklswykg teklepsdkf eisidgdrht lrvkncqlkd qgnyrlvcgp |
| 9661 | hiasakltvi epawerhlqd vtlkegqtct mtcqfsvpnv ksewfrngri lkpqgrhkte |
| 9721 | vehkvhklti advraedqgq ytckyedlet saelrieaep iqftkriqni vvsehqsatf |
| 9781 | ecevsfddai vtwykgptel tesqkynfrn dgrchymtih nvtpddegvy sviarleprg |
| 9841 | earstaelyl ttkeiklelk ppdipdsrvp iptmpiravp peeippvvap piplllptpe |
| 9901 | ekkpppkrie vtkkavkkda kkvvakpkem tpreeivkkp pppttlipak apeiidvssk |
| 9961 | aeevkimtit rkkevqkeke avyekkqavh kekrvfiesf eepydeleve pytepfeqpy |
| 10021 | yeepdedyee ikveakkevh eeweedfeeg qeyyereegy degeeeweea yqereviqvq |
| 10081 | kevyeesher kvpakvpekk appppkvikk pviekiekts rrmeeekvqv tkvpevskki |
| 10141 | vpqkpsrtpv qeevievkvp avhtkkmvis eekmffasht eeevsvtvpe vqkeivteek |
| 10201 | ihvavskrve pppkvpelpe kpapeevapv pipkkveppa pkvpevpkkp vpeekkpvpv |
| 10261 | pkkepaappk vpevpkkpvp eekipvpvak kkeappakvp evqkrvvtee kitivtqree |
| 10321 | spppavpeip kkkvpeerkp vprkeeevpp ppkvpalpkk pvpeekvavp vpvakkappp |
| 10381 | raevskktvv eekrfvaeek lsfavpqrve vtrhevsaee ewsyseeeeg vsisvyreee |
| 10441 | reeeeeaevt eyevmeepee yvveeklhii skrveaepae vterqekkiv lkpkipakie |
| 10501 | epppakvpea pkkivpekkv papvpkkekv pppkvpeepk kpvpekkvpp kvikmeeplp |
| 10561 | akvterhmqi tqeekvlvav tkkeappkar vpeepkravp eekvlklkpk reeeppakvt |
| 10621 | efrkrvvkee kvsieapkre pqpikevtim eekeraytle eeavsvqree eyeeyeeydy |
| 10681 | kefeeyepte eydqyeeyee reyeryeehe eyitepekpi pvkpvpeepv ptkpkappak |
| 10741 | vlkkavpeek vpvpipkklk ppppkvpeep kkvfeekiri sitkrekeqv tepaakvpmk |
| 10801 | pkrvvaeekv pvprkevapp vrvpevpkel epeevafeee vvthveeylv eeeeeyihee |
| 10861 | eefiteeevv pvipvkvpev prkpvpeekk pvpvpkkkea ppakvpevpk kpeekvpvli |
| 10921 | pkkekpppak vpevpkkpvp eekvpvpvpk kveappakvp evpkkpvpek kvpvpapkkv |
| 10981 | eappakvpev pkklipeekk ptpvpkkvea pppkvpkkre pvpvpvalpq eeevlfeeei |
| 11041 | vpeeevlpee eevlpeeeev lpeeeevlpe eeeippeeee vppeeeyvpe eeefvpeeev |
| 11101 | lpevkpkvpv papvpeikkk vtekkvvipk keeappakvp evpkkveekr iilpkeeevl |
| 11161 | pvevteepee episeeeipe eppsieevee vapprvpevi kkavpeaptp vpkkveappa |
| 11221 | kvskkipeek vpvpvqkkea ppakvpevpk kvpekkvlvp kkeavppakg rtvleekvsv |
| 11281 | afrqevvvke rlelevveae veeipeeeef heveeyfeeg efheveefik leqhrveeeh |
| 11341 | rvekvhrvie vfeaeevevf ekpkappkgp eisekiippk kpptkvvprk eppakvpevp |
| 11401 | kkivveekvr vpeeprvppt kvpdvlppke vvpekkvpvp pakkpeappp kvpeapkevv |
| 11461 | pekkvpvppp kkpevpptkv pevpkaavpe kkvpeaippk pespppevpe apkevvpekk |
| 11521 | vpaappkkpe vtpvkvpeap kevvpekkvp vpppkkpevp ptkvpevpkv avpekkvpea |
| 11581 | ippkpesppp evfeapeeva leeppaevve epepaappqv tvppkkpvpe kkapavvakk |
| 11641 | pelppvkvpe vpkevvpekk vplvvpkkpe appakvpevp kevvpekkva vpkkpevppa |
| 11701 | kvpevpkkpv leekpavpvp eraespppev yeepeeiape eeiapeeekp vpvaeeeepe |
| 11761 | vpppavpeep kkiipekkvp vikkpeappp kepepekvie kpklkprppp pppappkedv |
| 11821 | kekifqlkai pkkkvpekpq vpekveltpl kvpggekkvr kllperkpep keevvlksvl |
| 11881 | rkrpeeeepk vepkklekvk kpavpepppp kpveevevpt vtkrerkipe ptkvpeikpa |
| 11941 | iplpapepkp kpeaevktik pppvepeptp iaapvtvpvv gkkaeakapk eeaakpkgpi |
| 12001 | kgvpkktpsp ieaerrklrp gsggekppde apftyqlkav plkfvkeikd iiltesefvg |
| 12061 | ssaifeclvs pstaittwmk dgsnirespk hrfiadgkdr klhiidvqls dageytcvlr |
| 12121 | lgnkektsta klvveelpvr fvktleeevt vvkgqplyls celnkerdvv wrkdgkivve |
| 12181 | kpgrivpgvi glmraltind addtdagtyt vtvenannle csscvkvvev irdwlvkpir |
| 12241 | dqhvkpkgta ifacdiakdt pnikwfkgyd eipaepndkt eiirdgnhly lkiknamped |
| 12301 | iaeyaveieg krypakltlg erevellkpi edvtiyekes asfdaeisea dipgqwklkg |
| 12361 | ellrpsptce ikaeggkrfl tlrkvkldqa gevlyqalna ittailtvke ieldfavplk |
| 12421 | dvtvperrqa rfecvltrea nviwskgpdi ikssdkfdii adgkkhilvi ndsqfddegv |
| 12481 | ytaevegkkt sarlfvtgir lkfmspledq tvkegetatf vcelshekmh vvwfkndakl |
| 12541 | htsrtvliss egkthklemk evtlddisqi kaqvkelsst aqlkvleadp yftvklhdkt |
| 12601 | avekdeitlk cevskdvpvk wfkdgeeivp spkysikadg lrrilkikka dlkdkgeyvc |
| 12661 | dcgtdktkan vtvearlikv ekplygvevf vgetahfeie lsepdvhgqw klkgqpltas |
| 12721 | pdceiiedgk khililhncq lgmtgevsfq aanaksaanl kvkelplifi tplsdvkvfe |
| 12781 | kdeakfecev srepktfrwl kgtqeitgdd rfelikdgtk hsmviksaaf edeakymfea |
| 12841 | edkhtsgkli iegirlkflt plkdvtakek esavftvels hdnirvkwfk ndqrlhttrs |
| 12901 | vsmqdegkth sitfkdlsid dtsqirveam gmsseakltv legdpyftgk lqdytgvekd |
| 12961 | evilqceisk adapvkwfkd gkeikpskna vikadgkkrm lilkkalksd igqytcdcgt |
| 13021 | dktsgkldie dreiklvrpl hsvevmetet arfeteised dihanwklkg eallqtpdce |
| 13081 | ikeegkihsl vlhncrldqt ggvdfqaanv kssahlrvkp rvigllrplk dvtvtageta |
| 13141 | tfdcelsyed ipvewylkgk klepsdkvvp rsegkvhtlt lrdvkledag evqltakdfk |
| 13201 | thanlfvkep pveftkpled qtveegatav lecevsrena kvkwfkngte ilkskkyeiv |
| 13261 | adgrvrklvi hdctpedikt ytcdakdfkt scnlnvvpph veflrpltdl qvrekemarf |
| 13321 | ecelsrenak vkwfkdgaei kkykkydiis kgavrilvin kcllddeaey scevrtarts |
| 13381 | gmltvleeea vftknlanie vsetdtiklv cevskpgaev iwykgdeeii etgryeilte |
| 13441 | grkrilviqn ahledagnyn crlpssrtdg kvkvhelaae fiskpqnlei legekaefvc |
| 13501 | siskesfpvq wkrddktles gdkydviadg kkrvlvvkda tlqdmgtyvv mvgaaraaah |
| 13561 | ltvieklriv vplkdtrvke qqevvfncev ntegakakwf rneeaifdss kyiilqkdlv |
| 13621 | ytlrirdahl ddqanynvsl tnhrgenvks aanliveeed lriveplkdi etmekksvtf |
| 13681 | wckvnrlnvt lkwtkngeev pfdnrvsyrv dkykhmltik dcgfpdegey ivtagqdksv |
| 13741 | aelliieapt efvehledqt vtefddavfs cqlsrekanv kwyrngreik egkkykfekd |
| 13801 | gsihrliikd criddeceya cgvedrksra rlfveeipve iirppqdile apgadvvfla |
| 13861 | elnkdkvevq wlrnnmvvvq gdkhqmmseg kihrlqicdi kprdqgeyrf iakdkearak |
| 13921 | lelaaapkik tadqdlvvdv gkpltmvvpy daypkaeaew fkeneplstk tidttaeqts |
| 13981 | frileakkgd kgrykivlqn khgkaegfin lkvidvpgpv rnlevtetfd gevslaweep |
| 14041 | ltdggskiig yvverrdikr ktwvlatdra esceftvtgl qkggveylfr vsarnrvgtg |
| 14101 | epvetdnpve arskydvpgp plnvtitdvn rfgvsltwep peydggaeit nyvielrdkt |
| 14161 | sirwdtamtv raedlsatvt dvveggeysf rvraqnrigv gkpsaatpfv kvadpierps |
| 14221 | ppvnitssdq tqssvqlkwe pplkdggspi lgyiiercee gkdnwircnm klvpeltykv |
| 14281 | tglekgnkyl yrvsaenkag vsdpseilgp ltaddafvep tmdlsafkdg levivpnpit |
| 14341 | ilvpstgypr ptatwcfgdk vletgdrvkm ktlsayaelv ispsersdkg iytlklenrv |
| 14401 | ktisgeidvn viarpsapke lkfgditkds vhltweppdd dggspltgyv vekrevsrkt |
| 14461 | wtkvmdfvtd leftvpdlvq gkeylfkvca rnkcgpgepa yvdepvnmst patvpdppen |
| 14521 | vkwrdrtans ifltwdppkn dggsrikgyi vercprgsdk wvacgepvae tkmevtglee |
| 14581 | gkwyayrvka lnrqgaskps rpteeiqavd tqeapeifld vkllagltvk agtkielpat |
| 14641 | vtgkpepkit wtkadmilkq dkritienvp kkstvtivds krsdtgtyii eavnvcgrat |
| 14701 | avvevnvldk pgppaafdit dvtnescllt wnpprddggs kitnyvverr atdsevwhkl |
| 14761 | sstvkdtnfk atklipnkey ifrvaaenmy gvgepvqasp itakyqfdpp gpptrlepsd |
| 14821 | itkdavtltw cepdddggsp itgywverld pdtdkwvrcn kmpvkdttyr vkgltnkkky |
| 14881 | rfrvlaenla gpgkpskste pilikdpidp pwppgkptvk dvgktsvrln wtkpehdgga |
| 14941 | kiesyvieml ktgtdewvrv aegvpttqhl lpglmegqey sfrvravnka gesepsepsd |
| 15001 | pvlcreklyp pspprwlevi nitkntadlk wtvpekdggs pitnyivekr dvrrkgwqtv |
| 15061 | dttvkdtkct vtpltegsly vfrvaaenai gqsdyteied svlakdtftt pgppyalavv |
| 15121 | dvtkrhvdlk weppkndggr pigryviekk erlgtrwvka gktagpdcnf rvtdviegte |
| 15181 | vqfqvraene agvghpsept eilsiedpts ppsppldlhv tdagrkhiai awkppekngg |
| 15241 | spiigyhvem cpvgtekwmr vnsrpikdlk fkveegvvpd keyvlrvrav naigvsepse |
| 15301 | isenvvakdp dckptidlet hdiiviegek lsipvpfrav pvptvswhkd gkevkasdrl |
| 15361 | tmkndhisah levpksvrad agiytitlen klgsatasin vkviglpgpc kdikasditk |
| 15421 | ssckltwepp efdggtpilh yvlerreagr rtyipvmsge nklswtvkdl ipngeyffrv |
| 15481 | kavnkvggge yielknpvia gdpkgppdpp vdvevhnpta eamtitwkpp lydggskimg |
| 15541 | yiiekiakge erwkrcnehl vpiltytakg leegkeyqfr vraenaagis epsratpptk |
| 15601 | avdpidapkv ilrtslevkr gdeialdasi sgspyptitw ikdenvivpe eikkraaplv |
| 15661 | rrrkgevqee epfvlpltqr lsidnskkge sqlrvrdslr pdhglymikv endhgiakap |
| 15721 | ctvsvldtpg ppinfvfedi rktsvlckwe pplddggsei inytlekkdk tkpdsewivv |
| 15781 | tstlrhckys vtkliegkey lfrvraenrf gpgppcvskp lvakdpfgpp dapdkpived |
| 15841 | vtsnsmlvkw nepkdngspi igywlekrev nsthwsrvnk sllnalkanv dgllegltyv |
| 15901 | frvcaenaag pgkfsppsdp ktandpispp gppiprvtdt ssttielewe ppafngggei |
| 15961 | vgyfvdkqlv gtnewsrcte kmikvrqytv keiregadyk lrvsavnaag egppgetqpv |
| 16021 | tvaepqeppa veldvsvkgg igimagktlr ipavvtgrpv ptkvwtkeeg eldkdrvvid |
| 16081 | nvgtkselii kdalrkdhgr yvitatnscg skfaaarvev fdvpgpvldl kpvvtnrkmc |
| 16141 | llnwsdpedd ggseitgfii erkdakmhtw rqpietersk cditgllegq eykfrviakn |
| 16201 | kfgcgppvei gpilavdplg pptsperlty tertkstitl dwkeprsngg spiqgyiiek |
| 16261 | rrhdkpdfer vnkrlcptts flvenldehq myefrvkavn eigesepslp lnvviqddev |
| 16321 | pptiklrlsv rgdtikvkag epvhipadvt glpmpkiews knetviekpt dalqitkeev |
| 16381 | srseaktels ipkavredkg tytvtasnrl gsvfrnvhve vydrpspprn lavtdikaes |
| 16441 | cyltwdapld nggseithyv idkrdasrkk aeweevtnta vekrygiwkl ipngqyefrv |
| 16501 | ravnkygisd ecksdkvviq dpyrlpgppg kpkvlartkg smlvswtppl dnggspitgy |
| 16561 | wlekreegsp ywsrvsrapi tkvglkgvef nvprllegvk yqframaina agigppseps |
| 16621 | dpevagdpif ppgppscpev kdktkssisl gwkppakdgg spikgyivem qeegttdwkr |
| 16681 | vnepdklitt cecvvpnlke lrkyrfrvka vneageseps dttgeipatd iqeepevfid |
| 16741 | igaqdclvck agsqiripav ikgrptpkss wefdgkakka mkdgvhdipe daqletaens |
| 16801 | sviiipeckr shtgkysita knkagqktan crvkvmdvpg ppkdlkvsdi trgscrlswk |
| 16861 | mpdddggdri kgyviekrti dgkawtkvnp dcgsttfvvp dllseqqyff rvraenrfgi |
| 16921 | gppvetiqrt tardpiyppd ppiklkigli tkntvhlswk ppkndggspv thyiveclaw |
| 16981 | dptgtkkeaw rqcnkrdvee lqftvedlve ggeyefrvka vnaagvskps atvgpcdcqr |
| 17041 | pdmppsidlk efmeveegtn vnivakikgv pfptltwfka ppkkpdnkep vlydthvnkl |
| 17101 | vvddtctlvi pqsrrsdtgl ytitavnnlg taskemrlnv lgrpgppvgp ikfesysadq |
| 17161 | mtlswfppkd dggskitnyv iekreanrkt wvhvssepke ctytipklle gheyvfrima |
| 17221 | qnkygigepl dsepetarnl fsvpgapdkp tvssvtrnsm tvnweepeyd ggspvtgywl |
| 17281 | emkdttskrw krvnrdpika mtlgvsykvt gliegsdyqf rvyainaagv gpaslpsdpa |
| 17341 | tardpiappg ppfpkvtdwt kssadlewsp plkdggskvt gyiveykeeg keewekgkdk |
| 17401 | evrgtklvvt glkegafykf rvsavniagi gepgevtdvi emkdrlvspd lqldasvrdr |
| 17461 | ivvhaggvir iiayvsgkpp ptvtwnmner tlpqeatiet taisssmvik ncqrshqgvy |
| 17521 | sllakneage rkktiivdvl dvpgpvgtpf lahnitnesc kltwfspedd ggspitnyvi |
| 17581 | ekresdrraw tpvtytvtrq natvqgliqg kayffriaae nsigmgpfve tsealvirep |
| 17641 | itvperpedl evkevtkntv tltwnppkyd ggseiinyvl esrligtekf hkvtndnlls |
| 17701 | rkytvkglke gdtyeyrvsa vnivgqgkps fctkpitckd elapptlhld frdkltirvg |
| 17761 | eafaltgrys gkpkpkvswf kdeadvledd rthikttpat lalekikakr sdsykycvvv |
| 17821 | enstgsrkgf cqvnvvdrpg ppvgpvsfde vtkdymvisw kpplddggsk itnyiiekke |
| 17881 | vgkdvwmpvt sasakttckv skllegkdyi frihaenlyg isdplvsdsm kakdrfrvpd |
| 17941 | apdqpivtev tkdsalvtwn kphdggkpit nyilekretm skrwarvtkd pihpytkfrv |
| 18001 | pdllegcqye frvsaeneig igdpsppskp vfakdpiakp sppvnpeaid ttcnsvdltw |
| 18061 | qpprhdggsk ilgyiveyqk vgdeewrran htpescpetk ykvtglrdgq tykfrvlavn |
| 18121 | aagesdpahv pepvlvkdrl eppelildan mareqhikvg dtlrlsaiik gvpfpkvtwk |
| 18181 | kedrdaptka ridvtpvgsk leirnaahed ggiysltven pagsktvsvk vlvldkpgpp |
| 18241 | rdlevseirk dscyltwkep lddggsvitn yvverrdvas aqwsplsats kkkshfakhl |
| 18301 | negnqylfrv aaenqygrgp fvetpkpika ldplhppgpp kdlhhvdvdk tevslvwnkp |
| 18361 | drdggspitg ylveyqeegt qdwikfktvt nlecvvtglq qgktyrfrvk aenivglglp |
| 18421 | dttipiecqe klvppsveld vklieglvvk agttvrfpai irgvpvptak wttdgseikt |
| 18481 | dehytvetdn fssvltiknc lrrdtgeyqi tvsnaagskt vavhltvldv pgpptgpini |
| 18541 | ldvtpehmti swqppkddgg spvinyivek qdtrkdtwgv vssgssktkl kiphlqkgce |
| 18601 | yvfrvraenk igvgppldst ptvakhkfsp psppgkpvvt ditenaatvs wtlpksdggs |
| 18661 | pitgyymerr evtgkwvrvn ktpiadlkfr vtglyegnty efrvfaenla glskpspssd |
| 18721 | pikacrpikp pgppinpklk dksretadlv wtkplsdggs pilgyvvecq kpgtaqwnri |
| 18781 | nkdelirqca frvpgliegn eyrfrikaan ivgegeprel aesviakdil hppeveldvt |
| 18841 | crdvitvrvg qtirilarvk grpepditwt kegkvlvrek rvdliqdlpr velqikeavr |
| 18901 | adhgkyiisa knssghaqgs aivnvldrpg pcqnlkvtnv tkenctiswe npldnggsei |
| 18961 | tnfiveyrkp nqkgwsivas dvtkrlikan llanneyyfr vcaenkvgvg ptietktpil |
| 19021 | ainpidrpge penlhiadkg ktfvylkwrr pdydggspnl syhverrlkg sddwervhkg |
| 19081 | sikethymvd rcvenqiyef rvqtknegge sdwvkteevv vkedlqkpvl dlklsgvltv |
| 19141 | kagdtirlea gvrgkpfpev awtkdkdatd ltrsprvkid tradsskfsl tkakrsdggk |
| 19201 | yvvtatntag sfvayatvnv ldkpgpvrnl kivdvssdrc tvcwdppedd ggceiqnyil |
| 19261 | ekcetkrmvw stysatvltp gttvtrlieg neyifrvrae nkigtgppte skpviaktky |
| 19321 | dkpgrpdppe vtkvskeemt vvwnppeydg gksitgyfle kkekhstrwv pvnksaiper |
| 19381 | rmkvqnllpd heyqfrvkae neigigepsl psrpvvakdp ieppgpptnf rvvdttkhsi |
| 19441 | tlgwgkpvyd ggapiigyvv emrpkiadas pdegwkrcna aaqlvrkeft vtsldenqey |
| 19501 | efrvcaqnqv gigrpaelke aikpkeilep peidldasmr klvivragcp irlfaivrgr |
| 19561 | papkvtwrkv gidnvvrkgq vdlvdtmafl vipnstrdds gkysltlvnp agekavfvnv |
| 19621 | rvldtpgpvs dlkvsdvtkt schvswappe ndggsqvthy ivekreadrk twstvtpevk |
| 19681 | ktsfhvtnlv pgneyyfrvt avneygpgvp tdvpkpvlas dplsepdppr klevtemtkn |
| 19741 | satlawlppl rdggakidgy itsyreeeqp adrwteysvv kdlslvvtgl kegkkykfrv |
| 19801 | aarnavgvsl preaegvyea keqllppkil mpeqitikag kklrieahvy gkphptckwk |
| 19861 | kgedevvtss hlavhkadss siliikdvtr kdsgyyslta enssgtdtqk ikvvvmdapg |
| 19921 | ppqppfdisd idadacslsw hipledggsn itnyivekcd vsrgdwvtal asvtktscrv |
| 19981 | gklipgqeyi frvraenrfg isepltspkm vaqfpfgvps epknarvtkv nkdcifvawd |
| 20041 | rpdsdggspi igylierker nsllwvkand tlvrsteypc aglvegleys friyalnkag |
| 20101 | ssppskptey vtarmpvdpp gkpevidvtk stvsliwarp khdggskiig yfveacklpg |
| 20161 | dkwvrcntap hqipqeeyta tgleekaqyq fraiartavn isppsepsdp vtilaenvpp |
| 20221 | ridlsvamks lltvkagtnv cldatvfgkp mptvswkkdg tllkpaegik mamqrnlctl |
| 20281 | elfsvnrkds gdytitaens sgsksatikl kvldkpgppa svkinkmysd ramlsweppl |
| 20341 | edggseitny ivdkretsrp nwaqvsatvp itscsvekli egheyqfric aenkygvgdp |
| 20401 | vftepaiakn pydppgrcdp pvisnitkdh mtvswkppad dggspitgyl lekretqavn |
| 20461 | wtkvnrkpii ertikatglq egteyefrvt ainkagpgkp sdaskaayar dpqyppappa |
| 20521 | fpkvydttrs svslswgkpa ydggspiigy lvevkradsd nwvrcnlpqn lqktrfevtg |
| 20581 | lmedtqyqfr vyavnkigys dpsdvpdkhy pkdilippeg eldadlrktl ilragvtmrl |
| 20641 | yvpvkgrppp kitwskpnvn lrdrigldik stdfdtflrc envnkydagk yiltlpnscg |
| 20701 | kkeytivvkv ldtpgppvnv tvkeiskdsa yvtweppiid ggspiinyvv qkrdaerksw |
| 20761 | stvttecskt sfrvanleeg ksyffrvfae neygigdpge trdavkasqt pgpvvdlkvr |
| 20821 | svsksscsig wkkphsdggs riigyvvdfl teenkwqrvm kslslqysak dltegkeytf |
| 20881 | rvsaenenge gtpseitvva rddvvapdld lkglpdlcyl akensnfrlk ipikgkpaps |
| 20941 | vswkkgedpl atdtrvsves savnttlivy dcqksdagky titlknvagt kegtisikvv |
| 21001 | gkpgiptgpi kfdevtaeam tlkwappkdd ggseitnyil ekrdsvnnkw vtcasavqkt |
| 21061 | tfrvtrlheg meytfrvsae nkygvgeglk sepivarhpf dvpdappppn ivdvrhdsvs |
| 21121 | ltwtdpkktg gspitgyhle fkernsllwk ranktpirmr dfkvtglteg leyefrvmai |
| 21181 | nlagvgkpsl psepvvaldp idppgkpevi nitrnsvtli wtepkydggh kltgyivekr |
| 21241 | dlpskswmka nhvnvpecaf tvtdlveggk yefriraknt agaisapses tetiickdey |
| 21301 | eaptivldpt ikdgltikag dtivlnaisi lgkplpkssw skagkdirps ditqitstpt |
| 21361 | ssmltikyat rkdageytit atnpfgtkve hvkvtvldvp gppgpveisn vsaekatltw |
| 21421 | tppledggsp iksyilekre tsrllwtvvs ediqscrhva tkliqgneyi frvsavnhyg |
| 21481 | kgepvqsepv kmvdrfgppg ppekpevsnv tkntatvswk rpvddggsei tgyhverrek |
| 21541 | kslrwvraik tpvsdlrckv tglqegstye frvsaenrag igppseasds vlmkdaaypp |
| 21601 | gppsnphvtd ttkksaslaw gkphydggle itgyvvehqk vgdeawikdt tgtalritqf |
| 21661 | vvpdlqtkek ynfrisaind agvgepavip dveiverema pdfeldaelr rtlvvragls |
| 21721 | irifvpikgr papevtwtkd ninlknrani entesftlli ipecnrydtg kfvmtienpa |
| 21781 | gkksgfvnvr vldtpgpvln lrptditkds vtlhwdlpli dggsritnyi vekreatrks |
| 21841 | ystattkchk ctykvtglse gceyffrvma eneygigept ettepvkase apsppdslni |
| 21901 | mditkstvsl awpkpkhdgg skitgyviea qrkgsdqwth ittvkglecv vrnltegeey |
| 21961 | tfqvmavnsa grsapresrp vivkeqtmlp eldlrgiyqk lviakagdni kveipvlgrp |
| 22021 | kptvtwkkgd qilkqtqrvn fettatstil ninecvrsds gpypltarni vgevgdviti |
| 22081 | qvhdipgppt gpikfdevss dfvtfswdpp endggvpisn yvvemrqtds ttwvelattv |
| 22141 | irttykatrl ttgleyqfrv kaqnrygvgp gitsacivan ypfkvpgppg tpqvtavtkd |
| 22201 | smtiswhepl sdggspilgy hverkerngi lwqtvskalv pgnifkssgl tdgiayefrv |
| 22261 | iaenmagksk pskpsepmla ldpidppgkp vplnitrhtv tlkwakpeyt ggfkitsyiv |
| 22321 | ekrdlpngrw lkanfsnile neftvsglte daayefrvia knaagaispp sepsdaitcr |
| 22381 | ddveapkikv dvkfkdtvil kageafrlea dvsgrppptm ewskdgkele gtakleikia |
| 22441 | dfstnlvnkd strrdsgayt ltatnpggfa khifnvkvld rpgppegpla vtevtsekcv |
| 22501 | lswfpplddg gakidhyivq kretsrlawt nvasevqvtk lkvtkllkgn eyifrvmavn |
| 22561 | kygvgeples epvlavnpyg ppdppknpev ttitkdsmvv cwghpdsdgg seiinyiver |
| 22621 | rdkagqrwik cnkktltdlr ykvsgltegh eyefrimaen aagisapspt spfykacdtv |
| 22681 | fkpgppgnpr vldtsrssis iawnkpiydg gseitgymve ialpeedewq ivtppaglka |
| 22741 | tsytitglte nqeykiriya mnseglgepa lvpgtpkaed rmlppeield adlrkvvtir |
| 22801 | acctlrlfvp ikgrpapevk wardhgesld kasiestssy tllivgnvnr fdsgkyiltv |
| 22861 | enssgsksaf vnvrvldtpg ppqdlkvkev tktsvtltwd pplldggski knyivekres |
| 22921 | trkaystvat nchktswkvd qlwegcsyyf rvlaeneygi glpaetaesv kaserplppg |
| 22981 | kitlmdvtrn sbslswekpe hdggsrilgy ivemqtkgsd kwatcatvkv teatitgliq |
| 23041 | geeysfrvsa qnekgisdpr qlsvpviakd lvippafkll fntftvlage dlkvdvpfig |
| 23101 | rptpavtwhk dnvplkqttr vnaestenns lltikdacre dvghyvvklt nsageaietl |
| 23161 | nvivldkpgp ptgpvkmdev tadsitlswg ppkydggssi nnyivekrdt stttwqivsa |
| 23221 | tvarttikac rlktgceyqf riaaenrygk stylnseptv aqypfkvpgp pgtpvvtlss |
| 23281 | rdsmevqwne pisdggsrvi gyhlerkern silwvklnkt pipqtkfktt gleegveyef |
| 23341 | rvsaenivgi gkpskvsecy vardpcdppg rpeaiivtrn svtlqwkkpt ydggskitgy |
| 23401 | ivekkelpeg rwmkasftni idthfevtgl vedhryefrv iarnaagvfs epsestgait |
| 23461 | ardevdppri smdpkykdti vvhagesfkv dadiygkpip tigwikgdqe lsntarleik |
| 23521 | stdfatslsv kdavrvdsgn yilkaknvag ersvtvnvkv ldrpgppegp vvisgvtaek |
| 23581 | ctlawkpplq dggsdiinyi verretsrlv wtvvdanvqt lsckvtklle gneytfrima |
| 23641 | vnkygvgepl esepvvaknp fvvpdapkap evttvtkdsm ivvwerpasd ggseilgyvl |
| 23701 | ekrdkegirw trchkrlige lrlrvtglie nhdyefrvsa enaaglseps ppsayqkacd |
| 23761 | piykpgppnn pkviditrss vflswskpiy dggceiqgyi vekcdvsvge wtmctpptgi |
| 23821 | nktnievekl lekheynfri cainkagvge hadvpgpiiv eekleapdid ldlelrkiin |
| 23881 | iraggslrlf vpikgrptpe vkwgkvdgei rdaaiidvts sftslvldnv nrydsgkytl |
| 23941 | tlenssgtks afvtvrvldt psppvnlkvt eitkdsysit wepplldggs kiknyivekr |
| 24001 | eatrksyaav vtnchknswk idqlqegcsy yfrvtaeney giglpaqtad pikvaevpqp |
| 24061 | pgkitvddvt rnsyslswtk pehdggskii gyivemgakh sekwsecarv kslqavitnl |
| 24121 | tqgeeylfrv vavnekgrsd prslavpiva kdlviepdvk pafssysvqv gqdlkievpi |
| 24181 | sgrpkptitw tkdglplkqt trinvtdsld lttlsiketh kddggqygit vanvvgqkta |
| 24241 | sieivtldkp dppkgpvkfd dvsaesitls wnpplytggc qitnyivqkr dttttvwdvv |
| 24301 | satvarttlk vtklktgtey qfrifaenry gqsfalesdp ivaqypykep gppgtpfata |
| 24361 | iskdsmviqw hepvnnggsp vigyhlerke rnsilwtkvn ktiihdtqfk aqnleegiey |
| 24421 | efrvyaeniv gvgkasknse cyvardpcdp pgtpepimvk rneitiqwtk pvydggsmit |
| 24481 | gyivekrdlp dgrwmkasft nvietqftvs gltedqryef rviaknaaga iskpsdstgp |
| 24541 | itakdevelp rismdpkfrd tivvnagetf rleadvhgkp lptiewlrgd keieesarce |
| 24601 | ikntdfkall ivkdairidg gqyilrasnv agsksfpvnv kvldrpgppe gpvqvtgvts |
| 24661 | ekcsltwspp lqdggsdish yvvekretsr lawtvvasev vtnslkvtkl legneyvfri |
| 24721 | mavnkygvge plesapvlmk npfvlpgppk slevtniakd smtvcwnrpd sdggseiigy |
| 24781 | ivekrdrsgi rwikcnkrri tdlrlrvtgl tedheyefrv saenaagvge pspatvyyka |
| 24841 | cdpvfkpypp tnahivdttk nsitlawgkp iydggseilg yvveickade eewqivtpqt |
| 24901 | glrvtrfeis kltehqeyki rvcalnkvgl geatsvpgtv kpedkleape ldldselrkg |
| 24961 | ivvraggsar ihipfkgrpt peitwsreeg eftdkvqiek gvnytqlsid ncdrndagky |
| 25021 | ilklenssgs ksafvtvkvl dtpgppqnla vkevrkdsaf lvweppiidg gakvknyvid |
| 25081 | krestrkaya nvsskcskts fkvenltega iyyfrvmaen efgvgvpvet vdavkaaepp |
| 25141 | sppgkvtltd vsqtsaslmw ekpehdggsr vlgyvvemqp kgtekwsiva eskvcnavvt |
| 25201 | glssgqeyqf rvkaynekgk sdprvlgvpv iakdltiqps lklpfntysi qagedlkiei |
| 25261 | pvigrprpni swvkdgeplk qttrvnveet atstvlhike gnkddfgkyt vtatnsagta |
| 25321 | tenlsvivle kpgppvgpvr fdevsadfvv isweppaytg gcqisnyive krdtttttwh |
| 25381 | mvsatvartt ikitklktgt eyqfrifaen rygksaplds kavivqypfk epgppgtpfv |
| 25441 | tsiskdqmlv qwhepvndgg tkiigyhleq keknsilwvk lnktpiqdtk fkttgldegl |
| 25501 | eyefkvsaen ivgigkpskv secfvardpc dppgrpeaiv itrnnvtlkw kkpaydggsk |
| 25561 | itgyivekkd lpdgrwmkas ftnvleteft vsglvedqry efrviarnaa gnfsepsdss |
| 25621 | gaitardeid apnasldpky kdvivvhage tfvleadirg kpipdvvwsk dgkeleetaa |
| 25681 | rmeikstiqk ttlvvkdcir tdgggyilkl snvggtksip itvkvldrpg ppegplkvtg |
| 25741 | vtaekcylaw npplqdggan ithyiiekre tsrlswtqvs tevqalnykv tkllpgneyi |
| 25801 | frvmavnkyg igeplesgpv tacnpykppg ppstpevsai tkdsmvvtwa rpvddggtei |
| 25861 | egyilekrdk egvrwtkcnk ktltdlrlrv tglteghsye frvaaenaag vgepsepsvf |
| 25921 | yracdalypp gppsnpkvtd tsrssvslaw skpiydggap vkgyvvevke aaadewttct |
| 25981 | pptglqgkqf tvtklkente ynfricains egvgepatlp gsvvaqerie ppeieldadl |
| 26041 | rkvvvlrasa tlrlfvtikg rpepevkwek aegiltdraq ievtssftml vidnvtrfds |
| 26101 | grynitlenn sgsktafvnv rvldspsapv nltirevkkd svtlsweppl idggakitny |
| 26161 | ivekrettrk ayatitnnct kttfrienlq egcsyyfrvl asneygiglp aettepvkvs |
| 26221 | epplppgrvt lvdvtrntat ikwekpesdg gskitgyvve mqtkgsekws tctqvktlea |
| 26281 | tisgltagee yvfrvaavne kgrsdprqlg vpviardiei kpsvelpfht fnvkareqlk |
| 26341 | idvpfkgrpq atvnwrkdgq tlkettrvnv sssktvtsls ikeaskedvg tyelcvsnsa |
| 26401 | gsitvpitii vldrpgppgp iridevscds itiswnppey dggcqisnyi vekkettstt |
| 26461 | whivsqavar tsikivrltt gseyqfrvca enrygkssys essavvaeyp fsppgppgtp |
| 26521 | kvvhatkstm lvtwqvpvnd ggsrvigyhl eykerssilw skankiliad tqmkvsglde |
| 26581 | glmyeyrvya eniagigkcs kscepvpard pcdppggpev tnitrksysl kwskphydgg |
| 26641 | akitgyiver relpdgrwlk cnytniqety fevteltedq ryefrvfarn aadsvsepse |
| 26701 | stgpiivkdd vepprvmmdv kfrdvivvka gevlkinadi agrplpvisw akdgieieer |
| 26761 | arteiistdn htlltvkdci rrdtgqyvlt lknvagtrsv avnckvldkp gppagplein |
| 26821 | gltaekcsls wgrpqedgga didyyivekr etshlawtic egelqmtsck vtkllkgney |
| 26881 | ifrvtgvnky gvgeplesva ikaldpftvp spptsleits vtkesmtlcw srpesdggse |
| 26941 | isgyiierre knslrwvrvn kkpvydlrvk stglregcey eyrvyaenaa glslpsetsp |
| 27001 | liraedpvfl psppskpkiv dsgkttitia wvkplfdgga pitgytveyk ksddtdwkts |
| 27061 | iqslrgteyt isglttgaey vfrvksvnkv gasdpsdssd pqiakereee plfdidsemr |
| 27121 | ktlivkagas ftmtvpfrgr pvpnvlwskp dtdlrtrayv dttdsrtslt ienanrndsg |
| 27181 | kytltiqnvl saasltlvvk vldtpgpptn itvqdvtkes avlswdvpen dggapvknyh |
| 27241 | iekreaskka wvsvtnncnr lsykvtnlqe gaiyyfrvsg enefyvgipa etkegvkite |
| 27301 | kpsppeklgv tsiskdsysl twlkpehdgg srivhyvvea lekgqknwvk cavaksthhv |
| 27361 | vsglrensey ffrvfaenqa glsdprelll pvlikeqlep peidmknfps htvyvragsn |
| 27421 | lkvdipisgk plpkvtlsrd gvplkatmrf nteitaenlt inlkesvtad agryeitaan |
| 27481 | ssgttkafin ivvldrpgpp tgpvvisdit eesvtlkwep pkydggsqvt nyillkrets |
| 27541 | tavwtevsat vartmmkvmk lttgeeyqfr ikaenrfgis dhidsacvtv klpyttpgpp |
| 27601 | stpwvtnvtr esitvgwhep vsnggsavvg yhlemkdrns ilwqkanklv irtthfkvtt |
| 27661 | isagliyefr vyaenaagvg kpshpsepvl aidacepprn vritdiskns vslswqqpaf |
| 27721 | dggskitgyi verrdlpdgr wtkasftnvt etqfiisglt qnsqyefrvf arnavgsisn |
| 27781 | psevvgpitc idsyggpvid lpleytevvk yragtsvklr agisgkpapt iewykddkel |
| 27841 | qtnalvcven ttdlasilik dadrlnsgcy elklrnamgs asatirvqil dkpgppggpi |
| 27901 | efktvtaeki tllwrppadd ggakithyiv ekretsrvvw smvsehleec iitttkiikg |
| 27961 | neyifrvrav nkygigeple sdsvvaknaf vtpgppgipe vtkitknsmt vvwsrpiadg |
| 28021 | gsdisgyfle krdkkslgwf kvlketirdt rqkvtglten sdyqyrvcav naagqgpfse |
| 28081 | psefykaadp idppgppaki riadstkssi tlgwskpvyd ggsavtgyvv eirqgeeeew |
| 28141 | ttvstkgevr tteyvvsnlk pgvnyyfrvs avncagqgep iemnepvqak dileapeidl |
| 28201 | dvalrtsvia kagedvqvli pfkgrppptv twrkdeknlg sdarysient dssslltipq |
| 28261 | vtrndtgkyi ltiengvgep ksstvsvkvl dtpaacqklq vkhvsrgtvt llwdpplidg |
| 28321 | gspiinyvie krdatkrtws vvshkcssts fklidlsekt pfffrvlaen eigigepcet |
| 28381 | tepvkaaevp apirdlsmkd stktsvilsw tkpdfdggsv iteyvverkg kgeqtwshag |
| 28441 | isktceievs qlkegsvlef rvfaknekgl sdpvtigpit vkeliitpev dlsdipgaqv |
| 28501 | tvrighnvhl elpykgkpkp siswlkdglp lkesefvrfs ktenkitlsi knakkehggk |
| 28561 | ytvildnavc riavpitvit lgppskpkgp irfdeikads vilswdvped ngggeitcys |
| 28621 | iekretsqtn wkmvcssvar ttfkvpnlvk daeyqfrvra enrygvsqpl vssiivakhq |
| 28681 | fripgppgkp viynvtsdgm sltwdapvyd ggsevtgfhv ekkernsilw qkvntspisg |
| 28741 | reyratglve gldyqfrvya ensaglssps dpskftlavs pvdppgtpdy idvtretitl |
| 28801 | kwnpplrdgg skivgysiek rqgnerwvrc nftdvsecqy tvtglspgdr yefriiarna |
| 28861 | vgtisppsqs sgiimtrden vppivefgpe yfdgliiksg eslrikalvq grpvprvtwf |
| 28921 | kdgveiekrm nmeitdvlgs tslfvrdatr dhrgvytvea knasgsakae ikvkvqdtpg |
| 28981 | kvvgpirftn itgekmtlww daplndgcap ithyiiekre tsrlawalie dkceaqsyta |
| 29041 | iklingneyq frvsavnkfg vgrpldsdpv vaqiqytvpd apgipepsni tgnsitltwa |
| 29101 | rpesdggsei qqyilerrek kstrwvkvis krpisetrfk vtgltegney efhvmaenaa |
| 29161 | gvgpasgisr likcrepvnp pgpptvvkvt dtskttvsle wskpvfdggm eiigyiiemc |
| 29221 | kadlgdwhkv naeacvktry tvcdlqagee ykfrvsaing agkgdscevt gtikavdrlt |
| 29281 | apeldidanf kgthvvraga sirlflayqg rptptavwsk pdsnlslrad ihttdsfstl |
| 29341 | tvencnrnda gkytltvenn sgsksitftv kvldtpgppg pitfkdvtrg satlmwdapl |
| 29401 | ldggarihhy vvekreasrr swqvisekct rqifkvndla egvpyyfrvs avneygvgep |
| 29461 | yempepivat eqpapprrld vvdtskssav lawlkpdhdg gsritgylle mrqkgsdfwv |
| 29521 | eaghtkqltf tverlvekte yefrvkaknd agysepreaf ssviikepqi eptadltgit |
| 29581 | nqlitckags pftidvpisg rpapkvtwkl eemrlketdr vsitttkdrt tltvkdsmrg |
| 29641 | dsgryfltle ntagvktfsv tvvvigrpgp vtgpievssv saescvlswg epkdgggtei |
| 29701 | tnyivekres gttawqlvns svkrtgikvt hltkymeysf rvssenrfgv skplesapii |
| 29761 | aehpfvppsa ptrpevyhvs anamsirwee pyhdggskii gywvekkern tilwvkenkv |
| 29821 | pclecnykvt glvegleyqf rtyalnaagv skaseasrpi maqnpvdapg rpevtdvtrs |
| 29881 | tvsliwsapa ydggskvvgy iierkpvsev gdgrwlkcny tivsdnfftv talsegdtye |
| 29941 | frvlaknaag viskgsestg pvtcrdeyap pkaeldarlh gdlvtirags dlvldaavgg |
| 30001 | kpepkiiwtk gdkeldlcek vslqytgkra tavikfcdrs dsgkytltvk nasgtkavsv |
| 30061 | mvkvldspgp cgkltvsrvt gekctlawsl pqedggaeit hyiverrets rlnwvivege |
| 30121 | cptlsyvvtr liknneyifr vravnkygpg vpvesepiva rnsftipspp gipeevgtgk |
| 30181 | ehiiiqwtkp esdggneisn ylvdkrekks lrwtrvnkdy vvydtrlkvt slmegcdyqf |
| 30241 | rvtavnaagn sepseasnfi screpsytpg ppsaprvvdt tkhsislawt kpmydggtdi |
| 30301 | vgyvlemgek dtdqwyrvht natirnteft vpdlkmgqky sfrvaavnvk gmseysesia |
| 30361 | eiepveriei pdleladdlk ktvtiragas lrlmvsysgr pppvitwskq gidlasraii |
| 30421 | dttesyslli vdkvnrydag kytieaenqs gkksatvlvk vydtpgpcps vkvkevsrds |
| 30481 | vtitweipti dggapvnnyi vekreaamra fktvttkcsk tlyrisglve gtmyyfrvlp |
| 30541 | eniygigepc etsdavlvse vplvpaklev vdvtkstvtl awekplydgg srltgyvlea |
| 30601 | ckagterwmk vvtlkptvle htvtslnege qylfriraqn ekgvsepret vtavtvqdlr |
| 30661 | vlptidlstm pqktihvpag rpvelvipia grpppaaswf fagsklrese rvtvethtkv |
| 30721 | akltiretti rdtgeytlel knvtgttset ikviildkpg pptgpikide idatsitisw |
| 30781 | eppeldggap lsgyvveqrd ahrpgwlpvs esvtrstfkf trltegneyv frvaatnrfg |
| 30841 | igsylqsevi ecrssiripg ppetlqifdv srdgmtltwy ppeddggsqv tgyiverkev |
| 30901 | radrwvrvnk vpvtmtryrs tgltegleye hrvtainarg sgkpsrpskp ivamdpiapp |
| 30961 | gkpqnprvtd ttrtsyslaw svpedeggsk vtgyliemqk vdqhewtkcn ttptkireyt |
| 31021 | lthlpqgaey rfrvlacnag gpgepaevpg tvkvtemley pdyelderyq egifvrqggv |
| 31081 | irltipikgk pfpickwtke gqdiskrami atsethtelv ikeadrgdsg tydlvlenkc |
| 31141 | gkkavyikvr vigspnspeg pleyddiqvr svrvswrppa ddggadilgy ilerrevpka |
| 31201 | awytidsrvr gtslvvkglk enveyhfrvs aenqfgiskp lkseepvtpk tpinppepps |
| 31261 | nppevldvtk ssvslswsrp kddggsrvtg yyierketst dkwvrhnktq itttmytvtg |
| 31321 | lvpdaeyqfr iiagndvgls etspasepvv ckdpfdkpsq pgeleilsis kdsvtlqwek |
| 31381 | pecdggkeil gywveyrqsg dsawkksnke rikdkqftig glleateyef rvfaenetgl |
| 31441 | srprrtamsi ktkltsgeap girkemkdvt tklgeaaqls cqivgrplpd ikwyrfgkel |
| 31501 | iqsrkykmss dgrthtltvm teeqedegvy tciatnevge vetssklllq atpqfhpgyp |
| 31561 | lkekyygavg stlrlhvmyi grpvpamtwf hgqkllqnse nitientehy thlvmknvqr |
| 31621 | kthagkykvq lsnvfgtvda ildveiqdkp dkptgpivie allknsavis wkppaddggs |
| 31681 | witnyvvekc eakegaewql vssaisvttc rivnltenag yyfrvsaqnt fgisdplevs |
| 31741 | svviikspfe kpgapgkpti tavtkdscvv awkppasdgg akirnyylek rekkqnkwis |
| 31801 | vtteeiretv fsvknliegl eyefrvkcen lggesewsei sepitpksdv piqaphfkee |
| 31861 | lrnlnvryqs natlvckvtg hpkpivkwyr qgkeiiadgl kyriqefkgg yhqliiasvt |
| 31921 | dddatvyqvr atnqggsysg taslevevpa kihlpktleg mgavhalrge vvsikipfsg |
| 31981 | kpdpvitwqk gqdlidnngh yqvivtrsft slvfpngver kdagfyvvca knrfgidqkt |
| 32041 | veldvadvpd pprgvkvsdv srdsvnltwt epasdggski tnyivekcat taerwlrvgq |
| 32101 | aretrytvin lfgktsyqfr viaenkfgls kpsepsepti tkedktramn ydeevdetre |
| 32161 | vsmtkashss tkelyekymi aedlgrgefg ivhrcvetss kktymakfvk vkgtdqvlvk |
| 32221 | keisilniar hrnilhlhes fesmeelvmi fefisgldif erintsafel nereivsyvh |
| 32281 | qvcealqflh shnighfdir peniiyqtrr sstikiiefg qarqlkpgdn frllftapey |
| 32341 | yapevhqhdv vstatdmwsl gtivyvllsg inpflaetnq qiienimnae ytfdeeafke |
| 32401 | isieamdfvd rllvkerksr mtasealqhp wlkqkiervs tkvirtlkhr ryyhtlikkd |
| 32461 | lnmvvsaari scggairsqk gvsvakvkva sieigpvsgq imhavgeegg hvkyvckien |
| 32521 | ydqstqvtwy fgvrglense kyeityedgv ailyvkditk lddgtyrckv vndygedssy |
| 32581 | aelfvkgvre vydyycrrtm kkikrrtdtm rllerppeft lplynktayv genvrfgvti |
| 32641 | tvhpephvtw yksgqkikpg dndkkytfes dkglyqltin svttdddaey tvvarnkyge |
| 32701 | dsckakltvt lhppptdstl rpmfkrllan aecqegqsvc feirvsgipp ptlkwekdgq |
| 32761 | plslgpniei ihegldyyal hirdtlpedt gyyrvtatnt agstscqahl gverlrykkq |
| 32821 | efkskeeher hvqkqidktl rmaeilsgte svpltqvake alreaavlyk pavstktvkg |
| 32881 | efrleieekk eerklrmpyd vpeprkykqt tieedqrikq fvpmsdmkwy kkirdqyemp |
| 32941 | gkldrvvqkr pkrirlsrwe qfyvmplpri tdqyrpkwri pklsqddlei vrparrrtps |
| 33001 | pdydfyyrpr rrslgdisde elllpiddyl amkrteeerl rleeelelgf sasppsrspp |
| 33061 | hfelsslrys spqahvkvee trkdfrysty hiptkaeast syaelrerha qaayrqpkqr |
| 33121 | qrimaerede ellrpvtttq hlseykseld fmskeeksrk ksrrqrevte iteieeeyei |
| 33181 | skhaqresss sasrllrrrr slsptyielm rpvselirsr pqpaeeyedd terrsptper |
| 33241 | trprspspvs serslsrfer sarfdifsry esmkaalktq ktserkyevl sqqpftldha |
| 33301 | pritlrmrsh rvpcgqntrf ilnvqskpta evkwyhngve lqesskihyt ntsgvltlei |
| 33361 | ldchtddsgt yravctnykg easdyatldv tggdyttyas qrrdeevprs vfpeltrtea |
| 33421 | yavssfkkts emeasssvre vksqmtetre slssyehsas aemksaalee ksleeksttr |
| 33481 | kikttlaari ltkprsmtvy egesarfscd tdgepvptvt wlrkgqvlst sarhqvtttk |
| 33541 | ykstfeissv qasdegnysv vvensegkqe aeftltiqka rvtekavtsp prvkspeprv |
| 33601 | kspeavkspk rvkspepshp kavsptetkp tptekvqhlp vsappkitqf lkaeaskeia |
| 33661 | kltcvvessv lrakevtwyk dgkklkengh fqfhysadgt yelkinnlte sdqgeyvcei |
| 33721 | sgeggtsktn lqfmgqafks ihekvskise tkksdqktte stvtrktepk apepisskpv |
| 33781 | ivtglqdttv ssdsvakfav katgeprpta iwtkdgkait qggkyklsed kggffleihk |
| 33841 | tdtsdsglyt ctvknsagsv sssckltika ikdteaqkvs tqktseitpq kkavvqeeis |
| 33901 | qkalrseeik mseaksqekl alkeeaskvl iseevkksaa tsleksivhe eitktsqase |
| 33961 | evrthaeika fstqmsineg grlvlkania gatdvkwvln gveltnseey rygvsgsdqt |
| 34021 | ltikqashrd egiltciskt kegivkcqyd ltlskelsda pafisqprsq ninegqnvlf |
| 34081 | tceisgepsp eiewfknnlp isissnvsis rsrnvyslei rnasysdsgk ytikaknfrg |
| 34141 | qcsataslmv lplveepsre vvlrtsgdts lqgsfssqsv qmsaskqeas fssfssssas |
| 34201 | smtemkfasm saqsmssmqe sfvemssssf mgisnmtqle sstskmlkag irgippkiea |
| 34261 | lpsdisideg kvltvacaft geptpevtws cggrkihsqe ggrfhientd dlttliimdv |
| 34321 | qkqdgglytl slgnefgsds atvnihirsi |

| HBA1 (e.g., GenBank AccessionNumber P69905 (SEQ ID NO: 2): | |
|---|---|
| 1 | mvlspadktn vkaawgkvga hageygaeal ermflsfptt ktyfphfdls hgsaqvkghg |
| 61 | kkvadaltna vahvddmpna lsalsdlhah klrvdpvnfk llshcllvtl aahlpaeftp |
| 121 | avhasldkfl asvstvltsk yr |

| Insulin-like growth factor 1 receptor (IGF1R) | |
|---|---|
| (e.g., GenBank Accession Number F08069 (SEQ ID NO: 3): | |
| 1 | mksgsgggsp tslwgllfls aalslwptsg eicgpgidir ndyqqlkrle nctviegylh |
| 61 | illiskaedy rsyrfpkltv iteylllfrv agleslgdlf pnltvirgwk lfynyalvif |
| 121 | emtnlkdigl ynlrnitrga irieknadlc ylstvdwsli ldavsnnyiv gnkppkecgd |
| 181 | lcpgtmeekp mcekttinne ynyrcwttnr cqkmcpstcg kractennec chpeclgscs |
| 241 | apdndtacva crhyyyagvc vpacppntyr fegwrcvdrd fcanilsaes sdsegfvihd |
| 301 | gecmqecpsg firngsgsmy cipcegpcpk vceeekktkt idsvtsaqml qgctifkgnl |
| 361 | linirrgnni aselenfmgl ievvtgyvki rhshalvsls flknlrlilg eeqlegnysf |
| 421 | yvldnqnlqq lwdwdhrnlt ikagkmyfaf npklcvseiy rmeevtgtkq rqskgdintr |
| 481 | nngerasces dvlhftsttt sknriiitwh ryrppdyrdl isftvyykea pfknvteydg |
| 541 | qdacgsnswn mvdvdlppnk dvepgillhg lkpwtqyavy vkavtltmve ndhirgakse |
| 601 | ilyirtnasv psipldvlsa snsssqlivk wnppslpngn lsyyivrwqr qpqdgylyrh |
| 661 | nycskdkipi rkyadgtidi eevtenpkte vcggekgpcc acpkteaekq aekeeaeyrk |
| 721 | vfenflhnsi fvprperkrr dvmqvanttm ssrsrnttaa dtynitdpee leteypffes |
| 781 | rvdnkertvi snlrpftlyr idihscnhea eklgcsasnf vfartmpaeg addipgpvtw |
| 841 | eprpensifl kwpepenpng lilmyeikyg sqvedqrecv srqeyrkygg aklnrlnpgn |
| 901 | ytariqatsl sgngswtdpv ffyvqaktgy enfihliial pvavllivgg lvimlyvfhr |
| 961 | krnnsrlgng vlyasvnpey fsaadvyvpd ewevarekit msrelgqgsf gmvyegvakg |
| 1021 | vvkdepetrv aiktvneaas mrerieflne asvmkefnch hvvrllgvvs qgqptlvime |
| 1081 | lmtrgdlksy lrslrpemen npvlappsls kmiqmageia dgmaylnank fvhrdlaarn |
| 1141 | cmvaedftvk igdfgmtrdi yetdyyrkgg kgllpvrwms peslkdgvft tysdvwsfgv |
| 1201 | vlweiatlae qpyqglsneq vlrfvmeggl ldkpdncpdm lfelmrmcwq ynpkmrpsfl |
| 1261 | eiissikeem epgfrevsfy yseenklpep eeldlepenm esvpldpsas ssslplpdrh |
| 1321 | sghkaengpg pgvlvlrasf derqpyahmn ggrkneralp lpgsstc |

| Isoform 3 of zonadhesin precursor | |
|---|---|
| (e.g., GenBank Accession Number Q9Y493-1 (SEQ ID NO: 4): | |
| 1 | mvppvwtlll lvgaalfrke kppdqklvvr ssrdnyvltq cdfeddakpl cdwsqvsadd |
| 61 | edwvrasgps ptgstgapgg ypngegsylh mesnsfhrgg varllspdlw eqgplcvhfa |
| 121 | hhmfglswga qlrllllsge egrrpdvlwk hwntqrpswm Ittvtvpagf tlptrlmfeg |
| 181 | trgstayldi aldalsirrg scnrvcmmqt csfdipndlc dwtwiptasg akwtqkkgss |
| 241 | gkpgvgpdgd fsspgsgcym lldpknarpg qkavllspvs lssgclsfsf hyilrgqspg |
| 301 | aalhiyasvl gsirkhtlfs gqpgpnwqav svnytavgri qfavvgvfgk tpepavavda |
| 361 | tsiapcgegf pqcdfednah pfcdwvqtsg dgghwalghk ngpvhgmgpa ggfpnagghy |
| 421 | iyleadefsh aggsvrlvsr pfcapgdicv efayhmyglg egtmlelllg spagsppipl |
| 481 | wkrvgsqrpy wqntsvtvps ghqqpmqlif kgiqgsntas vvamgfilin pqtcpvkvlp |
| 541 | elppvspvss tgpsettglt enptistkkp tvsiekpsvt tekptvpkek ptiptekpti |
| 601 | stekptipse kpnmpsekpt ipsekptilt ekptipsekp tipsekptis tekptvptee |
| 661 | pttpteettt smeepvipte kpsiptekps iptekptism eetiistekp tispekptip |
| 721 | tekptiptek stispekptt ptekptipte kptispekpt tptekptisp ekltiptekp |
| 781 | tiptekptip tekptistee pttpteetti stekpsipme kptlpteett tsveettist |
| 841 | ekltipmekp tistekptip tekptispek ltipteklti ptekptipie ettisteklt |
| 901 | iptekptisp ekptistekp tiptekptip teettistek ltiptekpti spekltipte |
| 961 | kptistekpt iptekltipt ekptiptekp tiptekltal rpphpsptat glaalvmsph |
| 1021 | apstpmtsvi lgttttsrss tercppnary escacpasck sprpscgplc regcvcnpgf |
| 1081 | lfsdnhciqa sscncfynnd yyepgaewfs pnctehcrcw pgsrvecqis qcgthtvcql |
| 1141 | kngqygchpy agtatclvyg dphyvtfdgr hfgfmgkcty ilaqpcgnst dpffrvtakn |
| 1201 | eeqgqegvsc lskvyvtlpe stvtllkgrr tlyggqqvtl paipskgvfl gasgrfvelq |
| 1261 | tefglrvrwd gdqqlyvtvs stysgklcgl cgnydgnsdn dhlkldgspa gdkeelgnsw |
| 1321 | qtdqdedqec qkyqvvnsps cdsslqssms gpgfcgrlvd thgpfetcll hvkaasffds |
| 1381 | cmldmcgfqg lqhllcthms tmtttcqdag havkpwreph fcpmacppns kyslcakpcp |
| 1441 | dtchsgfsgm fcsdrcveac ecnpgfvlsg leciprsqcg clhpagsyfk vgerwykpgc |
| 1501 | kelcvcesnn rircqpwrcr aqefcgqqdg iygchaqgaa tctasgdphy ltfdgalhhf |
| 1561 | mgtctyvltr pcwsrsqdsy fvvsatnenr ggilevsyik avhvtvfdls isllrgckvm |
| 1621 | lnghrvalpv wlaqgrvtir lssnlvllyt nfglqvrydg shlvevtvps syggqlcglc |
| 1681 | gnynnnsldd nlrpdrklag dsmqlgaawk lpessepgcf lvggkpsscq ensmadawnk |
| 1741 | ncailinpqg pfsqchqvvp pgssfascvh gqcgtkgdtt alcrslqaya slcaqagqap |
| 1801 | awrnrtfcpm rcppgssysp csspcpdtcs sinnprdcpk alpcaescec qkghilsgts |
| 1861 | cvplgqcgct dpagsyhpvg erwytentct rlctcsvhnn itcfqstckp nqicwaldgl |
| 1921 | lhcrasgvgv cqlpgeshyv sfdgsnhsip dactivlvkv chpamalplf kisakhekee |
| 1981 | ggteafrlhe vyidiydaqv tlqkghrvli nskqvtipai sqipgvsvks ssiytivnik |
| 2041 | igvqvkfdgn hlleieiptt yygkvcgmcg nfndeeedel mmpsdevans dsefvnswkd |
| 2101 | kdidpscqsl pvdeqqipae qgenpsgncr aadlrrarek ceaalrapvw aqcasridlt |
| 2161 | pflvdcantl cefgglyqal cqalqafgat cqsqglkppl wrnssfcple cpayssytnc |
| 2221 | lpscspscwd ldgrcegakv psacaegcic qpgyvlsedk cvprsqcgck dahggsiplg |
| 2281 | kswvssgcte kcvctggaiq cgdfrcpsgs hcqltsdnsn sncvsdkseq csvygdpryl |
| 2341 | tfdgfsyrlq grmtyvlikt vdvlpegvep llvegrnkmd pprssiflqe vittvygykv |
| 2401 | qlqaglelvv nnqkmavpyr pnehlrvtlr gqrlylvtdf elvvsfygrk navislpsmy |
| 2461 | eglvsglcgn ydknrkndmm lpsgaltqnl ntfgnswevk tedallrfpr aipaeeegqg |
| 2521 | aelglrtglq vsecspeqla snstqacrvl adpqgpfaac hqtvapepfq ehcvldlcsa |
| 2581 | qdpreqeelr cqvlsghgvs sryhiselyd tlpsilcqpg rprglrgplr grlrqhprlc |
| 2641 | lqwhpeppla dcgctsngiy yqlgssflte dcsqrctcas srillcepfs cragevctlg |
| 2701 | nhtqgcfpes pclqnpcqnd gqcreggatf tcecevgygg glcmeprdap pprkpasnlv |
| 2761 | gvllgllvpv vvvllavtre ciyrtrrkre ktqegdrlar lvdtdtvldc ac |

| latent transforming growth factor beta binding | |
|---|---|
| protein 4 (LTBP4) (e.g., GenBank Accession Number | |
| A6NCG8 (SEQ ID NO: 5): | |
| mplanhrdde hgvasmvsvh vehpqeasvv vhqvervsgp weeadaeava | 50 |
| raeaaaraea aapytvlaqs apredgyoda sgfgycfrel rggecasplp | 100 |
| glrtqevccr gaglawgvhd cqlcserlgn servsapdgp cptgfervng | 150 |
| scedvdecat ggrcqhgeca ntrggytcvc pdgflldssr sscisqhvis | 200 |
| eakgpcfrvl rdggcslpil rnitkqiccc srvgkawgrg cqlcppfgse | 250 |
| gfreicpagp gyhysasdlr yntrplgqep prvslsqprt lpatsrpsag | 300 |
| flpthrlepr peprpdprpg pelplpsipa wtgpeipesg pssgmcqrnp | 350 |
| qvcgpgrcis rpsgytcacd sgfrispqgt rcidvdecrr vpppcapgrc | 400 |
| enspgsfrcv cgpgfragpr aaecldvdec hrvpppcdlg rcentpgsfl | 450 |
| cvcpagyqaa phgascqdvd ectqspglcg rgacknlpgs frcvcpagfr | 500 |
| gsaceedvde caqepppcgp grcdntagsf hcacpagfrs rgpgapcqdv | 550 |
| decarspppc tygrcenteg sfqcvcpmgf qpntagsece dvdecenhla | 600 |
| cpgqecvnsp gsfqcrtcps ghhlhrgrct dvdecssgap pcgphghctn | 650 |
| tegsfrcsca pgyrapsgrp gpcadvnecl egdfcfphge clntdgsfac | 700 |
| tcapgyrpgp rgascldvde cseedlcqsg ictntdgsfe cicppghrag | 750 |
| pdlascldvd ecrergpalc gsqrcenspg syrcvrdcdp gyhagpegtc | 800 |
| ddvdecqeyg peicgaqrce ntpgsyrctp acdpgyqptp gggcqdvdec | 850 |
| rnrsfcgaha vcqnlpgsfq clcdqyegar dgrhcvdvne cetlqgvcga | 900 |
| alcenvegsf lcvcpnspee fdpmtgrcvp prtsagtfpg sqpqapaspv | 950 |
| lparpppppl prrpstprqg pvgsgrrecy fdtaapdacd nilarnvtwq | 1000 |
| eccctvgegw gsgcriqqcp gtetaeyqsl cphgrgylap sgdlslrrdv | 1050 |
| decqlfrdqv cksgvcvnta pgyscycsng yyyhtqrlec idndecadee | 1100 |
| paceggrcvn tvgsyhctce pplvldgsqr rcvsnesqsl ddnlgvcwqe | 1350 |
| vgadlvcshp rldrqatyte ccclygeawg mdcalcpaqd sddfealcnv | 1200 |
| lrppaysppr pggfglpyey gpdlgppyqg lpygpelypp palpydpypp | 1250 |
| ppgpfarrea pygaprfdmp dfeddggpyg eseapappgp gtrwpyrsrd | 1300 |
| trrsfpepee ppeggsyags laepyeelea eecgildgct ngrcvrvpeg | 1350 |
| ftcrcfdgyr ldmtrmacvd inecdeaeaa splcvnarcl ntdgsfrcic | 1400 |
| rpgfapthqp hhcaparpra | 1420 |

| ASXL1 (additional sex combs like 1) (e.g., GenBank Accession | |
|---|---|
| Number Q8IXJ9-1 (SEQ ID NO: 6): | |
| 1 | mkdkqkkkke rtwaeaarlv lenysdapmt pkqilqviea eglkemrsgt splaclnaml |
| 61 | hsnsrggegl fyklpgrisl ftlkkdalqw srhpatvege epedtadves cgsneastvs |
| 121 | gendvsldet ssnascstes qsrplsnprd syrassgank qkkktgvmlp rvvltplkvn |
| 181 | gahvesasgf sgchadgesg spsssssgsl algsaairgq aevtqdpapl lrgfrkpatg |
| 241 | qmkrnrgeei dfetpgsilv ntnlralins rtfhalpshf qqqllfllpe vdrqvgtdgl |
| 301 | lrlsssalnn effthaaqsw rerladgeft hemqvrirqe mekekkveqw kekffedyyg |
| 361 | qklgltkees lqqnvgqeea eiksglcvpg esvriqrgpa trqrdghfkk rsrpdlrtra |
| 421 | rrnlykkqes eqagvakdak svasdvplyk dgeaktdpag lssphlpgts saapdlegpe |
| 481 | fpvesvasri qaepdnlara saspdripsl pgetvdqepk dqkrksfeqa asasfpekkp |
| 541 | rledrqsfrn tiesvhtekp qptkeepkvp piriqlsrik ppwvvkgqpt yqicpriipt |
| 601 | tesscrgwtg artladikar alqvrgargh hchreaatta igggggpggg gggatdeggg |
| 661 | rgsssgdgge acghpeprgg pstpgkctsd lqrtqllppy plngehtqag tamsrarred |
| 721 | lpslrkeesc llqratvglt dglgdasqlp vaptgdqpcq alpllssqts vaerlveqpq |
| 781 | lhpdvrtece sgttswesdd eeqgptvpad ngpipslvgd dtlekgtgqa ldshptmkdp |
| 841 | vnvtpsstpe ssptdclqnr afddelglgg scppmresdt rgenlktkal vsnsslhwip |
| 901 | ipsndevvkq pkpesrehip svepqvgeew ekaaptppal pgdltaeegl dpldsltslw |
| 961 | tvpsrggsds ngsycqqvdi eklkingdse alsphgestd tasdfeghlt edsseadtre |
| 1021 | aavtkgssvd kdekpnwnqs aplskvngdm rlvtrtdgmv apqswvsrvc avrqkipdsl |
| 1081 | llasteyqpr avclsmpgss veatnplvmq llqgslplek vlppanddsm sespqvpltk |
| 1141 | dqshgslrmg slhglgknsg mvdgsspssl ralkepllpd scetgtglar ieatqapgap |
| 1201 | qknckavpsf dslhpvtnpi tssrkleemd skeqfssfsc edqkevrams qdsnsnaapg |
| 1261 | kspgdlttsr tprfsspnvi sfgpeqtgra lgdqsnvtgq gkklfgsgnv aatlqrprpa |
| 1321 | dpmplpaeip pvfpsgklgp stnsmsggvq tpredwapkp hafvgsvkne ktfvggplka |
| 1381 | naenrkatgh splelvghle gmpfvmdlpf wklprepgkg lseplepssl paqlsikqaf |
| 1441 | yqklsklqls stsfnyssss ptfpkglags vvqlshkanf gashsaslsl qmftdsstve |
| 1501 | sislqcacsl kamimcqgcg afchddcigp sklcvlclvv r |

| beta globin (BBB) (e.g., GenBank Accession Number P68871 | |
|---|---|
| (SEQ ID NO: 7): | |
| 1 | mvhltpeeks avtalwgkvn vdevggealg rllvvypwtq rffesfgdls tpdavmgnpk |
| 61 | vkahgkkvlg afsdglahld nlkgtfatls elhcdklhvd penfrllgnv lvcvlahhfg |
| 121 | keftppvqaa yqkvvagvan alahkyh |

| BMP15-bone morphogenetic protein (e.g., GenBank Accession Number NM_005448.1 | |
|---|---|
| (see also, UniProt Accession Number O95972) (SEQ ID NO: 8): | |
| 1 | mvllsilril flcelvlfme hraqmaeggq ssiallaeap tlplieelle espgeqprkp |
| 61 | rllghslrym lelyrrsads hghprenrti gatmvrlvkp ltnvarphrg twhiqilgfp |
| 121 | lrpnrglyql vratvvyrhh lqltrfnlsc hvepwvqknp tnhfpssegd sskpslmsna |
| 181 | wkemditqlv qqrfwnnkgh rilrlrfmcq qqkdsgglel whgtssldia flllyfndth |
| 241 | ksirkakflp rgmeefmere sllrrtrqad gisaevtass skhsgpennq cslhpfqisf |
| 301 | rqlgwdhwii appfytpnyc kgtclrvlrd glnspnhaii qnlinqlvdq svprpscvpy |
| 361 | kyvpisvlmi eangsilyke yegmiaesct cr |

| TRIM49 (also known as RNF18; tripartite motif-containing 49) | |
|---|---|
| (e.g., GenBank Accession Number Q9NS80 (SEQ ID NO: 9): | |
| 1 | mnsgilqvfq gelicplcmn yfidpvtidc ghsfcrpcfy lnwqdipflv qcsectkste |
| 61 | qinlktnihl kkmaslarkv slwlflssee qmcgthretk kifcevdrsl lcllcsssqe |
| 121 | hryhrhrpie waaeehrekl lqkmqslwek acenhrnlnv ettrtrcwkd yvnlrleair |
| 181 | aeyqkmpafh heeekhnlem lkkkgkeifh rlhlskakma hrmeilrgmy eelnemchkp |
| 241 | dvellqafgd ilhrsesvll hmpqplnpel sagpitglrd rlngfrvhit lhheeanndi |
| 301 | flyeilrsmc igcdhqdvpy ftatprsfla wgvqtftsgk yywevhvgds wnwafgvcnm |
| 361 | yrkeknqnek idgkaglfll gcvkndiqcs lfttsplmlq yipkptsrvg lfldceaktv |
| 421 | sfvdvnqssl iytipncsfs pplrpifcci hf |

| DNAJ homolog subfamily B member 11 precursor (e.g., GenBank Accession | |
|---|---|
| Number Q9UBS4 (SEQ ID NO: 10): | |
| 1 | mapqnlstfc llllyligav iagrdfykil gvprsasikd ikkayrklal qlhpdrnpdd |
| 61 | pqaqekfqdl gaayevlsds ekrkqydtyg eeglkdghqs shgdifshff gdfgfmfggt |
| 121 | prqqdrnipr gsdiivdlev tleevyagnf vevvrnkpva rqapgkrkcn crqemrttql |
| 181 | gpgrfqmtqe vvcdecpnvk lvneertlev eiepgvrdgm eypfigegep hvdgepgdlr |
| 241 | frikvvkhpi ferrgddlyt nvtislvesl vgfemdithl dghkvhisrd kitrpgaklw |
| 301 | kkgeglpnfd nnnikgslii tfdvdfpkeq lteearegik qllkqgsvqk vynglqgy |

| uncharacterized hematopoietic stem/progenitor cells protein MDS027 | |
|---|---|
| (also known as MDS027 hHBrk1 HSPC300) (e.g., GenBank Accession Number | |
| Q9NZ47(SEQ ID NO: 11): | |
| 1 | mrgidtpsdr kkslkmslqa kwgpgldlsk strnwwvsnn ilwqphcqgm svltrtaphf |
| 61 | ppkvgrrqrl fteavqrq |

| uncharacterized protein ALB (e.g., GenBank Accession Number | |
|---|---|
| A6NBZ8 (SEQ ID NO: 12): | |
| mkwvtfisll flfssaysrg vfrrdahkse vahrfkdlge enfkalvlia | 50 |
| fagylqqcpf edhvklvnev tefaktcvad esaencdksl htlfgdklct | 100 |
| vatlretyge madccakqep ernecflqhk ddnpnlprlv rpevdvmcta | 150 |
| fhdneetflk kylyeiarrh pyfyapellf fakrykaaft eccqaadkaa | 200 |
| cllpkldelr degkassakq rlkcaslqkf gerafkawav arlsqrfpka | 250 |
| efaevsklvt dltkvhtecc hgdllecadd radlakyice nqdsissklk | 300 |
| eccekpllek shciaevend empadlpsla adfveskdvc knyaeakdvf | 350 |
| lgmflyeyar rhpdysvvll lrlaktyett lekccaaadp hecyakvfde | 400 |
| fkplveepqn likqncelfe qlgeykfqna llvrytkkvp qvstptlvev | 450 |
| srnlgkvgsk cckhpeakrm pcaedylsvv lnqlcvlhek tpvsdrvtkc | 500 |
| cteslvnrrp cfsalevdet yvpkefnaet ftfhadictl sekerqikkq | 550 |
| talvelvkhk pkatkeqlka vmddfaafve kcckaddket cfaeegqktc | 600 |
| cckssclrli tshlkasqpt mrirerk | 627 |

| isoform 3 of sushi, nidogen and EGF-like domain-containing protein 1 | |
|---|---|
| precursor (e.g., GenBank Accession Number Q8TER0-4 (SEQ ID NO: 13): | |
| 1 | mrhgvawall vaaalglgar gvrgavalad fypfgaergd avtpkqddgg sglrplsvpf |
| 61 | pffgaehsgl yvnnngiisf lkevsqftpv afpiakdrcv vaafwadvdn rragdvyyre |
| 121 | atdpamlrra tedvrhyfpe lldfnatwvf vatwyrvtff ggsssspvnt fqtvlitdgk |
| 181 | lsftifnyes ivwttgthas sggnatglgg iaaqagfnag dgqryfsipg srtadmaeve |
| 241 | tttnvgvpgr wafriddaqv rvggcghtts vclalrpcln ggkciddcvt gnpsytcscl |
| 301 | sgftgrrchl dvnecasqpc qnggtcthgi nsfrcqcpag fggptcetaq spcdtkecqh |
| 361 | ggqcqvengs avcvcqagyt gaacemdvdd cspdpclngg scvdlvgnyt clcaepfkgl |
| 421 | rcetgdhpvp daclsapchn ggtcvdadqg yvcecpegfm gldcrervpd dcecrnggrc |
| 481 | lganttlcqc plgffgllce feitampcnm ntqcpdggyc mehggsylcv chtdhnashs |
| 541 | lpspcdsdpc fnggscdahd dsytcecprg fhgkhcekar phlcssgpcr nggtckeagg |
| 601 | eyhcscpyrf tgrhceigkp dscasgpchn ggtcfhyigk ykcdcppgfs grhceiapsp |
| 661 | cfrspcvngg tcedrdtdff chcqagymgr rcqaevdcgp peevkhatlr fngtrlgava |
| 721 | lyacdrgysl sapsrirvcq phgvwseppq cleidecrsq pclhggscqd rvagylclcs |
| 781 | tgyegahcel erdecrahpc rnggscrnlp gayvcrcpag fvgvhcetev dacdsspcqh |
| 841 | ggrcesggga ylcvcpesff gyhcetvsdp cfsspcggrg yclasngshs ctckvgytge |
| 901 | dcakelfppt alkmervees gvsiswnppn gpaarqmldg yavtyvssdg syrrtdfvdr |
| 961 | trsshqlqal aagraynisv fsvkrnsnnk ndisrpavll artrprpveg fevtnvtast |
| 1021 | isvqwalhri rhatvsgvrv sirhpealrd qatdvdrsvd rftfrallpg krytiqlttl |
| 1081 | sglrgeehpt eslatapthv wtrplppanl taarvtatsa hvvwdaptpg slleayvinv |
| 1141 | ttsqstksry vpngklasyt vrdllpgrry qlsviavqst elgpqhsepa hlyiitsprd |
| 1201 | gadrrwhqgg hhprvlknrp pparlpelrl lndhsapetp tqpprfselv dgrgrvsarf |
| 1261 | ggspskaatv rsqptasaql enmeeapkrv slalqlpehg skdignvpgn csenpcqngg |
| 1321 | tcvpgadahs cdcgpgfkgr rcelacikvs rpctrlfset kafpvweggv chhvykrvyr |
| 1381 | vhqdicfkes cestslkktp nrkqsksqtl eks |

| isoform 2 of peripherin (e.g., GenBank Accession Number P41219-2 | |
|---|---|
| (SEQ ID NO: 14): | |
| 1 | mshhpsglra gfsstsyrrt fgpppslspg afsyssssrf sssrllgsas psssvrlgsf |
| 61 | rspragagal lrlpserldf smaealnqef latrsnekqe lqelndrfan fiekvrfleq |
| 121 | qnaalrgels qargqepara dqlcqqelre lrrelellgr erdrvqverd glaedlaalk |
| 181 | qrleeetrkr edaehnlvlf rkdvddatls rlelerkies lmdeieflkk lheeelrdlq |
| 241 | vsvesqqvqq veveatvkpe ltaalrdira qyesiaaknl qeaeewyksk yadlsdaanr |
| 301 | nhealrqakq emnesrrqiq sltcevdglr gtneallrql releeqfale aggyqagaar |
| 361 | leeelrqlke emarhlreyq ellnvkmald ieiatyrkll egeesrisvp vhsfaslnik |
| 421 | ttvpeveppq dshsrktvli ktietrngev vtesqkeqrs eldkssahsy |

| mitochondrial 28S ribosomal protein S22 (e.g., GenBank Accession | |
|---|---|
| Number P82650 (SEQ ID NO: 15): | |
| 1 | maplgttvll wsllrsspgv ervcfrariq pwhggllqpl pcsfemglpr rrfsseaaes |
| 61 | gspetkkptf mdeevqsilt kmtglnlqkt fkpaiqelkp ptyklmtqaq leeatrqave |
| 121 | aakvrlkmpp vleervpind vlaedkileg tettkyvftd isysiphrer fivvrepsgt |
| 181 | lrkasweerd rmiqvyfpke grkiltpiif keenlrtmys qdrhvdvlnl cfagfepdst |
| 241 | eyikvhhkty edidkrgkyd llrstryfgg mvwyfvnnkk idgllidqiq rdliddatnl |
| 301 | vqlyhvlhpd gqsaqgakdq aaeginlikv fakteaqkga yieltlqtyq ealsrhsaas |

| translation initiation factor EIF-2B subunit epsilon (e.g., GenBank | |
|---|---|
| Accession Number Q13144 (SEQ ID NO: 16): | |
| 1 | maapvvappg vvvsrankrs gagpggsggg gargaeeepp pplqavlvad sfdrrffpis |
| 61 | kdqprvllpl anvalidytl efltatgvqe tfvfccwkaa qikehllksk wcrptslnvv |
| 121 | riitselyrs lgdvlrdvda kalvrsdfll vygdvisnin itraleehrl rrkleknvsv |
| 181 | mtmifkessp shptrchedn vvvavdsttn rvlhfqktqg lrrfafplsl fqgssdgvev |
| 241 | rydlldchis icspqvaqlf tdnfdyqtrd dfvrgllvne eilgnqihmh vtakeygarv |
| 301 | snlhmysavc advirrwvyp ltpeanftds ttqscthsrh niyrgpevsl ghgsileenv |
| 361 | llgsgtvigs ncfitnsvig pgchigdnvv ldqtylwqgv rvaagaqihq sllcdnaevk |
| 421 | ervtlkprsv ltsqvvvgpn itlpegsvis lhppdaeede ddgefsddsg adqekdkvkm |
| 481 | kgynpaevga agkgylwkaa gmnmeeeeel qqnlwglkin meeesesese qsmdseepds |
| 541 | rggspqmddi kvfqnevlgt lqrgkeenis cdnlvleins lkyaynislk evmqvlshvv |
| 601 | lefplqqmds pldssrycal llpllkawsp vfrnyikraa dhlealaaie dfflehealg |
| 661 | ismakvlmaf yqleilaeet ilswfsqrdt tdkgqqlrkn qqlqrfiqwl keaeeessed |
| 721 | d |

| estradiol 17-beta-dehydrogenase 1 (e.g., GenBank Accession Number | |
|---|---|
| P14061 (SEQ ID NO: 17): | |
| 1 | martvvlitg cssgiglhla vrlasdpsqs fkvyatlrdl ktqgrlweaa ralacppgsl |
| 61 | etlqldvrds ksvaaarerv tegrvdvlvc naglgllgpl ealgedavas vldvnvvgtv |
| 121 | rmlqaflpdm krrgsgrvlv tgsvgglmgl pfndvycask faleglcesl avlllpfgvh |
| 181 | lsliecgpvh tafmekvlgs peevldrtdi htfhrfyqyl ahskqvfrea aqnpeevaev |
| 241 | fltalrapkp tlryftterf lpllrmrldd psgsnyvtam hrevfgdvpa kaeagaeagg |
| 301 | gagpgaedea grsavgdpel gdppaapq |

| XRCC6BP1 (e.g., GenBank Accession Number Q8N4L5 (SEQ ID NO: 18): | |
|---|---|
| 1 | magapderrr gpaageqlqq qhvscqvfpe rlaqgnpqqg ffssfftcnq kcqlrllktl |
| 61 | etsrshdlea vvpqngsetg warkglgntw pgasgsaqsl drlgimgagl ga |

| brain-specific angiogenesis inhibitor 1 precursor (e.g., GenBank | |
|---|---|
| Accession Number O14514(SEQ ID NO: 19): | |
| 1 | mrgqaaapgp vwilapllll llllgrrara aagadagpgp epcatlvqgk ffgyfsaaav |
| 61 | fpanasrcsw tlrnpdprry tlymkvakap vpcsgpgrvr tyqfdsfles trtylgvesf |
| 121 | devlrlcdps aplaflqask qflqmrrqqp pqhdglrpra gppgptddfs veylvvgnrn |
| 181 | psraacqmlc rwldaclags rsshpcgimq tpcaclggea ggpaagplap rgdvclrdav |
| 241 | aggpenclts ltqdrgghga tggwklwslw gectrdcggg lqtrtrtclp apgvegggce |
| 301 | gvleegrqcn reacgpagrt ssrsqslrst darrreelgd elqqfgfpap qtgdpaaeew |
| 361 | spwsvcsstc gegwqtrtrf cvsssystqc sgplreqrlc nnsavcpvhg awdewspwsl |
| 421 | csstcgrgfr drtrtcrppq fggnpcegpe kqtkfcnial cpgravdgnw newsswsacs |
| 481 | ascsqgrqqr trecngpsyg gaecqghwve trdcflqqcp vdgkwqawas wgscsvtcga |
| 541 | gsqrrervcs gpffggaacq gpqdeyrqcg tqrcpephei cdednfgavi wketpageva |
| 601 | avrcprnatg lilrrcelde egiayweppt yircvsidyr niqmmtrehl akaqrglpge |
| 661 | gvseviqtlv eisqdgtsys gdllstidvl rnmteifrra yysptpgdvq nfvqilsnll |
| 721 | aeenrdkwee aqlagpnake lfrlvedfvd vigfrmkdlr dayqvtdnlv lsihklpasg |
| 781 | atdisfpmkg wratgdwakv pedrvtvsks vfstgltead easvfvvgtv lyrnlgsfla |
| 841 | lqrnttvlns kvisvtvkpp prslrtplei efahmyngtt nqtcilwdet dvpsssappq |
| 901 | lgpwswrgcr tvpldalrtr clcdrlstfa ilaqlsadan mekatlpsvt livgcgvssl |
| 961 | tllmlviiyv svwryirser svilinfcls iissnalili gqtqtrnkvm ctlvaaflhf |
| 1021 | fflssfcwvl teawqsymav tghlrnrlir krflclgwgl palvvaisvg ftkakgystm |
| 1081 | nycwlslegg llyafvgpaa avvlvnmvig ilvfnklvsk dgitdkklke ragaslwssc |
| 1141 | vvlpllaltw msavlavtdr rsalfqilfa vfdslegfvi vmvhcilrre vqdavkcrvv |
| 1201 | drqeegngds ggsfqnghaq lmtdfekdvd lacrsvlnkd iaacrtatit gtlkrpslpe |
| 1261 | eeklklahak gpptnfnslp anvsklhlhg sprypggplp dfpnhsltlk rdkapkssfv |
| 1321 | gdgdifkkld selsraqeka ldtsyvilpt atatlrpkpk eepkysihid qmpqtrlihl |
| 1381 | stapeaslpa rsppsrqpps ggppeappaq pppppppppp ppqqplpppp nlepappslg |
| 1141 | dpgepaahpg pstgpstkne nvatlsvssl errksryael dfekimhtrk rhqdmfqdln |
| 1501 | rklqhaaekd kevlgpdskp ekqqtpnkrp weslrkahgt ptwvkkelep lqpsplelrs |
| 1561 | vewersgati plvgqdiidl qtev |

| isoform 2 of ring finger and CCCH-type zinc finger domain-containing | |
|---|---|
| protein 2 (e.g., GenBank Accession Number Q9HBD1-2 (SEQ ID NO: 20): | |
| 1 | mpvqaaqwte flscpicyne fdenvhkpis lgcshtvckt clnklhrkac pfdqtaintd |
| 61 | idvlpvnfal lqlvgaqvpd hqsiklsnlg enkhyevakk cvedlalylk plsggkyvas |
| 121 | lnqsalsrpm qrklvtlvnc qlveeegrvr amraarslge rtvtelilqh qnpqqlsanl |
| 181 | waavrargcq flgpamqeea lklvllaled gsalsrkvlv lfvvqrlepr fpqasktsig |
| 241 | hvvqllyras cfkvtkrded sslmqlkeef rsyealrreh daqivhiame aglrispeqw |
| 301 | ssllygdlah kshmqsiidk lqspesfaks vqeltivlqr tgdpanlnrl rphlellani |
| 361 | dpnpdavspt weqlenamva vktvvhglvd fiqnysrkgh etpqpqpnsk yktsmcrdlr |
| 421 | qqggcprgtn ctfahsqeel ekyrlrnkki natvrtfpll nkvgvnntvt ttagnvisvi |
| 481 | gstettgkiv pstngisnae nsvsqlisrs tdstlralet vkkvgkvgan gqnaagpsad |
| 541 | svtenkigsp pktpvsnvaa tsagpsnvgt elnsvpqkss pfltrvpvyp phseniqyfq |
| 601 | dprtqipfev pqypqtgyyp ppptvpagva pcvprfvrsn nvpesslppa smpyadhyst |
| 661 | fsprdrmnss pyqppppqpy gpvppvpsgm yapvydsrri wrppmyqrdd iirsnslppm |
| 721 | dvmhssvyqt slrerynsld gyysvacqpp seprttvplp repcghlkts ceeqirrkpd |
| 781 | qwaqyhtqka plvsstlpva tqsptppspl fsvdfradfs esvsgtkfee dhlshyspws |
| 841 | cgtigscina idsepkdvia nsnavlmdld sgdvkrrvhl fetqrrtkee dpiipfsdgp |
| 901 | iiskwgaisr ssrtgyhttd pvqatasqgs atkpisvsdy vpyvnavdsr wssygneats |
| 961 | sahyverdrf ivtdlsghrk hsstgdllsl elqqaksnsl llqreanala mqqkwnslde |
| 1021 | grhltlnlls keielrngel qsdytedatd tkpdrdiele lsaldtdepd gqsepieeil |
| 1081 | diqlgissqn dqllngmave nghpvqqhqk eppkqkkqsl gedhvileeq ktilpvtscf |
| 1141 | sqplpvsisn asclpittsv sagnlilkth vmsedkndfl kpvangkmvn s |

| hemoglobin subunit beta (e.g., GenBank Accession Number P68871 | |
|---|---|
| (SEQ ID NO: 21): | |
| 1 | mvhltpeeks avtalwgkvn vdevggealg rllvvypwtq rffesfgdls tpdavmgnpk |
| 61 | vkahgkkvlg afsdglahld nlkgtfatls elhcdklhvd penfrllgnv lvcvlahhfg |
| 121 | keftppvqaa yqkvvagvan alahkyh |

| isoform 1 of far upstream element-binding protein 1 (e.g., GenBank | |
|---|---|
| Accession Number Q96AE4-1 (SEQ ID NO: 22): | |
| 1 | madystvppp ssgsaggggg ggggggvnda fkdalqrarq iaakiggdag tslnsndygy |
| 61 | ggqkrpledg dqpdakkvap qndsfgtqlp pmhqqqsrsv mteeykvpdg mvgfiigrgg |
| 121 | egisriqqes gckiqiapds gglperscml tgtpesvqsa krlldqivek grpapgfhhg |
| 181 | dgpgnavqei mipaskaglv igkggetikq lqeragvkmv miqdgpqntg adkplritgd |
| 241 | pykvqqakem vlelirdqgg frevrneygs riggnegidv piprfavgiv igrngemikk |
| 301 | iqndagvriq fkpddgttpe riaqitgppd rcqhaaeiit dllrsvqagn pggpgpggrg |
| 361 | rgrgqgnwnm gppgglqefn fivptgktgl iigkggetik sisqqsgari elqrnpppna |
| 421 | dpnmklftir gtpqqidyar qlieekiggp vnplgppvph gphgvpgphg ppgppgpgtp |
| 481 | mgpynpapyn pgppgpaphg ppapyapqgw gnayphwqqq appdpakagt dpnsaawaay |
| 541 | yahyyqqqaq pppaapagap tttqtngqgd qqnpapagqv dytkaweeyy kkmgqavpap |
| 601 | tgappggqpd ysaawaeyyr qqaayyaqts pqgmpqhppa pqgq |

| GALECTIN-3 (e.g., GenBank Accession Number P17931 (SEQ ID NO: 23): | |
|---|---|
| 1 | madnfslhda lsgsgnpnpq gwpgawgnqp agaggypgas ypgaypgqap pgaypgqapp |
| 61 | gayhgapgay pgapapgvyp gppsgpgayp ssggpsapga ypatgpygap agplivpynl |
| 121 | plpggvvprm litilgtvkp nanrialdfq rgndvafhfn prfnennrry ivcntkldnn |
| 181 | wgreergsvf pfesgkpfki qvlvepdhfk vavndahllq ynhrvkklne isklgisgdi |
| 241 | dltsasytmi |

| lysozyme C precursor (e.g., GenBank Accession Number P61626 (SEQ ID NO: 24): | |
|---|---|
| 1 | mkalivlglv llsvtvqgkv fercelartl krlgmdgyrg islanwmcla kwesgyntra |
| 61 | tnynagdrst dygifqinsr ywcndgktpg avnachlscs allqdniada vacakrvvrd |
| 121 | pqgirawvaw rnrcqnrdvr qyvqgcgv |

| actin, alpha skeletal muscle (e.g., GenBank Accession Number | |
|---|---|
| P68133 (SEQ ID NO: 25): | |
| 1 | mcdedettal vcdngsglvk agfagddapr avfpsivgrp rhqgvmvgmg qkdsyvgdea |
| 61 | qskrgiltlk ypiehgiitn wddmekiwhh tfynelrvap eehptlltea plnpkanrek |
| 121 | mtqimfetfn vpamyvaiqa vlslyasgrt tgivldsgdg vthnvpiyeg yalphaimrl |
| 181 | dlagrdltdy lmkiltergy sfvttaerei vrdikeklcy valdfenema taasssslek |
| 241 | syelpdgqvi tignerfrcp etlfqpsfig mesagihett ynsimkcdid irkdlyannv |
| 301 | msggttmypg iadrmqkeit alapstmkik iiapperkys vwiggsilas lstfqqmwit |
| 361 | kqeydeagps ivhrkcf |

| isoform M2 of pyruvate kinase isozymes M1/M2 (e.g., GenBank Accession | |
|---|---|
| Number P14618-1 (SEQ ID NO: 26): | |
| 1 | mskphseagt afiqtqqlha amadtflehm crldidsppi tarntgiict igpasrsvet |
| 61 | lkemiksgmn varlnfshgt heyhaetikn vrtatesfas dpilyrpvav aldtkgpeir |
| 121 | tglikgsgta evelkkgatl kitldnayme kcdenilwld yknickvvev gskiyvddgl |
| 181 | islqvkqkga dflvteveng gslgskkgvn lpgaavdlpa vsekdiqdlk fgveqdvdmv |
| 241 | fasfirkasd vhevrkvlge kgknikiisk ienhegvrrf deileasdgi mvargdlgie |
| 301 | ipaekvflaq kmmigrcnra gkpvicatqm lesmikkprp traegsdvan avldgadcim |
| 361 | lsgetakgdy pleavrmqhl iareaeaaiy hlqlfeelrr lapitsdpte atavgaveas |
| 421 | fkccsgaiiv ltksgrsahq varyrprapi iavtrnpqta rqahlyrgif pvlckdpvqe |
| 481 | awaedvdlry nfamnvgkar gffkkgdvvi vltgwrpqsg ftntmrvvpv p |

| AGR2 (e.g., GenBank Accession Number O95994 (SEQ ID NO: 27): | |
|---|---|
| 1 | mekipvsafl llvalsytla rdttvkpgak kdtkdsrpkl pqtlsrgwgd qliwtqtyee |
| 61 | alyksktsnk plmiihhlde cphsqalkkv faenkeiqkl aeqfvllnlv yettdkhlsp |
| 121 | dgqyvprimf vdpsltvrad itgrysnrly ayepadtall ldnmkkalkl lktel |

| neutrophil defensin 1 precursor (e.g., GenBank Accession Number | |
|---|---|
| P59665 (SEQ ID NO: 28); | |
| 1 | mrtlailaai llvalqaqae plqaradeva aapeqiaadi pevvvslawd eslapkhpgs |
| 61 | rknmacycri paciagerry gtciyqgrlw afcc |

| myeloblastin precursor (e.g., GenBank Accession Number | |
|---|---|
| P24158 (SEQ ID NO: 29): | |
| 1 | mahrppspal asvllallls gaaraaeivg gheaqphsrp ymaslqmrgn pgshfcggtl |
| 61 | ihpsfvltaa hclrdipqrl vnvvlgahnv rtqeptqqhf svaqvflnny daenklndvl |
| 121 | liqlsspanl sasvatvqlp qqdqpvphgt qclamgwgrv gahdppaqvl qelnvtvvtf |
| 181 | fcrphnictf vprrkagicf gdsggplicd giiqgidsfv iwgcatrlfp dfftrvalyv |
| 241 | dwirstlrrv eakgrp |

| uncharacterized protein PSME2 (e.g., GenBank Accession Number | |
|---|---|
| Q9UL46 (SEQ ID NO: 30): | |
| makpcgvrls gearkqvevf rqnlfgeaee flyrflpqki iylnqllqed | 50 |
| slnvadltsl rapldipipd pppkddemet dkqekkevpk cgflpgnekv | 100 |
| lsllalvkpe vwtlkekcil vitwiqhlip kiedgndfgv aiqekvlerv | 150 |
| navktkveaf qttiskyfse rgdavakask ethvmdyral vherdeaayg | 200 |
| elramvldlr afyaelyhii ssnlekivnp kgeekpsmy | 239 |

| tubulin beta-2C chain (e.g., GenBank Accession Number | |
|---|---|
| P68371 (SEQ ID NO: 31): | |
| 1 | mreivhlqag qcgnqigakf wevisdehgi dptgtyhgds dlqlerinvy yneatggkyv |
| 61 | pravlvdlep gtmdsvrsgp fgqifrpdnf vfgqsgagnn wakghytega elvdsvldvv |
| 121 | rkeaescdcl qgfqlthslg ggtgsgmgtl liskireeyp drimntfsvv pspkvsdtvv |
| 181 | epynatlsvh qlventdety cidnealydi cfrtlklttp tygdlnhlvs atmsgvttcl |
| 241 | rfpgqlnadl rklavnmvpf prlhffmpgf apltsrgsqq yraltvpelt qqmfdaknmm |
| 301 | aacdprhgry ltvaavfrgr msmkevdeqm lnvqnknssy fvewipnnvk tavcdipprg |
| 361 | lkmsatfign staiqelfkr iseqftamfr rkaflhwytg egmdemefte aesnmndlvs |
| 421 | eyqqyqdata eeegefeeea eeeva |

| thiosulfate sulfurtransferase (e.g., GenBank Accession Number | |
|---|---|
| Q16762 (SEQ ID NO: 32): | |
| 1 | mvhqvlyral vstkwlaesi rtgklgpglr vldaswyspg trearkeyle rhvpgasffd |
| 61 | ieecrdtasp yemmlpseag faeyvgrlgi snhthvvvyd gehlgsfyap rvwwmfrvfg |
| 121 | hrtvsvlngg frnwlkeghp vtsepsrpep avfkatldrs llktyeqvle nleskrfqlv |
| 181 | dsrsqgrflg tepepdavgl dsghirgavn mpfmdflted gfekgpeelr alfqtkkvdl |
| 241 | sqpliatcrk gvtachvala aylcgkpdva vydgswsewf rrappesrvs qgkseka |

| heat shock 70 kDa protein 1 (e.g., GenBank Accession Number | |
|---|---|
| P08107 (SEQ ID NO: 33): | |
| 1 | makaaaigid lgttyscvgv fqhgkveiia ndqgnrttps yvaftdterl igdaaknqva |
| 61 | lnpqntvfda krligrkfgd pvvqsdmkhw pfqvindgdk pkvqvsykge tkafypeeis |
| 121 | smvltkmkei aeaylgypvt navitvpayf ndsqrqatkd agviaglnvl riineptaaa |
| 181 | iaygldrtgk gernvlifdl gggtfdvsil tiddgifevk atagdthlgg edfdnrlvnh |
| 241 | fveefkrkhk kdisqnkrav rrlrtacera krtlssstqa sleidslfeg idfytsitra |
| 301 | rfeelcsdlf rstlepveka lrdakldkaq ihdlvlvggs tripkvqkll qdffngrdln |
| 361 | ksinpdeava ygaavqaail mgdksenvqd lllldvapls lgletaggvm talikrnsti |
| 421 | ptkqtqiftt ysdnqpgvli qvyegeramt kdnnllgrfe lsgippaprg vpqievtfdi |
| 481 | dangilnvta tdkstgkank ititndkgrl skeeiermvq eaekykaede vqrervsakn |
| 541 | alesyafnmk savedeglkg kiseadkkkv ldkcqevisw ldantlaekd efehkrkele |
| 601 | qvcnpiisgl yqgaggpgpg gfgaqgpkgg sgsgptieev d |

| Ig kappa chain V-III region sie (e.g., GenBank Accession | |
|---|---|
| Number P01620 (SEQ ID NO: 34) | |
| 1 | eivitqspgt lslspgerat lscrasqsys nsylawyqqk pgqaprlliy gassratgip |
| 61 | drfsgsgsgt dftltisrle pddfavyycq qygsspqtfg qgskveikr |

| macrophage migration inhibitory factor (e.g., GenBank Accession | |
|---|---|
| Number P14174 (SEQ ID NO: 35): | |
| 1 | mpmfivntnv prasvpdgfl seltqqlaqa tgkppqyiav hvvpdqlmaf ggssepcalc |
| 61 | slhsigkigg aqnrsyskll cgllaerlri spdrvyinyy dmnaanvgwn nstfa |

| isoform 1 of ATP synthase subunit D, mitochondrial (e.g., GenBank | |
|---|---|
| Accession Number O75947-1 (SEQ ID NO: 36): | |
| 1 | magrklalkt idwvafaeii pqnqkaiass lkswnetlts rlaalpenpp aidwayykan |
| 61 | vakaglvddf ekkfnalkvp vpedkytaqv daeekedvks caewvslska riveyekeme |
| 121 | kmknlipfdq mtiedlneaf petkldkkky pywphqpien l |

| uncharacterized protein ENSP00000374051 (e.g., GenBank Accession | |
|---|---|
| Number A6NGM3 (SEQ ID NO: 37): | |
| mvvdknkrlt kggkkgakkk vvdpfskkdw ydvnapamfn irnigktlvt | 50 |
| rtqgtkiasd grvfevslad lqndevafrk fklitedvqg kncltnfhgv | 100 |
| dltsdkmcsm vkkwqtmiea hvdvkttdgy llrlfcvgft kkrnnqirkt | 150 |
| syaqhqqvlt sqirkkmmei mtrevqtndl kevvnklipd sigkdvekac | 200 |
| qsiyplhdvf vrkvkmlkkp kfelgklmel hgegcssgka tgdetgvkve | 250 |
| radgyelpvq esv | 263 |

| isocitrate dehydrogenase [NADP] cytoplasmic (e.g., GenBank Accession | |
|---|---|
| Number O75874 (SEQ ID NO: 38): | |
| 1 | mskkisggsv vemqgdemtr iiwelikekl ifpyveldlh sydlgienrd atndqvtkda |
| 61 | aeaikkhnvg vkcatitpde krveefklkq mwkspngtir nilggtvfre aiickniprl |
| 121 | vsgwvkpiii grhaygdqyr atdfvvpgpg kveitytpsd gtqkvtylvh nfeegggvam |
| 181 | gmynqdksie dfahssfqma lskgwplyls tkntilkkyd grfkdifqei ydkqyksqfe |
| 241 | aqkiwyehrl iddmvaqamk seggfiwack nydgdvqsds vaqgygslgm mtsvlvcpdg |
| 301 | ktveaeaahg tvtrhyrmyq kgqetstnpi asifawtrgl ahrakldnnk elaffanale |
| 361 | evsietieag fmtkdlaaci kglpnvqrsd ylntfefmdk lgenlkikla qakl |

| hemoglobin subunit delta (e.g., GenBank Accession Number P02042 | |
|---|---|
| (SEQ ID NO: 39): | |
| 1 | mvhltpeekt avnalwgkvn vdavggealg rllvvypwtq rffesfgdls spdavmgnpk |
| 61 | vkahgkkvlg afsdglahld nlkgtfsqls elhcdklhvd penfrllgnv lvcvlarnfg |
| 121 | keftpqmqaa yqkvvagvan alahkyh |

| isoform 1 of splicing factor, arginine/serine-rich 7 (e.g., GenBank | |
|---|---|
| Accession Number Q16629-1 (SEQ ID NO: 40): | |
| 1 | msrygrygge tkvyvgnlgt gagkgelera fsyygplrtv wiarnppgfa fvefedprda |
| 61 | edavrgldgk vicgsrvrve lstgmprrsr fdrpparrpf dpndrcyecg ekghyaydch |
| 121 | rysrrrrsrs rsrshsrsrg rrysrsrsrs rgrrsrsasp rrsrsislrr srsaslrrsr |
| 181 | sgsikgsryf qspsrsrsrs rsisrprssr sksrspspkr srspsgsprr saspermd |

| isoform 1 of mRNA-capping enzyme (e.g., GenBank Accession Number | |
|---|---|
| O60942-1 (SEQ ID NO: 41): | |
| 1 | mahnkipprw lncprrgqpv agrflplktm lgprydsqva eenrfhpsml snylkslkvk |
| 61 | mgllvdltnt srfydrndie kegikyiklq ckghgecptt entetfirlc erfnernppe |
| 121 | ligvhcthgf nrtgflicaf lvekmdwsie aavatfaqar ppgiykgdyl kelfrrygdi |
| 181 | eeappppllp dwcfeddede dededgkkes epgssasfgk rrkerlklga iflegvtvkg |
| 241 | vtqvttqpkl gevqqkchqf cgwegsgfpg aqpvsmdkqn iklldlkpyk vswkadgtry |
| 301 | mmlidgtnev fmidrdnsvf hvsnlefpfr kdlrmhlsnt lldgemiidr vngqavpryl |
| 361 | iydiikfnsq pvgdcdfnvr lqciereiis prhekmktgl idktqepfsv rnkpffdict |
| 421 | srkllegnfa kevshemdgl ifqptgkykp grcddilkwk ppslnsvdfr lkitrmggeg |
| 481 | llpqnvglly vggyerpfaq ikvtkelkqy dnkiieckfe nnswvfmrqr tdksfpnayn |
| 541 | tamavcnsis npvtkemlfe fidrctaasq gqkrkhhldp dtelmppppp krprplt |

| LON protease homolog, mitochondrial precursor (e.g., GenBank Accession | |
|---|---|
| Number P36776 (SEQ ID NO: 42): | |
| 1 | maastgyvrl wgaarcwvlr rpmlaaaggr vptaagawll rgqrtcdasp pwalwgrgpa |
| 61 | iggqwrgfwe assrgggafs ggedasegga eegaggaggs agagegpvit altpmtipdv |
| 121 | fphlpliait rnpvfprfik iievknkklv ellrrkvrla qpyvgvflkr ddsnesdvve |
| 181 | sldeiyhtgt faqihemqdl gdklrmivmg hrrvhisrql evepeepeae nkhkprrksk |
| 241 | rgkkeaedel sarhpaelam eptpelpaev lmvevenvvh edfqvteevk altaeivkti |
| 301 | rdiialnply resvlqmmqa gqrvvdnpiy lsdmgaaltg aeshelqdvl eetnipkrly |
| 361 | kalsllkkef elsklqqrlg reveekikqt hrkyllqeql kiikkelgle kddkdaieek |
| 421 | frerlkelvv pkhvmdvvde elsklglldn hssefnvtrn yldwltsipw gkysnenldl |
| 481 | araqavleed hygmedvkkr ilefiavsql rgstqgkilc fygppgvgkt siarsiaral |
| 541 | nreyfrfsvg gmtdvaeikg hrrtyvgamp gkiiqclkkt ktenplilid evdkigrgyq |
| 601 | gdpssallel ldpegnanfl dhyldvpvdl skvlfictan vtdtipeplr drmeminvsg |
| 661 | yvaqeklaia erylvpqara lcgldeskak lssdvltlli kqycresgvr nlqkqvekvl |
| 721 | rksaykivsg eaesvevtpe nlqdfvgkpv ftvermydvt ppgvvmglaw tamggstlfv |
| 781 | etslrrpqdk dakgdkdgsl evtgqlgevm kesariaytf araflmqhap andylvtshi |
| 841 | hlhvpegatp kdgpsagcti vtallslamg rpvrqnlamt gevsltgkil pvggikekti |
| 901 | aakragvtci vlpaenkkdf ydlaafiteg levhfvehyr eifdiafpde qaealaver |

| signal recognition particle 54 kDa protein (e.g., GenBank Accession | |
|---|---|
| Number P61011 (SEQ ID NO: 43): | |
| 1 | mvladlgrki tsalrslsna tiineevlna mlkevctall eadvniklvk qlrenvksai |
| 61 | dleemasgln krkmiqhavf kelvklvdpg vkawtptkgk qnvimfvglq gsgktttcsk |
| 121 | layyyqrkgw ktclicadtf ragafdqlkq natkaripfy gsytemdpvi iasegvekfk |
| 181 | nenfeiiivd tsgrhkqeds lfeemlqvan aiqpdnivyv mdasigqace aqakafkdkv |
| 241 | dvasvivtkl dghakgggal savaatkspi ifigtgehid dfepfktqpf iskllgmgdi |
| 301 | eglidkvnel klddnealie klkhgqftlr dmyeqfqnim kmgpfsqilg mipgfgtdfm |
| 361 | skgneqesma rlkklmtimd smndqeldst dgakvfskqp griqrvargs gvstrdvqel |
| 421 | ltqytkfaqm vkkmggikgl fkggdmsknv sqsqmaklnq qmakmmdprv lhhmggmagl |
| 481 | qsmmrqfqqg aagnmkgmmg fnnm |

| isoform long of galectin-9 (e.g., GenBank Accession Number O00182-1 | |
|---|---|
| (SEQ ID NO: 44): | |
| 1 | mafsgsqapy lspavpfsgt iqgglqdglq itvngtvlss sgtrfavnfq tgfsgndiaf |
| 61 | hfnprfedgg yvvcntrqng swgpeerkth mpfqkgmpfd lcflvqssdf kvmvngilfv |
| 121 | qyfhrvpfhr vdtisvngsv qlsyisfqnp rtvpvqpafs tvpfsqpvcf pprprgrrqk |
| 181 | ppgvwpanpa pitqtvihtv qsapgqmfst paippmmyph paypmpfitt ilgglypsks |
| 241 | illsgtvlps aqrfhinlcs gnhiafhlnp rfdenavvrn tqidnswgse erslprkmpf |
| 301 | vrgqsfsvwi lceahclkva vdgqhlfeyy hrlrnlptin rlevggdiql thvqt |

| integrin-linked protein kinase (e.g., GenBank Accession Number Q13418 | |
|---|---|
| (SEQ ID NO: 45): | |
| 1 | mddiftqcre gnavavrlwl dntendlnqg ddhgfsplhw acregrsavv emlimrgari |
| 61 | nvmnrgddtp lhlaashghr divqkllqyk adinavnehg nvplhyacfw gqdqvaedlv |
| 121 | angalvsicn kygempvdka kaplrellre raekmgqnln ripykdtfwk gttrtrprng |
| 181 | tlnkhsgidf kqlnfltkln enhsgelwkg rwqgndivvk vlkvrdwstr ksrdfneecp |
| 241 | rlrifshpnv lpvlgacqsp paphptlith wmpygslynv lhegtnfvvd qsqavkfald |
| 301 | margmaflht lepliprhal nsrsvmided mtarismadv kfsfqcpgrm yapawvapea |
| 361 | lqkkpedtnr rsadmwsfav llwelvtrev pfadlsnmei gmkvaleglr ptippgisph |
| 421 | vcklmkicmn edpakrpkfd mivpilekmq dk |

| bifunctional aminoacyl-tRNA synthetase (e.g., GenBank Accession Number | |
|---|---|
| P07814 (SEQ ID NO: 46): | |
| 1 | matlsltvns gdpplgalla vehvkddvsi sveegkenil hvsenviftd vnsilrylar |
| 61 | vattaglygs nlmehteidh wlefsatkls scdsftstin elnhclslrt ylvgnslsla |
| 121 | dlcvwatlkg naawqeqlkq kkapvhvkrw fgfleaqqaf qsvgtkwdvs ttkarvapek |
| 181 | kqdvgkfvel pgaemgkvtv rfppeasgyl highakaall nqhyqvnfkg klimrfddtn |
| 241 | pekekedfek viledvamlh ikpdqftyts dhfetimkya ekliqegkay vddtpaeqmk |
| 301 | aereqriesk hrknpieknl qmweemkkgs qfghscclra kidmssnngc mrdptlyrck |
| 361 | iqphprtgnk ynvyptydfa cpivdsiegv thalrtteyh drdeqfywii ealgirkpyi |
| 421 | weysrlnlnn tvlskrkltw fvneglvdgw ddprfptvrg vlrrgmtveg lkqfiaaqgs |
| 481 | srsvvnmewd kiwafnkkvi dpvapryval lkkevipvnv peaqeemkev akhpknpevg |
| 541 | lkpvwyspkv fiegadaetf segemvtfin wgnlnitkih knadgkiisl dakfnlenkd |
| 601 | ykkttkvtwl aetthalpip vicvtyehli tkpvlgkded fkqyvnknsk heelmlgdpc |
| 661 | lkdlkkgdii qlqrrgffic dqpyepvspy sckeapcvli yipdghtkem ptsgskektk |
| 721 | veatknetsa pfkerptpsl nnncttseds lvlynrvavq gdvvrelkak kapkedvdaa |
| 781 | vkqllslkae ykektgqeyk pgnppaeigq nissnssasi leskslydev aaqgevvrkl |
| 841 | kaekspkaki neavecllsl kaqykektgk eyipgqppls qssdssptrn sepagletpe |
| 901 | akvlfdkvas qgevvrklkt ekapkdqvdi avqellqlka qyksligvey kpvsatgaed |
| 961 | kdkkkkeken ksekqnkpqk qndgqrkdps knqggglsss gagegqgpkk qtrlgleakk |
| 1021 | eenladwysq vitksemiey hdisgcyilr pwayaiweai kdffdaeikk lgvencyfpm |
| 1081 | fvsqsaleke kthvadfape vawvtrsgkt elaepiairp tsetvmypay akwvqshrdl |
| 1141 | piklnqwcnv vrwefkhpqp flrtreflwq eghsafatme eaaeevlqil dlyaqvyeel |
| 1201 | laipvvkgrk tekekfaggd ytttieafis asgraiqggt shhlgqnfsk mfeivfedpk |
| 1261 | ipgekqfayq nswglttrti gvmtmvhgdn mglvlpprva cvqvviipcg itnalseedk |
| 1321 | ealiakcndy rrrllsvnir vradlrdnys pgwkfnhwel kgvpirlevg prdmkscqfv |
| 1381 | avrrdtgekl tvaeneaetk lqailediqv tlftrasedl kthmvvantm edfqkildsg |
| 1441 | kivqipfcge idcedwikkt tardqdlepg apsmgakslc ipfkplcelq pgakcvcgkn |
| 1501 | pakyytlfgr sy |

| isoform 1 of zinc finger protein 207 (e.g., GenBank Accession Number | |
|---|---|
| O43670-1 (SEQ ID NO: 47): | |
| 1 | mgrkkkkqlk pwcwycnrdf ddekiliqhq kakhfkchic hkklytgpgl aihcmqvhke |
| 61 | tidavpnaip grtdieleiy gmegipekdm derrrlleqk tqesqkkkqq ddsdeydddd |
| 121 | saastsfqpq pvqpqqgyip pmaqpglppv pgapgmppgi pplmpgvppl mpgmppvmpg |
| 181 | mppgmmpmgg mmppgpgipp lmpgmppgmp ppvprpgipp mtqaqavsap gilnrppapt |
| 241 | atvpapqppv tkplfpsagq mgtpvtssst assnseslsa sskalfpsta qaqaavqgpv |
| 301 | gtdfkplnst pattteppkp tfpaytqsta sttsttnsta akpaasitsk patltttsat |
| 361 | sklihpdedi sleerraqlp kyqrnlprpg qapignppvg piggmmppqp gipqqqgmrp |
| 421 | pmpphgqygg hhqgmpgylp gamppygqgp pmvppyqggp prppmgmrpp vmsqggry |

| inorganic pyrophosphatase (e.g., GenBank Accession Number Q15181 | |
|---|---|
| (SEQ ID NO: 48): | |
| 1 | msgfsteera apfsleyrvf lknekgqyis pfhdipiyad kdvfhmvvev prwsnakmei |
| 61 | atkdplnpik qdvkkgklry vanlfpykgy iwnygaipqt wedpghndkh tgccgdndpi |
| 121 | dvceigskvc argeiigvkv lgilamideg etdwkviain vddpdaanyn dindvkrlkp |
| 181 | gyleatvdwf rrykvpdgkp enefafnaef kdkdfaidii ksthdhwkal vtkktngkgi |
| 241 | scmnttlses pfkcdpdaar aivdalpppc esactvptdv dkwfhhqkn |

| calponin-2 (e.g., GenBank Accession Number Q99439 (SEQ ID NO: 49): | |
|---|---|
| 1 | msstqfnkgp syglsaevkn rllskydpqk eaelrtwieg ltglsigpdf qkglkdgtil |
| 61 | ctlmnklqpg svpkinrsmq nwhqlenlsn fikamvsygm npvdlfeand lfesgnmtqv |
| 121 | qvsllalagk aktkglqsgv digvkysekq ernfddatmk agqcviglqm gtnkcasqsg |
| 181 | mtaygtrrhl ydpknhilpp mdhstislqm gtnkcasqvg mtapgtrrhi ydtklgtdkc |
| 241 | dnssmslqmg ytqganqsgq vfglgrqiyd pkycpqgtva dgapsgtgdc pdpgevpeyp |
| 301 | pyyqeeagy |

| isoform 1 of muscleblind-like protein 3 (e.g., GenBank Accession | |
|---|---|
| Number Q9NUK0-1 (SEQ ID NO: 50): | |
| 1 | mtavnvalir dtkwltlevc refqrgtcsr adadckfahp prvchvengr vvacfdslkg |
| 61 | rctrenckyl hppphlktql eingrnnliq qktaaamfaq qmqlmlqnaq msslgsfpmt |
| 121 | psipanppma fnpyiphpgm glvpaelvpn tpvlipgnpp lampgavgpk lmrsdklevc |
| 181 | refqrgnctr gendcryahp tdasmieasd ntvticmdyi kgrcsrekck yfhppahlqa |
| 241 | rlkaahhqmn hsaasamalq pgtlqlipkr salekpngat pvfnptvfhc qqaltnlqlp |
| 301 | qpafipagpi lcmapasniv pmmhgatptt vsaattpats vpfaapttgn qlkf |

| cathepsin G precursor (e.g., GenBank Accession Number P08311 | |
|---|---|
| (SEQ ID NO: 51): | |
| 1 | mqplllllaf llptgaeage iiggresrph srpymaylqi qspagqsrcg gflvredfvl |
| 61 | taahcwgsni nvtlgahniq rrentqqhit arrairhpqy nqrtiqndim llqlsrrvrr |
| 121 | nrnvnpvalp raqeglrpgt lctvagwgrv smrrgtdtlr evqlrvqrdr qclrifgsyd |
| 181 | prrqicvgdr rerkaafkgd sggpllcnnv ahgivsygks sgvppevftr vssflpwirt |
| 241 | tmrsfklldq metpl |

| zinc finger and BTB domain-containing protein 34 (e.g., GenBank | |
|---|---|
| Accession Number Q8NCN2 (SEQ ID NO: 52): | |
| 1 | msvemdsssf iqfdvpeyss tvlsqlnelr lqgklcdiiv hiqgqpfrah kavlaasspy |
| 61 | frdhsalstm sglsisvikn pnvfeqllsf cytgrmslql kdvvsfltaa sflqmqcvid |
| 121 | kctqilesih skisvgdvds vtvgaeenpe srngvkdssf fanpveispp ycsqgrqpta |
| 181 | ssdlrmettp skalrsrlqe eghsdrgssg syseyeiqie gdheqgdllv resqitevkv |
| 241 | kmeksdrpsc sdssslgddg yhtemvdgeq vvavnvgsyg svlqhaysys qaasqptnvs |
| 301 | eafgslsnss psrsmlscfr ggrarqkral svhlhsdlqg lvqgsdseam mnnpgyessp |
| 361 | rersarghwy pynerliciy cgksfnqkgs ldrhmrlhmg icpfvckfcg kkytrkdqle |
| 421 | yhirghtddk pfrceicgkc fpfqgtlnqh lrknhpgvae vrsriesper tdvyveqkle |
| 481 | ndasasemgl dsrmeihtvs dapd |

| adenine phosphoribosyltransferase (e.g., GenBank Accession | |
|---|---|
| Number P07741 (SEQ ID NO: 53): | |
| 1 | madselqlve qrirsfpdfp tpgvvfrdis pvlkdpasfr aaigllarhl kathggridy |
| 61 | iagldsrgfl fgpslaqelg lgcvlirkrg klpgptlwas ysleygkael eiqkdalepg |
| 121 | qrvvvvddll atggtmnaac ellgrlqaev lecvslvelt slkgreklap vpffsllqye |

| 40S ribosomal protein S9 (e.g., GenBank Accession Number P46781 | |
|---|---|
| (SEQ ID NO: 54): | |
| 1 | mpvarswvcr ktyvtprrpf eksrldqelk ligeyglrnk revwrvkftl akirkaarel |
| 61 | ltldekdprr lfegnallrr lvrigvldeg kmkldyilgl kiedflerrl qtqvfklgla |
| 121 | ksihharvli rqrhirvrkq vvnipsfivr ldsqkhidfs lrspygggrp grvkrknakk |
| 181 | gqggagagdd eeed |

| TALIN-1 (e.g., GenBank Accession Number Q9Y490 (SEQ ID NO: 55): | |
|---|---|
| 1 | mvalslkisi gnvvktmqfe pstmvydacr iireripeap agppsdfglf lsdddpkkgi |
| 61 | wleagkaldy ymlrngdtme yrkkqrplki rmldgtvkti mvddsktvtd mlmticarig |
| 121 | itnhdeyslv relmeekkee itgtlrkdkt llrdekkmek lkqklhtdde lnwldhgrtl |
| 181 | reqgveehet lllrrkffys dqnvdsrdpv qlnllyvqar ddilngshpv sfdkacefag |
| 241 | fqcqiqfgph neqkhkagfl dlkdflpkey vkqkgerkif qahkncgqms eieakvryvk |
| 301 | larslktygv sfflvkekmk gknklvprll gitkecvmrv dektkeviqe wnltnikrwa |
| 361 | aspksftldf gdyqdgyysv qttegeqiaq liagyidiil kkkkskdhfg legdeestml |
| 421 | edsvspkkst vlqqqynrvg kvehgsvalp aimrsgasgp enfqvgsmpp aqqqitsgqm |
| 481 | hrghmpplts aqqaltgtin ssmqavqaaq atlddfdtlp plgqdaaska wrknkmdesk |
| 541 | heihsqvdai tagtasvvnl tagdpaetdy tavgcavtti ssnltemsrg vkllaalled |
| 601 | eggsgrpllq aakglagays ellrsaqpas aeprqnllqa agnvgqasge llqqigesdt |
| 661 | dphfqdalmq lakavasaaa alvlkaksva qrtedsglqt qviaaatqca lstsqlvact |
| 721 | kvvaptissp vcqeqlveag rlvakavegc vsasqaated gqllrgvgaa atavtqalne |
| 781 | llqhvkahat gagpagrydq atdtiltvte nifssmgdag emvrqarila qatsdlvnai |
| 841 | kadaegesdl ensrkllsaa kiladatakm veaakgaaah pdseeqqqrl reaaeglrma |
| 901 | tnaaaqnaik kklvqrleha akqaaasatq tiaaaqhaas tpkasagpqp llvqsckava |
| 961 | eqipllvqgv rgsqaqpdsp saqlaliaas qsflqpggkm vaaakasvpt iqdqasamql |
| 1021 | sqcaknlgta laelrtaaqk aqeacgplem dsalsvvqnl ekdlqevkaa ardgklkplp |
| 1081 | getmekctqd lgnstkavss aiaqllgeva qgnenyagia ardvagglrs laqaargvaa |
| 1141 | ltsdpavqai vldtasdvld kasslieeak kaaghpgdpe sqqrlaqvak avtqalnrcv |
| 1201 | sclpgqrdvd nalravgdas krllsdslpp stgtfqeaqs rlneaaagln qaatelvqas |
| 1261 | rgtpqdlara sgrfgqdfst fleagvemag qapsqedraq vvsnlkgism sssklllaak |
| 1321 | alstdpaapn lksqlaaaar avtdsinqli tmctqqapgq kecdnalrel etvrellenp |
| 1381 | vqpindmsyf gcldsvmens kvlgeamtgi sqnakngnlp efgdaistas kalcgfteaa |
| 1441 | aqaaylvgvs dpnsqagqqg lveptqfara nqaiqmacqs lgepgctqaq vlsaativak |
| 1501 | htsalcnscr lasarttnpt akrqfvqsak evanstanlv ktikaldgaf teenraqcra |
| 1561 | ataplleavd nlsafasnpe fssipaqisp egraamepiv isaktmlesa ggliqtaral |
| 1621 | avnprdppsw svlaghsrtv sdsikklits mrdkapgqle cetaiaalns clrdldqasl |
| 1681 | aavsqqlapr egisqealht qmltavqeis hlieplanaa raeasqlghk vsqmaqyfep |
| 1741 | ltlaavgaas ktlshpqqma lldqtktlae salqllytak eaggnpkqaa htqealeeav |
| 1801 | qmmteavedl tttlneaasa agvvggmvds itqainqlde gpmgepegsf vdyqttmvrt |
| 1861 | akaiavtvqe mvtksntspe elgplanqlt sdygrlasea kpaavaaene eigshikhrv |
| 1921 | qelghgcaal vtkagalqcs psdaytkkel iecarrvsek vshvlaalqa gnrgtqacit |
| 1981 | aasavsgiia dldttimfat agtlnregte tfadhregil ktakvlvedt kvlvqnaags |
| 2041 | qeklaqaaqs svatitrlad vvklgaaslg aedpetqvvl inavkdvaka lgdlisatka |
| 2101 | aagkvgddpa vwqlknsakv mvtnvtsllk tvkavedeat kgtraleatt ehirqelavf |
| 2161 | cspeppakts tpedfirmtk gitmatakav aagnscrqed viatanlsrr aiadmlrack |
| 2221 | eaayhpevap dvrlralhyg recangylel ldhvlltlqk pspelkqqlt ghskrvagsv |
| 2281 | teliqaaeam kgtewvdped ptviaenell gaaaaieaaa kkleqlkpra kpkeadesln |
| 2341 | feeqileaak siaaatsalv kaasaaqrel vaqgkvgaip analddgqws qglisaarmv |
| 2401 | aaatnnlcea anaavqghas qeklissakq vaastaqllv ackvkadqds eamkrlqaag |
| 2461 | navkrasdnl vkaaqkaaaf eeqenetvvv kekmvggiaq iiaaqeemlr kereleeark |
| 2521 | klaqirqqqy kflpselrde h |

| leucine-rich repeat-containing protein 59 (e.g., GenBank | |
|---|---|
| Accession Number Q96AG4 (SEQ ID NO: 56): | |
| 1 | mtkagskggn lrdkldgnel dlslsdlnev pvkelaalpk atildlscnk lttlpsdfcg |
| 61 | lthlvkldls knklqqlpad fgrlvnlqhl dllnnklvtl pvsfaqlknl kwldlkdnpl |
| 121 | dpvlakvagd cldekqckqc ankvlqhmka vqadqererq rrlevereae kkreakqrak |
| 181 | eaqerelrkr ekaeekerrr keydalkaak reqekkpkke anqapksksg srprkppprk |
| 241 | htrswavlkl llllllfgva gglvacrvte lqqqplctsv ntiydnavqg lrrheilqwv |
| 301 | lqtdsqq |

| ATP synthase subunit alpha, mitochondrial precursor (e.g., | |
|---|---|
| GenBank Accession Number P25705 (SEQ ID NO: 57): | |
| 1 | mlsvrvaaav vralprragl vsrnalgssf iaarnfhasn thlqktgtae mssileeril |
| 61 | gadtsvdlee tgrvlsigdg iarvhglrnv qaeemvefss glkgmslnle pdnvgvvvfg |
| 121 | ndklikegdi vkrtgaivdv pvgeellgrv vdalgnaidg kgpigsktrr rvglkapgii |
| 181 | prisvrepmq tgikavdslv pigrgqreli igdrqtgkts iaidtiinqk rfndgsdekk |
| 241 | klyciyvaig qkrstvaqlv krltdadamk ytivvsatas daaplqylap ysgcsmgeyf |
| 301 | rdngkhalii yddlskqava yrqmslllrr ppgreaypgd vfylhsrlle raakmndafg |
| 361 | ggsltalpvi etqagdvsay iptnvisitd gqifletelf ykgirpainv glsysrvgsa |
| 421 | aqtramkqva gtmklelaqy revaafaqfg sdldaatqql lsrgvrltel lkqgqyspma |
| 481 | ieeqvaviya gvrgyldkle pskitkfena flshvvsqhq allgtiradg kiseqsdakl |
| 541 | keivtnflag fea |

| isoform 7 of protein transport protein SEC31A (e.g., | |
|---|---|
| GenBank Accession Number O94979-7 (SEQ ID NO: 58): | |
| 1 | mklkevdrta mqawspagnh piylatgtsa qqldatfstn asleifeldl sdpsldmksc |
| 61 | atfssshryh kliwgpykmd skgdvsgvli aggengniil ydpskiiagd kevviaqndk |
| 121 | htgpvraldv nifqtnlvas ganeseiyiw dlnnfatpmt pgaktqpped isciawnrqv |
| 181 | qhilasasps gratvwdlrk nepiikvsdh snrmhcsgla whpdvatqmv laseddrlpv |
| 241 | iqmwdlrfas splrvlenha rgilaiawsm adpelllscg kdakilcsnp ntgevlyelp |
| 301 | tntqwcfdiq wcprnpavls aasfdgrisv ysimggstdg lrqkqvdkls ssfgnldpfg |
| 361 | tgqplpplqi pqqtaqhsiv lplkkppkwi rrpvgasfsf ggklvtfenv rmpshqgaeq |
| 421 | qqqqhhvfis qvvtekefls rsdqlqqavg sqgfinycqk kidasqtefe knvwsflkvn |
| 481 | feddsrgkyl ellgyrkedl gkkialalnk vdganvalkd sdqvaqsdge espaaeeqll |
| 541 | gehikeekee seflpssggt fnisvsgdid glitqalltg nfesavdlcl hdnrmadaii |
| 601 | laiaggqell artqkkyfak sqskitrlit avvmknwkei vescdlknwr ealaavltya |
| 661 | kpdefsalcd llgtrleneg dsllqtqacl cyicagnvek lvacwtkaqd gshplslqdl |
| 721 | iekvvilrka vqltqamdts tvgvllaakm sqyanllaaq gsiaaalafl pdntnqpnim |
| 781 | qlrdrlcraq gepvaghesp kipyekqqlp kyrpgpvagh hqmprvqtqq yyphgenppp |
| 841 | pgfimhgnvn pnaagqlpts pghmhtqvpp ypqpqpyqpa qpypfgtggs amyrpqqpva |
| 901 | pptsnaypnt pyissassyt gqsqlyaaqh gassptsspa tsfppppssg asfqhggpga |
| 961 | ppsssayalp pgttgtlpaa selpasqrtg pqngwndppa lnrvpkkkkm penfmppvpi |
| 1021 | tspimnplgd pqsqmlqqqp sapvplssqs sfpqphlpgg qpfhgvqqpl gqtgmppsfs |
| 1081 | kpniegapga pigntfqhvq slptkkitkk pipdehlilk ttfedliqrc lssatdpqtk |
| 1141 | rklddaskrl eflydklreq tlsptitsgl hniarsietr nysegltmht hivstsnfse |
| 1201 | tsafmpvlkv vltqanklgv |

| dihydroxyacetone kinase (e.g., GenBank Accession Number Q3LXA3 | |
|---|---|
| (SEQ ID NO: 59): | |
| 1 | mtskklvnsv agcaddalag lvacnpnlql lqghrvalrs dldslkgrva llsgggsghe |
| 61 | pahagfigkg mltgviagav ftspavgsil aairavaqag tvgtallvkn ytgdrlnfgl |
| 121 | areqaraegi pvemvvigdd saftvlkkag rrglcgtvli hkvagalaea gvgleeiakq |
| 181 | vnvvtkamgt lgvslsscsv pgskptfels adevelglgi hgeagvrrik matadeivkl |
| 241 | mldhmtnttn ashvpvqpgs svvmmvnnlg glsflelgii adatvrsleg rgvkiaralv |
| 301 | gtfmsalemp gisltlllvd epllklidae ttaaawpnva avsitgrkrs rvapaepqea |
| 361 | pdstaaggsa skrmalvler vcstllglee hlnaldraag dgdcgtthsr aaraiqewlk |
| 421 | egpppaspaq llsklsvlll ekmggssgal yglfltaaaq plkaktslpa wsaamdagle |
| 481 | amqkygkaap gdrtmldslw aagqelqawk spgadllqvl tkavksaeaa aeatknmeag |
| 541 | agrasyissa rleqpdpgav aaaailrail evlqs |

similar to heterogeneous nuclear ribonucleoproteins C1/C2 (HNRNP Cl/HNRNP
C2). ISOFORM 4 ENSEMBL Accession Number ENST0000342709 (SEQ ID NO: 60)(see
also, GenBank Accession No.: NM_004500.3 and UNIPARC Accession Number
IPI00868835):
masnvtnktdprsmnsrvfignlntlvvkksdveaifskygkivgcsvhkgfaffqyvnernaraavagedgrmiagq
vldinlaaepkvnrgkagykrsaaemygssfdldcdfqrdyydrmysyparvpppppiaraaikreltqikqkvdsfl
enlekiekeqskqavemnnvkseeeqssssvkkdetnvkmeseggaddsaeegdllddddnedggmtsws

18 kDa protein UNIPARC Accession Number IPI00796554 (SEQ ID NO: 61):
marsrtsssp aisqetevgg grkaiiifvp vpqlksfqki qvrlvrelek kfsgkhvvfi
aqrrilpkpt qksrtknkqk cprsrtltav hdafledlvf pseivgkrip vkldssrlik
vhldkaqqnn vehkvetfsg vykkltgkdv nfefpefql

| cold agglutinin FS-1 L-chain (e.g., GenBank Accession Number A2NB45: | |
|---|---|
| (SEQ ID NO: 62) | |
| divmtqspls lpvtpgepas iscrssqsll hsngfnylhw ylqkpgqspr | 50 |
| lliylgsnra sgvpdrfsgs gsgtdftlki srveaddvgi yycmqalqsp | 100 |
| ytfgqgtkle ikr | 113 |

| isoform 1 of heterogeneous nuclear ribonucleoprotein d0 (e.g., | |
|---|---|
| GenBank Accession Number Q14103-1 (SEQ ID NO: 63): | |
| 1 | mseeqfggdg aaaaataavg gsageqegam vaatqgaaaa agsgagtggg tasggteggs |
| 61 | aesegakida skneedeghs nssprhseaa taqreewkmf igglswdttk kdlkdyfskf |
| 121 | gevvdctlkl dpitgrsrgf gfvlfkeses vdkvmdqkeh klngkvidpk rakamktkep |
| 181 | vkkifvggls pdtpeekire yfggfgeves ielpmdnktn krrgfcfitf keeepvkkim |
| 241 | ekkyhnvgls kceikvamsk eqyqqqqqwg srggfagrar grgggpsqnw nqgysnywnq |
| 301 | gygnygynsq gyggyggydy tgynnyygyg dysnqqsgyg kvsrrgghqn sykpy |

| DAZAP1/MEF2D fusion protein (e.g., GenBank Accession Number | |
|---|---|
| Q5IRN2 (SEQ ID NO: 64): | |
| 1 | mnnsgadeig klfvggldws ttqetlrsyf sqygevvdcv imkdkttnqs rgfgfvkfkd |
| 61 | pncvgtvlas rphtldgrni dpkpctprgm qpertrpkeg wqkgprsdns ksnkifvggi |
| 121 | phncgetelr eyfkkfgvvt evvmiydaek qrprgngyvs araspgllpv angnslnkvi |
| 181 | paksppppth stqlgapsrk pdlrvitsqa gkglmhhlte dhldlnnaqr lgvsqsthsl |
| 241 | ttpvvsvatp sllsqglpfs smptayntdy qltsaelssl pafsspggls lgnvtawqqp |
| 301 | qqpqqpqqpq ppqqqppqpq qpqpqqpqqp qqppqqqshl vpvslsnlip gsplphvgaa |
| 361 | ltvtthphis iksepvspsr erspappppa vfpaarpepg dglsspaggs yetgdrddgr |
| 421 | gdfgptlgll rpapepeaeg savkrmrldt wtlk |

| POTE2 (e.g., GenBank Accession Number NP 001077007 (SEQ ID NO: 65)): | |
|---|---|
| 1 | mvvevdsmpa assvkkpfgl rskmgkwccr cfpcyresgk snvgtsgdhd dsamktlrsk |
| 61 | mgkwchhcfp ccrgsgksnv gasgdhddsa mktlrnkmgk wcchcfpccr gsgkskvgaw |
| 121 | gdyddsafme pryhvrgedl dklhraawwg kvprkdlivm lrdtdvnkkd kqkrtalhla |
| 181 | sangnsevvk llldrrcqln vldnkkrtal ikavqcqede calmllehgt dpnipdeygn |
| 241 | ttlhyaiyne dklmakalll ygadiesknk hgltplllgv heqkqqvvkf likkkanlna |
| 301 | ldrygrtali lavccgsasi vsllleqnid vssqdlsgqt areyavsshh hvicqllsdy |
| 361 | kekqmlkiss ensnpeqelk ltseeesqrf kgsensqpek msqeleinkd gdreveeemk |
| 421 | khesnnvgll enltngvtag ngdnglipqr ksrtpenqqf pdneseeyhr icellsdyke |
| 481 | kqmpkyssen snpeqdlklt seeesqrlkg sengqpekrs qepeinkdgd relenfmaie |
| 541 | emkkhgsthv gfpenltnga tagngddgli pprksrtpes qqfpdtenee yhsdeqndtq |
| 601 | kqfceeqntg ilhdeilihe ekqievvekm nselslsckk ekdvlhenst lreeiamlrl |
| 661 | eldtmkhqsq lrekkyledi esvkkkndnl lkalqlnelt mdddtavlvi dngsgmckag |
| 721 | fagddaprav fpsivgrprq qgmmggmhqk esyvgkeaqs krgiltlkyp mehgiitnwd |
| 781 | dmekiwhhtf ynelrvapee hpillteapl npkanrekmt qimfetfntp amyvaiqavp |
| 841 | slytsgrttg ivmdsgdgvt htvpiyegna lphatlrldl agrelpdylm kiltergyrf |
| 901 | ttmaereivr dikeklcyva ldfeqemata assssleksy elpdgqviti gnerfrcpea |
| 961 | lfqpcflgme scgihettfn simksdvdir kdlytntvls ggttmypgma hrmqkeiaal |
| 1021 | apsmmkirii appkrkysvw vggsilasls tfqqmwiskq eydesgpsiv hrkcf |

| Keratin 18 (KRT18) (e.g., GenBank Accession Number NP 000215 | |
|---|---|
| (SEQ ID NO: 66)): | |
| 1 | msfttrstfs tnyrslgsvq apsygarpvs saasvyagag gsgsrisvsr stsfrggmgs |
| 61 | gglatgiagg lagmggiqne ketmqslndr lasyldrvrs letenrrles kirehlekkg |
| 121 | pqvrdwshyf kiiedlraqi fantvdnari vlqidnarla addfrvkyet elamrqsven |
| 181 | dihglrkvid dtnitrlqle teiealkeel lfmkknheee vkglqaqias sgltvevdap |
| 241 | ksqdlakima diraqydela rknreeldky wsqqieestt vvttqsaevg aaettltelr |
| 301 | rtvqsleidl dsmrnlkasl enslrevear yalqmeqlng illhlesela qtraegqrqa |
| 361 | qeyeallnik vkleaeiaty rrlledgedf nlgdaldssn smqtiqkttt rrivdgkvvs |
| 421 | etndtkvlrh |

| PSME4 Isoform 1 of Proteasome activator complex subunit 4 (e.g., | |
|---|---|
| GenBank Accession Number NP 055429 (SEQ ID NO: 67)): | |
| 1 | mepaeragvg eppepggrpe pgprgfvpqk eivynkllpy aerldaesdl glaqikcnlg |
| 61 | ravqlqelwp gglfwtrkls tyirlygrkf skedhvlfik llyelvsipk leismmqgfa |
| 121 | rllinllkkk ellsradlel pwrplydmve rilysktehl glnwfpnsve nilktlvksc |
| 181 | rpyfpadata emleewrplm cpfdvtmqka ityfeiflpt slppelhhkg fklwfdelig |
| 241 | lwvsvqnlpq wegqlvnlfa rlatdnigyi dwdpyvpkif trilrslnlp vgssqvlvpr |
| 301 | fltnaydigh aviwitammg gpsklvqkhl aglfnsitsf yhpsnngrwl nklmkllqrl |
| 361 | pnsvvrrlhr erykkpswlt pvpdshkltd qdvtdfvqci iqpvllamfs ktgsleaaqa |
| 421 | lqnlalmrpe lvippvlert ypaletltep hqltatlscv igvarslvsg grwfpegpth |
| 481 | mlpllmralp gvdpndfskc mitfqfiatf stlvplvdcs svlqerndlt everelcsat |
| 541 | aefedfvlqf mdrcfglies stleqtreet etekmthles lvelglsstf stiltqcske |
| 601 | ifmvalqkvf nfstshifet rvagrmvadm craavkccpe eslklfvphc csvitqltmn |
| 661 | ddvlndeeld kellwnlqll seitrvdgrk lllyreqlvk ilqrtlhltc kqgytlscnl |
| 721 | lhhllrsttl iypteycsvp ggfdkppsey fpikdwgkpg dlwnlgiqwh vpsseevsfa |
| 781 | fylldsflqp elvklqhcgd gklemsrddi lqsltivhnc ligsgnllpp lkgepvtnlv |
| 841 | psmvsleetk lytgleydls renhreviat virkllnhil dnseddtksl fliikiigdl |
| 901 | lqfqgshkhe fdsrwksfnl vkksmenrlh gkkqhirall idrvmlghel rtltvegcey |
| 961 | kkihqdmird llrlstssys qvrnkaqqtf faalgaynfc crdiiplvle flrpdrqgvt |
| 1021 | qqqfkgalyc llgnhsgvcl anlhdwdciv qtwpaivssg lsqamslekp sivrlfddla |
| 1081 | ekihrqyeti gldftipksc veiaellqqs knpsinqill spekikegik rqqeknadal |
| 1141 | rnyenlvdtl ldgveqrnlp wkfehigigl lflllrddrv lplrairffv enlnhdaivv |
| 1201 | rkmaisavag ilkqlkrthk kltinpceis gcpkptqiia gdrpdnhwlh ydsktiprtk |
| 1261 | kewesscfve kthwgyytwp knmvvyagve eqpklgrsre dmteaeqiif dhfsdpkfve |
| 1321 | qlitflsled rkgkdkfnpr rfclfkgifr nfddaflpvl kphlehlvad shestqrcva |
| 1381 | eiiaglirgs khwtfekvek lwellcpllr talsnitvet yndwgaciat scesrdprkl |
| 1441 | hwlfellles plsgeggsfv dacrlyvlqg glaqqewrvp ellhrllkyl epkltqvykn |
| 1501 | vrerigsvlt yifmidvslp nttptisphv peftarilek lkplmdvdee iqnhvmeeng |
| 1561 | igeedertqg ikllktilkw lmasagrsfs tavteqlqll plffkiapve ndnsydelkr |
| 1621 | daklclslms qgllyphqvp lvlqvlkqta rssswharyt vltylqtmvf ynlfiflnne |
| 1681 | davkdirwlv islledeqle vremaattls gllqcnfltm dspmqihfeq lcktklpkkr |
| 1741 | krdpgsvgdt ipsaelvkrh agvlglgacv lsspydvptw mpqllmnlsa hlndpqpiem |
| 1801 | tvkktlsnfr rthhdnwqeh kqqftddqll vltdllvspc yya |

| Mitogen-activated protein kinase-activated protein kinase (MAPKAPK3) | |
|---|---|
| (e.g., GenBank Accession Number NP 004626 (SEQ ID NO: 68)): | |
| 1 | mdgetaeeqg gpvpppvapg gpglggapgg rrepkkyavt ddyqlskqvl glgvngkvle |
| 61 | cfhrrtgqkc alkllydspk arqevdhhwq asggphivci ldvyenmhhg krclliimec |
| 121 | meggelfsri qergdqafte reaaeimrdi gtaiqflhsh niahrdvkpe nllytskekd |
| 181 | avlkltdfgf akettqnalq tpcytpyyva pevlgpekyd kscdmwslgv imyillcgfp |
| 241 | pfysntgqai spgmkrrirl gqygfpnpew sevsedakql irlllktdpt erltitqfmn |
| 301 | hpwinqsmvv pqtplhtarv lqedkdhwde vkeemtsala tmrvdydqvk ikdlktsnnr |
| 361 | llnkrrkkqa gsssasqgcn nq |

| Complement component 1, s subcomponent (C1S) (e.g., | |
|---|---|
| GenBank Accession Number NP 001725 (SEQ ID NO: 69)): | |
| 1 | mwcivlfsll awvyaeptmy geilspnypq aypseveksw dievpegygi hlyfthldie |
| 61 | lsencaydsv qiisgdteeg rlcgqrssnn phspiveefq vpynklqvif ksdfsneerf |
| 121 | tgfaayyvat dinectdfvd vpcshfcnnf iggyfcscpp eyflhddmkn cgvncsgdvf |
| 181 | taligeiasp nypkpypens rceyqirlek gfqvvvtlrr edfdveaads agncldslvf |
| 241 | vagdrqfgpy cghgfpgpln ietksnaldi ifqtdltgqk kgwklryhgd pmpcpkedtp |
| 301 | nsvwepakak yvfrdvvqit cldgfevveg rvgatsfyst cqsngkwsns klkcqpvdcg |
| 361 | ipesiengkv edpestlfgs virytceepy yymengggge yhcagngswv nevlgpelpk |
| 421 | cvpvcgvpre pfeekqriig gsdadiknfp wqvffdnpwa ggalineywv ltaahvvegn |
| 481 | reptmyvgst svqtsrlaks kmltpehvfi hpgwkllevp egrtnfdndi alvrlkdpvk |
| 541 | mgptvspicl pgtssdynlm dgdlglisgw grtekrdrav rlkaarlpva plrkckevkv |
| 601 | ekptadaeay vftpnmicag gekgmdsckg dsggafavqd pndktkfyaa glvswgpqcg |
| 661 | tyglytrvkn yvdwimktmq enstpred |

| Lysozyme C precursor (LYZ) (e.g., GenBank Accession Number NP 000230 | |
|---|---|
| (SEQ ID NO: 70)): | |
| 1 | mkalivlglv llsvtvqgkv fercelartl krlgmdgyrg islanwmcla kwesgyntra |
| 61 | tnynagdrst dygifqinsr ywcndgktpg avnachlscs allqdniada vacakrvvrd |
| 121 | pqgirawvaw rnrcqnrdvr qyvqgcgv |

| Keritin Type Cytoskeletal 20 (KRT20) (e.g., GenBank Accession Number | |
|---|---|
| NP 061883 (SEQ ID NO: 71)): | |
| 1 | mdfsrrsfhr slssslqapv vstvgmqrlg ttpsvyggag grgirisnsr htvnygsdlt |
| 61 | gggdlfvgne kmamqnlndr lasylekvrt leqsnsklev qikqwyetna pragrdysay |
| 121 | yrqieelrsq ikdaqlqnar cvlqidnakl aaedfrlkye tergirltve adlqglnkvf |
| 181 | ddltlhktd1 eiqieelnkd lallkkehqe evdglhkhlg ntvnvevdaa pglnlgvimn |
| 241 | emrqkyevma qknlqeakeq ferqtavlqq qvtvnteelk gtevqltelr rtsqsleiel |
| 301 | qshlsmkesl ehtleetkar yssqlanlqs llssleaqlm girsnmerqn neyhilldik |
| 361 | trleqeiaty rrllegedvk tteyqlstle erdikktrki ktvvqevvdg kvvssevkev |
| 421 | eeni |

| RNASE3 (e.g., GenBank Accession Number NP 002926 (SEQ ID NO: 72)): | |
|---|---|
| 1 | mvpklftsqi clllllglmg vegslharpp qftraqwfai qhislnpprc tiamrainny |
| 61 | rwrcknqntf lrttfanvvn vcgnqsircp hnrtlnnchr srfrvpllhc dlinpgaqni |
| 121 | snctyadrpg rrfyvvacdn rdprdspryp vvpvhldtti |

| Aldehyde dehydrogenase X, mitochondrial precursor (ALDH1B1) (e.g., | |
|---|---|
| GenBank Accession Number NP 000683 (SEQ ID NO: 73)): | |
| 1 | mlrflaprll slqgrtarys saaalpspil npdipynqlf innewqdavs kktfptvnpt |
| 61 | tgevighvae gdradvdrav kaareafrlg spwrrmdase rgrllnrlad lverdrvyla |
| 121 | sletldngkp fqesyaldld evikvyryfa gwadkwhgkt ipmdgqhfcf trhepvgvcg |
| 181 | qiipwnfplv mqgwklapal atgntvvmkv aeqtplsaly laslikeagf ppgvvniitg |
| 241 | ygptagaaia qhvdvdkvaf tgstevghli qkaagdsnlk rvtlelggks psivladadm |
| 301 | ehaveqchea lffnmgqccc agsrtfvees iyneflertv ekakqrkvgn pfeldtqqgp |
| 361 | qvdkeqferv lgyiqlgqke gakllcgger fgergffikp tvfggvqddm riakeeifgp |
| 421 | vqplfkfkki eevverannt ryglaaavft rdldkamyft qalqagtvwv ntynivtcht |
| 481 | pfggfkesgn grelgedglk aytevktvti kvpqkns |

| CDNA FLJ25506 fis, clone CBR05185 (e.g., GenBank Accession Number | |
|---|---|
| Q8N7I6 (SEQ ID NO: 74)): | |
| 1 | mwicpggggg gggggggggg dredarpapl ccgrcwrsgc aarpprmvsi glrgavrgar |
| 61 | gchlgrpfsp svllcvgrpg saagaerghs lgsrefghrr gplpwcpanr rgspptagvp |
| 121 | rqppgfpaap aprgpgpltr llgrreagsk sqkllfrsar vqgggqfcps gsaflgvere |
| 181 | ptaglggaer rnarfwrger gqgrqakrpa psqpasplpg ggtwagcvgl vwmgtgfcga |
| 241 | pef |

| Isoform B of fibulin-1 precursor (FBLN1) (e.g., GenBank Accession | |
|---|---|
| Number P23142-2 (SEQ ID NO: 75)): | |
| 1 | meraapsrrv plpllllggl allaagvdad vlleaccadg hrmathqkdc slpyateske |
| 61 | crmvqeqcch sqleelhcat gislaneqdr catphgdnas leatfvkrcc hccllgraaq |
| 121 | aqgqsceysl mvgyqcgqvf raccvksqet gdldvgglqe tdkiieveee qedpylndrc |
| 181 | rgggpckqqc rdtgdevvcs cfvgyqllsd gvscedvnec itgshscrlg escintvgsf |
| 241 | rcqrdsscgt gyeltednsc kdidecesgi hnclpdficq ntlgsfrcrp klqcksgfiq |
| 301 | dalgncidin eclsisapcp ightcinteg sytcqknvpn cgrgyhlnee gtrcvdvdec |
| 361 | appaepcgkg hrcvnspgsf rcecktgyyf dgisrmcvdv necqrypgrl cghkcentlg |
| 421 | sylcscsvgf rlsvdgrsce dinecssspc sqecanvygs yqcycrrgyq lsdvdgvtce |
| 481 | didecalptg ghicsyrcin ipgsfqcscp ssgyrlapng rncqdidecv tgihncsine |
| 541 | tcfniqggfr clafecpeny rrsaatlqqe ktdtvrciks crpndvtcvf dpvhtishtv |
| 601 | islptfreft rpeeiiflra itpphpasqa niifditegn lrdsfdiikr ymdgmtvgvv |
| 661 | rqvrpivgpf havlklemny vvggvvshrn vvnvrifvse ywf |

| Nucleobindin 1 (NUCB1) (e.g., GenBank Accession Number NP 006175 | |
|---|---|
| (SEQ ID NO: 76)): | |
| 1 | mppsgprgtl lllpllllll lravlavple rgapnkeetp atespdtgly yhrylqevid |
| 61 | vletdghfre klqaanaedi ksgklsreld fvshhvrtkl delkrqevsr lrmllkakmd |
| 121 | aeqdpnvqvd hlnllkqfeh ldpqnqhtfe ardlelliqt atrdlaqyda ahheefkrye |
| 181 | mlkeherrry leslgeeqrk eaerkleeqq rrhrehpkvn vpgsqaqlke vweeldgldp |
| 241 | nrfnpktffi lhdinsdgvl deqelealft kelekvydpk needdmreme eerlrmrehv |
| 301 | mknvdtnqdr lvtleeflas tqrkefgdtg egwetvemhp ayteeelrrf eeelaareae |
| 361 | lnakaqrlsq etealgrsqg rleaqkrelq gavlhmeqrk qqqqqqqghk apaahpegql |
| 421 | kfhpdtddvp vpapagdqke vdtsekklle rlpevevpqh l |

| Histone cluster 2, H2ba (HIST2H2BA) (e.g., GenBank Accession Number | |
|---|---|
| NP 001019770 (SEQ ID NO: 77)): | |
| 1 | mpdpaksapa pkkgskkavt kvqkkdgkkr krsrkesysv yvykvlkqvh pdtgisskam |
| 61 | gimnsfvndi feriageasr lahynkrsti tsreiqtavr lllpgelakh avsegtkavt |
| 121 | kytssk |

| Tripartite motif-containing 28 (TRIM28) (e.g., GenBank Accession | |
|---|---|
| Number NP 005753 (SEQ ID NO: 78)): | |
| 1 | maasaaaasa aaasaasgsp gpgegsagge krstapsaaa sasasaaass pagggaeale |
| 61 | llehcgvcre rlrpereprl lpclhsacsa clgpaapaaa nssgdggaag dgtvvdcpvc |
| 121 | kqqcfskdiv enyfmrdsgs kaatdaqdan qcctscedna patsycvecs eplcetcvea |
| 181 | hqrvkytkdh tvrstgpaks rdgertvycn vhkheplvlf cescdtltcr dcqlnahkdh |
| 241 | qyqfledavr nqrkllaslv krlgdkhatl qkstkevrss irqvsdvqkr vqvdvkmail |
| 301 | qimkelnkrg rvlvndaqkv tegqqerler qhwtmtkiqk hqehilrfas walesdnnta |
| 361 | lllskkliyf qlhralkmiv dpvephgemk fqwdlnawtk saeafgkiva erpgtnstgp |
| 421 | apmapprapg plskqgsgss qpmevqegyg fgsgddpyss aephvsgvkr srsgegevsg |
| 481 | lmrkvprvsl erldldltad sqppvfkvfp gsttedynli viergaaaaa tgqpgtapag |
| 541 | tpgapplagm aivkeeetea aigapptate gpetkpvlma laegpgaegp rlaspsgsts |
| 601 | sglevvapeg tsapgggpgt lddsaticrv cqkpgdlvmc nqcefcfhld chlpalqdvp |
| 661 | geewscslch vlpdlkeedg slsldgadst gvvaklspan qrkcervlla lfchepcrpl |
| 721 | hqlatdstfs ldqpggtldl tlirarlqek lsppysspqe faqdvgrmfk qfnkltedka |
| 781 | dvqsiiglqr ffetrmneaf gdtkfsavlv epppmslpga glssqelsgg pgdgp |

| Peroxisomal D3, D2 enoyl-CoA isomerase (PECI) (e.g., GenBank | |
|---|---|
| Accession Number NP 006108 (SEQ ID NO: 79)): | |
| 1 | mnrtamrasq kdfensmnqv kllkkdpgne vklklyalyk qategpcnmp kpgvfdlink |
| 61 | akwdawnalg slpkeaarqn yvdlvsslsp slesssqvep gtdrkstgfe tlvvtsedgi |
| 121 | tkimfnrpkk knaintemyh eimralkaas kddsiitvlt gngdyyssgn dltnftdipp |
| 181 | ggveekaknn avllrefvgc fidfpkplia vvngpavgis vtllglfdav yasdratfht |
| 241 | pfshlgqspe gcssytfpki mspakateml ifgkkltage acaqglvtev fpdstfqkev |
| 301 | wtrlkafakl ppnalriske virkrerekl havnaeecnv lqgrwlsdec tnavvnflsr |
| 361 | kskl |

| Peptidylprolyl isomerase B (PPIB) (e.g., GenBank Accession | |
|---|---|
| Number NP 000933 (SEQ ID NO: 80)): | |
| 1 | mlrlsernmk vllaaaliag svfflllpgp saadekkkgp kvtvkvyfdl rigdedvgrv |
| 61 | ifglfgktvp ktvdnfvala tgekgfgykn skfhrvikdf miqggdftrg dgtggksiyg |
| 121 | erfpdenfkl khygpgwvsm anagkdtngs qffittvkta wldgkhvvfg kvlegmevvr |
| 181 | kvestktdsr dkplkdviia dcgkievekp faiake |

Similar to 40S ribosomal protein S17 (e.g.,
GenBank Accession Number IP00743305
(SEQ ID NO: 81)):
mgrvrtktykkaarviiekyytrlgndfhtnkrvceeiaiipskklrnk
ipeilgtdrrtsdwrgdqlscipvpfpnstmelakglqdnsrscvhssk
tccryhtvgppqlakigstgqvdqsgrprppnradlamepshaekdnhs
alstpeagqsthg

Eukaryotic translation elongation factor 1 gamma
(EEFlG) (e.g., GenBank Accession
Number IPI00747497 (SEQ ID NO: 82)):
avgtlytypenwrafkaliaaqysgaqvrvlsapphfhfgqtnrtpefl
rkfpagkvpafegddgfcvfesnaiayyvsneelrgstpeaaaqvvqwv
sfadsdivppastwvfptlgimhhnkqatenakeevrrilglldaylkt
rtflvgervtladitvvctllwlykqvlepsfrqafpntnrwfltcinq
pqfravlgevklcekmaqfdakkfaetqpkkdtprkekgsreekqkpqa
erkeekkaaapapeeemdeceqalaaepkakdpfahlpkstfvldefkr
kysnedtlsvalpyfwehfdkdqwslwyseyrfpeeltqtfmscnlitg
mfqrldklrknafasvilfgtnnsssisgvwvfrgqelafplspdwqvd
yesytwrkldpgseetqtlvreyfswegafqhvgkafnqgkifk

| Keratin 8 (KRT8) (e.g., GenBank Accession Number | |
|---|---|
| NP 002264 (SEQ ID NO: 83)): | |
| 1 | msirvtqksy kvstsgpraf ssrsytsgpg srissssfsr vgssnfrgql gggyggasgm |
| 61 | ggitavtvnq sllsplvlev dpniqavrtq ekeqiktlnn kfasfidkvr fleqqnkmle |
| 121 | tkwsllqqqk tarsnmdnmf esyinnlrrq letlgqeklk leaelgnmqg lvedfknkye |
| 181 | deinkrteme nefvlikkdv deaymnkvel esrlegltde inflrqlyee eirelqsqis |
| 241 | dtsvvlsmdn srsldmdsii aevkaqyedi anrsraeaes myqikyeelq slagkhgddl |
| 301 | rrtkteisem nrnisrlqae ieglkgqras leaaiadaeq rgelaikdan aklseleaal |
| 361 | qrakqdmarq lreyqelmnv klaldieiat yrkllegees rlesgmqnms ihtkttsgya |
| 421 | gglssayggl tspglsyslg ssfgsgagss sfsrtsssra vvvkkietrd gklvsessdv |
| 481 | lpk |

| Fibulin 2 (FBLN2) (e.g., GenBank Accession Number | |
|---|---|
| NP 001989 (SEQ ID NO: 84)): | |
| 1 | mvllwepaga wlalglalal gpsvaaaapr qdctgvecpp lencieeale pgaccatcvq |
| 61 | qgcacegyqy ydclqggfvr grvpagqsyf vdfgstecsc ppgggkiscq fmlcpeippn |
| 121 | cieavvvads cpqcgqvgcv haghkyaagh tvhlppcrac hcpdaggeli cyqlpgchgn |
| 181 | fsdaeegdpe rhyedpysyd qevaeveaat alggevqaga vqagaggppa algggsqpls |
| 241 | tiqappwpav lprptaaaal gppapvqaka rrvtedseee eeeeeereem avteqlaagg |
| 301 | hrgldglptt apagpslpiq eeraeagara eagarpeenl ildaqatsrs tgpegvthap |
| 361 | slgkaalvpt qavpgsprdp vkpsphnils tslpdaawip ptrevprkpq vlphshveed |
| 421 | tdpnsvhsip rsspegstkd lietccaagq qwaidndecl eipesgtedn vcrtaqrhcc |
| 481 | vsylqekscm agvlgakege tcgaedndsc gislykqccd ccglglrvra egqscesnpn |
| 541 | lgypcnhvml sccegeepli vpevrrppep aaaprrvsea emagrealsl gteaelpnsl |
| 601 | pgddqdecll lpgelcqhlc intvgsyhca cfpgfslqdd grtcrpeghp pqpeapqepa |
| 661 | lksefsqvas ntiplplpqp ntckdngpck qvcstvggsa icscfpgyai madgvscedi |
| 721 | necvtdlhtc srgehcvntl gsfhcykalt cepgyalkdg ecedvdecam gthtcqpgfl |
| 781 | cqntkgsfyc qarqrcmdgf lqdpegncvd inectslsep crpgfscint vgsytcqrnp |
| 841 | licargyhas ddgtkcvdvn ecetgvhrcg egqvchnlpg syrcdckagf qrdafgrgci |
| 901 | dvnecwaspg rlcqhtcent lgsyrcscas gfllaadgkr cedvneceaq rcsqecaniy |
| 961 | gsvqcycrqg yqlaedghtc tdidecaqga gilctfrcln vpgsyqcacp eqgytmtang |
| 1021 | rsckdvdeca lgthncseae tchniqgsfr clrfecppny vqvsktkcer ttchdflecq |
| 1081 | nsparithyq lnfqtgllvp ahifrigpap aftgdtialn iikgneegyf gtrrlnaytg |
| 1141 | vvylqravle prdfaldvem klwrqgsvtt flakmhifft tfal |

| VIM (e.g., GenBank Accession Number NP 003371 (SEQ ID NO: 85)): | |
|---|---|
| 1 | mstrsyssss yrrmfggpgt asrpsssrsy vttstrtysl gsalrpstsr slyasspggv |
| 61 | yatrssavrl rssvpgvrll qdsvdfslad aintefkntr tnekvelqel ndrfanyidk |
| 121 | vrfleqqnki llaeleqlkg qgksrlgdly eeemrelrrq vdqltndkar veverdnlae |
| 181 | dimrlreklq eemlqreeae ntlqsfrqdv dnaslarldl erkveslqee iaflkklhee |
| 241 | eigelqaqiq eqhvqidvdv skpdltaalr dvrqqyesva aknlqeaeew ykskfadlse |
| 301 | aanrnndalr qakqesteyr rqvqsltdev dalkgtnesl erqmremeen faveaanyqd |
| 361 | tigrlqdeiq nmkeemarhl reyqdllnvk maldieiaty rkllegeesr islplpnfss |
| 421 | lnlretnlds lplvdthskr tlliktvetr dgqvinetsq hhddle |

| Fibrinogen alpha chain (FGA) (e.g., GenBank Accession Number | |
|---|---|
| NP 000499 (SEQ ID NO: 86)): | |
| 1 | mfsmrivclv lsvvgtawta dsgegdflae gggvrgprvv erhqsackds dwpfcsdedw |
| 61 | nykcpsgcrm kglidevnqd ftnrinklkn slfeyqknnk dshslttnim eilrgdfssa |
| 121 | nnrdntynrv sedlrsriev lkrkviekvq hiqllqknvr aqlvdmkrle vdidikirsc |
| 181 | rgscsralar evdlkdyedq qkqleqviak dllpsrdrqh lplikmkpvp dlvpgnfksq |
| 241 | lqkvppewka ltdmpqmrme lerpggneit rggstsygtg setesprnps sagswnsgss |
| 301 | gpgstgnrnp gssgtggtat wkpgssgpgs tgswnsgssg tgstgnqnpg sprpgstgtw |
| 361 | npgssergsa ghwtsessvs gstgqwhses gsfrpdspgs gnarpnnpdw gtfeevsgnv |
| 421 | spgtrreyht eklvtskgdk elrtgkekvt sgsttttrrs csktvtktvi gpdghkevtk |
| 481 | evvtsedgsd cpeamdlgtl sgigtldgfr hrhpdeaaff dtastgktfp gffspmigef |
| 541 | vsetesrgse sgiftntkes sshhpgiaef psrgksssys kqftsstsyn rgdstfesks |
| 601 | ykmadeagse adhegthstk rghaksrpvr dcddvlqthp sgtqsgifni klpgsskifs |
| 661 | vycdqetslg gwlliqqrmd gslnfnrtwq dykrgfgsln degegefwlg ndylhlltqr |
| 721 | gsvlrveled wagneayaey hfrvgseaeg yalqvssyeg tagdaliegs veegaeytsh |
| 781 | nnmqfstfdr dadqweenca evygggwwyn ncqaanlngi yypggsydpr nnspyeieng |
| 841 | vvwvsfrgad yslravrmki rplvtq |

| Annexin A2 (ANXA2) (e.g., GenBank Accession Number NP 001002858 | |
|---|---|
| (SEQ ID NO: 87)): | |
| 1 | mgrqlagcgd agkkasfkms tvheilckls legdhstpps aygsvkaytn fdaerdalni |
| 61 | etaiktkgvd evtivniltn rsnaqrqdia fayqrrtkke lasalksals ghletvilgl |
| 121 | lktpaqydas elkasmkglg tdedslieii csrtnqelqe inrvykemyk tdlekdiisd |
| 181 | tsgdfrklmv alakgrraed gsvidyelid qdardlydag vkrkgtdvpk wisimtersv |
| 241 | phlqkvfdry ksyspydmle sirkevkgdl enaflnlvqc iqnkplyfad rlydsmkgkg |
| 301 | trdkvlirim vsrsevdmlk irsefkrkyg kslyyyiqqd tkgdyqkall ylcggdd |

| H2A histone family, member J (H2AFJ) (e.g., GenBank Accession Number | |
|---|---|
| NP 808760 (SEQ ID NO: 88)): | |
| 1 | msgrgkqggk vrakaksrss raglqfpvgr vhrllrkgny aervgagapv ylaavleylt |
| 61 | aeilelagna ardnkktrii prhlqlairn deelnkllgk vtiaqggvlp niqavllpkk |
| 121 | tesqktksk |

| Actin alpha, cardiac muscle 1 (ACTC1) (e.g., GenBank Accession Number | |
|---|---|
| NP 005150 (SEQ ID NO: 89)): | |
| 1 | mcddeettal vcdngsglvk agfagddapr avfpsivgrp rhqgvmvgmg qkdsyvgdea |
| 61 | qskrgiltlk ypiehgiitn wddmekiwhh tfynelrvap eehptlltea plnpkanrek |
| 121 | mtqimfetfn vpamyvaiqa vlslyasgrt tgivldsgdg vthnvpiyeg yalphaimrl |
| 181 | dlagrdltdy lmkiltergy sfvttaerei vrdikeklcy valdfenema taasssslek |
| 241 | syelpdgqvi tignerfrcp etlfqpsfig mesagihett ynsimkcdid irkdlyannv |
| 301 | lsggttmypg iadrmqkeit alapstmkik iiapperkys vwiggsilas lstfqqmwis |
| 361 | kqeydeagps ivhrkcf |

| Keratin 19 (KRT19) (e.g., GenBank Accession Number | |
|---|---|
| NP 002267 (SEQ ID NO: 90)): | |
| 1 | mtsysyrqss atssfgglgg gsvrfgpgva frapsihggs ggrgvsvssa rfvsssssga |
| 61 | ygggyggvlt asdgllagne kltmqnlndr lasyldkvra leaangelev kirdwyqkqg |
| 121 | pgpsrdyshy yttiqdlrdk ilgatiensr ivlqidnarl aaddfrtkfe teqalrmsve |
| 181 | adinglrrvl deltlartdl emqieglkee laylkknhee eistlrgqvg gqvsvevdsa |
| 241 | pgtdlakils dmrsqyevma eqnrkdaeaw ftsrteelnr evaghteqlq msrsevtdlr |
| 301 | rtlqgleiel qsqlsmkaal edtlaetear fgaqlahiqa lisgieaqlg dvradserqn |
| 361 | qeyqrlmdik srlegeiaty rsllegqedh ynnlsaskvl |

| Immunoglobin lambda locus(IGL@protein) (e.g., GenBank Accession Number | |
|---|---|
| Q6PIQ7 (SEQ ID NO: 91)): | |
| 1 | mawallllsl ltqgtgswaq saltqprsys gspgqsvtip ctgtssdvgn ynyvswyrqh |
| 61 | pgkapklmiy dvnkrpsgvp drfsgsksgn tasltisglq aedeadyycc syagtytfgv |
| 121 | fggqtkltvl gqpkaapsvt lfppsseelq ankatlvcli sdfypgavtv awkadsspvk |
| 181 | agvetttpsk qsnnkyaass ylsltpeqwk shksyscqvt hegstvektv aptecs |

| Immunoglobulin heavy constant mu (IGHM) (e.g., GenBank Accession | |
|---|---|
| Number Q8WUK1 (SEQ ID NO: 92)): | |
| 1 | mefglswvfl vallrgvqcq vglvesgggv vqpgrslrls caasgftfss ygmhwvrqap |
| 61 | gkglewvavi sydgsnkyya dsvkgrftis rdnskntlyl qmnslraedt avyycakdws |
| 121 | egvetfdiwg qgtmvtvssg sasaptlfpl vscenspsdt ssvavgclaq dflpdsitfs |
| 181 | wkyknnsdis strgfpsvlr ggkyaatsqv llpskdvmqg tdehvvckvq hpngnkeknv |
| 241 | plpviaelpp kvsvfvpprd gffgnprksk licqatgfsp rqiqvswlre gkqvgsgvtt |
| 301 | dqvqaeakes gpttykvtst ltikesdwls qsmftcrvdh rgltfqqnas smcvpdqdta |
| 361 | irvfaippsf asifltkstk ltclvtdltt ydsvtiswtr qngeavktht niseshpnat |
| 421 | fsavgeasic eddwnsgerf tctvthtdlp splkqtisrp kgvalhrpdv yllppareql |
| 481 | nlresatitc lvtgfspadv fvqwmqrgqp lspekyvtsa pmpepqapgr yfahsiltvs |
| 541 | eeewntgety tcvvahealp nrvtertvdk stegevsade egfenlwata stfivlflls |
| 601 | lfysttvtlf kvk |

| EGF-containing fibulin-like extracellular matrix protein 1 (EFEMP1) | |
|---|---|
| (e.g., GenBank Accession Number Q12805-3 (SEQ ID NO: 93)): | |
| 1 | mlkalfltml tlalvksqdt eetitytqct dgyewdpvrq qckdidecdi vpdackggmk |
| 61 | cvnhyggylc lpktaqiivn neqpqqetqp aegtsgattg vvaassmats gvlpgggfva |
| 121 | saaavagpem qtgrnnfvir rnpadpqrip snpshriqca agyeqsehnv cqdidectag |
| 181 | thncradqvc inlrgsfacq cppgyqkrge qcvdidecti ppychqrcvn tpgsfycqcs |
| 241 | pgfqlaanny tcvdinecda snqcaqqcyn ilgsficqcn qgyelssdrl ncedidecrt |
| 301 | ssylcqyqcv nepgkfscmc pqgyqvvrsr tcqdinecet tnecredemc wnyhggfrcy |
| 361 | prnpcqdpyi ltpenrcvcp vsnamcrelp qsivykymsi rsdrsvpsdi fqiqattiya |
| 421 | ntintfriks gnengefylr qtspvsamlv lvkslsgpre hivdlemltv ssigtfrtss |
| 481 | vlrltiivgp fsf |

| Tripartite motif-containing protein 34 (e.g., GenBank Accession Number | |
|---|---|
| NP 067629 (SEQ ID NO: 94)): | |
| 1 | maskillnvq eevtcpicle llteplsldc ghslcracit vsnkeavtsm ggksscpvcg |
| 61 | isysfehlqa nqhlaniver lkevklspdn gkkrdlcdhh geklllfcke drkvicwlce |
| 121 | rsqehrghht vlteevfkec qeklqavlkr lkkeeeeaek leadireekt swkyqvqter |
| 181 | qriqtefdql rsilnneeqr elqrleeeek ktldkfaeae delvqqkqlv relisdvecr |
| 241 | sqwstmellq dmsgimkwse iwrlkkpkmv skklktvfha pdlsrmlqmf reltavrcyw |
| 301 | vdvtlnsvnl nlnlvlsedq rqvisvpiwp fqcynygvlg sqyfssgkhy wevdvskkta |
| 361 | wilgvycrty srhmkyvvrr canrqnlytk yrplfgywvi glqnkckygv feeslssdpe |
| 421 | vltlsmavpp crvgvfldye agivsffnvt shgsliykfs kccfsqpvyp yfnpwncpap |
| 481 | mtlcppss |

| Isoform 3 of AP1-subunit Gamma Binding Protein 1 (e.g., GenBank Accession | |
|---|---|
| Number NP 542117(SEQ ID NO: 95)): | |
| 1 | malrpgagsg gggaagagag saggggfmfp vaggirppqa glmpmqqqgf pmvsvmqpnm |
| 61 | qgimgmnyss qmsqgpiamq agipmgpmpa agmpylgqap flgmrppgpq ytpdmqkqfa |
| 121 | eeqqkrfeqq qklleeerkr rgfeeqkqkl rllssvkpkt geksrddale aikgnldgfs |
| 181 | rdakmhptpa shpkkpgpsl eekflvscdi stsgqeqikl ntsevghkal gpgsskkyps |
| 241 | lmasngvavd gcvsgtttae aentsdqnls ieesgvgvfp sqdpaqprmp pwiyneslvp |
| 301 | daykkilett mtptgidtak lypilmssgl pretlgqiwa lanrttpgkl tkeelytvla |
| 361 | miavtqrgvp amspdalnqf paapiptlsg fsmtlptpvs qptvipsgpa gsmplslgqp |
| 421 | vmginlvgpv ggaaaqassg fiptypanqv vkpeeddfqd fqdasksgsl ddsfsdfqel |
| 481 | passktsnsq hgnsapsllm plpgtkalps mdkyavfkgi aadkssentv ppgdpgdkys |
| 541 | afrelegtae nkplgesfae frsagtddgf tdfktadsys plepptkdkt fppsfpsgti |
| 601 | qqkqqtqvkn plnladldmf ssvncssekp lsfsavfsts ksystpqstg saatmtalaa |
| 661 | tktssladdf gefslfgeys glapvgeqdd fadfmafsns sisseqkpdd kydalkeeas |
| 721 | pvpltsnvgs tvkggqnsta astkydvfrq lslegsglgv edlkdntpsg ksdddfadfh |
| 781 | sskfssinsd kslgekavaf rhtkedsasv ksldlpsigg ssvgkedsed alavqfdmkl |
| 841 | advggdlkhv msdssldlpt vsgqhppaad iedlkyaafg syssnfavst ltsydwsdrd |
| 901 | datqgrklsp fvlsagsgsp satsilqkke tsfgssenit mtslskvttf vsedalpett |
| 961 | fpalasfkdt ipqtseqkey enrdykdftk qdlptaersq eatcpspass gasqetpnec |
| 1021 | sddfgefqse kpkiskfdfl vatsqskmks seemiksela tfdlsvqgsh krslslgdke |
| 1081 | isrsspspal eqpfrdrsnt lnekpalpvi rdkykdltge veeneryaye wqrclgsaln |
| 1141 | vikkandtln gissssvcte viqsaggmey llgvvevyrv tkrvelgika tavcseklqq |
| 1201 | llkdidkvwn nligfmslat ltpdensldf sscmlrpgik naqelacgvc llnvdsrsra |
| 1261 | fnsetdsfkl aygghqyhas canfwincve pkppglvlpd ll |

| Proflin-1 (e.g., GenBank Accession Number NP 005013 (SEQ ID NO: 96)): | |
|---|---|
| 1 | magwnayidn lmadgtcqda aivgykdsps vwaavpgktf vnitpaevgv lvgkdrssfy |
| 61 | vngltlggqk csvirdsllq dgefsmdlrt kstggaptfn vtvtktdktl vllmgkegvh |
| 121 | gglinkkcye mashlrrsqy |

| Histone H4 (e.g., GenBank Accession Number | |
|---|---|
| NP 001029249 (SEQ ID NO: 97)): | |
| 1 | msgrgkggkg lgkggakrhr kvlrdniqgi tkpairrlar rggvkrisgl iyeetrgvlk |
| 61 | vflenvirda vtytehakrk tvtamdvvya lkrqgrtlyg fgg |

| Hemoglobin subunit alpha (e.g., GenBank Accession Number NP 000549 | |
|---|---|
| (SEQ ID NO: 98)): | |
| 1 | mvlspadktn vkaawgkvga hageygaeal ermflsfptt ktyfphfdls hgsaqvkghg |
| 61 | kkvadaltna vahvddmpna lsalsdlhah klrvdpvnfk llshcllvtl aahlpaeftp |
| 121 | avhasldkfl asvstvltsk yr |

| Transgelin (e.g., GenBank Accession Number NP 001001522 | |
|---|---|
| (SEQ ID NO: 99)): | |
| 1 | mankgpsygm srevqskiek kydeeleerl vewiivqcgp dvgrpdrgrl gfqvwlkngv |
| 61 | ilsklvnsly pdgskpvkvp enppsmvfkq meqvaqflka aedygviktd mfqtvdlfeg |
| 121 | kdmaavqrtl malgslavtk ndghyrgdpn wfmkkaqehk reftesqlqe gkhviglqmg |
| 181 | snrgasqagm tgygrprqii s |

| Lumican precursor (e.g., GenBank Accession Number NP 002336 | |
|---|---|
| (SEQ ID NO: 100)): | |
| 1 | mslsaftlfl aliggtsgqy ydydfplsiy gqsspncape cncpesypsa mycdelklks |
| 61 | vpmvppgiky lylrnnqidh idekafenvt dlqwlildhn llenskikgr vfsklkqlkk |
| 121 | lhinhnnlte svgplpksle dlqlthnkit klgsfeglvn ltfihlqhnr lkedavsaaf |
| 181 | kglksleyld lsfnqiarlp sglpvslltl yldnnkisni pdeyfkrfna lqylrlshne |
| 241 | ladsgipgns fnvsslveld lsynklknip tvnenlenyy levnqlekfd iksfckilgp |
| 301 | lsyskikhlr ldgnrisets lppdmyeclr vanevtln |

| Hemoglobin Beta (e.g., GenBank Accession Number NP 000509 | |
|---|---|
| (SEQ ID NO: 101)): | |
| 1 | mvhltpeeks avtalwgkvn vdevggealg rllvvypwtq rffesfgdls tpdavmgnpk |
| 61 | vkahgkkvlg afsdglahld nlkgtfatls elhcdklhvd penfrllgnv lvcvlahhfg |
| 121 | keftppvqaa yqkvvagvan alahkyh |

| Fibrinogen Beta Chain Precursor (e.g., GenBank Accession | |
|---|---|
| Number NP 005132 (SEQ ID NO: 102)): | |
| 1 | mkrmvswsfh klktmkhlll lllcvflvks qgvndneegf fsarghrpld kkreeapslr |
| 61 | papppisggg yrarpakaaa tqkkverkap daggclhadp dlgvlcptgc qlqeallqqe |
| 121 | rpirnsvdel nnnveavsqt ssssfqymyl lkdlwqkrqk qvkdnenvvn eysselekhq |
| 181 | lyidetvnsn iptnlrvlrs ilenirskiq klesdvsaqm eycrtpctvs cnipvvsgke |
| 241 | ceeiirkgge tsemyliqpd ssvkpyrvyc dmntenggwt viqnrqdgsv dfgrkwdpyk |
| 301 | qgfgnvatnt dgknycglpg eywlgndkis qltrmgptel liemedwkgd kvkahyggft |
| 361 | vqneankyqi svnkyrgtag nalmdgasql mgenrtmtih ngmffstydr dndgwltsdp |
| 421 | rkqcskedgg gwwynrchaa npngryywgg qytwdmakhg tddgvvwmnw kgswysmrkm |
| 481 | smkirpffpq q |

| Immunoglobulin kappa constant (IGKC) (e.g., GenBank Accession | |
|---|---|
| Number Q6GMX8 (SEQ ID NO: 103)): | |
| 1 | mdmrvpaqll gllllwfpgs rcdiqmtqsp ssvsasvgdr vtitcrasqg isswlawyqq |
| 61 | kpgkapklli yaasslqsgv psrfsgsgsg tdftltissl qpedfatyyc qqahsfpftf |
| 121 | gpgtkvdikr tvaapsvfif ppsdeqlksg tasvvcllnn fypreakvqw kvdnalqsgn |
| 181 | sqesvteqds kdstyslsst ltlskadyek hkvyacevth qglsspvtks fnrgec |

| Uncharacterized Protein ALB (e.g., GenBank Accession | |
|---|---|
| Number Q56G89 (SEQ ID NO: 104)): | |
| 1 | mkwvtfisll flfssaysrg vfrrdahkse vahrfkdlge enfkalvlia faqylqqcpf |
| 61 | edhvklvnev tefaktcvad esaencdksl htlfgdklct vatlretyge madccakqep |
| 121 | ernecflqhk ddnpnlprlv rpevdvmcta fhdneetflk kylyeiarrh pyfyapellf |
| 181 | fakrykaaft eccqaadkaa cllpkldelr degkassakq glkcaslqkf gerafkawav |
| 241 | arlsqrfpka efaevsklvt dltkvhtecc hgdllecadd radlakyice nqdsissklk |
| 301 | eccekpllek shciaevend empadlpsla adfvgskdvc knyaeakdvf lgmflyeyar |
| 361 | rhpdysvvll lrlaktyett lekccaaadp hecyakvfde fkplveepqn likqncelfe |
| 421 | qlgeykfqna llvrytkkvp qvstptlvev srnlgkvgsk cckhpeakrm pcaedclsvf |
| 481 | lnqlcvlhek tpvsdrvtkc cteslvngrp cfsalevdet yvpkefnaet ftfhadictl |
| 541 | sekerqikkg talvelvkhk pkatkeqlka vmddfaafve kcckaddket cfaeegkklv |
| 601 | aasqaalgl |

ApoA1 (e.g., GenBank Accession Number P02647 (SEQ ID NO: 105)):
MKAAVLTLAV LFLTGSQARH FWQQDEPPQS PWDRVKDLAT VYVDVLKDSG RDYVSQFEGS
ALGKQLNLKL LDNWDSVTST FSKLREQLGP VTQEFWDNLE KETEGLRQEM SKDLEEVKAK
VQPYLDDFQK KWQEEMELYR QKVEPLRAEL QEGARQKLHE LQEKLSPLGE EMRDRARAHV
DALRTHLAPY SDELRQRLAA RLEALKENGG ARLAEYHAKA TEHLSTLSEK AKPALEDLRQ
GLLPVLESFK VSFLSALEEY TKKLNTQ

C4A (e.g., GenBank Accession Number P0C0L4 (SEQ ID NO: 106)):
MRLLWGLIWA SSFFTLSLQK PRLLLFSPSV VHLGVPLSVG VQLQDVPRGQ VVKGSVFLRN
PSRNNVPCSP KVDFTLSSER DFALLSLQVP LKDAKSCGLH QLLRGPEVQL VAHSPWLKDS
LSRTTNIQGI NLLFSSRRGH LFLQTDQPIY NPGQRVRYRV FALDQKMRPS TDTITVMVEN
SHGLRVRKKE VYMPSSIFQD DFVIPDISEP GTWKISARFS DGLESNSSTQ FEVKKYVLPN
FEVKITPGKP YILTVPGHLD EMQLDIQARY IYGKPVQGVA YVRFGLLDED GKKTFFRGLE
SQTKLVNGQS HISLSKAEFQ DALEKLNMGI TDLQGLRLYV AAAIIESPGG EMEEAELTSW
YFVSSPFSLD LSKTKRHLVP GAPFLLQALV REMSGSPASG IPVKVSATVS SPGSVPEVQD
IQQNTDGSGQ VSIPIIIPQT ISELQLSVSA GSPHPAIARL TVAAPPSGGP GFLSIERPDS
RPPRVGDTLN LNLRAVGSGA TFSHYYYMIL SRGQIVFMNR EPKRTLTSVS VFVDHHLAPS
FYFVAFYYHG DHPVANSLRV DVQAGACEGK LELSVDGAKQ YRNGESVKLH LETDSLALVA
LGALDTALYA AGSKSHKPLN MGKVFEAMNS YDLGCGPGGG DSALQVFQAA GLAFSDGDQW
TLSRKRLSCP KEKTTRKKRN VNFQKAINEK LGQYASPTAK RCCQDGVTRL PMMRSCEQRA
ARVQQPDCRE PFLSCCQFAE SLRKKSRDKG QAGLQRALEI LQEEDLIDED DIPVRSFFPE
NWLWRVETVD RFQILTLWLP DSLTTWEIHG LSLSKTKGLC VATPVQLRVF REFHLHLRLP
MSVRRFEQLE LRPVLYNYLD KNLTVSVHVS PVEGLCLAGG GGLAQQVLVP AGSARPVAFS
VVPTAAAAVS LKVVARGSFE FPVGDAVSKV LQIEKEGAIH REELVYELNP LDHRGRTLEI
PGNSDPNMIP DGDFNSYVRV TASDPLDTLG SEGALSPGGV ASLLRLPRGC GEQTMIYLAP
TLAASRYLDK TEQWSTLPPE TKDHAVDLIQ KGYMRIQQFR KADGSYAAWL SRDSSTWLTA
FVLKVLSLAQ EQVGGSPEKL QETSNWLLSQ QQADGSFQDP CPVLDRSMQG GLVGNDETVA
LTAFVTIALH HGLAVFQDEG AEPLKQRVEA SISKANSFLG EKASAGLLGA HAAAITAYAL
SLTKAPVDLL GVAHNNLMAM AQETGDNLYW GSVTGSQSNA VSPTPAPRNP SDPMPQAPAL
WIETTAYALL HLLLHEGKAE MADQASAWLT RQGSFQGGFR STQDTVIALD ALSAYWIASH
TTEERGLNVT LSSTGRNGFK SHALQLNNRQ IRGLEEELQF SLGSKINVKV GGNSKGTLKV
LRTYNVLDMK NTTCQDLQIE VTVKGHVEYT MEANEDYEDY EYDELPAKDD PDAPLQPVTP
LQLFEGRRNR RRREAPKVVE EQESRVHYTV CIWRNGKVGL SGMAIADVTL LSGFHALRAD
LEKLTSLSDR YVSHFETEGP HVLLYFDSVP TSRECVGFEA VQEVPVGLVQ PASATLYDYY
NPERRCSVFY GAPSKSRLLA TLCSAEVCQC AEGKCPRQRR ALERGLQDED GYRMKFACYY
PRVEYGFQVK VLREDSRAAF RLFETKITQV LHFTKDVKAA ANQMRNFLVR ASCRLRLEPG
KEYLIMGLDG ATYDLEGHPQ YLLDSNSWIE EMPSERLCRS TRQRAACAQL NDFLQEYGTQ
GCQV

C3 187 kDa protein (e.g., GenBank Accession Number P01024
(SEQ ID NO: 107)):
MGPTSGPSLL LLLLTHLPLA LGSPMYSIIT PNILRLESEE TMVLEAHDAQ GDVPVTVTVH
DFPGKKLVLS SEKTVLTPAT NHMGNVTFTI PANREFKSEK GRNKFVTVQA TFGTQVVEKV
VLVSLQSGYL FIQTDKTIYT PGSTVLYRIF TVNHKLLPVG RTVMVNIENP EGIPVKQDSL
SSQNQLGVLP LSWDIPELVN MGQWKIRAYY ENSPQQVFST EFEVKEYVLP SFEVIVEPTE
KFYYIYNEKG LEVTITARFL YGKKVEGTAF VIFGIQDGEQ RISLPESLKR IPIEDGSGEV
VLSRKVLLDG VQNPRAEDLV GKSLYVSATV ILHSGSDMVQ AERSGIPIVT SPYQIHFTKT
PKYFKPGMPF DLMVFVTNPD GSPAYRVPVA VQGEDTVQSL TQGDGVAKLS INTHPSQKPL
SITVRTKKQE LSEAEQATRT MQALPYSTVG NSNNYLHLSV LRTELRPGET LNVNFLLRMD
RAHEAKIRYY TYLIMNKGRL LKAGRQVREP GQDLVVLPLS ITTDFIPSFR LVAYYTLIGA
SGQREVVADS VWVDVKDSCV GSLVVKSGQS EDRQPVPGQQ MTLKIEGDHG ARVVLVAVDK
GVFVLNKKNK LTQSKIWDVV EKADIGCTPG SGKDYAGVFS DAGLTFTSSS GQQTAQRAEL
QCPQPAARRR RSVQLTEKRM DKVGKYPKEL RKCCEDGMRE NPMRFSCQRR TRFISLGEAC
KKVFLDCCNY ITELRRQHAR ASHLGLARSN LDEDIIAEEN IVSRSEFPES WLWNVEDLKE
PPKNGISTKL MNIFLKDSIT TWEILAVSMS DKKGICVADP FEVTVMQDFF IDLRLPYSVV
RNEQVEIRAV LYNYRQNQEL KVRVELLHNP AFCSLATTKR RHQQTVTIPP KSSLSVPYVI
VPLKTGLQEV EVKAAVYHHF ISDGVRKSLK VVPEGIRMNK TVAVRTLDPE RLGREGVQKE
DIPPADLSDQ VPDTESETRI LLQGTPVAQM TEDAVDAERL KHLIVTPSGC GEQNMIGMTP
TVIAVHYLDE TEQWEKFGLE KRQGALELIK KGYTQQLAFR QPSSAFAAFV KRAPSTWLTA
YVVKVFSLAV NLIAIDSQVL CGAVKWLILE KQKPDGVFQE DAPVIHQEMI GGLRNNNEKD
MALTAFVLIS LQEAKDICEE QVNSLPGSIT KAGDFLEANY MNLQRSYTVA IAGYALAQMG
RLKGPLLNKF LTTAKDKNRW EDPGKQLYNV EATSYALLAL LQLKDFDFVP PVVRWLNEQR
YYGGGYGSTQ ATFMVFQALA QYQKDAPDHQ ELNLDVSLQL PSRSSKITHR IHWESASLLR
SEETKENEGF TVTAEGKGQG TLSVVTMYHA KAKDQLTCNK FDLKVTIKPA PETEKRPQDA
KNTMILEICT RYRGDQDATM SILDISMMTG FAPDTDDLKQ LANGVDRYIS KYELDKAFSD
RNTLIIYLDK VSHSEDDCLA FKVHQYFNVE LIQPGAVKVY AYYNLEESCT RFYHPEKEDG
KLNKLCRDEL CRCAEENCFI QKSDDKVTLE ERLDKACEPG VDYVYKTRLV KVQLSNDFDE
YIMAIEQTIK SGSDEVQVGQ QRTFISPIKC REALKLEEKK HYLMWGLSSD FWGEKPNLSY
IIGKDTWVEH WPEEDECQDE ENQKQCQDLG AFTESMVVFG CPN

Actin, Cytoplasmic 1 (actin beta)
(e.g., GenBank Accession Number
NP 001092 (SEQ ID NO: 108):
>refseqp|NP_001092|NP_001092 beta actin
[Homo sapiens].
MDDDIAALVVDNGSGMCKAGFAGDDAPRAVFPSIVGRPRHQGVMVGMGQ
KDSYVGDEAQSKRGILTLKYPIEHGIVTNWDDMEKIWHHTFYNELRVAP
EEHPVLLTEAPLNPKANREKMTQIMFETFNTPAMYVAIQAVLSLYASGR
TTGIVMDSGDGVTHTVPIYEGYALPHAILRLDLAGRDLTDYLMKILTER
GYSFTTTAEREIVRDIKEKLCYVALDFEQEMATAASSSSLEKSYELPDG
QVITIGNERFRCPEALFQPSFLGMESCGIHETTFNSIMKCDVDIRKDLY
ANTVLSGGTTMYPGIADRMQKEITALAPSTMKIKIIAPPERKYSVWIGG
SLASLSTFQQMWISKQEYDESGPSIVHRKCF

Hemoglobin beta (e.g., GenBank Accession
Number O95408 (SEQ ID NO: 109):
>uniprot|095408|095408_HUMAN Beta globin;
MVHLTPEEKSAVTALWGKVNVDEVGGEALGRLLVIYPWTQRFFESFGDL
STPDAVMG

Hemoglobin subunit alpha (e.g., GenBank Accession
Number P69905 (SEQ ID NO: 110):
>uniprot|P69905|HBA_HUMAN Hemoglobin
subunit alpha;
MVLSPADKTNVKAAWGKVGAHAGEYGAEALERMFLSFPTTKTYFPHFDL
SHGSAQVKGHGKKVADALTNAVAHVDDMPNALSALSDLHAHKLRVDPVN
FKLLSHCLLVTLAAHLPAEFTPAVHASLDKFLASVSTVLTSKYR

POTE-2 alpha actin (e.g., GenBank Accession
Number A5A3E0 (SEQ ID NO: 111):
>uniprot|A5A3E0|POTEF_HUMAN POTE ankyrin
domain family member F;
MVVEVDSMPAASSVKKPFGLRSKMGKWCCRCFPCCRESGKSNVGTSGDH
DDSAMKTLRSKMGKWCRHCFPCCRGSGKSNVGASGDHDDSAMKTLRNKM
GKWCCHCFPCCRGSSKSKVGAWGDYDDSAFMEPRYHVRGEDLDKLHRAA
WWGKVPRKDLIVMLRDTDVNKQDKQKRTALHLASANGNSEVVKLLLDRR
CQLNVLDNKKRTALIKAVQCQEDECALMLLEHGTDPNIPDEYGNTTLHY
AIYNEDKLMAKALLLYGADIESKNKHGLTPLLLGVHEQKQQVVKFLIKK
KANLNALDRYGRTALILAVCCGSASIVSLLLEQNIDVSSQDLSGQTARE
YAVSSHHHVICQLLSDYKEKQMLKISSENSNPEQDLKLTSEEESQRFKG
SENSQPEKMSQEPEINKDGDREVEEEMKKHESNNVGLLENLTNGVTAGN
GDNGLIPQRKSRTPENQQFPDNESEEYHRICELLSDYKEKQMPKYSSEN
SNPEQDLKLTSEEESQRLKGSENGQPEKRSQEPEINKDGDRELENFMAI
EEMKKHRSTHVGFPENLTNGATAGNGDDGLIPPRKSRTPESQQFPDTEN
EEYHSDEQNDTQKQFCEEQNTGILHDEILIHEEKQIEVVEKMNSELSLS
CKKEKDILHENSTLREEIAMLRLELDTMKHQSQLREKKYLEDIESVKKR
NDNLLKALQLNELTMDDDTAVLVIDNGSGMCKAGFAGDDAPRAVFPSIV
GRPRQQGMMGGMHQKESYVGKEAQSKRGILTLKYPMEHGIITNWDDMEK
IWHHTFYNELRVAPEEHPVLLTEATLNPKANREKMTQIMFETFNTPAMY
VAIQAVLSLYTSGRTTGIVMDSGDGVTHTVPIYEGNALPHATLRLDLAG
RELPDYLMKILTEHGYRFTTMAEREIVRDIKEKLCYVALDFEQEMATVA
SSSSLEKSYELPDGQVITIGNERFRCPEALFQPCFLGMESCGIHETTFN
SIMKSDVDIRKDLYTNTVLSGGTTMYPGMAHRMQKEIAALAPSMMKIRI
IAPPKRKYSVWVGGSILASLSTFQQMWISKQEYDESGPSIVHRKCL

SLC4A10 (e.g., GenBank Accession
Number Q6U841 (SEQ ID NO: 112):
>uniprot|Q6U841|S4A10_HUMAN Sodium-driven
chloride bicarbonate exchanger;
MEIKDQGAQMEPLLPTRNDEEAVVDRGGTRSILKTHFEKEDLEGHRTLF
IGVHVPLGGRKSHRRHRHRGHKHRKRDRERDSGLEDGRESPSFDTPSQR
VQFILGTEDDDEEHIPHDLFTELDEICWREGEDAEWRETARWLKFEEDV
EDGGERWSKPYVATLSLHSLFELRSCILNGTVLLDMHANTLEEIADMVL
DQQVSSGQLNEDVRHRVHEALMKQHHHQNQKKLTNRIPIVRSFADIGKK
QSEPNSMDKNAGQVVSPQSAPACVENKNDVSRENSTVDFSKGLGGQQKG
HTSPCGMKQRHEKGPPHQQEREVDLHFMKKIPPGAEASNILVGELEFLD
RTVVAFVRLSPAVLLQGLAEVPIPTRFLFILLGPLGKGQQYHEIGRSIA
TLMTDEVFHDVAYKAKDRNDLVSGIDEFLDQVTVLPPGEWDPSIRIEPP
KNVPSQEKRKIPAVPNGTAAHGEAEPHGGHSGPELQRTGRIFGGLILDI
KRKAPYFWSDFRDAFSLQCLASFLFLYCACMSPVITFGGLLGEATEGRI
SAIESLFGASMTGIAYSLFGGQPLTILGSTGPVLVFEKILFKFCKEYGL
SYLSLRASIGLWTATLCIILVATDASSLVCYITRFTEEAFASLICIIFI
YEALEKLFELSEAYPINMHNDLELLTQYSCNCVEPHNPSNGTLKEWRES
NISASDIIWENLTVSECKSLHGEYVGRACGHDHPYVPDVLFWSVILFFS
TVTLSATLKQFKTSRYFPTKVRSIVSDFAVFLTILCMVLIDYAIGIPSP
KLQVPSVFKPTRDDRGWFVTPLGPNPWWTVIAAIIPALLCTILIFMDQQ
ITAVIINRKEHKLKKGCGYHLDLLMVAVMLGVCSIMGLPWFVAATVLSI
THVNSLKLESECSAPGEQPKFLGIREQRVTGLMIFILMGSSVFMTSILK
FIPMPVLYGVFLYMGASSLKGIQFFDRIKLFWMPAKHQPDFIYLRHVPL
RKVHLFTIIQMSCLGLLWIIKVSRAAIVFPMMVLALVFVRKLMDLLFTK
RELSWLDDLMPESKKKKLEDAEKEEEQSMLAMEDEGTVQLPLEGHYRDD
PSVINISDEMSKTALWRNLLITADNSKDKESSFPSKSSPS

Ribonuclease P Protein Subunit P20 (POP7) (e.g.,
GenBank Accession Number Q75817 (SEQ ID NO: 113)
>uniprot|075817|POP7_HUMAN Ribonuclease P protein subunit p20;
MAENREPRGAVEAELDPVEYTLRKRLPSRLPRRPNDIYVNMKTDFKAQLARCQKLLDGGA
RGQNACSEIYIHGLGLAINRAINIALQLQAGSFGSLQVAANTSTVELVDELEPETDTREP
LTRIRNNSAIHIRVFRVTPK

Nuclear RNA export factor 1 (NXF1)(e.g., GenBank
Accession Number Q59E96 (SEQ ID NO: 114):
>uniprot|Q59E96|Q59E96_HUMAN Nuclear RNA export factor 1 variant;
RPAPEPALDLRCGMADEGKSYSEHDDERVNFPQRKKKGRGPFRWKYGEGNRRSGRGGSGI
RSSRLEEDDGDVAMSDAQDGPRVRYNPYTTRPNRRGDTWHDRDRIHVTVRRDRAPPERGG
AGTSQDGTSKNWFKITIPYGRKYDNAWLLSMIQSKCSVPFTPIEFHYENTRAQFFVEDAS
TASALEAVNYKILDRENRRISIIINSSAPPHTILNELKPEQVEQLKLIMSKRYDGSQQAL
DLKGLRSDPDLVAQNIDVVLNRRSCMAATLRIIEENIPELLSLNLSNNRLYRLDDMSSIV
QKAPNLKILNLSGNELKSERELDKIKGLKLEELWLDGNSLCDTFRDQSTYIRSVVACVSP
PGDLHPLGG

UVEAL Autoantigen With Coiled-Coil Domains And Ankyrin Repeats,
UACA (e.g., GenBank Accession Number Q05DB3 (SEQ ID NO: 115):
>uniprot|Q05DB3|Q05DB3_HUMAN UACA protein;
MMNCWFSCTPKNRHAADWNKYDDRLMKAAERGDVEKVTSILAKKGVNPGKLDVEGRSVFH
VVTSKGNLECLNAILIHGVDITTSDTAGRNALHLAAKYGHALCLQKLLQYNCPTEHADLQ
GRTALHDAAMADCPSSIQLLCDHGASVNAKDVDGRTPLVLATQMSRPTICQLLIDRGADV
NSRDKQNRTALMLGCEYGCRDAVEVLIKNGADISLLDALGHDSSYYARIGDNLDILTLLK
TASENTNKGRELWKKGPSLQQRNLTHMQDEVNVKSHQREHQNIQDLEIENEDLKERLRKI
QQEQRILLDKVNGLQLQLNEEVMVADDLESEREKLKSLLAAKEKQHEESLRTIEALKNRF
KYFESDHLGSGSHFSNRKEDMLLKQGQMYMADSQCTSPGIPAHMQSRSMLRPLELSLPSQ
TSYSENEILKKFLEAMRTFCESAKQDRLKLQNELAHKVAECKALALECERVKEDSDEQIK
QLEDALKDVQKRMYESEGKVKQMQTHFLALKEHLTSEAASGNHRLTEELKDQLKDLKVKY
EGASAEVGKLRNQIKQNEMIVEEFKRDEGKLIEENKRLQKELSMCEMEREKKGRKVTEME
GQAKELSAKLALSIPAEKFENMKSSLSNEVNEKAKKKK

Uncharacterized Protein C13ORF27 (e.g., GenBank
Accession Number Q5JUR7(SEQ ID NO: 116):
>uniprot|Q5JUR7|CM027_HUMAN Uncharacterized protein C13orf27;
MSHTEVKLKIPFGNKLLDAVCLVPNKSLTYGIILTHGASGDMNLPHLMSLASHLASHGFF
CLRFTCKGLNIVHRIKAYKSVLNYLKTSGEYKLAGVFLGGRSMGSRAAASVMCHIEPDDG
DDFVRGLICISYPLHHPKQQHKLRDEDLFRLKEPVLFVSGSADEMCEKNLLEKVAQKMQA
PHKIHWIEKANHSMAVKGRSTNDVFKEINTQILFWIQEITEMDKKCH

Isoform 3 of Sushi, Nidogen And EGF-Like Domain-Containing
Protein 1 Precursor (e.g., GenBank Accession Number
Q8TER0 (SEQ ID NO: 117):
>swissprot|Q8TER0|SNED1_HUMAN Sushi, nidogen
and EGF-like domain-containing protein 1;
MRHGVAWALLVAAALGLGARGVRGAVALADFYPFGAERGDAVTPKQDDGGSGLRPLSVPF
PFFGAEHSGLYVNNNGIISFLKEVSQFTPVAFPIAKDRCVVAAFWADVDNRRAGDVYYRE
ATDPAMLRRATEDVRHYFPELLDFNATWVFVATWYRVTFFGGSSSSPVNTFQTVLITDGK
LSFTIFNYESIVWTTGTHASSGGNATGLGGIAAQAGFNAGDGQRYFSIPGSRTADMAEVE
TTTNVGVPGRWAFRIDDAQVRVGGCGHTTSVCLALRPCLNGGKCIDDCVTGNPSYTCSCL
SGFTGRRCHLDVNECASQPCQNGGTCTHGINSFRCQCPAGFGGPTCETAQSPCDTKECQH
GGQCQVENGSAVCVCQAGYTGAACEMDVDDCSPDPCLNGGSCVDLVGNYTCLCAEPFKGL
RCETGDHPVPDACLSAPCHNGGTCVDADQGYVCECPEGFMGLDCRERVPDDCECRNGGRC
LGANTTLCQCPLGFFGLLCEFEITAMPCNMNTQCPDGGYCMEHGGSYLCVCHTDHNASHS
LPSPCDSDPCFNGGSCDAHDDSYTCECPRGFHGKHCEKARPHLCSSGPCRNGGTCKEAGG
EYHCSCPYRFTGRHCEIGKPDSCASGPCHNGGTCFHYIGKYKCDCPPGFSGRHCEIAPSP
CFRSPCVNGGTCEDRDTDFFCHCQAGYMGRRCQAEVDCGPPEEVKHATLRFNGTRLGAVA
LYACDRGYSLSAPSRIRVCQPHGVWSEPPQCLEIDECRSQPCLHGGSCQDRVAGYLCLCS
TGYEGAHCELERDECRAHPCRNGGSCRNLPGAYVCRCPAGFVGVHCETEVDACDSSPCQH
GGRCESGGGAYLCVCPESFFGYHCETVSDPCFSSPCGGRGYCLASNGSHSCTCKVGYTGE
DCAKELFPPTALKMERVEESGVSISWNPPNGPAARQMLDGYAVTYVSSDGSYRRTDFVDR
TRSSHQLQALAAGRAYNISVFSVKRNSNNKNDISRPAVLLARTRPRPVEGFEVTNVTAST
ISVQWALHRIRHATVSGVRVSIRHPEALRDQATDVDRSVDRFTFRALLPGKRYTIQLTTL
SGLRGEEHPTESLATAPTHVWTRPLPPANLTAARVTATSAHVVWDAPTPGSLLEAYVINV
TTSQSTKSRYVPNGKLASYTVRDLLPGRRYQLSVIAVQSTELGPQHSEPAHLYIITSPRD
GADRRWHQGGHHPRVLKNRPPPARLPELRLLNDHSAPETPTQPPRFSELVDGRGRVSARF
GGSPSKAATVRSQPTASAQLENMEEAPYRVSLALQLPEHGSKDIGNVPGNCSENPCQNGG
TCVPGADAHSCDCGPGFKGRRCELACIKVSRPCTRLFSETKAFPVWEGGVCHHVYKRVYR
VHQDICFKESCESTSLKKTPNRKQSKSQTLEKS

Isoform 1 Of Dynein Heavy Chain 10, Axonemal (DNAH10):
(e.g., GenBank Accession Number Q8IVF4 (SEQ ID NO: 118):
>uniprot|Q8IVF4|DYR10_HUMAN Dynein heavy chain 10, axonemal;
MVPEEVEVEIDEIPVLSEEGEEEEETYSQKVESVDKVRAKRVSLRTESLGQPLNREDEEM
DKEISEKLPSKRTAKHIMEKMHLHMLCTPLPEEFLDQNVVFFLRNTKEAISEATDMKEAM
EIMPETLEYGIINANVLHFLKNIICQVFLPALSFNQHRTSTTVGVTSGEVSNSSEHESDL
PPMPGEAVEYHSIQLIRDEFLMNVQKFASNIQRTMQQLEGELKLEMPIISVEGEVSDLAA
DPETVDILEQCVINWLNQISTAVEAQLKKTPQGKGPLAEIEFWRERNATLSALHEQTKLP
IVRKVLDVIKESDSMLVANLQPVFTELFKFHTEASDNVRFLSTVERYFKNITHGSGFHVV
LDTIPAMMSALRMVWIISRHYNKDERMIPLMERIAWEIAERVCRVVNLRTLFKENRASAQ
SKTLEARNTLRLWKKAYFDTRAKTEASGREDRWEFDRKRLFERTDYMATICQDLSDVLQV
LEEFYNIFGPELKAVTGDPKRIDDVLCRVDGLVTPMENLTFDPFSIKSSQFWKYVMDEFK
IEVLIDIINKIFVQNLENPPLYKNHPPVAGAIYWERSLFFRIKHTILRFQEVQEILDSDR
GQEVKQKYLEVGRTMKEYEDRKYEQWMEVTEQVLPALMKKSLLTKSSIATEEPSTLERGA
VFAINFSPALREIINETKYLEQLGFTVPELARNVALQEDKFLRYTAGIQRMLDHYHMLIG
TLNDAESVLLKDHSQELLRVFRSGYKRLNWNSLGTGDYITGCKQAIGKFESLVHQIHKNA
DDISSRLTLIEAINLFKYPAAKSEEELPGVKEFFEHIERERASDVDHMVRWYLAIGPLLT
KVEGLVVHTNTGKAPKLASYYKYWEKKIYEVLTKLILKNLQSFNSLILGNVPLFHTETIL
TAPEIILHPNTNEIDKMCFHCVRNCVEITKHFVRWMNGSCIECPPQKGEEEEVVIINFYN
DISLNPQIIEQAVMIPQNVHRILINLMKYLQKWKRYRPLWKLDKAIVMEKFAAKKPPCVA
YDEKLQFYSKIAYEVMRHPLIKDEHCIRLQLRHLANTVQENAKSWVISLGKLLNESAKEE
LYNLHEEMEHLAKNLRKIPNTLEDLKFVLATIAEIRSKSLVMELRYRDVQERYRTMAMYN
LFPPDAEKELVDKIESIWSNLFNDSVNVEHALGDIKRTFTELTRGEIMMYRVQIEEFAKR
FYSEGPGSVGDDLDKGVELLGVYERELARHEKSRQELANAEKLFDLPITMYPELLKVQKE
MSGLRMIYELYEGLKVAKEEWSQTLWINLNVQILQEGIEGFLRALRKLPRPVRGLSVTYY
LEAKMKAFKDSIPLLLDLKNEALRDRHWKELMEKTSVFFEMTETFTLENMFAMELHKHTD
VLNEIVTAAIKEVAIEKAVKEILDTWENMKFTVVKYCKGTQERGYILGSVDEIIQSLDDN
TFNLQSISGSRFVGPFLQTVHKWEKTLSLIGEVIEIWMLVQRKWMYLESIFIGGDIRSQL
PEEAKKFDNIDKVFKRIMGETLKDPVIKRCCEAPNRLSDLQNVSEGLEKCQKSLNDYLDS
KRNAFPRFFFISDDELLSILGSSDPLCVQEHMIKMYDNIASLRFNDGDSGEKLVSAMISA
EGEVMEFRKIVRAEGRVEDWMTAVLNEMRRTNRLITKEAIFRYCEDRSRVDWMLLYQGMV
VLAASQVWWTWEVEDVFHKAQKGEKQAMKNYGRKMHRQIDELVTRITMPLSKNDRKKYNT
VLIIDVHARDIVDSFIRGSILEAREFDWESQLRFYWDREPDELNIRQCTGTFGYGYEYMG
LNGRLVITPLTDRIYLTLTQALSMYLGGAPAGPAGTGKTETTKDLAKALGLLCVVTNCGE
GMDYRAVGKIFSGLAQCGAWGCFDEFNRIDASVLSVISSQIQTIRNALIHQLTTFQFEGQ
EISLDSRMGIFITMNPGYAGRTELPESVKALFRPVVVIVPDLQQICEIMLFSEGFLEAKT
LAKKMTVLYKLAREQLSKQYHYDFGLRALKSVLVMAGELKRGSSDLREDVVLMRALRDMN
LPKFVFEDVPLFLGLISDLFPGLDCPRVRYPDFNDAVEQVLEENGYAVLPIQVDKVVQMF
ETMLTRHTTMVVGPTRGGKSVVINTLCQAQTKLGLTTKLYILNPKAVSVIELYGILDPTT
RDWTDGVLSNIFREINKPTDKKERKYILFDGDVDALWVENMNSVMDDNRLLTLANGERIR
LQAHCALLFEVGDLQYASPATVSRCGMVYVDPKNLKYRPYWKKWVNQIPNKVEQYNLNSL
FEKYVPYLMDVIVEGIVDGRQAEKLKTIVPQTDLNMVTQLAKMLDALLEGEIEDLDLLEC
YFLEALYCSLGASLLEDGRMKFDEYIKRLASLSTVDTEGVWANPGELPGQLPTLYDFHFD
NKRNQWVPWSKLVPEYIHAPERKFINILVHTVDTTRTTWILEQMVKIKQPVIFVGESGTS
KTATTQNFLKNLSEETNIVLMVNFSSRTTSMDIQRNLEANVEKRTKDTYGPPMGKRLLVF
MDDMNMPRVDEYGTQQPIALLKLLLEKGYLYDRGKELNCKSIRDLGFIAAMGKAGGGRNE
VDPRFISLFSVFNVPFPSEESLHLIYSSILKGHTSTFHESIVAVSGKLTFCTLALYKNIV
QDLPPTPSKFHYIFNLRDLSRVFNGLVLTNPERFQTVAQMVRVWRNECLRVFHDRLISET
DKQLVQQHIGSLVVEHFKDDVEVVMRDPILFGDFQMALHEGEPRIYEDIQDYEAAKALFQ
EILEEYNESNTKMNLVLFDDALEHLTRVHRIIRMDRGHALLVGVGGSGKQSLSRLAAFTA
SCEVFEILLSRGYSENSFREDLKSLYLKLGIENKAMIFLFTDAHVAEEGFLELINNMLTS
GIVPALFSEEEKESILSQIGQEALKQGMGPAKESVWQYFVNKSANNLHIVLGMSPVGDTL
RTWCRNFPGMVNNTGIDWFMPWPPQALHAVAKSFLGYNPMIPAENIENVVKHVVLVHQSV
DHYSQQFLQKLRRSNYVTPKNYLDFINTYSKLLDEKTQCNIAQCKRLDGGLDKLKEATIQ
LDELNQKLAEQKIVLAEKSAACEALLEEIAVNTAVAEEKKKLAEEKAMEIEEQNKVIAME
KAEAETTLAEVMPILEAAKLELQKLDKSDVTEIRSFAKPPKQVQTVCECILIMKGYKELN
WKTAKGVMSDPNFLRSLMEIDFDSITQSQVKNIKGLLKTLNTTTEEMEAVSKAGLGMLKF
VEAVMGYCDVFREIKPKREKVARLERNFYLTKRELERIQNELAAIQKELETLGAKYEAAI
LEKQKLQEEAEIMERRLIAADKLISGLGSENIRWLNDLDELMHRRVKLLGDCLLCAAFLS
YEGAFTWEFRDEMVNRIWQNDILEREIPLSQPFRLESLLTDDVEISRWGSQGLPPDELSV
QNGILTTRASRFPLCIDPQQQALNWIKRKEEKNNLRVASFNDPDFLKQLEMSIKYGTPFL
FRDVDEYIDPVIDNVLEKNIKVSQGRQFIILGDKEVDYDSNFRLYLNTKLANPRYSPSVF
GKAMVINYTVTLKGLEDQLLSVLVAYERRELEEQREHLIQETSENKNLLKDLEDSLLREL
ATSTGNMLDNVDLVHTLEETKSKATEVSEKLKLAEKTALDIDRLRDGYRPAARRGAILFF
VLSEMALVNSMYQYSLIAFLEVFRLSLKKSLPDSILMKRLRNIMDTLTFSIYNHGCTGLF
ERHKLLFSFNMTIKIEQAEGRVPQEELDFFLKGNISLEKSKRKKPCAWLSDQGWEDIILL
SEMFSDNFGQLPDDVENNQTVWQEWYDLDSLEQFPVPLGYDNNITPFQKLLILRCFRVDR
VYRAVTDYVTVTMGEKYVQPPMISFEAIFEQSTPHSPIVFILSPGSDPATDLMKLAERSG
FGGNRLKFLAMGQGQEKVALQLLETAVARGQWLMLQNCHLLVKWLKDLEKSLERITKPHP
DFRLWLTTDPTKGFPIGILQKSLKVVTEPPNGLKLNMRATYFKISHEMLDQCPHPAFKPL
VYVLAFFHAVVQERRKFGKIGWNVYYDFNESDFQVCMEILNTYLTKAFQQRDPRIPWGSL
KYLIGEVMYGGRAIDSFDRRILTIYMDEYLGDFIFDTFQPFHFFRNKEVDYKIPVGDEKE
KFVEAIEALPLANTPEVFGLHPNAEIGYYTQAARDMWAHLLELQPQTGESSSGISRDDYI
GQVAKEIENKMPKVFDLDQVRKRLGTGLSPTSVVLLQELERFNKLVVRMTKSLAELQRAL
AGEVGMSNELDDVARSLFIGHIPNIWRRLAPDTLKSLGNWMVYFLRRFSQYMLWVTESEP
SVMWLSGLHIPESYLTALVQATCRKNGWPLDRSTLFTQVTKFQDADEVNERAGQGCFVSG
LYLEGADWDIEKGCLIKSKPKVLVVDLPILKIIPIEAHRLKLQNTFRTPVYTTSMRRNAM
GVGLVFEADLFTTRHISHWVLQGVCLTLNSD

Gap junction alpha-1 protein (GJA1/Connexion 43) (e.g.,
GenBank Accession Number P17302 (SEQ ID NO: 119):
>uniprot|P17302|CXA1_HUMAN Gap junction alpha-1 protein;
MGDWSALGKLLDKVQAYSTAGGKVWLSVLFIFRILLLGTAVESAWGDEQSAFRCNTQQPG
CENVCYDKSFPISHVRFWVLQIIFVSVPTLLYLAHVFYVMRKEEKLNKKEEELKVAQTDG
VNVDMHLKQIEIKKFKYGIEEHGKVKMRGGLLRTYIISILFKSIFEVAFLLIQWYIYGFS
LSAVYTCKRDPCPHQVDCFLSRPTEKTIFIIFMLVVSLVSLALNIIELFYVFFKGVKDRV
KGKSDPYHATSGALSPAKDCGSQKYAYFNGCSSPTAPLSPMSPPGYKLVTGDRNNSSCRN
YNKQASEQNWANYSAEQNRMGQAGSTISNSHAQPFDFPDDNQNSKKLAAGHELQPLAIVD
QRPSSRASSRASSRPRPDDLEI

Isoform 1 Of Kinesin-Like Protein KIF25(KIF25) (e.g.,
GenBank Accession Number Q5SZU8 (SEQ ID NO: 120):
>uniprot|Q5SZU8|Q5SZU8_HUMAN Kinesin family member 25;
CRAVGSASKLMELVHGGLQLRAKHPTLVHADSSRSHLIITVTLTTASCSDSTADQACSAT
LPREQTEAGRAGRSRRASQGALAPQLVPGNPAGHAEQVQARLQLVDSAGSECVGGDAKLL
VILCISPSQRHLAQTLQGLGFGIRARQVQRGPARKKPPSSQTEGKRRPD

GAPDH-Glyceraldehyde-3-Phosphate Dehydrogenase
(e.g., GenBank Accession Number P04406 (SEQ ID NO: 121):
>uniprot|P04406|G3P_HUMAN Glyceraldehyde-3-phosphate dehydrogenase;
MGKVKVGVNGFGRIGRLVTRAAFNSGKVDIVAINDPFIDLNYMVYMFQYDSTHGKFHGTV
KAENGKLVINGNPITIFQERDPSKIKWGDAGAEYVVESTGVFTTMEKAGAHLQGGAKRVI
ISAPSADAPMFVMGVNHEKYDNSLKIISNASCTTNCLAPLAKVIHDNFGIVEGLMTTVHA
ITATQKTVDGPSGKLWRDGRGALQNIIPASTGAAKAVGKVIPELNGKLTGMAFRVPTANV
SVVDLTCRLEKPAKYDDIKKVVKQASEGPLKGILGYTEHQVVSSDFNSDTHSSTFDAGAG
IALNDHFVKLISWYDNEFGYSNRVVDLMAHMASKE

Uncharacterized Protein ALB (e.g.,
GenBank Accession Number P02768 (SEQ ID NO: 122):
>uniprot|P02768|ALBU_HUMAN Serum albumin;
MKWVTFISLLFLFSSAYSRGVFRRDAHKSEVAHRFKDLGEENFKALVLIAFAQYLQQCPF
EDHVKLVNEVTEFAKTCVADESAENCDKSLHTLFGDKLCTVATLRETYGEMADCCAKQEP
ERNECFLQHKDDNPNLPRLVRPEVDVMCTAFHDNEETFLKKYLYEIARRHPYFYAPELLF
FAKRYKAAFTECCQAADKAACLLPKLDELRDEGKASSAKQRLKCASLQKFGERAFKAWAV
ARLSQRFPKAEFAEVSKLVTDLTKVHTECCHGDLLECADDRADLAKYICENQDSISSKLK
ECCEKPLLEKSHCIAEVENDEMPADLPSLAADFVESKDVCKNYAEAKDVFLGMFLYEYAR
RHPDYSVVLLLRLAKTYETTLEKCCAAADPHECYAKVFDEFKPLVEEPQNLIKQNCELFE
QLGEYKFQNALLVRYTKKVPQVSTPTLVEVSRNLGKVGSKCCKHPEAKRMPCAEDYLSVV
LNQLCVLHEKTPVSDRVTKCCTESLVNRRPCFSALEVDETYVPKEFNAETFTFHADICTL
SEKERQIKKQTALVELVKHKPKATKEQLKAVMDDFAAFVEKCCKADDKETCFAEEGKKLV
AASQAALGL

Galectin-3, LGALS3 (e.g., GenBank Accession
Number NP_002297(SEQ ID NO: 123)
>refseqp|NP_002297|NP_002297 galectin 3 [Homo sapiens].
MADNFSLHDALSGSGNPNPQGWPGAWGNQPAGAGGYPGASYPGAYPGQAPPGAYPGQAPP
GAYPGAPGAYPGAPAPGVYPGPPSGPGAYPSSGQPSATGAYPATGPYGAPAGPLIVPYNL
PLPGGVVPRMLITILGTVKPNANRIALDFQRGNDVAFHFNPRFNENNRRVIVCNTKLDNN
WGREERQSVFPFESGKPFKIQVLVEPDHFKVAVNDAHLLQYNHRVKKLNEISKLGISGDI
DLTSASYTMI

Similar To NAC-Alpha Domain-Containing Protein 1 (NACAD)
(e.g., GenBank Accession Number O15069 (SEQ ID NO: 124):
>uniprot|O15069|NACAD_HUMAN NAC-alpha domain-containing
protein 1;
MPGEAARAELLLPEADRPGPRTDLSCDAAAATTILGGDRREPCALTPGPSHLALTFLPSK
PGARPQPEGASWDAGPGGAPSAWADPGEGGPSPMLLPEGLSSQALSTEAPLPATLEPRIV
MGEETCQALLSPRAARTALRDQEGGHASPDPPPELCSQGDLSVPSPPPDPDSFFTPPSTP
TKTTYALLPACGPHGDARDSEAELRDELLDSPPASPSGSYITADGDSWASSPSCSLSLLA
PAEGLDFPSGWGLSPQGSMVDERELHPAGTPEPPSSESSLSADSSSSWGQEGHFFDLDFL
ANDPMIPAALLPFQGSLIFOVEAVEVTPLSPEEEEEEAVADPDPGGDLAGEGEEDSTSAS
FLQSLSDLSITEGMDEAFAFRDDTSAASSDSDSASYAEADDERLYSGEPHAQATLLQDSV
QKTEEESGGGAKGLQAQDGTVSWAVEAAPQTSDRGAYLSQRQELISEVTEEGLALGQEST
ATVTPHTLQVAPGLQVEVATRVTPQAGEEETDSTAGQESAAMAMPQPSQEGISEILGQES
VTAEKLPTPQEETSLTLCPDSPQNLKEEGGLDLPSGRKPVAAATIVPRQAKEDLTLPQDS
AMTPPLPLQDTDLSSAPKPVAAATIVSQQAEEGLTLPQDSVMTPPLPLQDTELSSAPKPV
AAATLVSQQAEEGLTLPQDSAMTPPLPLQDTDLSSAPKPVAAATLVSQQAEEGLTLPQDS
AMTPPLPLQDTDLSSAPKPVAAATLVSQQAEEGLTLPQDSAMTPPLPLQDTDLSSAPKPV
AAATIVSQQAEEGLTLPQDSAMTPPLPLQDTDLSSAPKPVAAATIVSQQAEEGLTLPQDS
AMTPPLPLQDTDLSSAPKPVAAATPVSQQAEEGLTLPQDSAMTPPLPLQDTDLSSAPKPV
AAATPVSQQAEEGLTLPQDSAMTAPLPLQDTGPTSGPEPLAVATPQTLQAEAGCAPGTEP
VATMAQQEVGEALGPRPAPEEKNAALPTVPEPAALDQVQQDDPQPAAEAGTPWAAQEDAD
STLGMEALSLPEPASGAGEEIAEALSRPGREACLEARAHTGDGAKPDSPQKETLEVENQQ
EGGLKLLAQEHGPRSALGGAREVPDAPPAACPEVSQARLLSPAREERGLSGKSTPEPTLP
SAVATEASLDSCPESSVGAVSSLDRGCPDAPAPTSAPTSQQPEPVLGLGSVEQPHEVPSV
LGTPLLQPPENLAKGQPSTPVDRPLGPDPSAPGTLAGAALPPLEPPAPCLCQDPQEDSVE
DEEPPGSLGLPPPQAGVQPAAAAVSGTTQPLGTGPRVSLSPHSPLLSPKVASMDAKDLAL
QILPPCQVPPPSGPQSPAGPQGLSAPEQQEDEDSLEEDSPRALGSGQHSDSHGESSAELD
EQDILAPQTVQCPAQAPAGGSEETIAKAKQSRSEKKARKAMSKLGLRQIQGVTRITIQKS
KNILFVIAKPDVFKSPASDTYVVFGEAKIEDLSQQVHKAAAEKFKVPSEPSALVPESAPR
PRVRLECKEEEEEEEEEVDEAGLELRDIELVMAQANVSRAKAVRALRDNHSDIVNAIMEL
TM

Acetyl-CoA Acetyltransferase, Mitochondrial , ACATI (e.g.,
GenBank Accession Number NP_000010 (SEQ ID NO: 125):
>refseqp|NP_000010|NP_000010 acetyl-Coenzyme
A acetyltransferase 1 precursor [Homo sapiens].
MAVLAALLRSGARSRSPLLRRLVQEIRYVERSYVSKPTLKEVVIVSATRTPIGSFLGSLS
LLPATKLGSIAIQGAIEKAGIPKEEVKEAYMGNVLQGGEGQAPTRQAVLGAGLPISTPCT
TINKVCASGMKAIMMASQSLMCGHQDVMVAGGMESMSNVPYVMNRGSTPYGGVKLEDLIV
KDGLTDVYNKIHMGSCAENTAKKLNIARNEQDAYAINSYTRSKAAWEAGKFGNEVIPVTV
TVKGQPDVVVKEDEEYKRVDFSKVPKLKTVFQKENGTVTAANASTLNDGAAALVLMTADA
AKRLNVTPLARIVAFADAAVEPIDFPIAPVYAASMVLKDVGLKKEDIAMWEVNEAFSLVV
LANIKMLEIDPQKVNINGGAVSLGHPIGMSGARIVGHLTHALKQGEYGLASICNGGGGAS
AMLIQKL

KH-Type Splicing Regulatory Protein, FUBP2 (e.g.,
GenBank Accession Number NP_003676 (SEQ ID NO: 126):
>refseqp|NP_003676|NP_003676 KH-type splicing regulatory
protein (FUSE binding protein 2) [Homo sapiens].
MSDYSTGGPPPGPPPPAGGGGGAGGAGGGPPPGPPGAGDRGGGGPGGGGPGGGSAGGPSQ
PPGGGGPGIRKDAFADAVQRARQIAAKIGGDAATTVNNSTPDFGFGGQKRQLEDGDQPES
KKLASQGDSISSQLGPIHPPPRTSMTEEYRVPDGMVGLIIGRGGEQINKIQQDSGCKVQI
SPDSGGLPERSVSLTGAPESVQKAKMMLDDIVSRGRGGPPGQFHDNANGGQNGTVQEIMI
PAGKAGLVIGKGGETIKQLQERAGVKMILIQDGSQNTNVDKPLRIIGDPYKVQQACEMVM
DILRERDQGGFGSRNEYGSRIGGGIDVPVPRHSVGVVIGRSGEMIKKIQNDAGVRIQFKQ
DDGTGPEKIAHIMGPPDRCEHAARIINDLLQSLRSGPPGPPGGPGMPPGGRGRGRGQGNW
GPPGGEMTFSIPTHKCGLVIGRGGENVKAINQQTGAFVEISRQLPPNGDPNFKLFIIRGS
PQQIDHAKQLIEEKIEGPLCPVGPGPGGPGPAGPMGPFNPGPFNQGPPGAPPHAGGPPPH
QYPPQGWGNTYPQWQPPAPHDPSKAAAAAADPNAAWAAYYSHYYQQPPGPVPGPAPAPAA
PPAQGEPPQPPPTGQSDYTKAWEEYYKKIGQQPQQPGAPPQQDYTKAWEEYYKKQAQVAT
GGGPGAPPGSQPDYSAAWAEYYRQQAAYYGQTPGPGGPQPPPTQQGQQQAQ

Profilin 1(PFN1) (e.g., GenBank Accession
Number NP_005013 (SEQ ID NO: 127):
>refseqp|NP_005013|NP_005013 profilin 1 [Homo sapiens].
MAGWNAYIDNLMADGTCQDAAIVGYKDSPSVWAAVPGKTFVNITPAEVGVLVGKDRSSFY
VNGLTLGGQKCSVIRDSLLQDGEFSMDLRTKSTGGAPTFNVTVTKTDKTLVLLMGKEGVH
GGLINKKCYEMASHLRRSQY

Chloride intracellular Channel Protein 1, CLIC1
(e.g., GenBank Accession Number NP_001279
(SEQ ID NO: 128):
>refseqp|NP_001279|NP_001279 chloride
intracellular channel 1 [Homo sapiens].
MAEEQPQVELFVKAGSDGAKIGNCPFSQRLFMVLWLKGVTFNVTTVDTK
RRTETVQKLCPGGQLPFLLYGTEVHTDTNKIEEFLEAVLCPPRYPKLAA
LNPESNTAGLDIFAKFSAYIKNSNPALNDNLEKGLLKALKVLDNYLTSP
LPEEVDETSAEDEGVSQRKFLDGNELTLADCNLLPKLHIVQVVCKKYRG
FTIPEAFRGVHRYLSNAYAREEFASTCPDDEEIELAYEQVAKALK

Zinc Finger Protein 831
(e.g., GenBank Accession Number NP_848552
(SEQ ID NO: 129):
>refseqp|NP_848552|NP_848552 zinc finger
protein 831 [Homo sapiens].
MEVPEPTCPAPPARDQPAPTPGPPGAPGGQASPHLTLGPVLLPPEQGLA
PPTVFLKALPIPLYHTVPPGGLQPRAPLVTGSLDGGNVPFILSPVLQPE
GPGPTQVGKPAAPTLTVNIVGTLPVLSPGLGPTLGSPGKVRNAGKYLCP
HCGRDCLKPSVLEKHIRSHTGERPFPCATCGIAFKTQSNLYKHRRTQTH
LNNSRLSSESEGAGGGLLEEGDKAGEPPRPEGRGESRCQGMHEGASERP
LSPGAHVPLLAKNLDVRTEAAPCPGSAFADREAPWDSAPMASPGLPAAS
TQPWRKLPEQKSPTAGKPCALQRQQATAAEKPWDAKAPEGRLRKCESTD
SGYLSRSDSAEQPHAPCSPLHSLSEHSAESEGEGGPGPGPGVAGAEPGA
REAGLELEKKRLEERIAQLISINQAVVDDAQLDNVRPRKTGLSKQGSID
LPTPYTYKDSFHFDIRALEPGRRRAPGPVRSTWTPPDKSRPLFFHSVPT
QLSTTVECVPVTRSNSLPFVEGSRTWLEPREPRDPWSRTQKPLSPRPGP
ARLGCRSGLSSTDVPSGHPRALVRQAAVEDLPGTPIGDALVPAEDTDAK
RTAAREAMAGKGRAGGRKCGQRRLKMFSQEKWQVYGDETFKRIYQKMKA
SPHGGKKAREVGMGSGAELGFPLQKEAAGSSGTVPTQDRRTPVHEDISA
GATPEPWGNPPALEASLVTEPTKHGETVARRGDSDRPRVEEAVSSPALG
GRDSPCSGSRSPLVSPNGRLELGWQMPPAPGPLKGGDVEAPRPVWPDPK
LEGGARGVGDVQETCLWAQTVLRWPSRGSGEDKLPSERKKLKVEDLHSW
KQPEPVSAETPGGPTQPASLSSQKQDADPGEVPGGSKESARQVGEPLES
SGASLAAASVALKRVGPRDKATPLHPAAPAPAEHPSLATPPQAPRVLSA
LADNAFSPKYLLRLPQAETPLPLPIPWGPRHSQDSLCSSGWPEERASFV
GSGLGTPLSPSPASGPSPGEADSILEDPSCSRPQDGRKGAQLGGDKGDR
MATSRPAARELPISAPGAPREATSSPPTPTCEAHLVQDMEGDSHRIHRL
CMGSTLARARLSGDVLNPWVPNWELGEPPGNAPEDPSSGPLVGPDPCSP
LQPGSFLTALTRPQGVPPGWPELALSSHSGTSRSHSTRSPHSTQNPFPS
LKAEPRLTWCCLSRSVPLPAEQKAKAASVYLAVHFPGSSLRDEGPNGPP
GSNGGWTWTSPGEGGPAQMSKFSYPTVPGVMPQHQVSEPEWKKGLPWRA
KMSRGNSKQRKLKINPKRYKGNFLQSCVQLRASRLRTPTWVRRRSRHPP
ALEGLKPCRTPGQTSSEIAGLNLQEEPSCATSESPPCCGKEEKKEGDCR
QTLGTLSLGTSSRIVREMDKRTVKDISPSAGEHGDCTTHSTAATSGLSL
QSDTCLAVVNDVPLPPGKGLDLGLLETQLLASQDSVSTDPKPYIFSDAQ
RPSSFGSKGTFPHHDIATSVAAVCISLPVRTDHIAQEIHSAESRDHSQT
AGRTLTSSSPDSKVTEEGRAQTLLPGRPSSGQRISDSVPLESTEKTHLE
IPASGPSSASSHHKEGRHKTFFPSRGQYGCGEMTVPCPSLGSDGRKRQV
SGLITRKDSVVPSKPEQPIEIPEAPSKSLKKRSLEGMRKQTRVEFSDTS
SDDEDRLVIEI

Endoplasmin (e.g., GenBank Accession Number
NP_003290 (SEQ ID NO: 130):
>refseqp|NP_003290|NP_003290 heat shock protein
90 kDa beta, member 1 [Homo sapiens].
MRALWVLGLCCVLLTFGSVRADDEVDVDGTVEEDLGKSREGSRTDDEVV
QREEEAIQLDGLNASQIRELREKSEKFAFQAEVNRMMKLIINSLYKNKE
IFLRELISNASDALDKIRLISLTDENALSGNEELTVKIKCDKEKNLLHV
TDTGVGMTREELVKNLGTIAKSGTSEFLNKMTEAQEDGQSTSELIGQFG
VGFYSAFLVADKVIVTSKHNNDTQHIWESDSNEFSVIADPRGNTLGRGT
TITLVLKEEASDYLELDTIKNLVKKYSQFINFPIYVWSSKTETVEEPME
EEEAAKEEKEESDDEAAVEEEEEEKKPKTKKVEKTVWDWELMNDIKPIW
QRPSKEVEEDEYKAFYKSFSKESDDPMAYIHFTAEGEVTFKSILFVPTS
APRGLFDEYGSKKSDYIKLYVRRVFITDDFHDMMPKYLNFVKGVVDSDD
LPLMVSRETLQQHKLLKVIRKKLVRKTLDMIKKIADDKYNDTFWKEFGT
NIKLGVIEDHSNRTRLAKLLRFQSSHHPTDITSLDQYVERMKEKQDKIY
FMAGSSRKEAESSPFVERLLKKGYEVIYLTEPVDEYCIQALPEFDGKRF
QNVAKEGVKFDESEKTKESREAVEKEFEPLLNWMKDKALKDKIEKAVVS
QRLTESPCALVASQYGWSGNMERIMKAQAYQTGKDISTNYYASQKKTFE
INPRHPLIRDMLRRIKEDEDDKTVLDLAVVLFETATLRSGYLLPDTKAY
GDRIERMLRLSLNIDPDAKVEEEPEEEPEETAEDTTEDTEQDEDEEMDV
GTDEEEETAKESTAEKDEL

Ribosomal Protein S10 (RPS10)
(e.g., GenBank Accession Number P46783
(SEQ ID NO: 131):
>uniprot|P46783|RS10_HUMAN 40S ribosomal
protein S10;
MLMPKKNRIAIYELLFKEGVMVAKKDVHMPKHPELADKNVPNLHVMKAM
QSLKSRGYVKEQFAWRHFYWYLTNEGIQYLRDYLHLPPEIVPATLRRSR
PETGRPRPKGLEGERPARLTRGEADRDTYRRSAVPPGADKKAEAGAGSA
TEFQFRGGFGRGRGQPPQ

Splicing Factor, Arginine/Serine-Rich 3
(e.g., GenBank Accession Number NP_003008
(SEQ ID NO: 132):
>refseqp|NP_003008|NP_003008 splicing factor,
arginine/serine-rich 3 [Homo sapiens].
MHRDSCPLDCKVYVGNLGNNGNKTELERAFGYYGPLRSVWVARNPPGFA
FVEFEDPRDAADAVRELDGRTLCGCRVRVELSNGEKRSRNRGPPPSWGR
RPRDDYRRRSPPPRRRSPRRRSFSRSRSRSLSRDRRRERSLSRERNHKP
SRSFSRSRSRSRSNERK

ACTA2 Protein ( alpha actin, smooth muscle)
(e.g., GenBank Accession Number P62736
(SEQ ID NO: 133):
>uniprot|P62736|ACTA_HUMAN Actin, aortic smooth
muscle;
MCEEEDSTALVCDNGSGLCKAGFAGDDAPRAVFPSIVGRPRHQGVMVGM
GQKDSYVGDEAQSKRGILTLKYPIEHGIITNWDDMEKIWHHSFYNELRV
APEEHPTLLTEAPLNPKANREKMTQIMFETFNVPAMYVAIQAVLSLYAS
GRTTGIVLDSGDGVTHNVPIYEGYALPHAIMRLDLAGRDLTDYLMKILT
ERGYSFVTTAEREIVRDIKEKLCYVALDFENEMATAASSSSLEKSYELP
DGQVITIGNERFRCPETLFQPSFIGMESAGIHETTYNSIMKCDIDIRKD
LYANNVLSGGTTMYPGIADRMQKEITALAPSTMKIKIIAPPERKYSVWI
GGSILASLSTFQQMWISKQEYDEAGPSIVHRKCF

Isoform 1 Of Sodium Channel Protein Type 8
Subunit Alpha, SCN8A (e.g., GenBank Accession
Number NP_055006 SEQ ID NO: 134):
>refseqp|NP_2355006|NP_055006 sodium channel,
voltage gated, type VIII, alpha [Homo sapiens].
MAARLLAPPGPDSFKPFTPESLANIERRIAESKLKKPPKADGSHREDDE
DSKPKPNSDLEAGKSLPFIYGDIPQGLVAVPLEDFDPYYLTQKTFVVLN
RGKTLFRFSATPALYILSPFNLIRRIAIKILIHSVFSMIIMCTILTNCV
FMTFSNPPDWSKNVEYTFTGIYTFESLVKIIARGFCIDGFTFLRDPWNW
LDFSVIMMAYITEFVNLGNVSALRTFRVLRALKTISVIPGLKTIVGALI
QSVKKLSDVMILTVFCLSVFALIGLQLFMGNLRNKCVVWPINFNESYLE
NGTKGFDWEEYINNKTNFYTVPGMLEPLLCGNSSDAGQCPEGYQCMKAG
RNPNYGYTSFDTFSWAFLALFRLMTQDYWENLYQLTLRAAGKTYMIFFV
LVIFVGSFYLVNLILAVVAMAYEEQNQATLEEAEQKEAEFKAMLEQLKK
QQEEAQAAAMATSAGTVSEDAIEEEGEEGGGSPRSSSEISKLSSKSAKE
RRNRRKKRKQKELSEGEEKGDPEKVFKSESEDGMRRKAFRLPDNRIGRK
FSIMNQSLLSIPGSPFLSRHNSKSSIFSFRGPGRFRDPGSENEFADDEH
STVEESEGRRDSLFIPIRARERRSSYSGYSGYSQGSRSSRIFPSLRRSV
KRNSTVDCNGVVSLIGGPGSHIGGRLLPEATTEVEIKKKGPGSLLVSMD
QLASYGRKDRINSIMSVVTNTLVEELEESQRKCPPCWYKFANTFLIWEC
HPYWIKLKEIVNLIVMDPFVDLAITICIVLNTLFMAMEHHPMTPQFEHV
LAVGNLVFTGIFTAEMFLKLIAMDPYYYFQEGWNIFDGFIVSLSLMELS
LADVEGLSVLRSFRLLRVFKLAKSWPTLNMLIKIIGNSVGALGNLTLVL
AIIVFIFAVVGMQLFGKSYKECVCKINQDCELPRWHMHDFFHSFLIVFR
VLCGEWIETMWDCMEVAGQAMCLIVFMMVMVIGNLVVLNLFLALLLSSF
SADNLAATDDDGEMNNLQISVIRIKKGVAWTKLKVHAFMQAHFKQREAD
EVKPLDELYEKKANCIANHTGADIHRNGDFQKNGNGTTSGIGSSVEKYI
IDEDHMSFINNPNLTVRVPIAVGESDFENLNTEDVSSESDPEGSKDKLD
DTSSSEGSTIDIKPEVEEVPVEQPEEYLDPDACFTEGCVQRFKCCQVNI
EEGLGKSWWILRKTCFLIVEHNWFETFIIFMILLSSGALAFEDIYIEQR
KTIRTILEYADKVFTYIFILEMLLKWTAYGFVKFFTNAWCWLDFLIVAV
SLVSLIANALGYSELGAIKSLRTLRALRPLRALSRFEGMRVVVNALVGA
IPSIMNVLLVCLIFWLIFSIMGVNLFAGKYHYCFNETSEIRFEIEDVNN
KTECEKLMEGNNTEIRWKNVKINFDNVGAGYLALLQVATFKGWMDIMYA
AVDSRKPDEQPKYEDNIYMYIYFVIFIIFGSFFTLNLFIGVIIDNFNQQ
KKKFGGQDIFMTEEQKKYYNAMKKLGSKKPQKPIPRPLNKIQGIVFDFV
TQQAFDIVIMMLICLNMVTMMVETDTQSKQMENILYWINLVFVIFFTCE
CVLKMFALRHYYFTIGWNIFDFVVVILSIVGMFLADIIEKYFVSPTLFR
VIRLARIGRILRLIKGAKGIRTLLFALMMSLPALFNIGLLLFLVMFIFS
IFGMSNFAYVKHEAGIDDMFNFETFGNSMICLFQITTSAGWDGLLLPIL
NRPPDCSLDKEHPGSGFKGDCGNPSVGIFFFVSYIIISFLIVVNMYIAI
ILENFSVATEESADPLSEDDFETFYEIWEKFDPDATQFIEYCKLADFAD
ALEHPLRVPKPNTIELIAMDLPMVSGDRIHCLDILFAFTKRVLGDSGEL
DILRQQMEERFVASNPSKVSYEPITTTLRRKQEEVSAVVLQRAYRGHLA
RRGFICKKTTSNKLENGGTHREKKESTPSTASLPSYDSVTKPEKEKQQR
AEEGRRERAKRQKEVRESKC

Isoform Long Of Galectin-9 (e.g., GenBank
Accession Number NP_033665 SEQ ID NO: 135):
>refseqp|NP_033665|NP_033665 galectin-9
isoform long [Homo sapiens].
MAFSGSQAPYLSPAVPFSGTIQGGLQDGLQITVNGTVLSSSGTRFAVNF
QTGFSGNDIAFHFNPRIEDGGYVVCNTRQNGSWGPEERKTHMPFQKGMP
FDLCFLVQSSDFKVMVNGILFVQYFHRVPFHRVDTISVNGSVQLSYISF
QNPRTVPVQPAFSTVPFSQPVCFPPRPRGRRQKPPGVWPANPAPITQTV
IHTVQSAFGQMFSTPAIPPMMYTHPAYPMPFITTILGGLYPSKSILLSG
TVLPSAQRFHINLCSGNHIAFHLNPRFDENAVVRNTQIDNSWGSEERSL
PRKMPFVRGQSFSVWILCEAHCLKVAVDGQHLFEYYHRLRNLPTINRLE
VGGDIQLTHVQT

T-Complex Protein 1 Subunit Epsilon, CCT5
(e.g., GenBank Accession Number NP_036205
(SEQ ID NO: 136):
>refseqp|NP_036205|NP_036205 chaperonin
containing TCP1, subunit 5 (epsilon)
[Homo sapiens].
MASMGTLAFDEYGRPFLIIKDQDRKSRLMGLEALKSHIMAAKAVANTMR
TSLGPNGLDKMMVDKDGDVTVTNDGATILSMMDVDHQIAKLMVELSKSQ
DDEIGDGTTGVVVLAGALLEEAEQLLDRGIHPIRIADGYEQAARVAIEH
LDKISDSVLVDIKDTEPLIQTAKTTLGSKVVNSCHRQMAEIAVNAVLTV
ADMERRDVDFELIKVEGKVGGRLEDTKLIKGVIVDKDFSHPQMPKKVED
YAKIAILTCPFEPPKPKTKHKLDVTSVEDKALQKYEKEKFEEMIQQIKE
TGANLAICQWGFDDEANHLLLQNNLPAVRWVGGPEIELIAIATGGRIVP
RFSELTAEKLGFAGINQEISFGTTKDKMLVIEQCKNSRAVTIFIRGGNK
MIIEEAKRSLHDALCVIRNLIRDNRVVYGGGAAEISCALAVSQEADKCP
TLEQYAMRAFADALEVIPMALSENSGMNPIQTMTEVRARQVKEMNPALG
IDCLHKGTNDMKQQHVIETLIGKKQQISLATQMVRMILKIDDIRKPGES
EE

Alpha-Enolase, Lung Specific
(e.g., GenBank Accession Number CALA47179
(SEQ ID NO: 137):
MSILKIIHARDIFESRGNPTVEVDLYTNKGGLFGRAAVPSGASTGIYEA
LLELRDNDKTRYMGGKGVSKAVEHIINKTIAPALISKNVNVVEQDKIDN
LMLDMDGSENKSKFGANAILGVSLAVCSNAGATAEKGVPLYRHIADLAG
NNPEVILPVPAFNVINGGSHAGNKLAMQEFMIPPCGADRFNDAIRIGAE
VYHNLKNVIKEKYGKDATNVGDEGGFAPNILENKEALELLKTAIGKAGY
SDKVVIGMDVAASEFYRDGKYDLDFNSPDDPSRYISPDQLADLYKGFVL
GHAVKNYPVGVSIEDPPFDQDDWGAWKKLFTGSLVGIQVVGDDLTVTKP
EARIAKAVEEVKACNCLLLLKVNQIGSVTESLQACKLAQSNGWGVMPVS
HRLSGETEDTFMADLVVGLCTGQIKTGPTCRSERLAKYNQLLRIEEAEA
GSKARFAGRNFRNPRIN

Proto-Oncogene Serine/Threonine-Protein Kinase
MOS (e.g., GenBank Accession Number NP_005363
(SEQ ID NO: 138):
>refseqp|NP_005363|NP_005363 v-mos Moloney murine
sarcoma viral oncogene homolog [Homo sapiens]
MPSPLALRPYLRSEFSPSVDARPCSSPSELPAKLLLGATLPRAPRLPRR
LAWCSIDWEQVCLLQRLGAGGFGSVYKATYRGVPVAIKQVNKCTKNRLA
SRRSFWAELNVARLRHDNIVRVVAASTRTPAGSNSLGTIIMEFGGNVTL
HQVIYGAAGHPEGDAGEPHCRTGGQLSLGKCLKYSLDVVNGLLFLHSQS
IVHLDLKPANILISEQDVCKISDFGCSEKLEDLLCFQTPSYPLGGTYTH
RAPELLKGEGVTPKADIYSFAITLWQMTTKQAPYSGERQHILYAVVAYD
LRPSLSAAVFEDSLPGQRLGDVIQRCWRPSAAQRPSARLLLVDLTSLKA
ELG

Isoform 1 Of Beta-Adducin (ADD2)
(e.g., GenBank Accession Number NP_001608
(SEQ ID NO: 139):
>refseqp|NP_001608|NP_001608 adducin 2 isoform a
[Homo sapiens].
MSEETVPEAASPPPPQGQPYFDRFSEDDPEYMRLRNRAADLRQDFNLME
PQKKRVTMILQSSFREELEGLIQEQMKKGNNSSNIWALRQIADFMASTS
HAVFPTSSMNVSMMTPINDLHTADSLNLAKGERLMRCKISSVYRLLDLY
GWAQLSDTYVTLRVSKEQDHFLISPKGVSCSEVTASSLIKVNILGEVVE
KGSSCFPVDTTGFCLHSAIYAARPDVRCIIHLHTPATAAVSAMKWGLLP
VSHNALLVGDMAYYDFNGEMEQEADRINLQKCLGPTCKILVLRNHGVVA
LGDTVEEAFYKIFHLQAACEDQVSALSSAGGVENLILLEQEKHRPHEVG
SVQWAGSTFGPMQKSRLGEHEFEALMRMLDNLGYRTGYTYRHPFVQEKT
KHKSEVEIPATVTAFVFEEDGAPVPALRQHAQKQQKEKTRWLNTPNTYL
RVNVADEVQRSMGSPRPKTTWMKADEVEKSSSGMPIRIENPNQFVPLYT
DPQEVLEMRNKIREQNRQDVKSAGPQSQLLASVIAEKSRSPSTESQLMS
KGDEDTKDDSEETVPNPFSQLTDQELEEYKKEVERKKLELDGEKETAPE
EPGSPAKSAPASPVQSPAKEAETKSPLVSPSKSLEEGTKETETSKAATT
EPETTQPEGVVVNGREEEQTAEEILSKGLSQMTTSADTDVDTSKDKTES
VTSGPMSPEGSPSKSPSKKKKKFRTPSFLKKSKKKEKVES

Apolipoprotein E (APOE) (e.g., GenBank Accession
Number NP_000032 SEQ ID NO : 140):
>refseqp|NP_00032|NP_000032 apolipoprotein E
precursor [Homo sapiens].
MKVLWAALLVTFLAGCQAKVEQAVETEPEPELRQQTEWQSGQRWELALG
RFWDYLRWVQTLSEQVQEELLSSQVTQELRALMDETMKELKAYKSELEE
QLTPVAEETRARLSKELQAAQARLGADMEDVCGRLVQYRGEVQAMLGQS
TEELRVRLASHLRKLRKRLLRDADDLQKRLAVYQAGAREGAERGLSAIR
ERLGPLVEQGRVRAATVGSLAGQPLQERAQAWGERLRARMEEMGSRTRD
RLDEVKEQVAEVRAKLEEQAQQIRLQAEAFQARLKSWFEPLVEDMQRQW
AGLVEKVQAAVGTSAAPVPSDNH

Ubiquilin-4 (UBQLN4)
(ataxin-1 ubiquitin-like interacting protein)
(e.g. , GenBank Accession Number NP_064516
(SEQ ID NO: 41):
>refseqp|NP_064516|NP_064516 ataxin-1 ubiquitin-
like interacting protein [Homo sapiens].
MAEPSGAETRPPIRVTVKTPKDKEEIVICDRASVKEFKEETSRRFKAQQ
DQLVLIFAGKILKDGDTLNQHGIKDGLTVHLVIKTPQKAQDPAAATASS
PSTPDPASAPSTTPASPATPAQPSTSGSASSDAGSGSRRSSGGGPSPGA
GEGSPSATASILSGFGGILGLGSLGLGSANFMELQQQMQRQLMSNPEML
SQIMENPLVQDMMSNPDLMRHMIMANPQMQQLMERNPEISHMLNNPELM
RQTMELARNPAMMQEMMRNQDRALSNLESIPGGYNALRRMYTDIQEPMF
SAAREQFGNMPFSSLAGNSDSSSSOPLRTENREPLPNPWSPSPPTSQAP
GSGGEGTGGSGTSQVHPTVSNPFGINAASLGSGMFNSPEMQALLQQISE
NPQLMQNVISAPYMRSMMQTLAQNPDFAAQMMVNVPLFAGNPQLQEQLR
LQLPVFLQQMQNPESLSILTNPRAMQALLQIQQGLQTLQTEAPGLVPSL
GSFGISRTPAPSAGSNAGSTPEAPTSSPATPATSSPTGASSAQQQLMQQ
MIQLLAGSGNSQVQTPEVRFQQQLEQLNSMGFINREANLQALIATGGDI
NAAIERLLGSQLS

Sumo-Conjugating Enzyme UB21
(URC9 homolog in yeast)
(e.g., GenBank Accession Number NP_003336
(SEQ ID NO: 142):
>refseqp|NP_003336|NP_003336 ubiquitin-
conjugating enzyme E2I [Homo sapiens].
MSGIALSRLAQERKAWRKDHPFGFVAVPTKNPDGTMNLMNWECAIPGKK
GTPWEGGLFKLRLMLFKDDYPSSPPKCKFEPPLFHPNVYPSGTVCLSIL
EEDKDWRPAITIKQILLGIQELLNEPNIQDPAQAEAYTIYCQNRVEYEK
RVRAQAKKFAPS

Myosin-15(MYH15) (e.g., GenBank Accession Number NP_055796
(SEQ ID NO: 143):
>refseqp|NP_055796|NP_055796 myosin, heavy polypeptide 15
[Homo sapiens].
MVESCLLTFRAFFWWIALIKMDLSDLGEAAAFLRRSEAELLLLQATALDGKKKCWIPDGE
NAYIEAEVKGSEDDGTVIVETADGESLSIKEDKIQQMNPPEFEMIEDMAMLTHLNEASVL
HTLKRRYGQWMIYTYSGLFCVTINPYKWLPVYQKEVMAAYKGKRRSEAPPHIFAVANNAF
QDMLHNRENQSILFTGESGAGKTVNSKHIIQYFATIAAMIESRKKQGALEDQIMQANTIL
EAFGNAKTLRNDNSSRFGKFIRMHFGARGMLSSVDIDIYLLEKSRVIFQQAGERNYHIFY
QILSGQKELHDLLLVSANPSDFHFCSCGAVTVESLDDAEELLATEQAMDILGFLPDEKYG
CYKLTGAIMHFGNMKFKQKPREEQLEADGTENADKAAFLMGINSSELVKCLIHPRIKVGN
EYVTRGQTIEQVTCAVGALSKSMYERMFKWLVARINRALDAKLSRQFFIGILDITGFEIL
EYNSLEQLCINFTNEKLQQFFNWHMFVLEQEEYKKESIEWVSIGFGLDLQACIDLIEKPM
GILSILEEECMFPKATDLTFKTKLFDNHFGKSVHLQKPKPDKKKFEAHFELVHYAGVVPY
NISGWLEKNKDLLNETVVAVFQKSSNRLLASLFENYMSTDSAIPFGEKKRKKGASFQTVA
SLHKENLNKLMTNLKSTAPHFVRCINPNVNKIPGILDPYLVLQQLRCNGVLEGTRICREG
FPNRLQYADFKQRYCILNPRTFPKSKFVSSRKAAEELLGSLEIDHTQYRFGITKVFFKAG
FLGQLEAIRDERLSKVFTLFQARAQGKLMRIKFQKILEERDALILIQWNIRAFMAVKNWP
WMRLFFKIKPLVKSSEVGEEVAGLKEECAQLQKALEKSEFQREELKAKQVSLTQEKNDLI
LQLQAEQETLANVEEQCEWLIKSKIQLEARVKELSERVEEEEEINSELTARGRKLEDECF
ELKKEIDDLETMLVKSEKEKRTTEHKVENLTEEVEFLNEDISKLNRAAEVVQEAHQQTLD
DLHMEEEKLSSLSKANLKLEQQVDELEGALEQERKARMNCERELHKLEGNLKLNRESMEN
LESSQRHLAEELRKKELELSQMNSKVENEKGLVAQLQKTVKELOTQIKDLKEKLEAERTT
RAKMERERADLTQDLADLNERLEEVGGSSLAQLEITKKQETKFQKLHRDMEEATLHFETT
SASLKKRHADSLAELEGQVENLQQVKQKLEKDKSDLQLEVDDLLTRVEQMTRAKANAEKL
CTLYEERLHEATAKLDKVTQLANDLAAQKTKLWSESGEFLRRLEEKEALINQLSREKSNF
TRQIEDLRGQLEKETKSQSALAHALQKAQRDCDLLREQYEEEQEVKAELHRTLSKVNAEM
VQWRMKYENNVIQRTEDLEDAKKELAIRLQEAAEAMGVANARNASLERARHQLQLELGDA
LSDLGKVRSAAARLDQKQLQSGKALADWKQKHEESQALLDASOKEVQALSTELLKLKNTY
EESIVGQETLRRENKNLQEEISNLTNQVREGTKNLTEMEKVKKLIEEEKTEVQVTLEETE
GALERNESKILHFQLELLEAKAELERKLSEKDEEIENFRRKQQCTIDSLQSSLDSEAKSR
IEVTRLKKKMEEDLNEMELQLSCANRQVSEATKSLGQLQIQIKDLQMQLDDSTQLNSDLK
EQVAVAERRNSLLQSELEDLRSLQEQTERGRRLSEEELLEATERINLFYTQNTSLLSQKK
KLEADVARMQKEAEEVVQECQNAEEKAKKAAIEAANLSEELKKKQDTIAHLERTRENMEQ
TITDLQKRLAEAEQMALMGSRKQIQKLESRVRELEGELEGEIRRSAEAQRGARRLERCIK
ELTYQAEEDKKNLSRMQTQMDKLQLKVQNYKQQVEVAETQANQYLSKYKKQQHELNEVKE
RAEVAESQVNKLKIKAREFGKKVQEE

FLJ93091, Homo Sapiens UMP-CMP Kinase (UMP-CMPK)
(e.g. , GenBank Accession Number NP_057392 (SEQ ID NO: 144):
refseqp|NP_057392|NP_057392 UMP-CMP kinase 1 isoform a
[Homo sapiens].
MLSRCRSGLLHVLGLSFLLQTRRPILLCSPRLMKPLVVFVLGGPGAGKGTQCARIVEKYG
YTHLSAGELLRDERKNPDSQYGELIEKYIKEGKIVPVEITISLLKREMDQTMAANAQKNK
FLIDGFPRNQDNLQGWNKTMDGKADVSFVLFFDCNNEICIERCLERGKSSGRSDDNRESL
EKRIQTYLQSTKPIIDLYEEMGKVKKIDASKSVDEVFDEVVQIFKEG

Intelectin-1 (ITLN1) (e.g., GenBank Accession
Number NP_060095 (SEQ ID NO: 145):
>refseqp|NP_060095|NP_060095 intelectin [Homo sapiens].
MNQLSFLLFLIATTRGWSTDEANTYFKEWTCSSSPSLPRSCKEIKDECPSAFDGLYFLRT
ENGVIYQTFCDMTSGGGGWTLVASVHENDMRGKCTVGDRWSSQQGSKAVYPEGDGNWANY
NTFGSAEAATSDDYKNPGYYDIQAKDLGIWHVPNKSPMQHWRNSSLLRYRTDTGFLQTLG
HNLFGIYQKYPVKYGEGKCWTDNGPVIPVVYDFGDAQKTASYYSPYGQREFTAGFVQFRV
FNNERAANALCAGMRVTGCNTEHHCIGGGGYFPEASPQQCGDFSGFDWSGYGTHVGYSSS
REITEAAVLLFYR

Apolipoprotein A-IV (APOA4) (e.g., GenBank Accession
Number Q13784 (SEQ ID NO: 146):
>uniprot|Q13784|Q13784_HUMAN APOA4 protein;
LEPYADQLRTQVNTQAEQLRRQLDPLAQRMERVLRENADSLQASLRPHADELKAKIDQNV
EELKGRLTPYADEFKVKIDQTVEELRRSLAPYAQDTQEKLNHQLEGLTPQMKKNAEELKA
RISASAEELRQRLAPLAEDVRGNLKGNTEGLQKSLAELGGHLDQQVEEFRRRVEPYGENF
NKALVQQMEQLRQKLGPHAGDVEGHLSFLEKDLRDKVNSFFSTFKEKESQDKTLSLPELE
QQQE

Mitochondrial pyruvate dehydrogenase (lipoamide) alpha 1 (PDHA1)
(e.g., GenBank Accession Number P0559 (SEQ ID NO: 147):
>uniprot|P08559|ODPA_HUMAN Pyruvate dehydrogenase E1 component
subunit alpha, somatic form, mitochondrial;
MRKMLAAVSRVLSGASQKPASRVLVASRNFANDATFEIKKCDLHRLEEGPPVTTVLTRED
GLKYYRMMQTVRRMELKADQLYKQKIIRGFCHLCDGQEACCVGLEAGINPTDHLITAYRA
HGFTFTRGLSVREILAELTGRKGGCAKGKGGSMHMYAKNFYGGNGIVGAQVPLGAGIALA
CKYNGKDEVCLTLYGDGAANQGQIFEAYNMAALWKLPCIFICENNRYGMGTSVERAAAST
DYYKRGDFIPGLRVDGMDILCVREATRFAAAYCRSGKGPILMELQTYRYHGHSMSDPGVS
YRTREEIQEVRSKSDPIMLLKDRMVNSNLASVEELKEIDVEVRKEIEDAAQFATADPEPP
LEELGYHIYSSDPPFEVRGANQWIKFKSVS

Leucine-Rich Repeat-Containing Protein 59 (LRRC59)
(e.g., GenBank Accession Number NP_060979 (SEQ ID NO: 148):
>refseqp|NP_060979|NP_060979 leucine rich repeat containing 59
[Homo sapiens]
MTKAGSKGGNLRDKLDGNELDLSLSDLNEVPVKELAALPKATILDLSCNKLTTLPSDFCG
LTHLVKLDLSKNKLQQLPADFGRLVNLQHLDLLNNKLVTLPVSFAQLKNLKWLDLKDNPL
DPVLAKVAGDCLDEKQCKQCANKVLQHMKAVQADQERERQRRLEVEREAEKKREAKQRAK
EAQERELRKREKAEEKERRRKEYDALKAAKREQEKKPKKEANQAPKSKSGSRPREPPPRK
HTRSWAVLKLLLLLLLFGVAGGLVACRVTELQQQPLCTSVNTIYDNAVQGLRRHEILQWV
LQTDSQQ

60S Ribosomal Protein L37A. (RPL37A)
(e.g., GenBank Accession Number NP_000989 (SEQ ID NO: 149):
>refseqp|NP_000989|NP_000989 ribosomal protein L37a
[Homo sapiens].
MAKRTKKVGIVGKYGTRYGASTRKMVKKIEISQHAKYTCSFCGKTKMKRRAVGIWHCGSC
MKTVAGGAWTYNTTSAVTVKSAIRRLKELKDQ

Uridine-Cytidine Kinase 1-like 1 (UCKL1).
(e.g., GenBank Accession Number Q53HM1 (SEQ ID NO: 150):
>uniprot|Q53HM1|Q53HM1_HUMAN Uridine kinase;
MAAPPARADADPSPTSPPTARDTPGRQAEKSETACEDRSNAESLDRLLPPVGTGRSPRKR
TTSQCKSEPPLLRTSKRTIYTAGRPPWYNEHGTQSKEAFAIGLGGGSASGKTTVARMIIE
ALDVPWVVLLSMDSFYKVLTEQQQEQAAHNNFNFDHPDAFDFDLIIFTLKKLKQGKSVKV
PIYDFTTHSRKKDWKTLYGANVIIFEGIMAFADKTLLELLDMKIFVDTDSDIRLVRRLRR
DISERGRDIEGVIKQYNKFVKPSFDQYIQPTMRLADIVVPRGSGNTVAIDLIVQHVHSQL
EERELSVRAALASAHQCHPLPRTLSVLKSTPQVRGMHTIIRDKETSRDEFIFYSKRLMRL
LIEHALSFLPFQDCVVQTPQGQDYAGKCYAGKQITGVSILRAGETMEPALRAVCKDVRIG
TILIQTNQLTGEPELHYLRLPKDISDDHVILMDCTVSTGAAAMMAVRVLLDHDVPEDKIF
LLSLLMAEMGVHSVAYAFPRVRIITTAVDKRVNDLFRIIPGIGNFGDRYFGTDAVPDGSD
EEEVAYTG

Aldehyde Dehydrogenase 9A1 (ALDH9A1)
(e.g., GenBank Accession Number NP_00687 (SEQ ID NO: 151):
>refseqp|NP_000687|NP_000687 aldehyde dehydrogenase 9A1
[homo sapiens].
MFLRAGLAALSPLLRSLRPSPVAAMSTGTFVVSQPLNYRGGARVEPADASGTEKAFEPAT
GRVIATFTCSGEKEVNLAVQNAKAAFKIWSQKSGMERCRILLEAARIIREREDEIATMEC
INNGKSIFEARLDIDISWQCLEYYAGLAASMAGEHIQLPGGSFGYTRREPLGVCVGIGAW
NYPFQIASWKSAPALACGNAMVFKPSPFTPVSALLLAEIYSEAGVPPGLFNVVQGGAATG
QFLCQHPDVAKVSFTGSVPTGMKIMEMSAKGIKPVTLELGGKSPLIIFSDCDMNNAVKGA
LMANFLTQGQVCCNGTRVFVQKEILDKFTEEVVKQTQRIKIGDPLLEDTRMGPLINRPHL
ERVLGFVKVAKEQGAKVLCGGDIYVPEDPKLKDGYYMRPCVLTNCRDDMTCVKEEIFGPV
MSILSFDTEAEVLERANDTTFGLAAGVFTRDIQRAHRVVAELQAGTCFINNYNVSPVELP
FGGYKKSGFGRENGRVTIEYYSQLKTVCVEMGDVESAF

Isoform 3 Of Thioredoxin Reductase 1, Cytoplasmic (TXNRD1)
(e.g., GenBank Accession Number Q16881 (SEQ ID NO: 152):
>uniprot|Q16881|TRXR1_HUMAN Thioredoxin reductase 1, cytoplasmic;
MGCAEGKAVAAAAPTELQTKGKNGDGRRRSAKDHHPGKTLPENPAGFTSTATADSRALLQ
AYIDGHSVVIFSRSTCTRCTEVKKLFKSLCVPYFVLELDQTEDGRALEGTLSELAAETDL
PVVFVKQRKIGGHGPTLKAYQEGRLQKLLKMNGPEDLPKSYDYDLIIIGGGSGGLAAAKE
AAQYGKKVMVLDFVTPTPLGTRWGLGGTCVNVGCIPKKLMHQAALLGQALQDSRNYGWKV
EETVKHDWDRMIEAVQNHIGSLNWGYRVALREKKVVYENAYGQFIGPHRIKATNNKGKEK
IYSAERFLIATGERPRYLGIPGDKEYCISSDDLFSLPYCPGKTLVVGASYVALECAGFLA
GIGLDVTVMVRSILLRGFDQDMANKIGEHMEEHGIKFIRQFVPIKVEQIEAGTPGRLRVV
AQSTNSEEIIEGEYNTVMLAIGRDACTRKIGLETVGVKINEKTGKIPVTDEEQTNVPYIY
AIGDILEDKVELTPVAIQAGRLLAQRLYAGSTVKCDYENVPTTVFTPLEYGACGLSEEKA
VEKFGEENIEVYHSYFWPLEWTIPSRDNNKCYAKIICNTKDNERVVGFHVLGPNAGEVTQ
GFAAALKCGLTKKQLDSTIGIHPVCAEVFTTLSVTKRSGASILQAGCUG

Nuclear Receptor Subfamily 2 Group E Member 1(NR2E1)
(e.g. , GenBank Accession Number NP_003260 (SEQ ID NO: 153):
>refseqp|NP_003260|NP_003260 nuclear receptor subfamily 2,
group E,member 1 [Homo sapiens].
MSKPAGSTSRILDIPCKVCGDRSSGKHYGVYACDGCSGFFKRSIRRNRTYVCKSGNQGGC
PVDKTHRNQCRACRLKKCLEVNMNKDAVQHERGPRTSTIRKQVALYFRGHKEENGAAAHF
PSAALPAPAFFTAVTQLEPHGLELAAVSTTPERQTLVSLAQPTPKYPHEVNGTPMYLYEV
ATESVCESAARLLFMSIKWAKSVPAFSTLSLQDQLMLLEDAWRELFVLGIAQWAIPVDAN
TLLAVSGMNGDNTDSQKLNKIISEIQALQEVVARFRQLRLDATEFACLKCIVTFKAVPTH
SGSELRSFRNAAAIAALQDEAQLTLNSYIHTRYPTQPCRFGKLLLLLPALRSISPSTIEE
VFFKKTIGNVPITRLLSDMYKSSDI

Cation Channel Sperm-Associated Protein 3 (CATSPER3)
(e.g., GenBank Accession Number NP 821138 (SEQ ID NO: 154):
>refseqp|NP_821138|NP_821138 cation channel, sperm associated 3
[Homo sapiens].
MSQHRHQRHSRVISSSPVDTTSVGFCPTFKKFKRNDDECRAFVKRVIMSRFFKIIMISTV
TSNAFFMALWTSYDIRYRLFRLLEFSEIFFVSICTSELSMKVYVDPINYWKNGYNLLDVI
IIIVMFLPYALRQLMGKQFTYLYIADGMQSLRILKLIGYSQGIRTLITAVGQTVYTVASV
LLLLFLLMYIFAILGFCLFGSPDNGDHDNWGNLAAAFFTLFSLATVDGWTDLQKQLDNRE
FALSRAFTIIFILLASFIFLNMFVGVMIMHTEDSIRKFERELMLEQQEMLMGEKQVILQR
QQEEISRLMHIQKNADCTSFSELVENFKKTLSHTDPMVLDDFGTSLPFIDIYFSTLDYQD
TTVHKLQELYYEIVHVLSLMLEDLPQEKPQSLEKVDEK

Transmembrane EMP24 Domain-Containing Protein 1 (TMED1 )
(e.g., GenBank Accession Number NP_006849 (SEQ ID NO: 155):
>refseqp|NP_006849|NP_006849 interleukin 1 receptor-like 1
ligand precursor [Homo sapiens]
MMAAGAALALALWLLMPPVEVGGAGPPPIQDGEFTFLLPAGRKQCFYQSAPANASLETEY
QVIGGAGLDVDFTLESPQGVLLVSESRKADGVHTVEPTEAGDYKLCFDNSFSTISEKLVF
FELIFDSLQDDEEVEGWAEAVEPEEMLDVKMEDIKESIETMRTRLERSIQMLTLLRAFEA
RDRNLQEGNLERVNFWSAVNVAVLLLVAVLQVCTLKRFFQDKRPVPT

Protein FAM154A(FAM154A)
(e.g., GenBank Accession Number NP_714218 (SEQ ID NO: 156):
>refseqp|NP_714918|NP_714918 hypothetical protein LOC158297
[Homo sapiens].
MKTKCICELCSCGRHHCPHLPTKIYDETEKPCLLSEYTENYPFYHSYLPRESFKPRREYQ
KGSIPMEGLTTSRRDFGPHKVAPVKVHQYDQFVPSEENMDLLTTYKKDYNPYPVCRVDPI
KPRDSKYPCSDKMECLPTYKADYLPWNQPRREPLRLEHKYQPASVRFDNRTTHQDDYPIK
GLVKTISCKPLAMPKLCNIPLEDVTNYKMSYVAHPVEKRFVHEAEKFRPCEIPFESLTTQ
KQSYRGLMGEPAKSLKPLARPPGLDMPFCNTTEFRDKYQAWPMPRMFSKAPITYVPPEDR
MDLLTTVQAHYTCPKGAPAQSCRPALQIKKCGRFEGSSTTKDDYKQWSSMRTEPVKPVPQ
LDLPTEPLDCLTTTRAHYVPHLPINTKSCKPHWSGPRGNVPVESQTTYTISFTPKEMGRC
LASYPEPPGYTFEEVDALGHRIYKPVSQAGSQQSSHLSVDDSENPNQRELEVLA

Isoform 1 Of Transcriptional Repressor NF-X1 (NFX1)
(e.g., GenBank Accession Number NP_002495 (SEQ ID NO: 157):
>refseqp|NP_002495|NP_002495 nuclear transcription factor,
X-box binding 1 isoform 1 [Homo sapiens].
MAEAPPVSGTFKFNTDAAEFIPQEKKNSGLNCGTQRRLDSNRIGRRNYSSPPPCHLSRQV
PYDEISAVHQHSYHPSGSKPKSQQTSFQSSPCNKSPKSHGLQNQPWQKLRNEKHHIRVKK
AQSLAEQTSDTAGLESSTRSESGTDLREHSPSESEKEVVGADPRGAKPKKATQFVYSYGR
GPKVKGKLKCEWSNRTTPKPEDAGPESTKPVGVFHPDSSEASSRKGVLDGYGARRNEQRR
YPQKRPPWEVEGARPRPGRNPPKQEGHRHTNAGHRNNMGPIPKDDLNERPAKSTCDSENL
AVINKSSRRVDQEKCTVRRQDPQVVSPFSRGKQNHVLKNVETHTGSLIEQLTTEKYECMV
CCELVRVTAPVWSCQSCYHVFHLNCIKKWARSPASQADGQSGWRCPACQNVSAHVPNTYT
CFCGKVKNPEWSRNEIPHSCGEVCRKKQPGQDCPHSCNLLCHPGPCPPCPAFMTKTCECG
RTRHTVRCGQAVSVHCSNPCENILNCGQHQCAELCHGGQCQPCQIILNQVCYCGSTSRDV
LCGTDVGKSDGFGDFSCLKICGKDLKCGNHTCSQVCHPQPCQQCPRLPQLVRCCPCGQTP
LSQLLELGSSSRKTCMDPVPSCGKVCGKPLPCGSLDFIHTCEKLCHEGDCGPCSRTSVIS
CRCSFRTKELPCTSLKSEDATFMCDKRCNKKRLCGRHKCNEICCVDKEHKCPLICGRKLR
CGLHRCEEPCHRGNCQTCWQASFDELTCHCGASVIYPPVPCGTRPPECTQTCARVHECDH
PVYHSCHSEEKCPPCTFLTQKWCMGKHEFRSNIPCHLVDISCGLPCSATLPCGMHKCQRL
CHKGECLVDEPCKQPCTTPRADCGHPCMAPCHTSSPCPVTACKAKVELQCECGRRKEMVI
CSEASSTYQRIAAISMASKITDMQLGGSVEISKLITKKEVHQARLECDEECSALERKKRL
AEAFHISEDSDPFNIRSSGSKFSDSLKEDARKDLKFVSDVEKEMETLVEAVNKGKNSKKS
HSFPPMNRDHRRIIHDLAQVYGLESVSYDSEPKRNVVVTAIRGKSVCPPTTLTGVLEREM
QARPPPPIPHHRHQSDKNPGSSNLQKITKEPIIDYFDVQD

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

### EXAMPLES

### Example 1

### Paired Autologous Colon Adenocarcinoma and Healthy Tissue Specimens.

Colon adenocarcinoma stages I-IV and autologous healthy tissue from regions of the large bowel adjacent to the tumors were obtained from the Asterand XpressBank (Detroit, Mich.). The samples provided by Asterand had been harvested and quick frozen to preserve intact any potential antigen that was present at the time of harvest. Minimal degradation of the tissues was confirmed by the RNA profile. The tissues were stored at −80° C. until used.

### Tissue and Sample Preparation for Generation of Polyclonal Antibodies in Chickens (YPAbs).

Approximately 50 mg of the frozen stages I-IV colon cancer tissue specimens were separately shaved, thawed on ice, and homogenized. The protein concentration of the samples were adjusted to 1 mg/ml, mixed with Freund's Complete Adjuvant and used to immunize and boost 2 chickens per sample. Colon cancer stages I-IV-specific immune YPAbs, obtained from the eggs three weeks following the following final boost, were tested for reactivity using western blotting against the corresponding stage-specific tumor tissue homogenate (data not shown). Strong and broadly reactive YPabs were purified from 6 eggs per chicken, aliquoted and stored at 4° C. until used. Only results for Stage IV colon cancer tissues are shown.

Assessment of Reactivity of Stage IV YPAbs with Pooled Sera of Patients Diagnosed with Stage IV Colon Cancer.

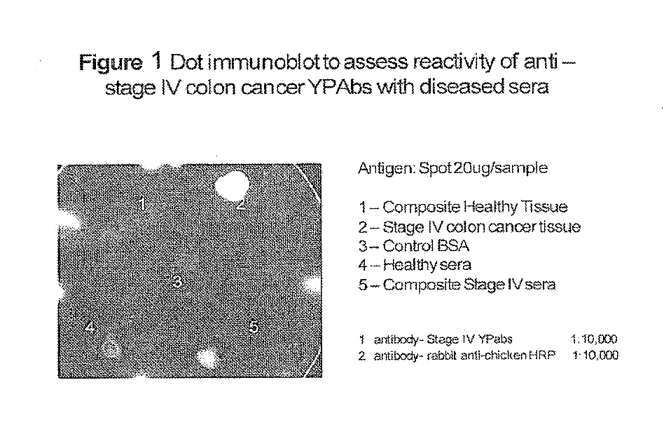

Reactivity was assessed using a dot immunoblot assay. The results, shown in FIG. 1, indicate differential reactivity of spotted pooled sera from stage IV colon cancer patients when compared with spots of control serum from age, gender and ethnicity-matched healthy patients (spot 4), BSA (spot 3), and homogenates of healthy tissue (spot 1). A homogenate of stage IV cancer tissue was the positive control (spot 2).

Subtraction of Antibodies Reactive with Proteins Expressed by Healthy Tissue.

The high titer, broadly reactive YPAbs elicited by homogenates of tumor tissue from each of the 4 stages of colon cancer were repeatedly adsorbed using homogenates of healthy bowel tissue obtained from the autologous host. The proteins in the homogenate were bound to a solid support and the YPAbs were allowed to incubate overnight with gentle rocking at 4° C. Unbound antibodies were recovered and the adsorption process was repeated twice more until ELISA and western blots showed essentially no reactivity with proteins present in healthy tissue. Remaining antibodies were recovered and purified for use in the following steps. Alternatively, in one study, antibodies raised against stage IV tumor tissue were subtracted with serum from healthy subjects. The subtraction was performed by binding the serum components to a solid support and treating the antibody preparation as described above.

### Change Mediated Antigen Capture and Protein Identification.

Unadsorbed antibodies were recovered, purified, and covalently bound to Dynabeads M-280 Tosyl-activated according to the manufacturer's (Dynal Biotech) directions to create “charged” magnetic beads. For immunocapture, homogenates (1 mg/ml) of the staged tumors were matched to their appropriately staged charged beads. Five ml of homogenates were incubated with 0.5 ml of charged beads for 1 h at 4° C. with tilt rotation. Following immunocapture, charged beads were washed with 10 bead volumes of wash buffer (PBS-0.2% NOG). Specifically bound proteins were elated with 1 M acetic acid. Many shed proteins were identified (sec SEQ ID NOs:1-157). The negative control consisted of elutants from an identical volume of uncharged beads used to immunocapture proteins from the homogenates. Proteins specifically bound by charged beads and controls were fractionated on 1D SDS-PAGE, stained with Coomassie blue, and sliced into sections. Protein bands contained in each gel slice were digested in-gel using the enzyme trypsin, eluted from the gel slice, and identified by GeLC-MS/MS and Mascot database searching (IP1 human protein database) at the University of Florida Interdisciplinary Center for Biotechnology Research (ICBR).

A similar format was used to pan serum of stage IV cancer patients for shed change mediated proteins. One ml of scrum from five patients (5 ml total) was pooled and incubated with 0.5 ml of charged beads for 1 h at 4° C. with tilt rotation. Following immunocapture, charged beads were washed with 10 bead volumes of wash buffer (PBS-0.2% NOG). Specifically bound proteins were eluted with 1 M acetic acid. Three shed proteins were identified, the details of which are shown in Table 1.

TABLE 1
PCMAT-identified shed proteins in pooled serum
of patients with stage IV colon carcinoma

| | | # of | |
|---|---|---|---|
| # | Protein | peptides | Comments |
| 1 | ApoA1 | 2 | Associated with colon adenocarcinoma |
| SEQ ID | | | progression, and a confirmed marker |
| NO: 105 | | | of aggression |
| 2 | C4A | 8 | Complement component 4A of the |
| SEQ ID | | | classical activation pathway |
| NO: 106 | | | |
| 3 | C3 187 | 7 | Complement component C3, which plays |
| SEQ ID | kDa | | a central role in activation of both |
| NO: 107 | protein | | the classical and alternate |
| | | | complement systems |

We claim:

1. A method of detecting cancer or a predisposition to developing cancer in a subject, comprising determining an expression level of a cancer-associated protein, polypeptide or polynucleotide selected from the group consisting of myeloblastin precursor (e.g., SEQ ID NO:29); Titin; HBA1; Insulin-like growth factor 1 receptor (IGF1R); Isoform 3 of zonadhesin precursor; latent transforming growth factor beta binding protein 4 (LTBP4); ASXL1 (additional sex combs like 1); beta globin (HBB); BMP15-bone morphogenetic protein; TRIM49; DNAJ homolog subfamily B member 11 precursor; uncharacterized hematopoietic stem/progenitor cells protein MDS027; uncharacterized protein ALB; isoform 3 of sushi, nidogen and EGF-like domain-containing protein 1 precursor; isoform 2 of peripherin; mitochondrial 28S ribosomal protein S22; translation initiation factor EIF-2B subunit epsilon; estradiol 17-beta-dehydrogenase 1; XRCC6BP1; brain-specific angiogenesis inhibitor 1 precursor; isoform 2 of ring finger and CCCH-type zinc finger domain-containing protein 2; hemoglobin subunit beta; isoform 1 of far upstream element-binding protein 1; GALECTIN-3; lysozyme C precursor; actin, alpha skeletal muscle; isoform M2 of pyruvate kinase isozymes M1/M2; AGR2; neutrophil defensin 1 precursor; uncharacterized protein PSME2; tubulin beta-2C chain; thiosulfate sulfurtransferase; heat shock 70 kDa protein 1; Ig kappa chain V-III region sie; macrophage migration inhibitory factor; isoform 1 of ATP synthase subunit D, mitochondrial; uncharacterized protein ENSP00000374051; isocitrate dehydrogenase [NADP] cytoplasmic; hemoglobin subunit delta; isoform 1 of splicing factor, arginine/serine-rich 7; isoform 1 of mRNA-capping enzyme; LON protease homolog, mitochondrial precursor; signal recognition particle 54 kDa protein; isoform long of galectin-9; integrin-linked protein kinase; bifunctional aminoacyl-tRNA synthetase; isoform 1 of zinc finger protein 207; inorganic pyrophosphatase; calponin-2; isoform 1 of muscleblind-like protein 3; cathepsin G precursor; zinc finger and BTB domain-containing protein 34; adenine phosphoribosyltransferase; 40S ribosomal protein S9; TALIN-1; leucine-rich repeat-containing protein 59; ATP synthase subunit alpha, mitochondrial precursor; isoform 7 of protein transport protein SEC31A; dihydroxyacetone kinase; protein similar to heterogeneous nuclear ribonucleoproteins C1/C2 (HNRNP C1/HNRNP C2) isoform 4; 18 kDa protein (e.g., UNIPARC Accession Number IP100796554; cold agglutinin FS-1 L-chain; isoform 1 of heterogeneous nuclear ribonucleoprotein d0; DAZAP1/MEF2D fusion protein; POTE2; Keratin 18 (KRT18); PSME4 Isoform 1 of Proteasome activator complex subunit; Mitogen-activated protein kinase-activated protein kinase (MAPKAPK33); Complement component 1, s subcomponent (C1S); Lysozyme C precursor (LYZ); Keritin Type Cytoskeletal 20 (KRT20); RNASE3; Aldehyde dehydrogenase X, mitochondrial precursor (ALDH1B1); CDNA FLJ25506 fis, clone CBR05185; Isoform B of fibulin-1 precursor (FBLN1); Nucleobindin 1 (NUCB1); Histone cluster 2, H2ba (HIST2H2BA); Tripartite motif-containing 28 (TRIM28); Peroxisomal D3, D2 enoyl-CoA isomerase (PECI); Peptidylprolyl isomerase B (PPIB); Similar to 40S ribosomal protein S17; Eukaryotic translation elongation factor 1 gamma (EEF1G); Keratin 8 (KRT8); Fibulin 2 (FBLN2); VIM; Fibrinogen alpha chain (FGA); Annexin A2 (ANXA2); H2A histone family, member J (H2AFJ); Actin alpha, cardiac muscle 1 (ACTC1); Keratin 19 (KRT19); Immunoglobin lambda locus (IGL@protein); Immunoglobulin heavy constant mu (IGHM); EGF-containing fibulin-like extracellular matrix protein 1 (EFEMP1); Tripartite motif-containing protein 34; Isoform 3 of AP1-subunit Gamma Binding Protein 1; Proflin-1; Histone H4; Hemoglobin subunit alpha; Transgelin); Lumican precursor; Hemoglobin Beta; Fibrinogen Beta Chain Precursor; Immunoglobulin kappa constant (IGKC); Uncharacterized Protein ALB; ApoA1; C4A; C3 187 kDa protein; Actin, Cytoplasmic 1 (actin beta); Hemoglobin beta; Hemoglobin subunit alpha; POTE-2 alpha actin; SLC4A10; Ribonuclease P Protein Subunit P20 (POP7); Nuclear RNA export factor 1 (NXF1); UVEAL Autoantigen With Coiled-Coil Domains And Ankyrin Repeats, UACA; Uncharacterized Protein C13ORF27; Isoform 3 of Sushi, Nidogen And EGF-Like Domain-Containing Protein 1 Precursor; Isoform 1 Of Dynein Heavy Chain 10, Axonemal (DNAH10); Gap junction alpha-1 protein (GJA1/Connexion 43); Isoform 1 Of Kinesin-Like Protein KIF25 (KIF25); GAPDH-Glyceraldehyde-3-Phosphate Dehydrogenase; Uncharacterized Protein ALB; Galectin-3, LGALS3; Similar to NAC-Alpha Domain-Containing Protein 1 (NACAD); Acetyl-CoA Acetyltransferase, Mitochondrial, ACAT1; KH-Type Splicing Regulatory Protein, FUBP2; Profilin 1(PFN1); Chloride Intracellular Channel Protein 1, CLIC1; Zinc Finger Protein 831; Endoplasmin; Ribosomal Protein S10 (RPS10); Splicing Factor, Arginine/Serine-Rich 3; ACTA2 Protein (alpha actin, smooth muscle); Isoform 1 of Sodium Channel Protein Type 8 Subunit Alpha, SCN8A; Isoform Long of Galectin-9; T-Complex Protein 1 Subunit Epsilon, CCT5; Alpha-Enolase, Lung Specific; Proto-Oncogene Serine/Threonine-Protein Kinase MOS; Isoform 1 Of Beta-Adducin (ADD2); Apolipoprotein E (APOE); Ubiquilin-4 (UBQLN4) (ataxin-1 ubiquitin-like interacting protein); Sumo-Conjugating Enzyme UB21 (UBC9 homolog in yeast); Myosin-15 (MYH15); FLJ93091, Homo Sapiens UMP-CMP Kinase (UMP-CMPK); Intelectin-1 (ITLN1); Apolipoprotein A-IV (APOA4); Mitochondrial pyruvate dehydrogenase (lipoamide) alpha 1 (PDHA1); Leucine-Rich Repeat-Containing Protein 59 (LRRC59); 60S Ribosomal Protein L37A (RPL37A); Uridine-Cytidine Kinase 1-like 1 (UCKL1); Aldehyde Dehydrogenase 9A1 (ALDH9A1); Isoform 3 of Thioredoxin Reductase 1, Cytoplasmic (TXNRD1); Nuclear Receptor Subfamily 2 Group E Member 1 (NR2E1); Cation Channel Sperm-Associated Protein 3 (CATSPER3); Transmembrane EMP24 Domain-Containing Protein 1 (TMED1); Protein FAM154A (FAM154A); and Isoform 1 of Transcriptional Repressor NF-X1 (NFX1) or any combinations thereof; in a biological sample from the subject, wherein an increase of the expression level of the cancer-associated protein, polypeptide, or polynucleotide in the biological sample as compared to a control sample indicates that the subject has cancer or has a predisposition to developing cancer.

2. The method of claim 1, wherein the protein or polypeptide comprises an amino acid sequence set forth as SEQ ID NO:1-157.

3. The method of claim 1, wherein the cancer is colorectal cancer.

4. The method of claim 1, wherein the method further comprises determining the expression level of two or more of the cancer-associated proteins, polypeptides, or polynucleotides.

5. The method of claim 1, wherein the expression level of the cancer-associated polynucleotide, polypeptide or protein is determined by a method selected from group consisting of: (a) detecting the presence or amount of the polypeptide, protein, or polynucleotide, (b) detecting mRNA of the cancer-associated polynucleotide, and (c) detecting the biological activity of the protein or polypeptide encoded by the cancer-associated polynucleotide.

6. The method of claim 1, wherein the biological sample comprises cells, cell extracts, tissue, bodily fluid, bodily fluid substantially lacking cells, serum, urine, tears, milk, seminal fluid, prostatic fluid, lung lavage fluid, saliva, mucosal cells, tumor cells, cancer cells, a biopsy sample, a lavage sample, a sputum sample, a serum sample, a plasma sample, a blood sample, a fecal sample, a lymph node sample, a bone marrow sample, a urine sample, a tissue sample, a colorectal tissue sample, or a pleural effusion sample.

7. The method of claim 5, wherein the expression level of the cancer-associated protein or polypeptide is determined by detecting the level of the polypeptide expression in the sample using an antibody or antigen-binding fragment thereof that specifically binds to the polypeptide.

8. An isolated antibody, or antigen-binding fragment thereof, that specifically binds to a protein or polypeptide selected from the group consisting of Titin; HBA1; Insulin-like growth factor 1 receptor (IGF1R); Isoform 3 of zonadhesin precursor; latent transforming growth factor beta binding protein 4 (LTBP4); ASXL1 (additional sex combs like 1); beta globin (HBB); BMP15-bone morphogenetic protein; TRIM49; DNAJ homolog subfamily B member 11 precursor; uncharacterized hematopoietic stem/progenitor cells protein MDS027; uncharacterized protein ALB; isoform 3 of sushi, nidogen and EGF-like domain-containing protein 1 precursor; isoform 2 of peripherin; mitochondrial 28S ribosomal protein S22; translation initiation factor EIF-2B subunit epsilon; estradiol 17-beta-dehydrogenase 1; XRCC6BP1; brain-specific angiogenesis inhibitor 1 precursor; isoform 2 of ring finger and CCCH-type zinc finger domain-containing protein 2; hemoglobin subunit beta; isoform 1 of far upstream element-binding protein 1; GALECTIN-3; lysozyme C precursor; actin, alpha skeletal muscle; isoform M2 of pyruvate kinase isozymes M1/M2; AGR2; neutrophil defensin 1 precursor; myeloblastin precursor; uncharacterized protein PSME2; tubulin beta-2C chain; thiosulfate sulfurtransferase; heat shock 70 kDa protein 1; Ig kappa chain V-III region sie; macrophage migration inhibitory factor; isoform 1 of ATP synthase subunit D, mitochondrial; uncharacterized protein ENSP00000374051; isocitrate dehydrogenase [NADP] cytoplasmic; hemoglobin subunit delta; isoform 1 of splicing factor, arginine/serine-rich 7; isoform 1 of mRNA-capping enzyme; LON protease homolog, mitochondrial precursor; signal recognition particle 54 kDa protein; isoform long of galectin-9; integrin-linked protein kinase; bifunctional aminoacyl-tRNA synthetase; isoform 1 of zinc finger protein 207; inorganic pyrophosphatase; calponin-2; isoform 1 of muscleblind-like protein 3; cathepsin G precursor; zinc finger and BTB domain-containing protein 34; adenine phosphoribosyltransferase; 40S ribosomal protein S9; TALIN-1; leucine-rich repeat-containing protein 59; ATP synthase subunit alpha, mitochondrial precursor; isoform 7 of protein transport protein SEC31A; dihydroxyacetone kinase; protein similar to heterogeneous nuclear ribonucleoproteins C1/C2 (HNRNP C1/HNRNP C2) isoform 4; 18 kDa protein (e.g., UNIPARC Accession Number IP100796554; cold agglutinin FS-1 L-chain; isoform 1 of heterogeneous nuclear ribonucleoprotein d0; DAZAP1/MEF2D fusion protein; POTE2; Keratin 18 (KRT18); PSME4 Isoform 1 of Proteasome activator complex subunit; Mitogen-activated protein kinase-activated protein kinase (MAPKAPK33); Complement component 1, s subcomponent (C1S); Lysozyme C precursor (LYZ); Keritin Type Cytoskeletal 20 (KRT20); RNASE3; Aldehyde dehydrogenase X, mitochondrial precursor (ALDH1B1); CDNA FLJ25506 fis, clone CBR05185; Isoform B of fibulin-1 precursor (FBLN1); Nucleobindin 1 (NUCB1); Histone cluster 2, H2ba (HIST2H2BA); Tripartite motif-containing 28 (TRIM28); Peroxisomal D3, D2 enoyl-CoA isomerase (PECI); Peptidylprolyl isomerase B (PPIB); Similar to 40S ribosomal protein S17; Eukaryotic translation elongation factor 1 gamma (EEF1G); Keratin 8 (KRT8); Fibulin 2 (FBLN2); VIM; Fibrinogen alpha chain (FGA); Annexin A2 (ANXA2); H2A histone family, member J (H2AFJ); Actin alpha, cardiac muscle 1 (ACTC1); Keratin 19 (KRT19); Immunoglobin lambda locus (IGL@protein); Immunoglobulin heavy constant mu (IGHM); EGF-containing fibulin-like extracellular matrix protein 1 (EFEMP1); Tripartite motif-containing protein 34; Isoform 3 of AP1-subunit Gamma Binding Protein 1; Proflin-1; Histone H4; Hemoglobin subunit alpha; Transgelin); Lumican precursor; Hemoglobin Beta; Fibrinogen Beta Chain Precursor; Immunoglobulin kappa constant (IGKC); Uncharacterized Protein ALB; ApoA1; C4A; C3 187 kDa protein; Actin, Cytoplasmic 1 (actin beta); Hemoglobin beta; Hemoglobin subunit alpha; POTE-2 alpha actin; SLC4A10; Ribonuclease P Protein Subunit P20 (POP7); Nuclear RNA export factor 1 (NXF1); UVEAL Autoantigen With Coiled-Coil Domains And Ankyrin Repeats, UACA; Uncharacterized Protein C13ORF27; Isoform 3 of Sushi, Nidogen And EGF-Like Domain-Containing Protein 1 Precursor; Isoform 1 Of Dynein Heavy Chain 10, Axonemal (DNAH10); Gap junction alpha-1 protein (GJA1/Connexion 43); Isoform 1 Of Kinesin-Like Protein KIF25 (KIF25); GAPDH-Glyceraldehyde-3-Phosphate Dehydrogenase; Uncharacterized Protein ALB; Galectin-3, LGALS3; Similar to NAC-Alpha Domain-Containing Protein 1 (NACAD); Acetyl-CoA Acetyltransferase, Mitochondrial, ACAT1; KH-Type Splicing Regulatory Protein, FUBP2; Profilin 1 (PFN1); Chloride Intracellular Channel Protein 1, CLIC1; Zinc Finger Protein 831; Endoplasmin; Ribosomal Protein S10 (RPS10); Splicing Factor, Arginine/Serine-Rich 3; ACTA2 Protein (alpha actin, smooth muscle); Isoform 1 of Sodium Channel Protein Type 8 Subunit Alpha, SCN8A; Isoform Long of Galectin-9; T-Complex Protein 1 Subunit Epsilon, CCT5; Alpha-Enolase, Lung Specific; Proto-Oncogene Serine/Threonine-Protein Kinase MOS; Isoform 1 Of Beta-Adducin (ADD2); Apolipoprotein E (APOE); Ubiquilin-4 (UBQLN4) (ataxin-1 ubiquitin-like interacting protein); Sumo-Conjugating Enzyme UB21 (UBC9 homolog in yeast); Myosin-15 (MYH15); FLJ93091, Homo Sapiens UMP-CMP Kinase (UMP-CMPK); Intelectin-1 (ITLN1); Apolipoprotein A-IV (APOA4); Mitochondrial pyruvate dehydrogenase (lipoamide) alpha 1 (PDHA1); Leucine-Rich Repeat-Containing Protein 59 (LRRC59); 60S Ribosomal Protein L37A (RPL37A); Uridine-Cytidine Kinase 1-like 1 (UCKL1); Aldehyde Dehydrogenase 9A1 (ALDH9A1); Isoform 3 of Thioredoxin Reductase 1, Cytoplasmic (TXNRD1); Nuclear Receptor Subfamily 2 Group E Member 1 (NR2E1); Cation Channel Sperm-Associated Protein 3 (CATSPER3); Transmembrane EMP24 Domain-Containing Protein 1 (TMED1); Protein FAM154A (FAM154A); and Isoform 1 of Transcriptional Repressor NF-X1 (NFX1) or any combination thereof.

9. The isolated antibody of claim 8, wherein the protein or polypeptide comprises an amino acid sequence set forth as SEQ ID NO:1-157.

10. The isolated antibody of claim 8, wherein the antibody is a monoclonal antibody, a polyclonal antibody, a single-chain antibody, a monospecific single-chain antibody, a bispecific single-chain antibody, a bivalent single-chain antibody, a tetravalent single-chain antibody, a chimeric antibody, an antigen-binding fragment of an antibody, or a humanized antibody.

11. (canceled)

12. (canceled)

13. (canceled)

14. (canceled)

15. A kit for the detection of cancer in a mammal, the kit comprising (a) an antibody or antigen-binding fragment thereof, wherein in the antibody or antigen-binding fragment thereof specifically binds an epitope of a protein or polypeptide selected from the group consisting of Titin; HBA1; Insulin-like growth factor 1 receptor (IGF1R); Isoform 3 of zonadhesin precursor; latent transforming growth factor beta binding protein 4 (LTBP4); ASXL1 (additional sex combs like 1); beta globin (HBB); BMP15-bone morphogenetic protein; TRIM49; DNAJ homolog subfamily B member 11 precursor; uncharacterized hematopoietic stem/progenitor cells protein MDS027; uncharacterized protein ALB; isoform 3 of sushi, nidogen and EGF-like domain-containing protein 1 precursor; isoform 2 of peripherin; mitochondrial 28S ribosomal protein S22; translation initiation factor EIF-2B subunit epsilon; estradiol 17-beta-dehydrogenase 1; XRCC6BP1; brain-specific angiogenesis inhibitor 1 precursor; isoform 2 of ring finger and CCCH-type zinc finger domain-containing protein 2; hemoglobin subunit beta; isoform 1 of far upstream element-binding protein 1; GALECTIN-3; lysozyme C precursor; actin, alpha skeletal muscle; isoform M2 of pyruvate kinase isozymes M1/M2; AGR2; neutrophil defensin 1 precursor; myeloblastin precursor; uncharacterized protein PSME2; tubulin beta-2C chain; thiosulfate sulfurtransferase; heat shock 70 kDa protein 1; Ig kappa chain V-III region sie; macrophage migration inhibitory factor; isoform 1 of ATP synthase subunit D, mitochondrial; uncharacterized protein ENSP00000374051; isocitrate dehydrogenase [NADP] cytoplasmic; hemoglobin subunit delta; isoform 1 of splicing factor, arginine/serine-rich 7; isoform 1 of mRNA-capping enzyme; LON protease homolog, mitochondrial precursor; signal recognition particle 54 kDa protein; isoform long of galectin-9; integrin-linked protein kinase; bifunctional aminoacyl-tRNA synthetase; isoform 1 of zinc finger protein 207; inorganic pyrophosphatase; calponin-2; isoform 1 of muscleblind-like protein 3; cathepsin G precursor; zinc finger and BTB domain-containing protein 34; adenine phosphoribosyltransferase; 40S ribosomal protein S9; TALIN-1; leucine-rich repeat-containing protein 59; ATP synthase subunit alpha, mitochondrial precursor; isoform 7 of protein transport protein SEC31A; dihydroxyacetone kinase; protein similar to heterogeneous nuclear ribonucleoproteins C1/C2 (HNRNP C1/HNRNP C2) isoform 4; 18 kDa protein (e.g., UNIPARC Accession Number IP100796554; cold agglutinin FS-1 L-chain; isoform 1 of heterogeneous nuclear ribonucleoprotein d0; DAZAP1/MEF2D fusion protein; POTE2; Keratin 18 (KRT18); PSME4 Isoform 1 of Proteasome activator complex subunit; Mitogen-activated protein kinase-activated protein kinase (MAPKAPK33); Complement component 1, s subcomponent (C1S); Lysozyme C precursor (LYZ); Keritin Type Cytoskeletal 20 (KRT20); RNASE3; Aldehyde dehydrogenase X, mitochondrial precursor (ALDH1B1); CDNA FLJ25506 fis, clone CBR05185; Isoform B of fibulin-1 precursor (FBLN1); Nucleobindin 1 (NUCB1); Histone cluster 2, H2ba (HIST2H2BA); Tripartite motif-containing 28 (TRIM28); Peroxisomal D3, D2 enoyl-CoA isomerase (PECI); Peptidylprolyl isomerase B (PPIB); Similar to 40S ribosomal protein S17; Eukaryotic translation elongation factor 1 gamma (EEF1G); Keratin 8 (KRT8); Fibulin 2 (FBLN2); VIM; Fibrinogen alpha chain (FGA); Annexin A2 (ANXA2); H2A histone family, member J (H2AFJ); Actin alpha, cardiac muscle 1 (ACTC1); Keratin 19 (KRT19); Immunoglobin lambda locus (IGL@protein); Immunoglobulin heavy constant mu (IGHM); EGF-containing fibulin-like extracellular matrix protein 1 (EFEMP1); Tripartite motif-containing protein 34; Isoform 3 of AP1-subunit Gamma Binding Protein 1; Proflin-1; Histone H4; Hemoglobin subunit alpha; Transgelin); Lumican precursor; Hemoglobin Beta; Fibrinogen Beta Chain Precursor; Immunoglobulin kappa constant (IGKC); Uncharacterized Protein ALB; ApoA1; C4A; C3 187 kDa protein; Actin, Cytoplasmic 1 (actin beta); Hemoglobin beta; Hemoglobin subunit alpha; POTE-2 alpha actin; SLC4A10; Ribonuclease P Protein Subunit P20 (POP7); Nuclear RNA export factor 1 (NXF1); UVEAL Autoantigen With Coiled-Coil Domains And Ankyrin Repeats, UACA; Uncharacterized Protein C13ORF27; Isoform 3 of Sushi, Nidogen And EGF-Like Domain-Containing Protein 1 Precursor; Isoform 1 Of Dynein Heavy Chain 10, Axonemal (DNAH10); Gap junction alpha-1 protein (GJA1/Connexion 43); Isoform 1 Of Kinesin-Like Protein KIF25 (KIF25); GAPDH-Glyceraldehyde-3-Phosphate Dehydrogenase; Uncharacterized Protein ALB; Galectin-3, LGALS3; Similar to NAC-Alpha Domain-Containing Protein 1 (NACAD); Acetyl-CoA Acetyltransferase, Mitochondrial, ACAT1; KH-Type Splicing Regulatory Protein, FUBP2; Profilin 1 (PFN1); Chloride Intracellular Channel Protein 1, CLIC1; Zinc Finger Protein 831; Endoplasmin; Ribosomal Protein S10 (RPS10); Splicing Factor, Arginine/Serine-Rich 3; ACTA2 Protein (alpha actin, smooth muscle); Isoform 1 of Sodium Channel Protein Type 8 Subunit Alpha, SCN8A; Isoform Long of Galectin-9; T-Complex Protein 1 Subunit Epsilon, CCT5; Alpha-Enolase, Lung Specific; Proto-Oncogene Serine/Threonine-Protein Kinase MOS; Isoform 1 Of Beta-Adducin (ADD2); Apolipoprotein E (APOE); Ubiquilin-4 (UBQLN4) (ataxin-1 ubiquitin-like interacting protein); Sumo-Conjugating Enzyme UB21 (UBC9 homolog in yeast); Myosin-15 (MYH15); FLJ93091, Homo Sapiens UMP-CMP Kinase (UMP-CMPK); Intelectin-1 (ITLN1); Apolipoprotein A-IV (APOA4); Mitochondrial pyruvate dehydrogenase (lipoamide) alpha 1 (PDHA1); Leucine-Rich Repeat-Containing Protein 59 (LRRC59); 60S Ribosomal Protein L37A (RPL37A); Uridine-Cytidine Kinase 1-like 1 (UCKL1); Aldehyde Dehydrogenase 9A1 (ALDH9A1); Isoform 3 of Thioredoxin Reductase 1, Cytoplasmic (TXNRD1); Nuclear Receptor Subfamily 2 Group E Member 1 (NR2E1); Cation Channel Sperm-Associated Protein 3 (CATSPER3); Transmembrane EMP24 Domain-Containing Protein 1 (TMED1); Protein FAM154A (FAM154A); and Isoform 1 of Transcriptional Repressor NF-X1 (NFX1) or any combinations thereof; and (b) one or more reagents for detecting a binding reaction between the antibody and the polypeptide.

16. The kit of claim 15, wherein the protein or polypeptide comprises an amino acid sequence set forth as SEQ ID NO:1-157.

17.-50. (canceled)

51. The isolated antibody or antigen-binding fragment thereof of claim 8, wherein the isolated antibody or antigen-binding fragment thereof specifically binds to the protein or polypeptide with a binding affinity Kₐ of 10⁷ l/mol or more.

52. The isolated antibody or antigen-binding fragment thereof of claim 8, wherein the isolated antibody or antigen-binding fragment thereof is bound to a support.

53. The isolated antibody or antigen-binding fragment thereof of claim 8, wherein the isolated antibody or antigen-binding fragment is labeled.

54. The method of claim 1, wherein the biological sample is obtained from the subject before, during, or after treatment for cancer.

55. The method of claim 7, wherein the antibody or antigen-binding fragment thereof is labeled.

56. The method of claim 7, wherein the antibody or antigen-binding fragment thereof is bound to a support.

57. The method of claim 7, wherein the antibody or antigen-binding fragment thereof specifically binds to the polypeptide with a binding affinity Kₐ of 10⁷ l/mol or more.

58. The method of claim 1, wherein the expression level of the cancer-associated protein or polypeptide is detected using mass spectrometry, an ELISA, an immunohistochemical assay, an immunocytochemical assay, or a flow cytometry assay of antibody-labeled cells.
